SEQUENCE LISTING (1181 sequences)

SEQ ID NO: 1 - DNA, 35 bp, synthetic construct
  source 1..35: mol_type = other DNA
gaacaagcaatttttccgtttttattttcaaaaac

SEQ ID NO: 2 - DNA, 40 bp, synthetic construct
  source 1..40: mol_type = other DNA
agcagcccgatctaaatcattaccgcgcgattttccctta

SEQ ID NO: 3 - DNA, 42 bp, synthetic construct
  source 1..42: mol_type = other DNA
tgcgttatacgttgctattttatgaggaagtttagtatcata

SEQ ID NO: 4 - DNA, 35 bp, synthetic construct
  source 1..35: mol_type = other DNA
aacgctcaactttttgtagggcttaattgactaga

SEQ ID NO: 5 - DNA, 35 bp, synthetic construct
  source 1..35: mol_type = other DNA
gacaaaaggttttttaagtaattctgtccataatt

SEQ ID NO: 6 - DNA, 35 bp, synthetic construct
  source 1..35: mol_type = other DNA
ataatatcccttttttcctaatttacgagcgctta

SEQ ID NO: 7 - DNA, 42 bp, synthetic construct
  source 1..42: mol_type = other DNA
taacaacctgcaacaaacaaaattaattccaaaaaaataatt

SEQ ID NO: 8 - DNA, 35 bp, synthetic construct
  source 1..35: mol_type = other DNA
aaacgtagaatttttatacatacataaaggaacgg

SEQ ID NO: 9 - DNA, 56 bp, synthetic construct
  source 1..56: mol_type = other DNA
ttttgctcagtaaagacaccagaacctagtttgccccagcagacagcttggaaacg

SEQ ID NO: 10 - DNA, 39 bp, synthetic construct
  source 1..39: mol_type = other DNA
gagactcctccaccgcctggccctgtcgcccaaagtaag

SEQ ID NO: 11 - DNA, 35 bp, synthetic construct
  source 1..35: mol_type = other DNA
agacgttggcaacagctgattattcggttaccgaa

SEQ ID NO: 12 - DNA, 59 bp, synthetic construct
  source 1..59: mol_type = other DNA
ccgcctcaccggaaaataggtgtatcaccgtacttttttaggaggtttagtaccaatag

SEQ ID NO: 13 - DNA, 42 bp, synthetic construct
  source 1..42: mol_type = other DNA
caaatatattacccaagggaaccgaactggccgctgcaagaa

SEQ ID NO: 14 - DNA, 42 bp, synthetic construct
  source 1..42: mol_type = other DNA
gcggattaggcttgtccatgttacttagcatcggagagagat

SEQ ID NO: 15 - DNA, 35 bp, synthetic construct
  source 1..35: mol_type = other DNA
gccgccaggcggtttgcgtatcatgttttgctgaa

SEQ ID NO: 16 - DNA, 42 bp, synthetic construct
  source 1..42: mol_type = other DNA
tttgcctagagccgatcggccaacgcgcaagtacggcggatg

SEQ ID NO: 17 - DNA, 35 bp, synthetic construct
  source 1..35: mol_type = other DNA
tagtaagagctttttacactatcataacccttagg

SEQ ID NO: 18 - DNA, 42 bp, synthetic construct
  source 1..42: mol_type = other DNA
cgccaggctacaaaaagattcaaaagggcgaacgatcatcag

SEQ ID NO: 19 - DNA, 42 bp, synthetic construct
  source 1..42: mol_type = other DNA
tggcgaacaagagacctcatatattttaatggtcataaaacg

SEQ ID NO: 20 - DNA, 35 bp, synthetic construct
  source 1..35: mol_type = other DNA
tcctcatactcacattaattgacacaacgaaattg

SEQ ID NO: 21 - DNA, 42 bp, synthetic construct
  source 1..42: mol_type = other DNA
ttagagcaaagcgcgggtgcctaatgagcataaagggtcata

SEQ ID NO: 22 - DNA, 42 bp, synthetic construct
  source 1..42: mol_type = other DNA
caacattaaaattgtaaagccacgtcaaagggcagctttcat

SEQ ID NO: 23 - DNA, 35 bp, synthetic construct
  source 1..35: mol_type = other DNA
ggattctccgtttttgggaacaaacggcggctggc

SEQ ID NO: 24 - DNA, 42 bp, synthetic construct
  source 1..42: mol_type = other DNA
tggattaacgaccaaacaatattaccgctagcggtaaaataa

SEQ ID NO: 25 - DNA, 42 bp, synthetic construct
  source 1..42: mol_type = other DNA
attatcaacccttcagtagaagaactcaatgcgccttgttaa

SEQ ID NO: 26 - DNA, 35 bp, synthetic construct
  source 1..35: mol_type = other DNA
aataagtatagcccgagatagcgcctgaacatcgg

SEQ ID NO: 27 - DNA, 42 bp, synthetic construct
  source 1..42: mol_type = other DNA
agacaaatgccttgaatcccttataaataagttactatacag

SEQ ID NO: 28 - DNA, 42 bp, synthetic construct
  source 1..42: mol_type = other DNA
ttgagatagaatgacatatttaacaacgtaaacacacgaagg

SEQ ID NO: 29 - DNA, 50 bp, synthetic construct
  source 1..50: mol_type = other DNA
tatgcgatcattgatcgagccagtaatattgaaattaaaacactgcatca

SEQ ID NO: 30 - DNA, 42 bp, synthetic construct
  source 1..42: mol_type = other DNA
tatgtacaggctgcacaataaacaacatctttttccatacag

SEQ ID NO: 31 - DNA, 50 bp, synthetic construct
  source 1..50: mol_type = other DNA
agattgtgcttctgatcaacaatagatatgctgatcctcagagctttcct

SEQ ID NO: 32 - DNA, 42 bp, synthetic construct
  source 1..42: mol_type = other DNA
ttttgaatatcattaaccaatcaataatagactactaaaggg

SEQ ID NO: 33 - DNA, 45 bp, synthetic construct
  source 1..45: mol_type = other DNA
tgaatttttttgtttaattttttcgtcaaaaatgccagttacaaa

SEQ ID NO: 34 - DNA, 55 bp, synthetic construct
  source 1..55: mol_type = other DNA
ttttcaccaactttttagagccgtcaatggcggtcacaatttcatttgattattt

SEQ ID NO: 35 - DNA, 41 bp, synthetic construct
  source 1..41: mol_type = other DNA
tgagcatatcagcaactggcatgattaagactgataattgt

SEQ ID NO: 36 - DNA, 29 bp, synthetic construct
  source 1..29: mol_type = other DNA
aatactttttccaaaagttgctttcgagg

SEQ ID NO: 37 - DNA, 42 bp, synthetic construct
  source 1..42: mol_type = other DNA
gatattccgcgttttcagaaccgccaccaataagatttgcgg

SEQ ID NO: 38 - DNA, 45 bp, synthetic construct
  source 1..45: mol_type = other DNA
agcaaacatcaagagtattttttatcttgacatttgaaagaggac

SEQ ID NO: 39 - DNA, 55 bp, synthetic construct
  source 1..55: mol_type = other DNA
tataatgagtacctcggcattttcggtcctcagagcagggtggtttttcacagac

SEQ ID NO: 40 - DNA, 56 bp, synthetic construct
  source 1..56: mol_type = other DNA
acctgtttcgtgccagctgcacattccaaactaatgcagatacataattgtgtctg

SEQ ID NO: 41 - DNA, 42 bp, synthetic construct
  source 1..42: mol_type = other DNA
gagagatgttttccccagacgacgataagaaagatgtagatt

SEQ ID NO: 42 - DNA, 42 bp, synthetic construct
  source 1..42: mol_type = other DNA
gagcaaaagggggaaagaagttttgccaacgttaaataacct

SEQ ID NO: 43 - DNA, 45 bp, synthetic construct
  source 1..45: mol_type = other DNA
ccaagcttaatgccggattttttgagggtagcggccggagacagt

SEQ ID NO: 44 - DNA, 55 bp, synthetic construct
  source 1..55: mol_type = other DNA
ttatccgtagaggaattagcaaggccggtgatattctcactgcccgcttatatga

SEQ ID NO: 45 - DNA, 42 bp, synthetic construct
  source 1..42: mol_type = other DNA
cagtcactacttctgtaatgggataggtgccatcacacgctg

SEQ ID NO: 46 - DNA, 45 bp, synthetic construct
  source 1..45: mol_type = other DNA
aaaattatggattatttttttttacattggcatgcaacaggaaaa

SEQ ID NO: 47 - DNA, 55 bp, synthetic construct
  source 1..55: mol_type = other DNA
gagaaacaagaaatgaaggtaaatattgatgatactgttgttccagttttaccta

SEQ ID NO: 48 - DNA, 41 bp, synthetic construct
  source 1..41: mol_type = other DNA
ccgaaagcgaattgctgaacctcaaatatcaaaaaaaatcg

SEQ ID NO: 49 - DNA, 46 bp, synthetic construct
  source 1..46: mol_type = other DNA
tgagtttgaacaaattttttgtcagaggaggcttttgaggactaaa

SEQ ID NO: 50 - DNA, 54 bp, synthetic construct
  source 1..54: mol_type = other DNA
aaatcaggtcttttaccctgactattaaccagaactttttgagtagtaactgat

SEQ ID NO: 51 - DNA, 42 bp, synthetic construct
  source 1..42: mol_type = other DNA
ataagaaccaacatcagttcagaaaacgggtttaaaacggag

SEQ ID NO: 52 - DNA, 55 bp, synthetic construct
  source 1..55: mol_type = other DNA
attctactaaatttaatggtagagaatatcgtcataaatatttttaagtctttga

SEQ ID NO: 53 - DNA, 46 bp, synthetic construct
  source 1..46: mol_type = other DNA
ccaataaccagtcattttttggacgtttcatttggtggcgcgagct

SEQ ID NO: 54 - DNA, 54 bp, synthetic construct
  source 1..54: mol_type = other DNA
gaagggcgatcttttgcgggcctcttcatcgtaaatttttactagcatgatttc

SEQ ID NO: 55 - DNA, 42 bp, synthetic construct
  source 1..42: mol_type = other DNA
gagaaaagttcagcgccattcgccattccccggttacttttg

SEQ ID NO: 56 - DNA, 55 bp, synthetic construct
  source 1..55: mol_type = other DNA
cgttagtaactatatgtaaaagtcctgagctttccggcaccataagcaaaagcta

SEQ ID NO: 57 - DNA, 46 bp, synthetic construct
  source 1..46: mol_type = other DNA
tcgcacaaacgttattttttatattttttgctttgtacgagcacgt

SEQ ID NO: 58 - DNA, 54 bp, synthetic construct
  source 1..54: mol_type = other DNA
agaaaccaccatttaggagcggaattaaagaatactttttgtggcacagttctt

SEQ ID NO: 59 - DNA, 22 bp, synthetic construct
  source 1..22: mol_type = other DNA
ctgtccaacaggaaatcatagg

SEQ ID NO: 60 - DNA, 55 bp, synthetic construct
  source 1..55: mol_type = other DNA
gagtgatagattaagacgctagtaccgaactcgtattaaattcgccataatcagt

SEQ ID NO: 61 - DNA, 54 bp, synthetic construct
  source 1..54: mol_type = other DNA
tttagaagtattttgactttacaaacagaaccacctttttagcagaagattttt

SEQ ID NO: 62 - DNA, 44 bp, synthetic construct
  source 1..44: mol_type = other DNA
aaggaactaaagcatttttttcaccttattcattttcaattacc

SEQ ID NO: 63 - DNA, 54 bp, synthetic construct
  source 1..54: mol_type = other DNA
tttgccatctttttcataatcaaaatccctcagagtttttccgccaccccagtg

SEQ ID NO: 64 - DNA, 54 bp, synthetic construct
  source 1..54: mol_type = other DNA
catcgatagcatttaccgtaatcagtaaggaggtttttttgaggcaggtgggaa

SEQ ID NO: 65 - DNA, 54 bp, synthetic construct
  source 1..54: mol_type = other DNA
aggtgaattattttccgtcaccgacttccgttccatttttgtaagcgtcgagtc

SEQ ID NO: 66 - DNA, 48 bp, synthetic construct
  source 1..48: mol_type = other DNA
atatggtataaacatttttgttaatgcctcctgtttgatttggtggtt

SEQ ID NO: 67 - DNA, 55 bp, synthetic construct
  source 1..55: mol_type = other DNA
atagctttttgatagctataaccttgcttctgtaaatcgtccctaatttgaaaat

SEQ ID NO: 68 - DNA, 55 bp, synthetic construct
  source 1..55: mol_type = other DNA
aaaagtttttcctgtttccattaaacgggtaaaatacgtaatcatcgcattgggc

SEQ ID NO: 69 - DNA, 55 bp, synthetic construct
  source 1..55: mol_type = other DNA
tcatctttttttctgacctaatagtagtagcattaacatccagccttttcaatca

SEQ ID NO: 70 - DNA, 42 bp, synthetic construct
  source 1..42: mol_type = other DNA
gaacgcgaagacaatagcaaaattaagctgtaccaaacagga

SEQ ID NO: 71 - DNA, 55 bp, synthetic construct
  source 1..55: mol_type = other DNA
ggttgtttttggttataaatcagagcgggagctaaacaggagcaatacacaatat

SEQ ID NO: 72 - DNA, 42 bp, synthetic construct
  source 1..42: mol_type = other DNA
taagtatttaagaacgcataaccgatatgcccttcaccggaa

SEQ ID NO: 73 - DNA, 55 bp, synthetic construct
  source 1..55: mol_type = other DNA
caatatttttgctatctcgctgaggcttgcagggagttaaagaccaacagaaccg

SEQ ID NO: 74 - DNA, 42 bp, synthetic construct
  source 1..42: mol_type = other DNA
ctcctttgcttaattaaatatgcaactaggggagaccagaac

SEQ ID NO: 75 - DNA, 55 bp, synthetic construct
  source 1..55: mol_type = other DNA
aatactttttcacattctataacagttgattcccaattctgtgagaaatattttt

SEQ ID NO: 76 - DNA, 42 bp, synthetic construct
  source 1..42: mol_type = other DNA
agaggctggaacaagatacatttcgcaaaatgcaagagtctg

SEQ ID NO: 77 - DNA, 55 bp, synthetic construct
  source 1..55: mol_type = other DNA
cttccttttttgtagccgaaaaaccgtctatcaggcaagtgcagccatgattcac

SEQ ID NO: 78 - DNA, 21 bp, synthetic construct
  source 1..21: mol_type = other DNA
gaacccatgtttttgtaacac

SEQ ID NO: 79 - DNA, 46 bp, synthetic construct
  source 1..46: mol_type = other DNA
atttgtatgccactcggaatcataattagaatcgcccataaatcaa

SEQ ID NO: 80 - DNA, 21 bp, synthetic construct
  source 1..21: mol_type = other DNA
atgtttagacttttatagcgt

SEQ ID NO: 81 - DNA, 46 bp, synthetic construct
  source 1..46: mol_type = other DNA
cgggagaaataaataaatatattttagtgacgacggcaactgttgg

SEQ ID NO: 82 - DNA, 21 bp, synthetic construct
  source 1..21: mol_type = other DNA
cagtatcggcttttaggaaga

SEQ ID NO: 83 - DNA, 46 bp, synthetic construct
  source 1..46: mol_type = other DNA
cgttgtaggccgatctttttaacctccgatgtagattgcggaacaa

SEQ ID NO: 84 - DNA, 46 bp, synthetic construct
  source 1..46: mol_type = other DNA
tgctaaagtatccaaataagaaacgatttctgtatacatttgagga

SEQ ID NO: 85 - DNA, 42 bp, synthetic construct
  source 1..42: mol_type = other DNA
cgctgagatctttaaatagaaagccttattacgcaaggttat

SEQ ID NO: 86 - DNA, 21 bp, synthetic construct
  source 1..21: mol_type = other DNA
gttggcaaatttttcagttga

SEQ ID NO: 87 - DNA, 56 bp, synthetic construct
  source 1..56: mol_type = other DNA
aagctgcgaggcgcagacggtaccctcagagttaagcccaatctcagaaaagactt

SEQ ID NO: 88 - DNA, 56 bp, synthetic construct
  source 1..56: mol_type = other DNA
aaattttgtgtcgaaatccgcggctacaggtaattgagcgctgatagcaaagcaaa

SEQ ID NO: 89 - DNA, 46 bp, synthetic construct
  source 1..46: mol_type = other DNA
attagagctcataggctggctgaccttctccaacaccttattagcg

SEQ ID NO: 90 - DNA, 42 bp, synthetic construct
  source 1..42: mol_type = other DNA
caccaccttagcgtaggtcatttcgccaaaaggaaaatcaag

SEQ ID NO: 91 - DNA, 56 bp, synthetic construct
  source 1..56: mol_type = other DNA
aacgcttaaggataaaaattttatatttgggaagaaaaatctgagggggcgccagc

SEQ ID NO: 92 - DNA, 46 bp, synthetic construct
  source 1..46: mol_type = other DNA
tcgactcctaccgttctagctgataaattgcatgcaccaatgaaac

SEQ ID NO: 93 - DNA, 56 bp, synthetic construct
  source 1..56: mol_type = other DNA
attctggggccttgctggtaaccaccactaaccaataggaaccacgttggattgtt

SEQ ID NO: 94 - DNA, 56 bp, synthetic construct
  source 1..56: mol_type = other DNA
tgattttagtaataacatcacactatgggttaaaattcgcatgtttgagattcctg

SEQ ID NO: 95 - DNA, 46 bp, synthetic construct
  source 1..46: mol_type = other DNA
agaaataaagacgctcaatcgtctgaaatttgcacttattcattaa

SEQ ID NO: 96 - DNA, 46 bp, synthetic construct
  source 1..46: mol_type = other DNA
caataattggcaacagtttattttgtcacaatcaattttaaaattc

SEQ ID NO: 97 - DNA, 42 bp, synthetic construct
  source 1..42: mol_type = other DNA
gactttttcgcttaactgaacaccctcgtcacgtataaagcc

SEQ ID NO: 98 - DNA, 42 bp, synthetic construct
  source 1..42: mol_type = other DNA
gaaaaggtgcaaactggctcattatctgcggaataaagtacc

SEQ ID NO: 99 - DNA, 42 bp, synthetic construct
  source 1..42: mol_type = other DNA
ataacgtgcataatatttaaattgttccagccaacaagaaaa

SEQ ID NO: 100 - DNA, 59 bp, synthetic construct
  source 1..59: mol_type = other DNA
aatggttaaacagtacataaatcaatattaaaaataccgaacattcgaccactcatcga

SEQ ID NO: 101 - DNA, 50 bp, synthetic construct
  source 1..50: mol_type = other DNA
gaatccttgtcgtaggaagttttgtcgtctttccagacttttttagtaaa

SEQ ID NO: 102 - DNA, 48 bp, synthetic construct
  source 1..48: mol_type = other DNA
ataaacagccataattaccttaaaacagaggtgaagataatgggattt

SEQ ID NO: 103 - DNA, 45 bp, synthetic construct
  source 1..45: mol_type = other DNA
atcggttaaagaagagcaaatgaaaaatttgaggagtatgttagc

SEQ ID NO: 104 - DNA, 47 bp, synthetic construct
  source 1..47: mol_type = other DNA
tgaatttcttaagcgaaaaccctgcctatttcgcggaataatataaa

SEQ ID NO: 105 - DNA, 42 bp, synthetic construct
  source 1..42: mol_type = other DNA
gccctttagcccggccagagccacaagagaaggatgttgata

SEQ ID NO: 106 - DNA, 45 bp, synthetic construct
  source 1..45: mol_type = other DNA
tagcaacgacctgcccctgacgagaaactagtcagagcccaatag

SEQ ID NO: 107 - DNA, 48 bp, synthetic construct
  source 1..48: mol_type = other DNA
acaatgagccaccctaattcgagcttcaaagcgaacttttagaccgga

SEQ ID NO: 108 - DNA, 48 bp, synthetic construct
  source 1..48: mol_type = other DNA
agatgaacggtgtttttcactcagaaccgccaccatagcccggtcagg

SEQ ID NO: 109 - DNA, 45 bp, synthetic construct
  source 1..45: mol_type = other DNA
gaagtttttaatgaccgccagcattgacgcgacagttacgaggca

SEQ ID NO: 110 - DNA, 45 bp, synthetic construct
  source 1..45: mol_type = other DNA
gtttagcttagaacatcgatgaacggtagctattataatagtaaa

SEQ ID NO: 111 - DNA, 48 bp, synthetic construct
  source 1..48: mol_type = other DNA
ttgagattcgtttacagtcacgacgttgtaaaacgattttggccagtg

SEQ ID NO: 112 - DNA, 48 bp, synthetic construct
  source 1..48: mol_type = other DNA
caaatcaccatcatccagtccagacgattggcctaaacgtcctgcagg

SEQ ID NO: 113 - DNA, 59 bp, synthetic construct
  source 1..59: mol_type = other DNA
cactattttaaagaacgtggactccatgagtctctgaatttagagccatacaacccgtc

SEQ ID NO: 114 - DNA, 45 bp, synthetic construct
  source 1..45: mol_type = other DNA
ggcgcgtttgcctgtgacctgaaagcgttcatcatgggacgacga

SEQ ID NO: 115 - DNA, 48 bp, synthetic construct
  source 1..48: mol_type = other DNA
ttcgcgtattgaccgaataatggaagggttagaaccttttaccatatc

SEQ ID NO: 116 - DNA, 48 bp, synthetic construct
  source 1..48: mol_type = other DNA
acgctcatggaaaggaacaaatacatggcttttgacggaaagtaaaac

SEQ ID NO: 117 - DNA, 45 bp, synthetic construct
  source 1..45: mol_type = other DNA
cgcagagtcggcaaagtaacagtgcccgtttaccatatctggtca

SEQ ID NO: 118 - DNA, 35 bp, synthetic construct
  source 1..35: mol_type = other DNA
aaacagggaatcttaccaacgggttttgttagcga

SEQ ID NO: 119 - DNA, 42 bp, synthetic construct
  source 1..42: mol_type = other DNA
cagatagcgagaggtaggattagcgggggaaagtatgcagca

SEQ ID NO: 120 - DNA, 45 bp, synthetic construct
  source 1..45: mol_type = other DNA
gtgccgtccgaacaaatgacaacaaccaagagagtttaagaggct

SEQ ID NO: 121 - DNA, 35 bp, synthetic construct
  source 1..35: mol_type = other DNA
tgcgaataaaggctccaaaagatcaagaagtgcca

SEQ ID NO: 122 - DNA, 29 bp, synthetic construct
  source 1..29: mol_type = other DNA
atcccatgaaaatctacatttaagtatta

SEQ ID NO: 123 - DNA, 50 bp, synthetic construct
  source 1..50: mol_type = other DNA
taaaacacctttgccgggtattaaaccagagaagaagtgtttttatatgt

SEQ ID NO: 124 - DNA, 35 bp, synthetic construct
  source 1..35: mol_type = other DNA
ccccaaaaaaacattatgaccaaagaatagaacgc

SEQ ID NO: 125 - DNA, 35 bp, synthetic construct
  source 1..35: mol_type = other DNA
attgtgaaccaagcgcgaaacctaaaacgcgttaa

SEQ ID NO: 126 - DNA, 35 bp, synthetic construct
  source 1..35: mol_type = other DNA
tatcaaacggtacgccagaatccgagtaatagccc

SEQ ID NO: 127 - DNA, 21 bp, synthetic construct
  source 1..21: mol_type = other DNA
ttattaacgaactgaaagagt

SEQ ID NO: 128 - DNA, 28 bp, synthetic construct
  source 1..28: mol_type = other DNA
ctaaaatagccagcatgatgaaacaaac

SEQ ID NO: 129 - DNA, 21 bp, synthetic construct
  source 1..21: mol_type = other DNA
acctcccccaatagcatagtt

SEQ ID NO: 130 - DNA, 43 bp, synthetic construct
  source 1..43: mol_type = other DNA
agcgtaatttacaggcgtctttccagaggctattaattaactt

SEQ ID NO: 131 - DNA, 41 bp, synthetic construct
  source 1..41: mol_type = other DNA
tctgagcggctgtagtttgagtaacattggctataattaac

SEQ ID NO: 132 - DNA, 56 bp, synthetic construct
  source 1..56: mol_type = other DNA
tgcctgatgcctgagtaatgtaccattacattattacaggtaaaaccaatgggtaa

SEQ ID NO: 133 - DNA, 21 bp, synthetic construct
  source 1..21: mol_type = other DNA
aatcggtaataaaggcaaatc

SEQ ID NO: 134 - DNA, 42 bp, synthetic construct
  source 1..42: mol_type = other DNA
aactacagggagaaacccagctacaattgattagtattctaa

SEQ ID NO: 135 - DNA, 42 bp, synthetic construct
  source 1..42: mol_type = other DNA
gtgaatagcatcaaccctcattttcaggaatatcaacgaggg

SEQ ID NO: 136 - DNA, 42 bp, synthetic construct
  source 1..42: mol_type = other DNA
agatagagatgatgccgtgcatctgccataaatttgctacag

SEQ ID NO: 137 - DNA, 42 bp, synthetic construct
  source 1..42: mol_type = other DNA
atcagataggcgttaagccttaaatcaattatcctgaagcgc

SEQ ID NO: 138 - DNA, 42 bp, synthetic construct
  source 1..42: mol_type = other DNA
aggcagaaaataaggaaagaggcaaaagcgattatattacct

SEQ ID NO: 139 - DNA, 42 bp, synthetic construct
  source 1..42: mol_type = other DNA
cctcagaaagaattgcagcgaaagacagccggaactcattca

SEQ ID NO: 140 - DNA, 42 bp, synthetic construct
  source 1..42: mol_type = other DNA
gtaccgacctgtgtatacgagccggaagtgagctataaagcc

SEQ ID NO: 141 - DNA, 42 bp, synthetic construct
  source 1..42: mol_type = other DNA
attttcaaccttttttgctttgaatacccaaaagatttaacg

SEQ ID NO: 142 - DNA, 42 bp, synthetic construct
  source 1..42: mol_type = other DNA
attagacacgcctggcttatccgaaattcttaccacagtaca

SEQ ID NO: 143 - DNA, 42 bp, synthetic construct
  source 1..42: mol_type = other DNA
agaatggcagcaaattcgtaatctgtgagcgagtattgggaa

SEQ ID NO: 144 - DNA, 42 bp, synthetic construct
  source 1..42: mol_type = other DNA
gggtcagagggcgacgtcagatgaccctcaatcaagcgccaa

SEQ ID NO: 145 - DNA, 42 bp, synthetic construct
  source 1..42: mol_type = other DNA
tgggcgccatttttacccgccgcgcttaaactatcccaacag

SEQ ID NO: 146 - DNA, 21 bp, synthetic construct
  source 1..21: mol_type = other DNA
gtaccgacctgtgtatacgag

SEQ ID NO: 147 - DNA, 21 bp, synthetic construct
  source 1..21: mol_type = other DNA
aggcagaaaataaggaaagag

SEQ ID NO: 148 - DNA, 21 bp, synthetic construct
  source 1..21: mol_type = other DNA
tctgtgagcgagtattgggaa

SEQ ID NO: 149 - DNA, 21 bp, synthetic construct
  source 1..21: mol_type = other DNA
attttcaaccttttttgcttt

SEQ ID NO: 150 - DNA, 21 bp, synthetic construct
  source 1..21: mol_type = other DNA
atcagataggcgttaagcctt

SEQ ID NO: 151 - DNA, 21 bp, synthetic construct
  source 1..21: mol_type = other DNA
tgaccctcaatcaagcgccaa

SEQ ID NO: 152 - DNA, 21 bp, synthetic construct
  source 1..21: mol_type = other DNA
tgggcgccatttttacccgcc

SEQ ID NO: 153 - DNA, 21 bp, synthetic construct
  source 1..21: mol_type = other DNA
tctgccataaatttgctacag

SEQ ID NO: 154 - DNA, 21 bp, synthetic construct
  source 1..21: mol_type = other DNA
tttcaggaatatcaacgaggg

SEQ ID NO: 155 - DNA, 22 bp, synthetic construct
  source 1..22: mol_type = other DNA
cctcagaaagaattgcagcgaa

SEQ ID NO: 156 - DNA, 62 bp, synthetic construct
  source 1..62: mol_type = other DNA
gaaattcttaccacagtacaaactacagggagaaacccagctacaattgattagtattctaa

SEQ ID NO: 157 - DNA, 67 bp, synthetic construct
  source 1..67: mol_type = other DNA
tcacttcatactctacgttgttgttgttgttgttgttgttgttgttgcaaaagcgattatattacct

SEQ ID NO: 158 - DNA, 87 bp, synthetic construct
  source 1..87: mol_type = other DNA
tcacttcatactctacgttgttgttgttgttgttgttgttgttgttagacagccggaactcattcagtgaatagcatcaaccctcat

SEQ ID NO: 159 - DNA, 88 bp, synthetic construct
  source 1..88: mol_type = other DNA
tcacttcatactctacgttgttgttgttgttgttgttgttgttgttgaatacccaaaagatttaacggggtcagagggcgacgtcaga

SEQ ID NO: 160 - DNA, 88 bp, synthetic construct
  source 1..88: mol_type = other DNA
tcacttcatactctacgttgttgttgttgttgttgttgttgttgttccggaagtgagctataaagccagaatggcagcaaattcgtaa

SEQ ID NO: 161 - DNA, 88 bp, synthetic construct
  source 1..88: mol_type = other DNA
tcacttcatactctacgttgttgttgttgttgttgttgttgttgttgcgcttaaactatcccaacagagatagagatgatgccgtgca

SEQ ID NO: 162 - DNA, 89 bp, synthetic construct
  source 1..89: mol_type = other DNA
tcacttcatactctacgttgttgttgttgttgttgttgttgttgttaaatcaattatcctgaagcgcattagacacgcctggcttatcc

SEQ ID NO: 163 - DNA, 33 bp, synthetic construct
  source 1..33: mol_type = other DNA
tatgaagtgatggatgatgatgatgatgatgat

SEQ ID NO: 164 - DNA, 42 bp, synthetic construct
  source 1..42: mol_type = other DNA
gatcgtccaatcataaatcaacgtaacaagcccgaccgccac

SEQ ID NO: 165 - DNA, 42 bp, synthetic construct
  source 1..42: mol_type = other DNA
agcggtccgccgacaagttaccagaaggcaccaggcggataa

SEQ ID NO: 166 - DNA, 28 bp, synthetic construct
  source 1..28: mol_type = other DNA
gaacgcgatagaagtagcattccacaga

SEQ ID NO: 167 - DNA, 28 bp, synthetic construct
  source 1..28: mol_type = other DNA
acaccgctaatagacaacagtttcagcg

SEQ ID NO: 168 - DNA, 28 bp, synthetic construct
  source 1..28: mol_type = other DNA
atcagctatcgtaagcaattcatcaata

SEQ ID NO: 169 - DNA, 42 bp, synthetic construct
  source 1..42: mol_type = other DNA
gaggccacctgagagtcaatagtgaattccaagaaccgaacg

SEQ ID NO: 170 - DNA, 50 bp, synthetic construct
  source 1..50: mol_type = other DNA
catttttaacggattggttgagaggagtgtactggtagggaggtgcgtag

SEQ ID NO: 171 - DNA, 35 bp, synthetic construct
  source 1..35: mol_type = other DNA
gagccttacaactaaaggaatgagtgagggagcac

SEQ ID NO: 172 - DNA, 42 bp, synthetic construct
  source 1..42: mol_type = other DNA
caccaacaaagtactttcaactttaatcctttaaagtaattt

SEQ ID NO: 173 - DNA, 41 bp, synthetic construct
  source 1..41: mol_type = other DNA
gcgggactaacgaagagaataacataacagccctcaagcaa

SEQ ID NO: 174 - DNA, 42 bp, synthetic construct
  source 1..42: mol_type = other DNA
gcaaggcctgtaatgataatcagaaaaggcaaagctaatgca

SEQ ID NO: 175 - DNA, 49 bp, synthetic construct
  source 1..49: mol_type = other DNA
attttagtcacgcatagtctttaatgcgttttaaactttccttatcatt

SEQ ID NO: 176 - DNA, 28 bp, synthetic construct
  source 1..28: mol_type = other DNA
aacccacgccaccaaaagattaagagga

SEQ ID NO: 177 - DNA, 49 bp, synthetic construct
  source 1..49: mol_type = other DNA
cccccagaatacacaccgaccgtgtgatggcatttatccccctcaaatg

SEQ ID NO: 178 - DNA, 50 bp, synthetic construct
  source 1..50: mol_type = other DNA
caggcggtagctcaatgggcgcccaccaccctcagattttcatttaattg

SEQ ID NO: 179 - DNA, 42 bp, synthetic construct
  source 1..42: mol_type = other DNA
tagtttggtaggtaggctatcaggtcatattaagtaatagcg

SEQ ID NO: 180 - DNA, 28 bp, synthetic construct
  source 1..28: mol_type = other DNA
gcttagatgataagcagactgtagcgcg

SEQ ID NO: 181 - DNA, 28 bp, synthetic construct
  source 1..28: mol_type = other DNA
gctgtttgctcgaaatcaccagtagcac

SEQ ID NO: 182 - DNA, 49 bp, synthetic construct
  source 1..49: mol_type = other DNA
agaaacgaaaccgagataccgatagttgcacgctgttattctgaaacat

SEQ ID NO: 183 - DNA, 28 bp, synthetic construct
  source 1..28: mol_type = other DNA
taacagtggtttaacattcaaccgattg

SEQ ID NO: 184 - DNA, 28 bp, synthetic construct
  source 1..28: mol_type = other DNA
aactaactttgcaatgtgctgcaaggcg

SEQ ID NO: 185 - DNA, 28 bp, synthetic construct
  source 1..28: mol_type = other DNA
caatcgccctgtttgtgccggaaaccag

SEQ ID NO: 186 - DNA, 50 bp, synthetic construct
  source 1..50: mol_type = other DNA
tattcacacaattcccgttgcgcacaaacaaataaacattacctccccgg

SEQ ID NO: 187 - DNA, 42 bp, synthetic construct
  source 1..42: mol_type = other DNA
cgcgtaatatccaggtaataaaagggactaatcctgtgtaga

SEQ ID NO: 188 - DNA, 56 bp, synthetic construct
  source 1..56: mol_type = other DNA
gatcgtccaatcataaatcaacgtaacaagcccgaccgccacttttttacgcatct

SEQ ID NO: 189 - DNA, 56 bp, synthetic construct
  source 1..56: mol_type = other DNA
agcggtccgccgacaagttaccagaaggcaccaggcggataattttttgagtgtgt

SEQ ID NO: 190 - DNA, 42 bp, synthetic construct
  source 1..42: mol_type = other DNA
gaacgcgatagaagtagcattccacagattttttacaaggca

SEQ ID NO: 191 - DNA, 42 bp, synthetic construct
  source 1..42: mol_type = other DNA
acaccgctaatagacaacagtttcagcgttttttgttctggt

SEQ ID NO: 192 - DNA, 42 bp, synthetic construct
  source 1..42: mol_type = other DNA
atcagctatcgtaagcaattcatcaatattttttacgcatct

SEQ ID NO: 193 - DNA, 56 bp, synthetic construct
  source 1..56: mol_type = other DNA
gaggccacctgagagtcaatagtgaattccaagaaccgaacgttttttgagtgtgt

SEQ ID NO: 194 - DNA, 64 bp, synthetic construct
  source 1..64: mol_type = other DNA
catttttaacggattggttgagaggagtgtactggtagggaggtgcgtagttttttacaaggca

SEQ ID NO: 195 - DNA, 49 bp, synthetic construct
  source 1..49: mol_type = other DNA
gagccttacaactaaaggaatgagtgagggagcacttttttgttctggt

SEQ ID NO: 196 - DNA, 51 bp, synthetic construct
  source 1..51: mol_type = other DNA
caccaacaaagtactttcaactttaatcctttaaagtaattttacgcatct

SEQ ID NO: 197 - DNA, 55 bp, synthetic construct
  source 1..55: mol_type = other DNA
gcgggactaacgaagagaataacataacagccctcaagcaattttttgagtgtgt

SEQ ID NO: 198 - DNA, 56 bp, synthetic construct
  source 1..56: mol_type = other DNA
gcaaggcctgtaatgataatcagaaaaggcaaagctaatgcattttttacaaggca

SEQ ID NO: 199 - DNA, 58 bp, synthetic construct
  source 1..58: mol_type = other DNA
attttagtcacgcatagtctttaatgcgttttaaactttccttatcatttgttctggt

SEQ ID NO: 200 - DNA, 42 bp, synthetic construct
  source 1..42: mol_type = other DNA
aacccacgccaccaaaagattaagaggattttttacgcatct

SEQ ID NO: 201 - DNA, 63 bp, synthetic construct
  source 1..63: mol_type = other DNA
cccccagaatacacaccgaccgtgtgatggcatttatccccctcaaatgttttttgagtgtgt

SEQ ID NO: 202 - DNA, 64 bp, synthetic construct
  source 1..64: mol_type = other DNA
caggcggtagctcaatgggcgcccaccaccctcagattttcatttaattgttttttacaaggca

SEQ ID NO: 203 - DNA, 56 bp, synthetic construct
  source 1..56: mol_type = other DNA
tagtttggtaggtaggctatcaggtcatattaagtaatagcgttttttgttctggt

SEQ ID NO: 204 - DNA, 42 bp, synthetic construct
  source 1..42: mol_type = other DNA
gcttagatgataagcagactgtagcgcgttttttacgcatct

SEQ ID NO: 205 - DNA, 42 bp, synthetic construct
  source 1..42: mol_type = other DNA
gctgtttgctcgaaatcaccagtagcacttttttgagtgtgt

SEQ ID NO: 206 - DNA, 58 bp, synthetic construct
  source 1..58: mol_type = other DNA
agaaacgaaaccgagataccgatagttgcacgctgttattctgaaacattacaaggca

SEQ ID NO: 207 - DNA, 42 bp, synthetic construct
  source 1..42: mol_type = other DNA
taacagtggtttaacattcaaccgattgttttttgttctggt

SEQ ID NO: 208 - DNA, 42 bp, synthetic construct
  source 1..42: mol_type = other DNA
aactaactttgcaatgtgctgcaaggcgttttttacgcatct

SEQ ID NO: 209 - DNA, 42 bp, synthetic construct
  source 1..42: mol_type = other DNA
caatcgccctgtttgtgccggaaaccagttttttgagtgtgt

SEQ ID NO: 210 - DNA, 64 bp, synthetic construct
  source 1..64: mol_type = other DNA
tattcacacaattcccgttgcgcacaaacaaataaacattacctccccggttttttacaaggca

SEQ ID NO: 211 - DNA, 56 bp, synthetic construct
  source 1..56: mol_type = other DNA
cgcgtaatatccaggtaataaaagggactaatcctgtgtagattttttgttctggt

SEQ ID NO: 212 - DNA, 56 bp, synthetic construct
  source 1..56: mol_type = other DNA
gatcgtccaatcataaatcaacgtaacaagcccgaccgccactttttagatgcgta

SEQ ID NO: 213 - DNA, 42 bp, synthetic construct
  source 1..42: mol_type = other DNA
gaacgcgatagaagtagcattccacagatttttacacactca

SEQ ID NO: 214 - DNA, 56 bp, synthetic construct
  source 1..56: mol_type = other DNA
agcggtccgccgacaagttaccagaaggcaccaggcggataattttttgccttgta

SEQ ID NO: 215 - DNA, 42 bp, synthetic construct
  source 1..42: mol_type = other DNA
acaccgctaatagacaacagtttcagcgtttttaccagaaca

SEQ ID NO: 216 - DNA, 63 bp, synthetic construct
  source 1..63: mol_type = other DNA
gatcgtccaatcataaatcaacgtaacaagcccgaccgccactttttttttttttacaaggca

SEQ ID NO: 217 - DNA, 63 bp, synthetic construct
  source 1..63: mol_type = other DNA
agcggtccgccgacaagttaccagaaggcaccaggcggataatttttttttttttgttctggt

SEQ ID NO: 218 - DNA, 49 bp, synthetic construct
  source 1..49: mol_type = other DNA
gaacgcgatagaagtagcattccacagatttttttttttttgagtgtgt

SEQ ID NO: 219 - DNA, 49 bp, synthetic construct
  source 1..49: mol_type = other DNA
acaccgctaatagacaacagtttcagcgtttttttttttttacgcatct

SEQ ID NO: 220 - DNA, 49 bp, synthetic construct
  source 1..49: mol_type = other DNA
atcagctatcgtaagcaattcatcaatatttttttttttttgttctggt

SEQ ID NO: 221 - DNA, 63 bp, synthetic construct
  source 1..63: mol_type = other DNA
gaggccacctgagagtcaatagtgaattccaagaaccgaacgtttttttttttttacgcatct

SEQ ID NO: 222 - DNA, 71 bp, synthetic construct
  source 1..71: mol_type = other DNA
catttttaacggattggttgagaggagtgtactggtagggaggtgcgtagtttttttttttttacaaggca

SEQ ID NO: 223 - DNA, 56 bp, synthetic construct
  source 1..56: mol_type = other DNA
gagccttacaactaaaggaatgagtgagggagcactttttttttttttgagtgtgt

SEQ ID NO: 224 - DNA, 63 bp, synthetic construct
  source 1..63: mol_type = other DNA
caccaacaaagtactttcaactttaatcctttaaagtaattttttttttttttttgttctggt

SEQ ID NO: 225 - DNA, 62 bp, synthetic construct
  source 1..62: mol_type = other DNA
gcgggactaacgaagagaataacataacagccctcaagcaatttttttttttttacgcatct

SEQ ID NO: 226 - DNA, 63 bp, synthetic construct
  source 1..63: mol_type = other DNA
gcaaggcctgtaatgataatcagaaaaggcaaagctaatgcatttttttttttttacaaggca

SEQ ID NO: 227 - DNA, 70 bp, synthetic construct
  source 1..70: mol_type = other DNA
attttagtcacgcatagtctttaatgcgttttaaactttccttatcatttttttttttttttgagtgtgt

SEQ ID NO: 228 - DNA, 63 bp, synthetic construct
  source 1..63: mol_type = other DNA
gatcgtccaatcataaatcaacgtaacaagcccgaccgccacttttttttttttaccagaaca

SEQ ID NO: 229 - DNA, 63 bp, synthetic construct
  source 1..63: mol_type = other DNA
agcggtccgccgacaagttaccagaaggcaccaggcggataatttttttttttttgccttgta

SEQ ID NO: 230 - DNA, 49 bp, synthetic construct
  source 1..49: mol_type = other DNA
gaacgcgatagaagtagcattccacagattttttttttttagatgcgta

SEQ ID NO: 231 - DNA, 49 bp, synthetic construct
  source 1..49: mol_type = other DNA
acaccgctaatagacaacagtttcagcgttttttttttttacacactca

SEQ ID NO: 232 - DNA, 49 bp, synthetic construct
  source 1..49: mol_type = other DNA
aactaactttgcaatgtgctgcaaggcgtttttttttttttgccttgta

SEQ ID NO: 233 - DNA, 49 bp, synthetic construct
  source 1..49: mol_type = other DNA
caatcgccctgtttgtgccggaaaccagttttttttttttacacactca

SEQ ID NO: 234 - DNA, 71 bp, synthetic construct
  source 1..71: mol_type = other DNA
tattcacacaattcccgttgcgcacaaacaaataaacattacctccccggttttttttttttaccagaaca

SEQ ID NO: 235 - DNA, 63 bp, synthetic construct
  source 1..63: mol_type = other DNA
cgcgtaatatccaggtaataaaagggactaatcctgtgtagattttttttttttagatgcgta

SEQ ID NO: 236 - DNA, 63 bp, synthetic construct
  source 1..63: mol_type = other DNA
gatcgtccaatcataaatcaacgtaacaagcccgaccgccactttttttttttttaggcatca

SEQ ID NO: 237 - DNA, 63 bp, synthetic construct
  source 1..63: mol_type = other DNA
agcggtccgccgacaagttaccagaaggcaccaggcggataatttttttttttttatgaggca

SEQ ID NO: 238 - DNA, 49 bp, synthetic construct
  source 1..49: mol_type = other DNA
gaacgcgatagaagtagcattccacagatttttttttttttgttgttcg

SEQ ID NO: 239 - DNA, 49 bp, synthetic construct
  source 1..49: mol_type = other DNA
acaccgctaatagacaacagtttcagcgtttttttttttttaattcgcg

SEQ ID NO: 240 - DNA, 49 bp, synthetic construct
  source 1..49: mol_type = other DNA
atcagctatcgtaagcaattcatcaatatttttttttttttacaccaca

SEQ ID NO: 241 - DNA, 63 bp, synthetic construct
  source 1..63: mol_type = other DNA
gaggccacctgagagtcaatagtgaattccaagaaccgaacgtttttttttttttagccgtta

SEQ ID NO: 242 - DNA, 71 bp, synthetic construct
  source 1..71: mol_type = other DNA
catttttaacggattggttgagaggagtgtactggtagggaggtgcgtagtttttttttttttgctcgata

SEQ ID NO: 243 - DNA, 56 bp, synthetic construct
  source 1..56: mol_type = other DNA
gagccttacaactaaaggaatgagtgagggagcactttttttttttttatcgacga

SEQ ID NO: 244 - DNA, 63 bp, synthetic construct
  source 1..63: mol_type = other DNA
caccaacaaagtactttcaactttaatcctttaaagtaattttttttttttttttgatgccta

SEQ ID NO: 245 - DNA, 62 bp, synthetic construct
  source 1..62: mol_type = other DNA
gcgggactaacgaagagaataacataacagccctcaagcaattttttttttttcgaacaaca

SEQ ID NO: 246 - DNA, 63 bp, synthetic construct
  source 1..63: mol_type = other DNA
gcaaggcctgtaatgataatcagaaaaggcaaagctaatgcatttttttttttttgcctcata

SEQ ID NO: 247 - DNA, 70 bp, synthetic construct
  source 1..70: mol_type = other DNA
attttagtcacgcatagtctttaatgcgttttaaactttccttatcattttttttttttttcgcgaatta

SEQ ID NO: 248 - DNA, 49 bp, synthetic construct
  source 1..49: mol_type = other DNA
aacccacgccaccaaaagattaagaggattttttttttttacattcacg

SEQ ID NO: 249 - DNA, 70 bp, synthetic construct
  source 1..70: mol_type = other DNA
cccccagaatacacaccgaccgtgtgatggcatttatccccctcaaatgttttttttttttgcaagaaca

SEQ ID NO: 250 - DNA, 71 bp, synthetic construct
  source 1..71: mol_type = other DNA
caggcggtagctcaatgggcgcccaccaccctcagattttcatttaattgtttttttttttttcaactcca

SEQ ID NO: 251 - DNA, 63 bp, synthetic construct
  source 1..63: mol_type = other DNA
tagtttggtaggtaggctatcaggtcatattaagtaatagcgtttttttttttttccaagtca

SEQ ID NO: 252 - DNA, 49 bp, synthetic construct
  source 1..49: mol_type = other DNA
gcttagatgataagcagactgtagcgcgtttttttttttttccaagtca

SEQ ID NO: 253 - DNA, 49 bp, synthetic construct
  source 1..49: mol_type = other DNA
gctgtttgctcgaaatcaccagtagcactttttttttttttcaactcca

SEQ ID NO: 254 - DNA, 70 bp, synthetic construct
  source 1..70: mol_type = other DNA
agaaacgaaaccgagataccgatagttgcacgctgttattctgaaacatttttttttttttgcaagaaca

SEQ ID NO: 255 - DNA, 49 bp, synthetic construct
  source 1..49: mol_type = other DNA
taacagtggtttaacattcaaccgattgttttttttttttacattcacg

SEQ ID NO: 256 - DNA, 49 bp, synthetic construct
  source 1..49: mol_type = other DNA
aactaactttgcaatgtgctgcaaggcgtttttttttttttcaactcca

SEQ ID NO: 257 - DNA, 49 bp, synthetic construct
  source 1..49: mol_type = other DNA
caatcgccctgtttgtgccggaaaccagttttttttttttacattcacg

SEQ ID NO: 258 - DNA, 71 bp, synthetic construct
  source 1..71: mol_type = other DNA
tattcacacaattcccgttgcgcacaaacaaataaacattacctccccggtttttttttttttccaagtca

SEQ ID NO: 259 - DNA, 63 bp, synthetic construct
  source 1..63: mol_type = other DNA
cgcgtaatatccaggtaataaaagggactaatcctgtgtagattttttttttttgcaagaaca

SEQ ID NO: 260 - DNA, 63 bp, synthetic construct
  source 1..63: mol_type = other DNA
gatcgtccaatcataaatcaacgtaacaagcccgaccgccactttttttttttttagtgagca

SEQ ID NO: 261 - DNA, 63 bp, synthetic construct
  source 1..63: mol_type = other DNA
agcggtccgccgacaagttaccagaaggcaccaggcggataatttttttttttttccttgtca

SEQ ID NO: 262 - DNA, 49 bp, synthetic construct
  source 1..49: mol_type = other DNA
gaacgcgatagaagtagcattccacagattttttttttttgcaatcaca

SEQ ID NO: 263 - DNA, 49 bp, synthetic construct
  source 1..49: mol_type = other DNA
acaccgctaatagacaacagtttcagcgtttttttttttttaacaagcg

SEQ ID NO: 264 - DNA, 49 bp, synthetic construct
  source 1..49: mol_type = other DNA
atcagctatcgtaagcaattcatcaatatttttttttttttcaactcca

SEQ ID NO: 265 - DNA, 63 bp, synthetic construct
  source 1..63: mol_type = other DNA
gaggccacctgagagtcaatagtgaattccaagaaccgaacgttttttttttttacattcacg

SEQ ID NO: 266 - DNA, 71 bp, synthetic construct
  source 1..71: mol_type = other DNA
catttttaacggattggttgagaggagtgtactggtagggaggtgcgtagtttttttttttttccaagtca

SEQ ID NO: 267 - DNA, 56 bp, synthetic construct
  source 1..56: mol_type = other DNA
gagccttacaactaaaggaatgagtgagggagcacttttttttttttgcaagaaca

SEQ ID NO: 268 - DNA, 63 bp, synthetic construct
  source 1..63: mol_type = other DNA
caccaacaaagtactttcaactttaatcctttaaagtaattttttttttttttttgctcacta

SEQ ID NO: 269 - DNA, 62 bp, synthetic construct
  source 1..62: mol_type = other DNA
gcgggactaacgaagagaataacataacagccctcaagcaatttttttttttttgtgattgc

SEQ ID NO: 270 - DNA, 63 bp, synthetic construct
  source 1..63: mol_type = other DNA
gcaaggcctgtaatgataatcagaaaaggcaaagctaatgcatttttttttttttgacaagga

SEQ ID NO: 271 - DNA, 70 bp, synthetic construct
  source 1..70: mol_type = other DNA
attttagtcacgcatagtctttaatgcgttttaaactttccttatcattttttttttttttcgcttgtta

SEQ ID NO: 272 - DNA, 49 bp, synthetic construct
  source 1..49: mol_type = other DNA
aacccacgccaccaaaagattaagaggatttttttttttttcgtcgata

SEQ ID NO: 273 - DNA, 70 bp, synthetic construct
  source 1..70: mol_type = other DNA
cccccagaatacacaccgaccgtgtgatggcatttatccccctcaaatgtttttttttttttaacggcta

SEQ ID NO: 274 - DNA, 71 bp, synthetic construct
  source 1..71: mol_type = other DNA
caggcggtagctcaatgggcgcccaccaccctcagattttcatttaattgtttttttttttttatcgagca

SEQ ID NO: 275 - DNA, 63 bp, synthetic construct
  source 1..63: mol_type = other DNA
tagtttggtaggtaggctatcaggtcatattaagtaatagcgtttttttttttttgtggtgta

SEQ ID NO: 276 - DNA, 49 bp, synthetic construct
  source 1..49: mol_type = other DNA
gcttagatgataagcagactgtagcgcgttttttttttttgcaagaaca

SEQ ID NO: 277 - DNA, 49 bp, synthetic construct
  source 1..49: mol_type = other DNA
gctgtttgctcgaaatcaccagtagcactttttttttttttccaagtca

SEQ ID NO: 278 - DNA, 70 bp, synthetic construct
  source 1..70: mol_type = other DNA
agaaacgaaaccgagataccgatagttgcacgctgttattctgaaacatttttttttttttacattcacg

SEQ ID NO: 279 - DNA, 49 bp, synthetic construct
  source 1..49: mol_type = other DNA
taacagtggtttaacattcaaccgattgtttttttttttttcaactcca

SEQ ID NO: 280 - DNA, 49 bp, synthetic construct
  source 1..49: mol_type = other DNA
aactaactttgcaatgtgctgcaaggcgttttttttttttacattcacg

SEQ ID NO: 281 - DNA, 49 bp, synthetic construct
  source 1..49: mol_type = other DNA
caatcgccctgtttgtgccggaaaccagttttttttttttgcaagaaca

SEQ ID NO: 282 - DNA, 71 bp, synthetic construct
  source 1..71: mol_type = other DNA
tattcacacaattcccgttgcgcacaaacaaataaacattacctccccggtttttttttttttcaactcca

SEQ ID NO: 283 - DNA, 63 bp, synthetic construct
  source 1..63: mol_type = other DNA
cgcgtaatatccaggtaataaaagggactaatcctgtgtagatttttttttttttccaagtca

SEQ ID NO: 284 - DNA, 63 bp, synthetic construct
  source 1..63: mol_type = other DNA
gatcgtccaatcataaatcaacgtaacaagcccgaccgccactttttttttttttatctcgca

SEQ ID NO: 285 - DNA, 63 bp, synthetic construct
  source 1..63: mol_type = other DNA
agcggtccgccgacaagttaccagaaggcaccaggcggataattttttttttttagttccgta

SEQ ID NO: 286 - DNA, 49 bp, synthetic construct
  source 1..49: mol_type = other DNA
gaacgcgatagaagtagcattccacagatttttttttttttatcgctca

SEQ ID NO: 287 - DNA, 49 bp, synthetic construct
  source 1..49: mol_type = other DNA
acaccgctaatagacaacagtttcagcgtttttttttttttacgttcct

SEQ ID NO: 288 - DNA, 49 bp, synthetic construct
  source 1..49: mol_type = other DNA
atcagctatcgtaagcaattcatcaatatttttttttttttaccgcata

SEQ ID NO: 289 - DNA, 63 bp, synthetic construct
  source 1..63: mol_type = other DNA
gaggccacctgagagtcaatagtgaattccaagaaccgaacgtttttttttttttatggcgta

SEQ ID NO: 290 - DNA, 71 bp, synthetic construct
  source 1..71: mol_type = other DNA
catttttaacggattggttgagaggagtgtactggtagggaggtgcgtagtttttttttttttgtctgtga

SEQ ID NO: 291 - DNA, 56 bp, synthetic construct
  source 1..56: mol_type = other DNA
gagccttacaactaaaggaatgagtgagggagcacttttttttttttacgatctca

SEQ ID NO: 292 - DNA, 63 bp, synthetic construct
  source 1..63: mol_type = other DNA
caccaacaaagtactttcaactttaatcctttaaagtaattttttttttttttttggagttga

SEQ ID NO: 293 - DNA, 62 bp, synthetic construct
  source 1..62: mol_type = other DNA
gcgggactaacgaagagaataacataacagccctcaagcaatttttttttttttgacttgga

SEQ ID NO: 294 - DNA, 63 bp, synthetic construct
  source 1..63: mol_type = other DNA
gcaaggcctgtaatgataatcagaaaaggcaaagctaatgcattttttttttttcgtgaatgt

SEQ ID NO: 295 - DNA, 70 bp, synthetic construct
  source 1..70: mol_type = other DNA
attttagtcacgcatagtctttaatgcgttttaaactttccttatcatttttttttttttttgttcttgc

SEQ ID NO: 296 - DNA, 49 bp, synthetic construct
  source 1..49: mol_type = other DNA
aacccacgccaccaaaagattaagaggatttttttttttttggtcaaga

SEQ ID NO: 297 - DNA, 70 bp, synthetic construct
  source 1..70: mol_type = other DNA
cccccagaatacacaccgaccgtgtgatggcatttatccccctcaaatgtttttttttttttgaactgga

SEQ ID NO: 298 - DNA, 71 bp, synthetic construct
  source 1..71: mol_type = other DNA
caggcggtagctcaatgggcgcccaccaccctcagattttcatttaattgttttttttttttgctgcatta

SEQ ID NO: 299 - DNA, 63 bp, synthetic construct
  source 1..63: mol_type = other DNA
tagtttggtaggtaggctatcaggtcatattaagtaatagcgtttttttttttttgcttatgc

SEQ ID NO: 300 - DNA, 49 bp, synthetic construct
  source 1..49: mol_type = other DNA
aactaactttgcaatgtgctgcaaggcgtttttttttttttgagatcgt

SEQ ID NO: 301 - DNA, 49 bp, synthetic construct
  source 1..49: mol_type = other DNA
caatcgccctgtttgtgccggaaaccagtttttttttttttacgccata

SEQ ID NO: 302 - DNA, 71 bp, synthetic construct
  source 1..71: mol_type = other DNA
tattcacacaattcccgttgcgcacaaacaaataaacattacctccccggtttttttttttttcacagaca

SEQ ID NO: 303 - DNA, 63 bp, synthetic construct
  source 1..63: mol_type = other DNA
cgcgtaatatccaggtaataaaagggactaatcctgtgtagatttttttttttttatgcggta

SEQ ID NO: 304 - DNA, 49 bp, synthetic construct
  source 1..49: mol_type = other DNA
atcagctatcgtaagcaattcatcaatatttttttttttttgagcgata

SEQ ID NO: 305 - DNA, 63 bp, synthetic construct
  source 1..63: mol_type = other DNA
gaggccacctgagagtcaatagtgaattccaagaaccgaacgttttttttttttaggaacgta

SEQ ID NO: 306 - DNA, 71 bp, synthetic construct
  source 1..71: mol_type = other DNA
catttttaacggattggttgagaggagtgtactggtagggaggtgcgtagtttttttttttttgcgagata

SEQ ID NO: 307 - DNA, 56 bp, synthetic construct
  source 1..56: mol_type = other DNA
gagccttacaactaaaggaatgagtgagggagcactttttttttttttacggaact

SEQ ID NO: 308 - DNA, 63 bp, synthetic construct
  source 1..63: mol_type = other DNA
caccaacaaagtactttcaactttaatcctttaaagtaatttttttttttttttagaatcgga

SEQ ID NO: 309 - DNA, 62 bp, synthetic construct
  source 1..62: mol_type = other DNA
gcgggactaacgaagagaataacataacagccctcaagcaatttttttttttttagcacgta

SEQ ID NO: 310 - DNA, 63 bp, synthetic construct
  source 1..63: mol_type = other DNA
gcaaggcctgtaatgataatcagaaaaggcaaagctaatgcatttttttttttttacgaacct

SEQ ID NO: 311 - DNA, 70 bp, synthetic construct
  source 1..70: mol_type = other DNA
attttagtcacgcatagtctttaatgcgttttaaactttccttatcatttttttttttttttacgcacta

SEQ ID NO: 312 - DNA, 49 bp, synthetic construct
  source 1..49: mol_type = other DNA
gcttagatgataagcagactgtagcgcgttttttttttttcgcaacata

SEQ ID NO: 313 - DNA, 49 bp, synthetic construct
  source 1..49: mol_type = other DNA
gctgtttgctcgaaatcaccagtagcactttttttttttttacagagca

SEQ ID NO: 314 - DNA, 70 bp, synthetic construct
  source 1..70: mol_type = other DNA
agaaacgaaaccgagataccgatagttgcacgctgttattctgaaacatttttttttttttcgcaatgta

SEQ ID NO: 315 - DNA, 49 bp, synthetic construct
  source 1..49: mol_type = other DNA
taacagtggtttaacattcaaccgattgtttttttttttttactcagca

SEQ ID NO: 316 - DNA, 49 bp, synthetic construct
  source 1..49: mol_type = other DNA
aactaactttgcaatgtgctgcaaggcgtttttttttttttaatgcagc

SEQ ID NO: 317 - DNA, 49 bp, synthetic construct
  source 1..49: mol_type = other DNA
caatcgccctgtttgtgccggaaaccagttttttttttttgcataagca

SEQ ID NO: 318 - DNA, 71 bp, synthetic construct
  source 1..71: mol_type = other DNA
tattcacacaattcccgttgcgcacaaacaaataaacattacctccccggtttttttttttttcttgacca

SEQ ID NO: 319 - DNA, 63 bp, synthetic construct
  source 1..63: mol_type = other DNA
cgcgtaatatccaggtaataaaagggactaatcctgtgtagatttttttttttttccagttca

SEQ ID NO: 320 - DNA, 49 bp, synthetic construct; identical to SEQ ID NO: 304
  source 1..49: mol_type = other DNA

SEQ ID NO: 321 - DNA, 63 bp, synthetic construct; identical to SEQ ID NO: 305
  source 1..63: mol_type = other DNA

SEQ ID NO: 322 - DNA, 71 bp, synthetic construct; identical to SEQ ID NO: 306
  source 1..71: mol_type = other DNA

SEQ ID NO: 323 - DNA, 56 bp, synthetic construct; identical to SEQ ID NO: 307
  source 1..56: mol_type = other DNA

SEQ ID NO: 324 - DNA, 63 bp, synthetic construct
  source 1..63: mol_type = other DNA
caccaacaaagtactttcaactttaatcctttaaagtaattttttttttttttttacgtgcta

SEQ ID NO: 325 - DNA, 62 bp, synthetic construct
  source 1..62: mol_type = other DNA
gcgggactaacgaagagaataacataacagccctcaagcaatttttttttttttccgattct

SEQ ID NO: 326 - DNA, 63 bp, synthetic construct
  source 1..63: mol_type = other DNA
gcaaggcctgtaatgataatcagaaaaggcaaagctaatgcatttttttttttttagtgcgta

SEQ ID NO: 327 - DNA, 70 bp, synthetic construct
  source 1..70: mol_type = other DNA
attttagtcacgcatagtctttaatgcgttttaaactttccttatcattttttttttttttaggttcgta

SEQ ID NO: 328 - DNA, 49 bp, synthetic construct; identical to SEQ ID NO: 312
  source 1..49: mol_type = other DNA

SEQ ID NO: 329 - DNA, 49 bp, synthetic construct; identical to SEQ ID NO: 313
  source 1..49: mol_type = other DNA

SEQ ID NO: 330 - DNA, 70 bp, synthetic construct; identical to SEQ ID NO: 314
  source 1..70: mol_type = other DNA

SEQ ID NO: 331 - DNA, 49 bp, synthetic construct; identical to SEQ ID NO: 315
  source 1..49: mol_type = other DNA

SEQ ID NO: 332 - DNA, 49 bp, synthetic construct; identical to SEQ ID NO: 316
  source 1..49: mol_type = other DNA

SEQ ID NO: 333 - DNA, 49 bp, synthetic construct; identical to SEQ ID NO: 317
  source 1..49: mol_type = other DNA

SEQ ID NO: 334 - DNA, 71 bp, synthetic construct; identical to SEQ ID NO: 318
  source 1..71: mol_type = other DNA

SEQ ID NO: 335 - DNA, 63 bp, synthetic construct; identical to SEQ ID NO: 319
  source 1..63: mol_type = other DNA

SEQ ID NO: 336 - DNA, 49 bp, synthetic construct
  source 1..49: mol_type = other DNA
atcagctatcgtaagcaattcatcaatatttttttttttttgctctgta

SEQ ID NO: 337 - DNA, 63 bp, synthetic construct
  source 1..63: mol_type = other DNA
gaggccacctgagagtcaatagtgaattccaagaaccgaacgtttttttttttttatgttgcg

SEQ ID NO: 338 - DNA, 71 bp, synthetic construct
  source 1..71: mol_type = other DNA
catttttaacggattggttgagaggagtgtactggtagggaggtgcgtagtttttttttttttgctgagta

SEQ ID NO: 339 - DNA, 56 bp, synthetic construct
  source 1..56: mol_type = other DNA
gagccttacaactaaaggaatgagtgagggagcactttttttttttttacattgcg

SEQ ID NO: 340 - DNA, 49 bp, synthetic construct
  source 1..49: mol_type = other DNA
gcttagatgataagcagactgtagcgcgtttttttttttttacattgcg

SEQ ID NO: 341 - DNA, 49 bp, synthetic construct
  source 1..49: mol_type = other DNA
gctgtttgctcgaaatcaccagtagcactttttttttttttgctgagta

SEQ ID NO: 342 - DNA, 70 bp, synthetic construct
  source 1..70: mol_type = other DNA
agaaacgaaaccgagataccgatagttgcacgctgttattctgaaacattttttttttttttatgttgcg

SEQ ID NO: 343 - DNA, 49 bp, synthetic construct
  source 1..49: mol_type = other DNA
taacagtggtttaacattcaaccgattgtttttttttttttgctctgta

SEQ ID NO: 344 - DNA, 49 bp, synthetic construct
  source 1..49: mol_type = other DNA
aactaactttgcaatgtgctgcaaggcgtttttttttttttatgttgcg

SEQ ID NO: 345 - DNA, 49 bp, synthetic construct
  source 1..49: mol_type = other DNA
caatcgccctgtttgtgccggaaaccagtttttttttttttacattgcg

SEQ ID NO: 346 - DNA, 71 bp, synthetic construct
  source 1..71: mol_type = other DNA
tattcacacaattcccgttgcgcacaaacaaataaacattacctccccggtttttttttttttgctctgta

SEQ ID NO: 347 - DNA, 63 bp, synthetic construct
  source 1..63: mol_type = other DNA
cgcgtaatatccaggtaataaaagggactaatcctgtgtagatttttttttttttgctgagta

SEQ ID NO: 348 - DNA, 26 bp, synthetic construct
  source 1..26: mol_type = other DNA
tgatgcctaaaaaaaaaacgtatgga

SEQ ID NO: 349 - DNA, 26 bp, synthetic construct
  source 1..26: mol_type = other DNA
cgaacaacaaaaaaaaatacagcgta

SEQ ID NO: 350 - DNA, 26 bp, synthetic construct
  source 1..26: mol_type = other DNA
tgcctcataaaaaaaaatatctgcga

SEQ ID NO: 351 - DNA, 26 bp, synthetic construct
  source 1..26: mol_type = other DNA
cgcgaattaaaaaaaaagcatgtgaa

SEQ ID NO: 352 - DNA, 26 bp, synthetic construct
  source 1..26: mol_type = other DNA
tgctcactaaaaaaaaatgaaggaca

SEQ ID NO: 353 - DNA, 26 bp, synthetic construct
  source 1..26: mol_type = other DNA
tgtgattgcaaaaaaaatactgacgt

SEQ ID NO: 354 - DNA, 26 bp, synthetic construct
  source 1..26: mol_type = other DNA
tgacaaggaaaaaaaaatacgagtgt

SEQ ID NO: 355 - DNA, 26 bp, synthetic construct
  source 1..26: mol_type = other DNA
cgcttgttaaaaaaaaatccacttga

SEQ ID NO: 356 - DNA, 26 bp, synthetic construct
  source 1..26: mol_type = other DNA
tgagatcgtaaaaaaaatgaagtcca

SEQ ID NO: 357 - DNA, 26 bp, synthetic construct
  source 1..26: mol_type = other DNA
tacgccataaaaaaaaatgtcttcca

SEQ ID NO: 358 - DNA, 26 bp, synthetic construct
  source 1..26: mol_type = other DNA
tcacagacaaaaaaaaatgttctcca

SEQ ID NO: 359 - DNA, 26 bp, synthetic construct
  source 1..26: mol_type = other DNA
tatgcggtaaaaaaaaatgtctagca

SEQ ID NO: 360 - DNA, 26 bp, synthetic construct
  source 1..26: mol_type = other DNA
tccatacgttttttttttaggcatca

SEQ ID NO: 361 - DNA, 26 bp, synthetic construct
  source 1..26: mol_type = other DNA
tacgctgtatttttttttgttgttcg

SEQ ID NO: 362 - DNA, 26 bp, synthetic construct
  source 1..26: mol_type = other DNA
tcgcagatatttttttttatgaggca

SEQ ID NO: 363 - DNA, 26 bp, synthetic construct
  source 1..26: mol_type = other DNA
ttcacatgctttttttttaattcgcg

SEQ ID NO: 364 - DNA, 26 bp, synthetic construct
  source 1..26: mol_type = other DNA
tgtccttcatttttttttagtgagca

SEQ ID NO: 365 - DNA, 26 bp, synthetic construct
  source 1..26: mol_type = other DNA
acgtcagtattttttttgcaatcaca

SEQ ID NO: 366 - DNA, 26 bp, synthetic construct
  source 1..26: mol_type = other DNA
acactcgtatttttttttccttgtca

SEQ ID NO: 367 - DNA, 26 bp, synthetic construct
  source 1..26: mol_type = other DNA
tcaagtggatttttttttaacaagcg

SEQ ID NO: 368 - DNA, 26 bp, synthetic construct
  source 1..26: mol_type = other DNA
tggacttcattttttttacgatctca

SEQ ID NO: 369 - DNA, 26 bp, synthetic construct
  source 1..26: mol_type = other DNA
tggaagacatttttttttatggcgta

SEQ ID NO: 370 - DNA, 26 bp, synthetic construct
  source 1..26: mol_type = other DNA
tggagaacatttttttttgtctgtga

SEQ ID NO: 371 - DNA, 26 bp, synthetic construct
  source 1..26: mol_type = other DNA
tgctagacatttttttttaccgcata

SEQ ID NO: 372 - DNA, 56 bp, synthetic construct
  source 1..56: mol_type = other DNA
gatcgtccaatcataaatcaacgtaacaagcccgaccgccacttttttacggaact

SEQ ID NO: 373 - DNA, 56 bp, synthetic construct
  source 1..56: mol_type = other DNA
agcggtccgccgacaagttaccagaaggcaccaggcggataattttttacggaact

SEQ ID NO: 374 - DNA, 42 bp, synthetic construct
  source 1..42: mol_type = other DNA
gaacgcgatagaagtagcattccacagattttttacggaact

SEQ ID NO: 375 - DNA, 42 bp, synthetic construct
  source 1..42: mol_type = other DNA
acaccgctaatagacaacagtttcagcgttttttacggaact

SEQ ID NO: 376 - DNA, 42 bp, synthetic construct
  source 1..42: mol_type = other DNA
aactaactttgcaatgtgctgcaaggcgttttttacggaact

SEQ ID NO: 377 - DNA, 42 bp, synthetic construct
  source 1..42: mol_type = other DNA
caatcgccctgtttgtgccggaaaccagttttttacggaact

SEQ ID NO: 378 - DNA, 64 bp, synthetic construct
  source 1..64: mol_type = other DNA
tattcacacaattcccgttgcgcacaaacaaataaacattacctccccggttttttacggaact

SEQ ID NO: 379 - DNA, 56 bp, synthetic construct
  source 1..56: mol_type = other DNA
cgcgtaatatccaggtaataaaagggactaatcctgtgtagattttttacggaact

SEQ ID NO: 380 - DNA, 42 bp, synthetic construct
  source 1..42: mol_type = other DNA
aacccacgccaccaaaagattaagaggattttttacggaact

SEQ ID NO: 381 - DNA, 63 bp, synthetic construct
  source 1..63: mol_type = other DNA
cccccagaatacacaccgaccgtgtgatggcatttatccccctcaaatgttttttacggaact

SEQ ID NO: 382 - DNA, 64 bp, synthetic construct
  source 1..64: mol_type = other DNA
caggcggtagctcaatgggcgcccaccaccctcagattttcatttaattgttttttacggaact

SEQ ID NO: 383 - DNA, 56 bp, synthetic construct
  source 1..56: mol_type = other DNA
tagtttggtaggtaggctatcaggtcatattaagtaatagcgttttttacggaact

SEQ ID NO: 384 - DNA, 42 bp, synthetic construct
  source 1..42: mol_type = other DNA
atcagctatcgtaagcaattcatcaatattttttacaccaca

SEQ ID NO: 385 - DNA, 56 bp, synthetic construct
  source 1..56: mol_type = other DNA
gaggccacctgagagtcaatagtgaattccaagaaccgaacgttttttaacggcta

SEQ ID NO: 386 - DNA, 64 bp, synthetic construct
  source 1..64: mol_type = other DNA
catttttaacggattggttgagaggagtgtactggtagggaggtgcgtagttttttacagagca

SEQ ID NO: 387 - DNA, 49 bp, synthetic construct
  source 1..49: mol_type = other DNA
gagccttacaactaaaggaatgagtgagggagcactttttgctgcatta

SEQ ID NO: 388 - DNA, 56 bp, synthetic construct
  source 1..56: mol_type = other DNA
gatcgtccaatcataaatcaacgtaacaagcccgaccgccacttttttatcgctca

SEQ ID NO: 389 - DNA, 56 bp, synthetic construct
  source 1..56: mol_type = other DNA
agcggtccgccgacaagttaccagaaggcaccaggcggataattttttatcgagca

SEQ ID NO: 390 - DNA, 42 bp, synthetic construct
  source 1..42: mol_type = other DNA
gaacgcgatagaagtagcattccacagattttttatctcgca

SEQ ID NO: 391 - DNA, 42 bp, synthetic construct
  source 1..42: mol_type = other DNA
acaccgctaatagacaacagtttcagcgttttttatcgacga

SEQ ID NO: 392 - DNA, 42 bp, synthetic construct
  source 1..42: mol_type = other DNA
atcagctatcgtaagcaattcatcaatattttttaggcatca

SEQ ID NO: 393 - DNA, 56 bp, synthetic construct
  source 1..56: mol_type = other DNA
gaggccacctgagagtcaatagtgaattccaagaaccgaacgttttttagtgagca

SEQ ID NO: 394 - DNA, 64 bp, synthetic construct
  source 1..64: mol_type = other DNA
catttttaacggattggttgagaggagtgtactggtagggaggtgcgtagttttttactcagca

SEQ ID NO: 395 - DNA, 49 bp, synthetic construct
  source 1..49: mol_type = other DNA
gagccttacaactaaaggaatgagtgagggagcacttttttacgtgcta

SEQ ID NO: 396 - DNA, 42 bp, synthetic construct
  source 1..42: mol_type = other DNA
aacccacgccaccaaaagattaagaggattttttacgtgcta

SEQ ID NO: 397 - DNA, 63 bp, synthetic construct
  source 1..63: mol_type = other DNA
cccccagaatacacaccgaccgtgtgatggcatttatccccctcaaatgttttttactcagca

SEQ ID NO: 398 - DNA, 64 bp, synthetic construct
  source 1..64: mol_type = other DNA
caggcggtagctcaatgggcgcccaccaccctcagattttcatttaattgttttttagtgagca

SEQ ID NO: 399 - DNA, 56 bp, synthetic construct
  source 1..56: mol_type = other DNA
tagtttggtaggtaggctatcaggtcatattaagtaatagcgttttttaggcatca

SEQ ID NO: 400 - DNA, 42 bp, synthetic construct
  source 1..42: mol_type = other DNA
gcttagatgataagcagactgtagcgcgttttttatgaggca

SEQ ID NO: 401 - DNA, 42 bp, synthetic construct
  source 1..42: mol_type = other DNA
gctgtttgctcgaaatcaccagtagcacttttttcaactcca

SEQ ID NO: 402 - DNA, 63 bp, synthetic construct
  source 1..63: mol_type = other DNA
agaaacgaaaccgagataccgatagttgcacgctgttattctgaaacatttttttatctgcga

SEQ ID NO: 403 - DNA, 42 bp, synthetic construct
  source 1..42: mol_type = other DNA
taacagtggtttaacattcaaccgattgttttttcacagaca

SEQ ID NO: 404 - DNA, 56 bp, synthetic construct
  source 1..56: mol_type = other DNA
gatcgtccaatcataaatcaacgtaacaagcccgaccgccacttttttccagttca

SEQ ID NO: 405 - DNA, 56 bp, synthetic construct
  source 1..56: mol_type = other DNA
agcggtccgccgacaagttaccagaaggcaccaggcggataattttttccaagtca

SEQ ID NO: 406 - DNA, 42 bp, synthetic construct
  source 1..42: mol_type = other DNA
gaacgcgatagaagtagcattccacagattttttccttgtca

SEQ ID NO: 407 - DNA, 42 bp, synthetic construct
  source 1..42: mol_type = other DNA
acaccgctaatagacaacagtttcagcgttttttcttgacca

SEQ ID NO: 408 - DNA, 42 bp, synthetic construct
  source 1..42: mol_type = other DNA
atcagctatcgtaagcaattcatcaatattttttactcagca

SEQ ID NO: 409 - DNA, 56 bp, synthetic construct
  source 1..56: mol_type = other DNA
gaggccacctgagagtcaatagtgaattccaagaaccgaacgttttttaggcatca

SEQ ID NO: 410 - DNA, 64 bp, synthetic construct
  source 1..64: mol_type = other DNA
catttttaacggattggttgagaggagtgtactggtagggaggtgcgtagttttttacgtgcta

SEQ ID NO: 411 - DNA, 49 bp, synthetic construct
  source 1..49: mol_type = other DNA
gagccttacaactaaaggaatgagtgagggagcacttttttagtgagca

SEQ ID NO: 412 - DNA, 42 bp, synthetic construct
  source 1..42: mol_type = other DNA
aacccacgccaccaaaagattaagaggattttttactcagca

SEQ ID NO: 413 - DNA, 63 bp, synthetic construct
  source 1..63: mol_type = other DNA
cccccagaatacacaccgaccgtgtgatggcatttatccccctcaaatgttttttaggcatca

SEQ ID NO: 414 - DNA, 64 bp, synthetic construct
  source 1..64: mol_type = other DNA
caggcggtagctcaatgggcgcccaccaccctcagattttcatttaattgttttttacgtgcta

SEQ ID NO: 415 - DNA, 56 bp, synthetic construct
  source 1..56: mol_type = other DNA
tagtttggtaggtaggctatcaggtcatattaagtaatagcgttttttagtgagca

SEQ ID NO: 416 - DNA, 56 bp, synthetic construct
  source 1..56: mol_type = other DNA
gatcgtccaatcataaatcaacgtaacaagcccgaccgccacttttttaggcatca

SEQ ID NO: 417 - DNA, 56 bp, synthetic construct
  source 1..56: mol_type = other DNA
agcggtccgccgacaagttaccagaaggcaccaggcggataattttttagtgagca

SEQ ID NO: 418 - DNA, 42 bp, synthetic construct
  source 1..42: mol_type = other DNA
gaacgcgatagaagtagcattccacagattttttactcagca

SEQ ID NO: 419 - DNA, 42 bp, synthetic construct
  source 1..42: mol_type = other DNA
acaccgctaatagacaacagtttcagcgttttttacgtgcta

SEQ ID NO: 420 - DNA, 42 bp, synthetic construct
  source 1..42: mol_type = other DNA
aactaactttgcaatgtgctgcaaggcgttttttaggcatca

SEQ ID NO: 421 - DNA, 42 bp, synthetic construct
  source 1..42: mol_type = other DNA
caatcgccctgtttgtgccggaaaccagttttttagtgagca

SEQ ID NO: 422 - DNA, 64 bp, synthetic construct
  source 1..64: mol_type = other DNA
tattcacacaattcccgttgcgcacaaacaaataaacattacctccccggttttttactcagca

SEQ ID NO: 423 - DNA, 56 bp, synthetic construct
  source 1..56: mol_type = other DNA
cgcgtaatatccaggtaataaaagggactaatcctgtgtagattttttacgtgcta

SEQ ID NO: 424 - DNA, 42 bp, synthetic construct
  source 1..42: mol_type = other DNA
atcagctatcgtaagcaattcatcaatattttttacgctgta

SEQ ID NO: 425 - DNA, 56 bp, synthetic construct
  source 1..56: mol_type = other DNA
gaggccacctgagagtcaatagtgaattccaagaaccgaacgttttttatgcggta

SEQ ID NO: 426 - DNA, 64 bp, synthetic construct
  source 1..64: mol_type = other DNA
catttttaacggattggttgagaggagtgtactggtagggaggtgcgtagttttttatggcgta

SEQ ID NO: 427 - DNA, 49 bp, synthetic construct
  source 1..49: mol_type = other DNA
gagccttacaactaaaggaatgagtgagggagcacttttttagtgcgta

SEQ ID NO: 428 - DNA, 56 bp, synthetic construct
  source 1..56: mol_type = other DNA
gatcgtccaatcataaatcaacgtaacaagcccgaccgccacttttttagcacgta

SEQ ID NO: 429 - DNA, 56 bp, synthetic construct
  source 1..56: mol_type = other DNA
agcggtccgccgacaagttaccagaaggcaccaggcggataattttttgctcacta

SEQ ID NO: 430 - DNA, 42 bp, synthetic construct
  source 1..42: mol_type = other DNA
gaacgcgatagaagtagcattccacagattttttgctgagta

SEQ ID NO: 431 - DNA, 42 bp, synthetic construct
  source 1..42: mol_type = other DNA
acaccgctaatagacaacagtttcagcgttttttgatgccta

SEQ ID NO: 432 - DNA, 42 bp, synthetic construct
  source 1..42: mol_type = other DNA
atcagctatcgtaagcaattcatcaatattttttccgattct

SEQ ID NO: 433 - DNA, 56 bp, synthetic construct
  source 1..56: mol_type = other DNA
gaggccacctgagagtcaatagtgaattccaagaaccgaacgttttttccgattct

SEQ ID NO: 434 - DNA, 64 bp, synthetic construct
  source 1..64: mol_type = other DNA
catttttaacggattggttgagaggagtgtactggtagggaggtgcgtagttttttccgattct

SEQ ID NO: 435 - DNA, 49 bp, synthetic construct
  source 1..49: mol_type = other DNA
gagccttacaactaaaggaatgagtgagggagcacttttttccgattct

SEQ ID NO: 436 - DNA, 56 bp, synthetic construct
  source 1..56: mol_type = other DNA
gatcgtccaatcataaatcaacgtaacaagcccgaccgccactttttagttccgta

SEQ ID NO: 437 - DNA, 56 bp, synthetic construct
  source 1..56: mol_type = other DNA
agcggtccgccgacaagttaccagaaggcaccaggcggataatttttagttccgta

SEQ ID NO: 438 - DNA, 42 bp, synthetic construct
  source 1..42: mol_type = other DNA
gaacgcgatagaagtagcattccacagatttttagttccgta

SEQ ID NO: 439 - DNA, 42 bp, synthetic construct
  source 1..42: mol_type = other DNA
acaccgctaatagacaacagtttcagcgtttttagttccgta

SEQ ID NO: 440 - DNA, 42 bp, synthetic construct
  source 1..42: mol_type = other DNA
aactaactttgcaatgtgctgcaaggcgttttttccgattct

SEQ ID NO: 441 - DNA, 42 bp, synthetic construct
  source 1..42: mol_type = other DNA
caatcgccctgtttgtgccggaaaccagttttttccgattct

SEQ ID NO: 442 - DNA, 64 bp, synthetic construct
  source 1..64: mol_type = other DNA
tattcacacaattcccgttgcgcacaaacaaataaacattacctccccggttttttccgattct

SEQ ID NO: 443 - DNA, 56 bp, synthetic construct
  source 1..56: mol_type = other DNA
cgcgtaatatccaggtaataaaagggactaatcctgtgtagattttttccgattct

SEQ ID NO: 444 - DNA, 56 bp, synthetic construct
  source 1..56: mol_type = other DNA
gatcgtccaatcataaatcaacgtaacaagcccgaccgccacttttttacgtgcta

SEQ ID NO: 445 - DNA, 56 bp, synthetic construct
  source 1..56: mol_type = other DNA
agcggtccgccgacaagttaccagaaggcaccaggcggataattttttactcagca

SEQ ID NO: 446 - DNA, 42 bp, synthetic construct
  source 1..42: mol_type = other DNA
gaacgcgatagaagtagcattccacagattttttagtgagca

SEQ ID NO: 447 - DNA, 42 bp, synthetic construct
  source 1..42: mol_type = other DNA
acaccgctaatagacaacagtttcagcgttttttaggcatca

SEQ ID NO: 448 - DNA, 42 bp, synthetic construct
  source 1..42: mol_type = other DNA
aactaactttgcaatgtgctgcaaggcgttttttgctcgata

SEQ ID NO: 449 - DNA, 42 bp, synthetic construct
  source 1..42: mol_type = other DNA
caatcgccctgtttgtgccggaaaccagttttttgagcgata

SEQ ID NO: 450 - DNA, 64 bp, synthetic construct
  source 1..64: mol_type = other DNA
tattcacacaattcccgttgcgcacaaacaaataaacattacctccccggttttttcgtcgata

SEQ ID NO: 451 - DNA, 56 bp, synthetic construct
  source 1..56: mol_type = other DNA
cgcgtaatatccaggtaataaaagggactaatcctgtgtagattttttgcgagata

SEQ ID NO: 452 - DNA, 56 bp, synthetic construct
  source 1..56: mol_type = other DNA
gatcgtccaatcataaatcaacgtaacaagcccgaccgccacttttttgtggtgta

SEQ ID NO: 453 - DNA, 56 bp, synthetic construct
  source 1..56: mol_type = other DNA
agcggtccgccgacaagttaccagaaggcaccaggcggataattttttgctctgta

SEQ ID NO: 454 - DNA, 42 bp, synthetic construct
  source 1..42: mol_type = other DNA
gaacgcgatagaagtagcattccacagattttttagccgtta

SEQ ID NO: 455 - DNA, 42 bp, synthetic construct
  source 1..42: mol_type = other DNA
acaccgctaatagacaacagtttcagcgttttttaatgcagc

SEQ ID NO: 456 - DNA, 42 bp, synthetic construct
  source 1..42: mol_type = other DNA
aactaactttgcaatgtgctgcaaggcgttttttacgcacta

SEQ ID NO: 457 - DNA, 42 bp, synthetic construct
  source 1..42: mol_type = other DNA
caatcgccctgtttgtgccggaaaccagttttttaccgcata

SEQ ID NO: 458 - DNA, 64 bp, synthetic construct
  source 1..64: mol_type = other DNA
tattcacacaattcccgttgcgcacaaacaaataaacattacctccccggttttttacgccata

SEQ ID NO: 459 - DNA, 56 bp, synthetic construct
  source 1..56: mol_type = other DNA
cgcgtaatatccaggtaataaaagggactaatcctgtgtagattttttacagcgta

SEQ ID NO: 460 - DNA, 56 bp, synthetic construct
  source 1..56: mol_type = other DNA
gatcgtccaatcataaatcaacgtaacaagcccgaccgccacttttttcgcagata

SEQ ID NO: 461 - DNA, 56 bp, synthetic construct
  source 1..56: mol_type = other DNA
agcggtccgccgacaagttaccagaaggcaccaggcggataattttttgtctgtga

SEQ ID NO: 462 - DNA, 42 bp, synthetic construct
  source 1..42: mol_type = other DNA
gaacgcgatagaagtagcattccacagattttttgcctcata

SEQ ID NO: 463 - DNA, 42 bp, synthetic construct
  source 1..42: mol_type = other DNA
acaccgctaatagacaacagtttcagcgttttttggagttga

SEQ ID NO: 464 - DNA, 42 bp, synthetic construct
  source 1..42: mol_type = other DNA
aactaactttgcaatgtgctgcaaggcgttttttgacttgga

SEQ ID NO: 465 - DNA, 42 bp, synthetic construct
  source 1..42: mol_type = other DNA
caatcgccctgtttgtgccggaaaccagttttttgaactgga

SEQ ID NO: 466 - DNA, 64 bp, synthetic construct
  source 1..64: mol_type = other DNA
tattcacacaattcccgttgcgcacaaacaaataaacattacctccccggttttttggtcaaga

SEQ ID NO: 467 - DNA, 56 bp, synthetic construct
  source 1..56: mol_type = other DNA
cgcgtaatatccaggtaataaaagggactaatcctgtgtagattttttgacaagga

SEQ ID NO: 468 - DNA, 56 bp, synthetic construct
  source 1..56: mol_type = other DNA
gatcgtccaatcataaatcaacgtaacaagcccgaccgccactttttagaatcgga

SEQ ID NO: 469 - DNA, 56 bp, synthetic construct
  source 1..56: mol_type = other DNA
agcggtccgccgacaagttaccagaaggcaccaggcggataatttttagaatcgga

SEQ ID NO: 470 - DNA, 42 bp, synthetic construct
  source 1..42: mol_type = other DNA
gaacgcgatagaagtagcattccacagatttttagaatcgga

SEQ ID NO: 471 - DNA, 42 bp, synthetic construct
  source 1..42: mol_type = other DNA
acaccgctaatagacaacagtttcagcgtttttagaatcgga

SEQ ID NO: 472 - DNA, 55 bp, synthetic construct
  source 1..55: mol_type = other DNA
gatcgtccaatcataaatcaacgtaacaagcccgaccgccactttttacttgacg

SEQ ID NO: 473 - DNA, 55 bp, synthetic construct
  source 1..55: mol_type = other DNA
agcggtccgccgacaagttaccagaaggcaccaggcggataatttttacttgacg

SEQ ID NO: 474 - DNA, 41 bp, synthetic construct
  source 1..41: mol_type = other DNA
gaacgcgatagaagtagcattccacagatttttacttgacg

SEQ ID NO: 475 - DNA, 41 bp, synthetic construct
  source 1..41: mol_type = other DNA
acaccgctaatagacaacagtttcagcgtttttacttgacg

SEQ ID NO: 476 - DNA, 41 bp, synthetic construct
  source 1..41: mol_type = other DNA
atcagctatcgtaagcaattcatcaatatttttacttgacg

SEQ ID NO: 477 - DNA, 55 bp, synthetic construct
  source 1..55: mol_type = other DNA
gaggccacctgagagtcaatagtgaattccaagaaccgaacgtttttacttgacg

SEQ ID NO: 478 - DNA, 63 bp, synthetic construct
  source 1..63: mol_type = other DNA
catttttaacggattggttgagaggagtgtactggtagggaggtgcgtagtttttacttgacg

SEQ ID NO: 479 - DNA, 48 bp, synthetic construct
  source 1..48: mol_type = other DNA
gagccttacaactaaaggaatgagtgagggagcactttttacttgacg

SEQ ID NO: 480 - DNA, 55 bp, synthetic construct
  source 1..55: mol_type = other DNA
caccaacaaagtactttcaactttaatcctttaaagtaattttttttacttgacg

SEQ ID NO: 481 - DNA, 54 bp, synthetic construct
  source 1..54: mol_type = other DNA
gcgggactaacgaagagaataacataacagccctcaagcaatttttacttgacg

SEQ ID NO: 482 - DNA, 55 bp, synthetic construct
  source 1..55: mol_type = other DNA
gcaaggcctgtaatgataatcagaaaaggcaaagctaatgcatttttacttgacg

SEQ ID NO: 483 - DNA, 62 bp, synthetic construct
  source 1..62: mol_type = other DNA
attttagtcacgcatagtctttaatgcgttttaaactttccttatcatttttttacttgacg

SEQ ID NO: 484 - DNA, 41 bp, synthetic construct
  source 1..41: mol_type = other DNA
aacccacgccaccaaaagattaagaggatttttacttgacg

SEQ ID NO: 485 - DNA, 62 bp, synthetic construct
  source 1..62: mol_type = other DNA
cccccagaatacacaccgaccgtgtgatggcatttatccccctcaaatgtttttacttgacg

SEQ ID NO: 486 - DNA, 63 bp, synthetic construct
  source 1..63: mol_type = other DNA
caggcggtagctcaatgggcgcccaccaccctcagattttcatttaattgtttttacttgacg

SEQ ID NO: 487 - DNA, 55 bp, synthetic construct
  source 1..55: mol_type = other DNA
tagtttggtaggtaggctatcaggtcatattaagtaatagcgtttttacttgacg

SEQ ID NO: 488 - DNA, 41 bp, synthetic construct
  source 1..41: mol_type = other DNA
gcttagatgataagcagactgtagcgcgtttttacttgacg

SEQ ID NO: 489 - DNA, 41 bp, synthetic construct
  source 1..41: mol_type = other DNA
gctgtttgctcgaaatcaccagtagcactttttacttgacg

SEQ ID NO: 490 - DNA, 62 bp, synthetic construct
  source 1..62: mol_type = other DNA
agaaacgaaaccgagataccgatagttgcacgctgttattctgaaacattttttacttgacg

SEQ ID NO: 491 - DNA, 41 bp, synthetic construct
  source 1..41: mol_type = other DNA
taacagtggtttaacattcaaccgattgtttttacttgacg

SEQ ID NO: 492 - DNA, 41 bp, synthetic construct
  source 1..41: mol_type = other DNA
aactaactttgcaatgtgctgcaaggcgtttttacttgacg

SEQ ID NO: 493 - DNA, 41 bp, synthetic construct
  source 1..41: mol_type = other DNA
caatcgccctgtttgtgccggaaaccagtttttacttgacg

SEQ ID NO: 494 - DNA, 63 bp, synthetic construct
  source 1..63: mol_type = other DNA
tattcacacaattcccgttgcgcacaaacaaataaacattacctccccggtttttacttgacg

SEQ ID NO: 495 - DNA, 55 bp, synthetic construct
  source 1..55: mol_type = other DNA
cgcgtaatatccaggtaataaaagggactaatcctgtgtagatttttacttgacg

SEQ ID NO: 496 - DNA, 55 bp, synthetic construct
  source 1..55: mol_type = other DNA
gatcgtccaatcataaatcaacgtaacaagcccgaccgccactttttcgtcaagt

SEQ ID NO: 497 - DNA, 55 bp, synthetic construct
  source 1..55: mol_type = other DNA
agcggtccgccgacaagttaccagaaggcaccaggcggataatttttcgtcaagt

SEQ ID NO: 498 - DNA, 41 bp, synthetic construct
  source 1..41: mol_type = other DNA
gaacgcgatagaagtagcattccacagatttttcgtcaagt

SEQ ID NO: 499 - DNA, 41 bp, synthetic construct
  source 1..41: mol_type = other DNA
acaccgctaatagacaacagtttcagcgtttttcgtcaagt

SEQ ID NO: 500 - DNA, 41 bp, synthetic construct
  source 1..41: mol_type = other DNA
atcagctatcgtaagcaattcatcaatatttttcgtcaagt

SEQ ID NO: 501 - DNA, 55 bp, synthetic construct
  source 1..55: mol_type = other DNA
gaggccacctgagagtcaatagtgaattccaagaaccgaacgtttttcgtcaagt

SEQ ID NO: 502 - DNA, 63 bp, synthetic construct
  source 1..63: mol_type = other DNA
catttttaacggattggttgagaggagtgtactggtagggaggtgcgtagtttttcgtcaagt

SEQ ID NO: 503 - DNA, 48 bp, synthetic construct
  source 1..48: mol_type = other DNA
gagccttacaactaaaggaatgagtgagggagcactttttcgtcaagt

SEQ ID NO: 504 - DNA, 55 bp, synthetic construct
  source 1..55: mol_type = other DNA
caccaacaaagtactttcaactttaatcctttaaagtaattttttttcgtcaagt

SEQ ID NO: 505 - DNA, 54 bp, synthetic construct
  source 1..54: mol_type = other DNA
gcgggactaacgaagagaataacataacagccctcaagcaatttttcgtcaagt

SEQ ID NO: 506 - DNA, 55 bp, synthetic construct
  source 1..55: mol_type = other DNA
gcaaggcctgtaatgataatcagaaaaggcaaagctaatgcatttttcgtcaagt

SEQ ID NO: 507 - DNA, 62 bp, synthetic construct
  source 1..62: mol_type = other DNA
attttagtcacgcatagtctttaatgcgttttaaactttccttatcatttttttcgtcaagt

SEQ ID NO: 508 - DNA, 41 bp, synthetic construct
  source 1..41: mol_type = other DNA
aacccacgccaccaaaagattaagaggatttttcgtcaagt

SEQ ID NO: 509 - DNA, 62 bp, synthetic construct
  source 1..62: mol_type = other DNA
cccccagaatacacaccgaccgtgtgatggcatttatccccctcaaatgtttttcgtcaagt

SEQ ID NO: 510 - DNA, 63 bp, synthetic construct
  source 1..63: mol_type = other DNA
caggcggtagctcaatgggcgcccaccaccctcagattttcatttaattgtttttcgtcaagt

SEQ ID NO: 511 - DNA, 55 bp, synthetic construct
  source 1..55: mol_type = other DNA
tagtttggtaggtaggctatcaggtcatattaagtaatagcgtttttcgtcaagt

SEQ ID NO: 512 - DNA, 41 bp, synthetic construct
  source 1..41: mol_type = other DNA
gcttagatgataagcagactgtagcgcgtttttcgtcaagt

SEQ ID NO: 513 - DNA, 41 bp, synthetic construct
  source 1..41: mol_type = other DNA
gctgtttgctcgaaatcaccagtagcactttttcgtcaagt

SEQ ID NO: 514 - DNA, 62 bp, synthetic construct
  source 1..62: mol_type = other DNA
agaaacgaaaccgagataccgatagttgcacgctgttattctgaaacattttttcgtcaagt

SEQ ID NO: 515 - DNA, 41 bp, synthetic construct
  source 1..41: mol_type = other DNA
taacagtggtttaacattcaaccgattgtttttcgtcaagt

SEQ ID NO: 516 - DNA, 41 bp, synthetic construct
  source 1..41: mol_type = other DNA
aactaactttgcaatgtgctgcaaggcgtttttcgtcaagt

SEQ ID NO: 517 - DNA, 41 bp, synthetic construct
  source 1..41: mol_type = other DNA
caatcgccctgtttgtgccggaaaccagtttttcgtcaagt

SEQ ID NO: 518 - DNA, 63 bp, synthetic construct
  source 1..63: mol_type = other DNA
tattcacacaattcccgttgcgcacaaacaaataaacattacctccccggtttttcgtcaagt

SEQ ID NO: 519 - DNA, 55 bp, synthetic construct
  source 1..55: mol_type = other DNA
cgcgtaatatccaggtaataaaagggactaatcctgtgtagatttttcgtcaagt

SEQ ID NO: 520 - DNA, 65 bp, synthetic construct
  source 1..65: mol_type = other DNA
gatcgtccaatcataaatcaacgtaacaagcccgaccgccactttttttttttttttacttgacg

SEQ ID NO: 521 - DNA, 65 bp, synthetic construct
  source 1..65: mol_type = other DNA
agcggtccgccgacaagttaccagaaggcaccaggcggataatttttttttttttttacttgacg

SEQ ID NO: 522 - DNA, 51 bp, synthetic construct
  source 1..51: mol_type = other DNA
gaacgcgatagaagtagcattccacagatttttttttttttttacttgacg

SEQ ID NO: 523 - DNA, 51 bp, synthetic construct
  source 1..51: mol_type = other DNA
acaccgctaatagacaacagtttcagcgtttttttttttttttacttgacg

SEQ ID NO: 524 - DNA, 51 bp, synthetic construct
  source 1..51: mol_type = other DNA
atcagctatcgtaagcaattcatcaatatttttttttttttttacttgacg

SEQ ID NO: 525 - DNA, 65 bp, synthetic construct
  source 1..65: mol_type = other DNA
gaggccacctgagagtcaatagtgaattccaagaaccgaacgtttttttttttttttacttgacg

SEQ ID NO: 526 - DNA, 73 bp, synthetic construct
  source 1..73: mol_type = other DNA
catttttaacggattggttgagaggagtgtactggtagggaggtgcgtagtttttttttttttttacttgacg

SEQ ID NO: 527 - DNA, 58 bp, synthetic construct
  source 1..58: mol_type = other DNA
gagccttacaactaaaggaatgagtgagggagcactttttttttttttttacttgacg

SEQ ID NO: 528 - DNA, 65 bp, synthetic construct
  source 1..65: mol_type = other DNA
caccaacaaagtactttcaactttaatcctttaaagtaattttttttttttttttttacttgacg

SEQ ID NO: 529 - DNA, 64 bp, synthetic construct
  source 1..64: mol_type = other DNA
gcgggactaacgaagagaataacataacagccctcaagcaatttttttttttttttacttgacg

SEQ ID NO: 530 - DNA, 65 bp, synthetic construct
  source 1..65: mol_type = other DNA
gcaaggcctgtaatgataatcagaaaaggcaaagctaatgcatttttttttttttttacttgacg

SEQ ID NO: 531 - DNA, 72 bp, synthetic construct
  source 1..72: mol_type = other DNA
attttagtcacgcatagtctttaatgcgttttaaactttccttatcatttttttttttttttttacttgacg

SEQ ID NO: 532 - DNA, 51 bp, synthetic construct
  source 1..51: mol_type = other DNA
aacccacgccaccaaaagattaagaggatttttttttttttttacttgacg

SEQ ID NO: 533 - DNA, 72 bp, synthetic construct
  source 1..72: mol_type = other DNA
cccccagaatacacaccgaccgtgtgatggcatttatccccctcaaatgtttttttttttttttacttgacg

SEQ ID NO: 534 - DNA, 73 bp, synthetic construct
  source 1..73: mol_type = other DNA
caggcggtagctcaatgggcgcccaccaccctcagattttcatttaattgtttttttttttttttacttgacg

SEQ ID NO: 535 - DNA, 65 bp, synthetic construct
  source 1..65: mol_type = other DNA
tagtttggtaggtaggctatcaggtcatattaagtaatagcgtttttttttttttttacttgacg

SEQ ID NO: 536 - DNA, 51 bp, synthetic construct
  source 1..51: mol_type = other DNA
gcttagatgataagcagactgtagcgcgtttttttttttttttacttgacg

SEQ ID NO: 537 - DNA, 51 bp, synthetic construct
  source 1..51: mol_type = other DNA
gctgtttgctcgaaatcaccagtagcactttttttttttttttacttgacg

SEQ ID NO: 538 - DNA, 72 bp, synthetic construct
  source 1..72: mol_type = other DNA
agaaacgaaaccgagataccgatagttgcacgctgttattctgaaacattttttttttttttttacttgacg

SEQ ID NO: 539 - DNA, 51 bp, synthetic construct
  source 1..51: mol_type = other DNA
taacagtggtttaacattcaaccgattgtttttttttttttttacttgacg

SEQ ID NO: 540 - DNA, 51 bp, synthetic construct
  source 1..51: mol_type = other DNA
aactaactttgcaatgtgctgcaaggcgtttttttttttttttacttgacg

SEQ ID NO: 541 - DNA, 51 bp, synthetic construct
  source 1..51: mol_type = other DNA
caatcgccctgtttgtgccggaaaccagtttttttttttttttacttgacg

SEQ ID NO: 542 - DNA, 73 bp, synthetic construct
  source 1..73: mol_type = other DNA
tattcacacaattcccgttgcgcacaaacaaataaacattacctccccggtttttttttttttttacttgacg

SEQ ID NO: 543 - DNA, 65 bp, synthetic construct
  source 1..65: mol_type = other DNA
cgcgtaatatccaggtaataaaagggactaatcctgtgtagatttttttttttttttacttgacg

SEQ ID NO: 544 - DNA, 65 bp, synthetic construct
  source 1..65: mol_type = other DNA
gatcgtccaatcataaatcaacgtaacaagcccgaccgccactttttttttttttttcgtcaagt

SEQ ID NO: 545 - DNA, 65 bp, synthetic construct
  source 1..65: mol_type = other DNA
agcggtccgccgacaagttaccagaaggcaccaggcggataatttttttttttttttcgtcaagt

SEQ ID NO: 546 - DNA, 51 bp, synthetic construct
  source 1..51: mol_type = other DNA
gaacgcgatagaagtagcattccacagatttttttttttttttcgtcaagt

SEQ ID NO: 547 - DNA, 51 bp, synthetic construct
  source 1..51: mol_type = other DNA
acaccgctaatagacaacagtttcagcgtttttttttttttttcgtcaagt

SEQ ID NO: 548 - DNA, 51 bp, synthetic construct
  source 1..51: mol_type = other DNA
atcagctatcgtaagcaattcatcaatatttttttttttttttcgtcaagt

SEQ ID NO: 549 - DNA, 65 bp, synthetic construct
  source 1..65: mol_type = other DNA
gaggccacctgagagtcaatagtgaattccaagaaccgaacgtttttttttttttttcgtcaagt

SEQ ID NO: 550 - DNA, 73 bp, synthetic construct
  source 1..73: mol_type = other DNA
catttttaacggattggttgagaggagtgtactggtagggaggtgcgtagtttttttttttttttcgtcaagt

SEQ ID NO: 551 - DNA, 58 bp, synthetic construct
  source 1..58: mol_type = other DNA
gagccttacaactaaaggaatgagtgagggagcactttttttttttttttcgtcaagt

SEQ ID NO: 552 - DNA, 65 bp, synthetic construct
  source 1..65: mol_type = other DNA
caccaacaaagtactttcaactttaatcctttaaagtaattttttttttttttttttcgtcaagt

SEQ ID NO: 553 - DNA, 64 bp, synthetic construct
  source 1..64: mol_type = other DNA
gcgggactaacgaagagaataacataacagccctcaagcaatttttttttttttttcgtcaagt

SEQ ID NO: 554 - DNA, 65 bp, synthetic construct
  source 1..65: mol_type = other DNA
gcaaggcctgtaatgataatcagaaaaggcaaagctaatgcatttttttttttttttcgtcaagt

SEQ ID NO: 555 - DNA, 72 bp, synthetic construct
  source 1..72: mol_type = other DNA
attttagtcacgcatagtctttaatgcgttttaaactttccttatcatttttttttttttttttcgtcaagt

SEQ ID NO: 556 - DNA, 51 bp, synthetic construct
  source 1..51: mol_type = other DNA
aacccacgccaccaaaagattaagaggatttttttttttttttcgtcaagt

SEQ ID NO: 557 - DNA, 72 bp, synthetic construct
  source 1..72: mol_type = other DNA
cccccagaatacacaccgaccgtgtgatggcatttatccccctcaaatgtttttttttttttttcgtcaagt

SEQ ID NO: 558 - DNA, 73 bp, synthetic construct
  source 1..73: mol_type = other DNA
caggcggtagctcaatgggcgcccaccaccctcagattttcatttaattgtttttttttttttttcgtcaagt

SEQ ID NO: 559 - DNA, 65 bp, synthetic construct
  source 1..65: mol_type = other DNA
tagtttggtaggtaggctatcaggtcatattaagtaatagcgtttttttttttttttcgtcaagt

SEQ ID NO: 560 - DNA, 51 bp, synthetic construct
  source 1..51: mol_type = other DNA
gcttagatgataagcagactgtagcgcgtttttttttttttttcgtcaagt

SEQ ID NO: 561 - DNA, 41 bp, synthetic construct; identical to SEQ ID NO: 513
  source 1..41: mol_type = other DNA

SEQ ID NO: 562 - DNA, 72 bp, synthetic construct
  source 1..72: mol_type = other DNA
agaaacgaaaccgagataccgatagttgcacgctgttattctgaaacattttttttttttttttcgtcaagt

SEQ ID NO: 563 - DNA, 51 bp, synthetic construct
  source 1..51: mol_type = other DNA
taacagtggtttaacattcaaccgattgtttttttttttttttcgtcaagt

SEQ ID NO: 564 - DNA, 51 bp, synthetic construct
  source 1..51: mol_type = other DNA
aactaactttgcaatgtgctgcaaggcgtttttttttttttttcgtcaagt

SEQ ID NO: 565 - DNA, 51 bp, synthetic construct
  source 1..51: mol_type = other DNA
caatcgccctgtttgtgccggaaaccagtttttttttttttttcgtcaagt

SEQ ID NO: 566 - DNA, 73 bp, synthetic construct
  source 1..73: mol_type = other DNA
tattcacacaattcccgttgcgcacaaacaaataaacattacctccccggtttttttttttttttcgtcaagt

SEQ ID NO: 567 - DNA, 65 bp, synthetic construct
  source 1..65: mol_type = other DNA
cgcgtaatatccaggtaataaaagggactaatcctgtgtagatttttttttttttttcgtcaagt

SEQ ID NO: 568 - DNA, 72 bp, synthetic construct
  source 1..72: mol_type = other DNA
gatcgtccaatcataaatcaacgtaacaagcccgaccgccactttttttttttttacaaggcaacctcaaca

SEQ ID NO: 569 - DNA, 72 bp, synthetic construct
  source 1..72: mol_type = other DNA
agcggtccgccgacaagttaccagaaggcaccaggcggataatttttttttttttgttctggtagcgatgta

SEQ ID NO: 570 - DNA, 58 bp, synthetic construct
  source 1..58: mol_type = other DNA
gaacgcgatagaagtagcattccacagatttttttttttttgagtgtgtatcgtgcta

SEQ ID NO: 571 - DNA, 58 bp, synthetic construct
  source 1..58: mol_type = other DNA
acaccgctaatagacaacagtttcagcgtttttttttttttacgcatctattcgccta

SEQ ID NO: 572 - DNA, 63 bp, synthetic construct; identical to SEQ ID NO: 228
  source 1..63: mol_type = other DNA

SEQ ID NO: 573 - DNA, 63 bp, synthetic construct; identical to SEQ ID NO: 229
  source 1..63: mol_type = other DNA

SEQ ID NO: 574 - DNA, 49 bp, synthetic construct; identical to SEQ ID NO: 230
  source 1..49: mol_type = other DNA

SEQ ID NO: 575 - DNA, 49 bp, synthetic construct; identical to SEQ ID NO: 231
  source 1..49: mol_type = other DNA

SEQ ID NO: 576 - DNA, 72 bp, synthetic construct
  source 1..72: mol_type = other DNA
gatcgtccaatcataaatcaacgtaacaagcccgaccgccactttttttttttttacatcgctaccagaaca

SEQ ID NO: 577 - DNA, 72 bp, synthetic construct
  source 1..72: mol_type = other DNA
agcggtccgccgacaagttaccagaaggcaccaggcggataatttttttttttttgttgaggttgccttgta

SEQ ID NO: 578 - DNA, 58 bp, synthetic construct
  source 1..58: mol_type = other DNA
gaacgcgatagaagtagcattccacagatttttttttttttaggcgaatagatgcgta

SEQ ID NO: 579 - DNA, 58 bp, synthetic construct
  source 1..58: mol_type = other DNA
acaccgctaatagacaacagtttcagcgtttttttttttttagcacgatacacactca

SEQ ID NO: 580 - DNA, 49 bp, synthetic construct
  source 1..49: mol_type = other DNA
aactaactttgcaatgtgctgcaaggcgtttttttttttttaacagcca

SEQ ID NO: 581 - DNA, 49 bp, synthetic construct
  source 1..49: mol_type = other DNA
caatcgccctgtttgtgccggaaaccagtttttttttttttaagccaca

SEQ ID NO: 582 - DNA, 71 bp, synthetic construct
  source 1..71: mol_type = other DNA
tattcacacaattcccgttgcgcacaaacaaataaacattacctccccggtttttttttttttaccaagca

SEQ ID NO: 583 - DNA, 63 bp, synthetic construct
  source 1..63: mol_type = other DNA
cgcgtaatatccaggtaataaaagggactaatcctgtgtagatttttttttttttaggttgca

SEQ ID NO: 584 - DNA, 63 bp, synthetic construct
  source 1..63: mol_type = other DNA
caccaacaaagtactttcaactttaatcctttaaagtaattttttttttttttttggctgtta

SEQ ID NO: 585 - DNA, 62 bp, synthetic construct
  source 1..62: mol_type = other DNA
gcgggactaacgaagagaataacataacagccctcaagcaatttttttttttttgcttggta

SEQ ID NO: 586 - DNA, 63 bp, synthetic construct
  source 1..63: mol_type = other DNA
gcaaggcctgtaatgataatcagaaaaggcaaagctaatgcatttttttttttttgtggctta

SEQ ID NO: 587 - DNA, 70 bp, synthetic construct
  source 1..70: mol_type = other DNA
attttagtcacgcatagtctttaatgcgttttaaactttccttatcatttttttttttttttgcaaccta

SEQ ID NO: 588 - DNA, 49 bp, synthetic construct
  source 1..49: mol_type = other DNA
aacccacgccaccaaaagattaagaggattttttttttttaccagacat

SEQ ID NO: 589 - DNA, 70 bp, synthetic construct
  source 1..70: mol_type = other DNA
cccccagaatacacaccgaccgtgtgatggcatttatccccctcaaatgttttttttttttatacagcac

SEQ ID NO: 590 - DNA, 71 bp, synthetic construct
  source 1..71: mol_type = other DNA
caggcggtagctcaatgggcgcccaccaccctcagattttcatttaattgttttttttttttactgtccat

SEQ ID NO: 591 - DNA, 63 bp, synthetic construct
  source 1..63: mol_type = other DNA
tagtttggtaggtaggctatcaggtcatattaagtaatagcgttttttttttttacaggtcat

SEQ ID NO: 592 - DNA, 49 bp, synthetic construct
  source 1..49: mol_type = other DNA
gcttagatgataagcagactgtagcgcgttttttttttttacaggtcat

SEQ ID NO: 593 - DNA, 49 bp, synthetic construct
  source 1..49: mol_type = other DNA
gctgtttgctcgaaatcaccagtagcacttttttttttttactgtccat

SEQ ID NO: 594 - DNA, 70 bp, synthetic construct
  source 1..70: mol_type = other DNA
agaaacgaaaccgagataccgatagttgcacgctgttattctgaaacatttttttttttttatacagcac

SEQ ID NO: 595 - DNA, 49 bp, synthetic construct
  source 1..49: mol_type = other DNA
taacagtggtttaacattcaaccgattgttttttttttttaccagacat

SEQ ID NO: 596 - DNA, 49 bp, synthetic construct
  source 1..49: mol_type = other DNA
aactaactttgcaatgtgctgcaaggcgttttttttttttactgtccat

SEQ ID NO: 597 - DNA, 49 bp, synthetic construct
  source 1..49: mol_type = other DNA
caatcgccctgtttgtgccggaaaccagttttttttttttaccagacat

SEQ ID NO: 598 - DNA, 71 bp, synthetic construct
  source 1..71: mol_type = other DNA
tattcacacaattcccgttgcgcacaaacaaataaacattacctccccggttttttttttttacaggtcat

SEQ ID NO: 599 - DNA, 63 bp, synthetic construct
  source 1..63: mol_type = other DNA
cgcgtaatatccaggtaataaaagggactaatcctgtgtagattttttttttttatacagcac

SEQ ID NO: 600 - DNA, 63 bp, synthetic construct
  source 1..63: mol_type = other DNA
gatcgtccaatcataaatcaacgtaacaagcccgaccgccacttttttttttttgtgctgtat

SEQ ID NO: 601 - DNA, 63 bp, synthetic construct
  source 1..63: mol_type = other DNA
agcggtccgccgacaagttaccagaaggcaccaggcggataattttttttttttatgtctggt

SEQ ID NO: 602 - DNA, 49 bp, synthetic construct
  source 1..49: mol_type = other DNA
gaacgcgatagaagtagcattccacagattttttttttttatgacctgt

SEQ ID NO: 603 - DNA, 49 bp, synthetic construct
  source 1..49: mol_type = other DNA
acaccgctaatagacaacagtttcagcgttttttttttttatggacagt

SEQ ID NO: 604 - DNA, 49 bp, synthetic construct
  source 1..49: mol_type = other DNA
aacccacgccaccaaaagattaagaggattttttttttttagatgaacg

SEQ ID NO: 605 - DNA, 70 bp, synthetic construct
  source 1..70: mol_type = other DNA
cccccagaatacacaccgaccgtgtgatggcatttatccccctcaaatgttttttttttttactaatgcg

SEQ ID NO: 606 - DNA, 71 bp, synthetic construct
  source 1..71: mol_type = other DNA
caggcggtagctcaatgggcgcccaccaccctcagattttcatttaattgttttttttttttaaggtctgt

SEQ ID NO: 607 - DNA, 63 bp, synthetic construct
  source 1..63: mol_type = other DNA
tagtttggtaggtaggctatcaggtcatattaagtaatagcgttttttttttttacttgatcg

SEQ ID NO: 608 - DNA, 49 bp, synthetic construct
  source 1..49: mol_type = other DNA
gcttagatgataagcagactgtagcgcgttttttttttttatattgccg

SEQ ID NO: 609 - DNA, 49 bp, synthetic construct
  source 1..49: mol_type = other DNA
gctgtttgctcgaaatcaccagtagcacttttttttttttactggaact

SEQ ID NO: 610 - DNA, 70 bp, synthetic construct
  source 1..70: mol_type = other DNA
agaaacgaaaccgagataccgatagttgcacgctgttattctgaaacattttttttttttttatgtccga

SEQ ID NO: 611 - DNA, 49 bp, synthetic construct
  source 1..49: mol_type = other DNA
taacagtggtttaacattcaaccgattgttttttttttttactaacacg

SEQ ID NO: 612 - DNA, 49 bp, synthetic construct
  source 1..49: mol_type = other DNA
aactaactttgcaatgtgctgcaaggcgttttttttttttagctctcat

SEQ ID NO: 613 - DNA, 49 bp, synthetic construct
  source 1..49: mol_type = other DNA
caatcgccctgtttgtgccggaaaccagttttttttttttataagtgcg

SEQ ID NO: 614 - DNA, 71 bp, synthetic construct
  source 1..71: mol_type = other DNA
tattcacacaattcccgttgcgcacaaacaaataaacattacctccccggttttttttttttacgttacag

SEQ ID NO: 615 - DNA, 63 bp, synthetic construct
  source 1..63: mol_type = other DNA
cgcgtaatatccaggtaataaaagggactaatcctgtgtagattttttttttttagcaggtta

SEQ ID NO: 616 - DNA, 49 bp, synthetic construct
  source 1..49: mol_type = other DNA
aacccacgccaccaaaagattaagaggattttttttttttaactaggca

SEQ ID NO: 617 - DNA, 70 bp, synthetic construct
  source 1..70: mol_type = other DNA
cccccagaatacacaccgaccgtgtgatggcatttatccccctcaaatgttttttttttttattgtctcg

SEQ ID NO: 618 - DNA, 71 bp, synthetic construct
  source 1..71: mol_type = other DNA
caggcggtagctcaatgggcgcccaccaccctcagattttcatttaattgttttttttttttattggatcg

SEQ ID NO: 619 - DNA, 63 bp, synthetic construct
  source 1..63: mol_type = other DNA
tagtttggtaggtaggctatcaggtcatattaagtaatagcgttttttttttttagttaacgc

SEQ ID NO: 620 - DNA, 49 bp, synthetic construct
  source 1..49: mol_type = other DNA
gcttagatgataagcagactgtagcgcgtttttttttttttaacctgct

SEQ ID NO: 621 - DNA, 49 bp, synthetic construct
  source 1..49: mol_type = other DNA
gctgtttgctcgaaatcaccagtagcacttttttttttttcgcacttat

SEQ ID NO: 622 - DNA, 70 bp, synthetic construct
  source 1..70: mol_type = other DNA
agaaacgaaaccgagataccgatagttgcacgctgttattctgaaacatttttttttttttctgtaacgt

SEQ ID NO: 623 - DNA, 49 bp, synthetic construct
  source 1..49: mol_type = other DNA
taacagtggtttaacattcaaccgattgttttttttttttatgagagct

SEQ ID NO: 624 - DNA, 49 bp, synthetic construct
  source 1..49: mol_type = other DNA
aactaactttgcaatgtgctgcaaggcgttttttttttttaggtcgtta

SEQ ID NO: 625 - DNA, 49 bp, synthetic construct
  source 1..49: mol_type = other DNA
caatcgccctgtttgtgccggaaaccagttttttttttttatacgatgc

SEQ ID NO: 626 - DNA, 71 bp, synthetic construct
  source 1..71: mol_type = other DNA
tattcacacaattcccgttgcgcacaaacaaataaacattacctccccggttttttttttttaggccaata

SEQ ID NO: 627 - DNA, 63 bp, synthetic construct
  source 1..63: mol_type = other DNA
cgcgtaatatccaggtaataaaagggactaatcctgtgtagattttttttttttatagttgcg

SEQ ID NO: 628 - DNA, 63 bp, synthetic construct
  source 1..63: mol_type = other DNA
gatcgtccaatcataaatcaacgtaacaagcccgaccgccacttttttttttttcgagacaat

SEQ ID NO: 629 - DNA, 63 bp, synthetic construct
  source 1..63: mol_type = other DNA
agcggtccgccgacaagttaccagaaggcaccaggcggataatttttttttttttgcctagtt

SEQ ID NO: 630 - DNA, 49 bp, synthetic construct
  source 1..49: mol_type = other DNA
gaacgcgatagaagtagcattccacagattttttttttttgcgttaact

SEQ ID NO: 631 - DNA, 49 bp, synthetic construct
  source 1..49: mol_type = other DNA
acaccgctaatagacaacagtttcagcgttttttttttttcgatccaat

SEQ ID NO: 632 - DNA, 49 bp, synthetic construct
  source 1..49: mol_type = other DNA
gcttagatgataagcagactgtagcgcgttttttttttttcagattcgt

SEQ ID NO: 633 - DNA, 49 bp, synthetic construct
  source 1..49: mol_type = other DNA
gctgtttgctcgaaatcaccagtagcacttttttttttttcgccataat

SEQ ID NO: 634 - DNA, 70 bp, synthetic construct
  source 1..70: mol_type = other DNA
agaaacgaaaccgagataccgatagttgcacgctgttattctgaaacatttttttttttttcgttgtagt

SEQ ID NO: 635 - DNA, 49 bp, synthetic construct
  source 1..49: mol_type = other DNA
taacagtggtttaacattcaaccgattgtttttttttttttaagacggt

SEQ ID NO: 636 - DNA, 49 bp, synthetic construct
  source 1..49: mol_type = other DNA
aactaactttgcaatgtgctgcaaggcgttttttttttttacgtaccat

SEQ ID NO: 637 - DNA, 49 bp, synthetic construct
  source 1..49: mol_type = other DNA
caatcgccctgtttgtgccggaaaccagttttttttttttagagtgcat

SEQ ID NO: 638 - DNA, 71 bp, synthetic construct
  source 1..71: mol_type = other DNA
tattcacacaattcccgttgcgcacaaacaaataaacattacctccccggttttttttttttaccagaact

SEQ ID NO: 639 - DNA, 63 bp, synthetic construct
  source 1..63: mol_type = other DNA
cgcgtaatatccaggtaataaaagggactaatcctgtgtagattttttttttttactgttcct

SEQ ID NO: 640 - DNA, 49 bp, synthetic construct
  source 1..49: mol_type = other DNA
aacccacgccaccaaaagattaagaggattttttttttttatggtacgt

SEQ ID NO: 641 - DNA, 70 bp, synthetic construct
  source 1..70: mol_type = other DNA
cccccagaatacacaccgaccgtgtgatggcatttatccccctcaaatgttttttttttttagttctggt

SEQ ID NO: 642 - DNA, 71 bp, synthetic construct
  source 1..71: mol_type = other DNA
caggcggtagctcaatgggcgcccaccaccctcagattttcatttaattgttttttttttttatgcactct

SEQ ID NO: 643 - DNA, 63 bp, synthetic construct
  source 1..63: mol_type = other DNA
tagtttggtaggtaggctatcaggtcatattaagtaatagcgttttttttttttaggaacagt

SEQ ID NO: 644 - DNA, 49 bp, synthetic construct
  source 1..49: mol_type = other DNA
gcttagatgataagcagactgtagcgcgttttttttttttagcatgagt

SEQ ID NO: 645 - DNA, 49 bp, synthetic construct
  source 1..49: mol_type = other DNA
gctgtttgctcgaaatcaccagtagcacttttttttttttattcctcgt

SEQ ID NO: 646 - DNA, 70 bp, synthetic construct
  source 1..70: mol_type = other DNA
agaaacgaaaccgagataccgatagttgcacgctgttattctgaaacattttttttttttttaaggacgt

SEQ ID NO: 647 - DNA, 49 bp, synthetic construct
  source 1..49: mol_type = other DNA
taacagtggtttaacattcaaccgattgtttttttttttttaacggtct

SEQ ID NO: 648 - DNA, 49 bp, synthetic construct
  source 1..49: mol_type = other DNA
aactaactttgcaatgtgctgcaaggcgttttttttttttgcaacatct

SEQ ID NO: 649 - DNA, 49 bp, synthetic construct
  source 1..49: mol_type = other DNA
caatcgccctgtttgtgccggaaaccagttttttttttttgcagtgaat

SEQ ID NO: 650 - DNA, 71 bp, synthetic construct
  source 1..71: mol_type = other DNA
tattcacacaattcccgttgcgcacaaacaaataaacattacctccccggttttttttttttgcgacttat

SEQ ID NO: 651 - DNA, 63 bp, synthetic construct
  source 1..63: mol_type = other DNA
cgcgtaatatccaggtaataaaagggactaatcctgtgtagatttttttttttttattccggt

SEQ ID NO: 652 - DNA, 63 bp, synthetic construct
  source 1..63: mol_type = other DNA
gatcgtccaatcataaatcaacgtaacaagcccgaccgccacttttttttttttaccggaata

SEQ ID NO: 653 - DNA, 63 bp, synthetic construct
  source 1..63: mol_type = other DNA
agcggtccgccgacaagttaccagaaggcaccaggcggataattttttttttttattcactgc

SEQ ID NO: 654 - DNA, 49 bp, synthetic construct
  source 1..49: mol_type = other DNA
gaacgcgatagaagtagcattccacagattttttttttttataagtcgc

SEQ ID NO: 655 - DNA, 49 bp, synthetic construct
  source 1..49: mol_type = other DNA
acaccgctaatagacaacagtttcagcgttttttttttttagatgttgc

SEQ ID NO: 656 - DNA, 49 bp, synthetic construct
  source 1..49: mol_type = other DNA
gcttagatgataagcagactgtagcgcgttttttttttttgccatgaat

SEQ ID NO: 657 - DNA, 49 bp, synthetic construct
  source 1..49: mol_type = other DNA
gctgtttgctcgaaatcaccagtagcacttttttttttttcacacgtat

SEQ ID NO: 658 - DNA, 70 bp, synthetic construct
  source 1..70: mol_type = other DNA
agaaacgaaaccgagataccgatagttgcacgctgttattctgaaacatttttttttttttcgatgacat

SEQ ID NO: 659 - DNA, 49 bp, synthetic construct
  source 1..49: mol_type = other DNA
taacagtggtttaacattcaaccgattgttttttttttttcgaacatgt

SEQ ID NO: 660 - DNA, 49 bp, synthetic construct
  source 1..49: mol_type = other DNA
aactaactttgcaatgtgctgcaaggcgttttttttttttgcatcgtat

SEQ ID NO: 661 - DNA, 49 bp, synthetic construct
  source 1..49: mol_type = other DNA
caatcgccctgtttgtgccggaaaccagtttttttttttttaacgacct

SEQ ID NO: 662 - DNA, 71 bp, synthetic construct
  source 1..71: mol_type = other DNA
tattcacacaattcccgttgcgcacaaacaaataaacattacctccccggttttttttttttcgcaactat

SEQ ID NO: 663 - DNA, 63 bp, synthetic construct
  source 1..63: mol_type = other DNA
cgcgtaatatccaggtaataaaagggactaatcctgtgtagatttttttttttttattggcct

SEQ ID NO: 664 - DNA, 49 bp, synthetic construct
  source 1..49: mol_type = other DNA
atcagctatcgtaagcaattcatcaatattttttttttttacgaggaat

SEQ ID NO: 665 - DNA, 63 bp, synthetic construct
  source 1..63: mol_type = other DNA
gaggccacctgagagtcaatagtgaattccaagaaccgaacgttttttttttttactcatgct

SEQ ID NO: 666 - DNA, 71 bp, synthetic construct
  source 1..71: mol_type = other DNA
catttttaacggattggttgagaggagtgtactggtagggaggtgcgtagttttttttttttagaccgtta

SEQ ID NO: 667 - DNA, 56 bp, synthetic construct
  source 1..56: mol_type = other DNA
gagccttacaactaaaggaatgagtgagggagcacttttttttttttacgtcctta

SEQ ID NO: 668 - DNA, 49 bp, synthetic construct
  source 1..49: mol_type = other DNA
aacccacgccaccaaaagattaagaggattttttttttttatagaacgc

SEQ ID NO: 669 - DNA, 70 bp, synthetic construct
  source 1..70: mol_type = other DNA
cccccagaatacacaccgaccgtgtgatggcatttatccccctcaaatgttttttttttttataaccacg

SEQ ID NO: 670 - DNA, 71 bp, synthetic construct
  source 1..71: mol_type = other DNA
caggcggtagctcaatgggcgcccaccaccctcagattttcatttaattgttttttttttttattgaaccg

SEQ ID NO: 671 - DNA, 63 bp, synthetic construct
  source 1..63: mol_type = other DNA
tagtttggtaggtaggctatcaggtcatattaagtaatagcgttttttttttttaccatgtct

SEQ ID NO: 672 - DNA, 49 bp, synthetic construct
  source 1..49: mol_type = other DNA
aacccacgccaccaaaagattaagaggattttttttttttgcgttctat

SEQ ID NO: 673 - DNA, 70 bp, synthetic construct
  source 1..70: mol_type = other DNA
cccccagaatacacaccgaccgtgtgatggcatttatccccctcaaatgttttttttttttcggttcaat

SEQ ID NO: 674 - DNA, 71 bp, synthetic construct
  source 1..71: mol_type = other DNA
caggcggtagctcaatgggcgcccaccaccctcagattttcatttaattgttttttttttttcgtggttat

SEQ ID NO: 675 - DNA, 63 bp, synthetic construct
  source 1..63: mol_type = other DNA
tagtttggtaggtaggctatcaggtcatattaagtaatagcgttttttttttttagacatggt

SEQ ID NO: 676 - DNA, 49 bp, synthetic construct
  source 1..49: mol_type = other DNA
gcttagatgataagcagactgtagcgcgttttttttttttagacatggt

SEQ ID NO: 677 - DNA, 49 bp, synthetic construct
  source 1..49: mol_type = other DNA
gctgtttgctcgaaatcaccagtagcacttttttttttttcgtggttat

SEQ ID NO: 678 - DNA, 70 bp, synthetic construct
  source 1..70: mol_type = other DNA
agaaacgaaaccgagataccgatagttgcacgctgttattctgaaacatttttttttttttcggttcaat

SEQ ID NO: 679 - DNA, 49 bp, synthetic construct
  source 1..49: mol_type = other DNA
taacagtggtttaacattcaaccgattgttttttttttttgcgttctat

SEQ ID NO: 680 - DNA, 49 bp, synthetic construct
  source 1..49: mol_type = other DNA
aactaactttgcaatgtgctgcaaggcgttttttttttttcgtggttat

SEQ ID NO: 681 - DNA, 49 bp, synthetic construct
  source 1..49: mol_type = other DNA
caatcgccctgtttgtgccggaaaccagttttttttttttgcgttctat

SEQ ID NO: 682 - DNA, 71 bp, synthetic construct
  source 1..71: mol_type = other DNA
tattcacacaattcccgttgcgcacaaacaaataaacattacctccccggttttttttttttagacatggt

SEQ ID NO: 683 - DNA, 63 bp, synthetic construct
  source 1..63: mol_type = other DNA
cgcgtaatatccaggtaataaaagggactaatcctgtgtagattttttttttttcggttcaat

SEQ ID NO: 684 - DNA, 49 bp, synthetic construct
  source 1..49: mol_type = other DNA
aacccacgccaccaaaagattaagaggattttttttttttattctccgt

SEQ ID NO: 685 - DNA, 70 bp, synthetic construct
  source 1..70: mol_type = other DNA
cccccagaatacacaccgaccgtgtgatggcatttatccccctcaaatgtttttttttttttgcggatat

SEQ ID NO: 686 - DNA, 71 bp, synthetic construct
  source 1..71: mol_type = other DNA
caggcggtagctcaatgggcgcccaccaccctcagattttcatttaattgtttttttttttttaactgtgc

SEQ ID NO: 687 - DNA, 63 bp, synthetic construct
  source 1..63: mol_type = other DNA
tagtttggtaggtaggctatcaggtcatattaagtaatagcgttttttttttttatcgtctct

SEQ ID NO: 688 - DNA, 49 bp, synthetic construct
  source 1..49: mol_type = other DNA
gcttagatgataagcagactgtagcgcgttttttttttttatatgcagc

SEQ ID NO: 689 - DNA, 49 bp, synthetic construct
  source 1..49: mol_type = other DNA
gctgtttgctcgaaatcaccagtagcacttttttttttttatcatacgc

SEQ ID NO: 690 - DNA, 70 bp, synthetic construct
  source 1..70: mol_type = other DNA
agaaacgaaaccgagataccgatagttgcacgctgttattctgaaacatttttttttttttaggtgtcat

SEQ ID NO: 691 - DNA, 49 bp, synthetic construct
  source 1..49: mol_type = other DNA
taacagtggtttaacattcaaccgattgttttttttttttaccactgat

SEQ ID NO: 692 - DNA, 49 bp, synthetic construct
  source 1..49: mol_type = other DNA
aactaactttgcaatgtgctgcaaggcgttttttttttttgcagttgat

SEQ ID NO: 693 - DNA, 49 bp, synthetic construct
  source 1..49: mol_type = other DNA
caatcgccctgtttgtgccggaaaccagttttttttttttgcaaggaat

SEQ ID NO: 694 - DNA, 71 bp, synthetic construct
  source 1..71: mol_type = other DNA
tattcacacaattcccgttgcgcacaaacaaataaacattacctccccggttttttttttttgcatggatt

SEQ ID NO: 695 - DNA, 63 bp, synthetic construct
  source 1..63: mol_type = other DNA
cgcgtaatatccaggtaataaaagggactaatcctgtgtagattttttttttttagcctgatt

SEQ ID NO: 696 - DNA, 63 bp, synthetic construct
  source 1..63: mol_type = other DNA
gatcgtccaatcataaatcaacgtaacaagcccgaccgccacttttttttttttagagacgat

SEQ ID NO: 697 - DNA, 63 bp, synthetic construct
  source 1..63: mol_type = other DNA
agcggtccgccgacaagttaccagaaggcaccaggcggataattttttttttttatatccgca

SEQ ID NO: 698 - DNA, 49 bp, synthetic construct
  source 1..49: mol_type = other DNA
gaacgcgatagaagtagcattccacagattttttttttttgcacagtta

SEQ ID NO: 699 - DNA, 49 bp, synthetic construct
  source 1..49: mol_type = other DNA
acaccgctaatagacaacagtttcagcgttttttttttttacggagaat

SEQ ID NO: 700 - DNA, 49 bp, synthetic construct
  source 1..49: mol_type = other DNA
gcttagatgataagcagactgtagcgcgttttttttttttaccgtctta

SEQ ID NO: 701 - DNA, 49 bp, synthetic construct
  source 1..49: mol_type = other DNA
gctgtttgctcgaaatcaccagtagcacttttttttttttattatggcg

SEQ ID NO: 702 - DNA, 70 bp, synthetic construct
  source 1..70: mol_type = other DNA
agaaacgaaaccgagataccgatagttgcacgctgttattctgaaacatttttttttttttactacaacg

SEQ ID NO: 703 - DNA, 49 bp, synthetic construct
  source 1..49: mol_type = other DNA
taacagtggtttaacattcaaccgattgttttttttttttacgaatctg

SEQ ID NO: 704 - DNA, 49 bp, synthetic construct
  source 1..49: mol_type = other DNA
aactaactttgcaatgtgctgcaaggcgttttttttttttattgtagcg

SEQ ID NO: 705 - DNA, 49 bp, synthetic construct
  source 1..49: mol_type = other DNA
caatcgccctgtttgtgccggaaaccagttttttttttttagcagagat

SEQ ID NO: 706 - DNA, 71 bp, synthetic construct
  source 1..71: mol_type = other DNA
tattcacacaattcccgttgcgcacaaacaaataaacattacctccccggttttttttttttaggacacat

SEQ ID NO: 707 - DNA, 63 bp, synthetic construct
  source 1..63: mol_type = other DNA
cgcgtaatatccaggtaataaaagggactaatcctgtgtagattttttttttttagttcttgc

SEQ ID NO: 708 - DNA, 49 bp, synthetic construct
  source 1..49: mol_type = other DNA
aacccacgccaccaaaagattaagaggattttttttttttaggctgtta

SEQ ID NO: 709 - DNA, 70 bp, synthetic construct
  source 1..70: mol_type = other DNA
cccccagaatacacaccgaccgtgtgatggcatttatccccctcaaatgttttttttttttagtgaccat

SEQ ID NO: 710 - DNA, 71 bp, synthetic construct
  source 1..71: mol_type = other DNA
caggcggtagctcaatgggcgcccaccaccctcagattttcatttaattgttttttttttttaatacgtcg

SEQ ID NO: 711 - DNA, 63 bp, synthetic construct
  source 1..63: mol_type = other DNA
tagtttggtaggtaggctatcaggtcatattaagtaatagcgttttttttttttaagatccgt

SEQ ID NO: 712 - DNA, 49 bp, synthetic construct
  source 1..49: mol_type = other DNA
gcttagatgataagcagactgtagcgcgttttttttttttatatgcgtc

SEQ ID NO: 713 - DNA, 49 bp, synthetic construct
  source 1..49: mol_type = other DNA
gctgtttgctcgaaatcaccagtagcacttttttttttttaacaggact

SEQ ID NO: 714 - DNA, 70 bp, synthetic construct
  source 1..70: mol_type = other DNA
agaaacgaaaccgagataccgatagttgcacgctgttattctgaaacatttttttttttttacttggact

SEQ ID NO: 715 - DNA, 49 bp, synthetic construct
  source 1..49: mol_type = other DNA
taacagtggtttaacattcaaccgattgttttttttttttaccagctta

SEQ ID NO: 716 - DNA, 49 bp, synthetic construct
  source 1..49: mol_type = other DNA
aactaactttgcaatgtgctgcaaggcgttttttttttttcgctacaat

SEQ ID NO: 717 - DNA, 49 bp, synthetic construct
  source 1..49: mol_type = other DNA
caatcgccctgtttgtgccggaaaccagttttttttttttatgtgtcct

SEQ ID NO: 718 - DNA, 71 bp, synthetic construct
  source 1..71: mol_type = other DNA
tattcacacaattcccgttgcgcacaaacaaataaacattacctccccggttttttttttttatctctgct

SEQ ID NO: 719 - DNA, 63 bp, synthetic construct
  source 1..63: mol_type = other DNA
cgcgtaatatccaggtaataaaagggactaatcctgtgtagattttttttttttgcaagaact

SEQ ID NO: 720 - DNA, 63 bp, synthetic construct
  source 1..63: mol_type = other DNA
gatcgtccaatcataaatcaacgtaacaagcccgaccgccacttttttttttttatgatagcg

SEQ ID NO: 721 - DNA, 63 bp, synthetic construct
  source 1..63: mol_type = other DNA
agcggtccgccgacaagttaccagaaggcaccaggcggataattttttttttttgcatgtaca

SEQ ID NO: 722 - DNA, 49 bp, synthetic construct
  source 1..49: mol_type = other DNA
gaacgcgatagaagtagcattccacagattttttttttttactggttct

SEQ ID NO: 723 - DNA, 49 bp, synthetic construct
  source 1..49: mol_type = other DNA
acaccgctaatagacaacagtttcagcgttttttttttttatacaaccg

SEQ ID NO: 724 - DNA, 49 bp, synthetic construct
  source 1..49: mol_type = other DNA
gcttagatgataagcagactgtagcgcgttttttttttttattcatggc

SEQ ID NO: 725 - DNA, 49 bp, synthetic construct
  source 1..49: mol_type = other DNA
gctgtttgctcgaaatcaccagtagcacttttttttttttatgtcatcg

SEQ ID NO: 726 - DNA, 70 bp, synthetic construct
  source 1..70: mol_type = other DNA
agaaacgaaaccgagataccgatagttgcacgctgttattctgaaacatttttttttttttatacgtgtg

SEQ ID NO: 727 - DNA, 49 bp, synthetic construct
  source 1..49: mol_type = other DNA
taacagtggtttaacattcaaccgattgttttttttttttacatgttcg

SEQ ID NO: 728 - DNA, 49 bp, synthetic construct
  source 1..49: mol_type = other DNA
aactaactttgcaatgtgctgcaaggcgtttttttttttttaacagcct

SEQ ID NO: 729 - DNA, 49 bp, synthetic construct
  source 1..49: mol_type = other DNA
caatcgccctgtttgtgccggaaaccagttttttttttttcgacgtatt

SEQ ID NO: 730 - DNA, 71 bp, synthetic construct
  source 1..71: mol_type = other DNA
tattcacacaattcccgttgcgcacaaacaaataaacattacctccccggttttttttttttatggtcact

SEQ ID NO: 731 - DNA, 63 bp, synthetic construct
  source 1..63: mol_type = other DNA
cgcgtaatatccaggtaataaaagggactaatcctgtgtagattttttttttttacggatctt

SEQ ID NO: 732 - DNA, 49 bp, synthetic construct
  source 1..49: mol_type = other DNA
aacccacgccaccaaaagattaagaggattttttttttttaggttcgta

SEQ ID NO: 733 - DNA, 70 bp, synthetic construct
  source 1..70: mol_type = other DNA
cccccagaatacacaccgaccgtgtgatggcatttatccccctcaaatgttttttttttttatagctcga

SEQ ID NO: 734 - DNA, 71 bp, synthetic construct
  source 1..71: mol_type = other DNA
caggcggtagctcaatgggcgcccaccaccctcagattttcatttaattgtttttttttttttatcagcga

SEQ ID NO: 735 - DNA, 63 bp, synthetic construct
  source 1..63: mol_type = other DNA
tagtttggtaggtaggctatcaggtcatattaagtaatagcgttttttttttttataccaacg

SEQ ID NO: 736 - DNA, 49 bp, synthetic construct
  source 1..49: mol_type = other DNA
gcttagatgataagcagactgtagcgcgttttttttttttatgtgatcg

SEQ ID NO: 737 - DNA, 49 bp, synthetic construct
  source 1..49: mol_type = other DNA
gctgtttgctcgaaatcaccagtagcacttttttttttttatacggcta

SEQ ID NO: 738 - DNA, 70 bp, synthetic construct
  source 1..70: mol_type = other DNA
agaaacgaaaccgagataccgatagttgcacgctgttattctgaaacatttttttttttttacattgtcg

SEQ ID NO: 739 - DNA, 49 bp, synthetic construct
  source 1..49: mol_type = other DNA
taacagtggtttaacattcaaccgattgttttttttttttagcatatcg

SEQ ID NO: 740 - DNA, 49 bp, synthetic construct
  source 1..49: mol_type = other DNA
aactaactttgcaatgtgctgcaaggcgttttttttttttcggttgtat

SEQ ID NO: 741 - DNA, 49 bp, synthetic construct
  source 1..49: mol_type = other DNA
caatcgccctgtttgtgccggaaaccagtttttttttttttgtacatgc

SEQ ID NO: 742 - DNA, 71 bp, synthetic construct
  source 1..71: mol_type = other DNA
tattcacacaattcccgttgcgcacaaacaaataaacattacctccccggttttttttttttagaaccagt

SEQ ID NO: 743 - DNA, 63 bp, synthetic construct
  source 1..63: mol_type = other DNA
cgcgtaatatccaggtaataaaagggactaatcctgtgtagattttttttttttcgctatcat

SEQ ID NO: 744 - DNA, 63 bp, synthetic construct
  source 1..63: mol_type = other DNA
gatcgtccaatcataaatcaacgtaacaagcccgaccgccacttttttttttttcgttggtat

SEQ ID NO: 745 - DNA, 63 bp, synthetic construct
  source 1..63: mol_type = other DNA
agcggtccgccgacaagttaccagaaggcaccaggcggataatttttttttttttcgagctat

SEQ ID NO: 746 - DNA, 49 bp, synthetic construct
  source 1..49: mol_type = other DNA
gaacgcgatagaagtagcattccacagatttttttttttttcgctgata

SEQ ID NO: 747 - DNA, 49 bp, synthetic construct
  source 1..49: mol_type = other DNA
acaccgctaatagacaacagtttcagcgtttttttttttttacgaacct

SEQ ID NO: 748 - DNA, 49 bp, synthetic construct
  source 1..49: mol_type = other DNA
gcttagatgataagcagactgtagcgcgttttttttttttcgcattagt

SEQ ID NO: 749 - DNA, 49 bp, synthetic construct
  source 1..49: mol_type = other DNA
gctgtttgctcgaaatcaccagtagcacttttttttttttcgttcatct

SEQ ID NO: 750 - DNA, 70 bp, synthetic construct
  source 1..70: mol_type = other DNA
agaaacgaaaccgagataccgatagttgcacgctgttattctgaaacatttttttttttttcgatcaagt

SEQ ID NO: 751 - DNA, 49 bp, synthetic construct
  source 1..49: mol_type = other DNA
taacagtggtttaacattcaaccgattgttttttttttttacagacctt

SEQ ID NO: 752 - DNA, 49 bp, synthetic construct
  source 1..49: mol_type = other DNA
aactaactttgcaatgtgctgcaaggcgttttttttttttaccattacg

SEQ ID NO: 753 - DNA, 49 bp, synthetic construct
  source 1..49: mol_type = other DNA
caatcgccctgtttgtgccggaaaccagttttttttttttacaagatcg

SEQ ID NO: 754 - DNA, 71 bp, synthetic construct
  source 1..71: mol_type = other DNA
tattcacacaattcccgttgcgcacaaacaaataaacattacctccccggttttttttttttagtggcata

SEQ ID NO: 755 - DNA, 63 bp, synthetic construct
  source 1..63: mol_type = other DNA
cgcgtaatatccaggtaataaaagggactaatcctgtgtagattttttttttttatatcgtgc

SEQ ID NO: 756 - DNA, 49 bp, synthetic construct
  source 1..49: mol_type = other DNA
aacccacgccaccaaaagattaagaggattttttttttttaacacatcg

SEQ ID NO: 757 - DNA, 70 bp, synthetic construct
  source 1..70: mol_type = other DNA
cccccagaatacacaccgaccgtgtgatggcatttatccccctcaaatgttttttttttttatgatgtcg

SEQ ID NO: 758 - DNA, 71 bp, synthetic construct
  source 1..71: mol_type = other DNA
caggcggtagctcaatgggcgcccaccaccctcagattttcatttaattgttttttttttttacatcaagc

SEQ ID NO: 759 - DNA, 63 bp, synthetic construct
  source 1..63: mol_type = other DNA
tagtttggtaggtaggctatcaggtcatattaagtaatagcgttttttttttttataacaccg

SEQ ID NO: 760 - DNA, 49 bp, synthetic construct
  source 1..49: mol_type = other DNA
gcttagatgataagcagactgtagcgcgttttttttttttaaggcagat

SEQ ID NO: 761 - DNA, 49 bp, synthetic construct
  source 1..49: mol_type = other DNA
gctgtttgctcgaaatcaccagtagcacttttttttttttagattgacg

SEQ ID NO: 762 - DNA, 70 bp, synthetic construct
  source 1..70: mol_type = other DNA
agaaacgaaaccgagataccgatagttgcacgctgttattctgaaacatttttttttttttatatgagcg

SEQ ID NO: 763 - DNA, 49 bp, synthetic construct
  source 1..49: mol_type = other DNA
taacagtggtttaacattcaaccgattgttttttttttttacggttact

SEQ ID NO: 764 - DNA, 49 bp, synthetic construct
  source 1..49: mol_type = other DNA
aactaactttgcaatgtgctgcaaggcgttttttttttttcgtaatggt

SEQ ID NO: 765 - DNA, 49 bp, synthetic construct
  source 1..49: mol_type = other DNA
caatcgccctgtttgtgccggaaaccagtttttttttttttatgccact

SEQ ID NO: 766 - DNA, 71 bp, synthetic construct
  source 1..71: mol_type = other DNA
tattcacacaattcccgttgcgcacaaacaaataaacattacctccccggttttttttttttcgatcttgt

SEQ ID NO: 767 - DNA, 63 bp, synthetic construct
  source 1..63: mol_type = other DNA
cgcgtaatatccaggtaataaaagggactaatcctgtgtagattttttttttttgcacgatat

SEQ ID NO: 768 - DNA, 63 bp, synthetic construct
  source 1..63: mol_type = other DNA
gatcgtccaatcataaatcaacgtaacaagcccgaccgccacttttttttttttaatcaggct

SEQ ID NO: 769 - DNA, 63 bp, synthetic construct
  source 1..63: mol_type = other DNA
agcggtccgccgacaagttaccagaaggcaccaggcggataattttttttttttattccttgc

SEQ ID NO: 770 - DNA, 49 bp, synthetic construct
  source 1..49: mol_type = other DNA
gaacgcgatagaagtagcattccacagattttttttttttaatccatgc

SEQ ID NO: 771 - DNA, 49 bp, synthetic construct
  source 1..49: mol_type = other DNA
acaccgctaatagacaacagtttcagcgttttttttttttatcaactgc

SEQ ID NO: 772 - DNA, 49 bp, synthetic construct
  source 1..49: mol_type = other DNA
gcttagatgataagcagactgtagcgcgttttttttttttcggcaatat

SEQ ID NO: 773 - DNA, 49 bp, synthetic construct
  source 1..49: mol_type = other DNA
gctgtttgctcgaaatcaccagtagcactttttttttttttcggacata

SEQ ID NO: 774 - DNA, 70 bp, synthetic construct
  source 1..70: mol_type = other DNA
agaaacgaaaccgagataccgatagttgcacgctgttattctgaaacatttttttttttttagttccagt

SEQ ID NO: 775 - DNA, 49 bp, synthetic construct
  source 1..49: mol_type = other DNA
taacagtggtttaacattcaaccgattgttttttttttttcgtgttagt

SEQ ID NO: 776 - DNA, 49 bp, synthetic construct
  source 1..49: mol_type = other DNA
aactaactttgcaatgtgctgcaaggcgttttttttttttcgatgtgtt

SEQ ID NO: 777 - DNA, 49 bp, synthetic construct
  source 1..49: mol_type = other DNA
caatcgccctgtttgtgccggaaaccagttttttttttttgcttgatgt

SEQ ID NO: 778 - DNA, 71 bp, synthetic construct
  source 1..71: mol_type = other DNA
tattcacacaattcccgttgcgcacaaacaaataaacattacctccccggttttttttttttcgacatcat

SEQ ID NO: 779 - DNA, 63 bp, synthetic construct
  source 1..63: mol_type = other DNA
cgcgtaatatccaggtaataaaagggactaatcctgtgtagattttttttttttcggtgttat

SEQ ID NO: 780 - DNA, 49 bp, synthetic construct
  source 1..49: mol_type = other DNA
atcagctatcgtaagcaattcatcaatattttttttttttcgatcacat

SEQ ID NO: 781 - DNA, 63 bp, synthetic construct
  source 1..63: mol_type = other DNA
gaggccacctgagagtcaatagtgaattccaagaaccgaacgttttttttttttcgacaatgt

SEQ ID NO: 782 - DNA, 71 bp, synthetic construct
  source 1..71: mol_type = other DNA
catttttaacggattggttgagaggagtgtactggtagggaggtgcgtagtttttttttttttagccgtat

SEQ ID NO: 783 - DNA, 56 bp, synthetic construct
  source 1..56: mol_type = other DNA
gagccttacaactaaaggaatgagtgagggagcacttttttttttttcgatatgct

SEQ ID NO: 784 - DNA, 49 bp, synthetic construct
  source 1..49: mol_type = other DNA
aacccacgccaccaaaagattaagaggattttttttttttatgacacct

SEQ ID NO: 785 - DNA, 70 bp, synthetic construct
  source 1..70: mol_type = other DNA
cccccagaatacacaccgaccgtgtgatggcatttatccccctcaaatgttttttttttttatcagtggt

SEQ ID NO: 786 - DNA, 71 bp, synthetic construct
  source 1..71: mol_type = other DNA
caggcggtagctcaatgggcgcccaccaccctcagattttcatttaattgttttttttttttgctgcatat

SEQ ID NO: 787 - DNA, 63 bp, synthetic construct
  source 1..63: mol_type = other DNA
tagtttggtaggtaggctatcaggtcatattaagtaatagcgttttttttttttgcgtatgat

SEQ ID NO: 788 - DNA, 49 bp, synthetic construct
  source 1..49: mol_type = other DNA
atcagctatcgtaagcaattcatcaatattttttttttttgacgcatat

SEQ ID NO: 789 - DNA, 63 bp, synthetic construct
  source 1..63: mol_type = other DNA
gaggccacctgagagtcaatagtgaattccaagaaccgaacgttttttttttttagtccaagt

SEQ ID NO: 790 - DNA, 71 bp, synthetic construct
  source 1..71: mol_type = other DNA
catttttaacggattggttgagaggagtgtactggtagggaggtgcgtagttttttttttttagtcctgtt

SEQ ID NO: 791 - DNA, 56 bp, synthetic construct
  source 1..56: mol_type = other DNA
gagccttacaactaaaggaatgagtgagggagcactttttttttttttaagctggt

SEQ ID NO: 792 - DNA, 49 bp, synthetic construct
  source 1..49: mol_type = other DNA
aacccacgccaccaaaagattaagaggattttttttttttagtccaagt

SEQ ID NO: 793 - DNA, 70 bp, synthetic construct
  source 1..70: mol_type = other DNA
cccccagaatacacaccgaccgtgtgatggcatttatccccctcaaatgtttttttttttttaagctggt

SEQ ID NO: 794 - DNA, 71 bp, synthetic construct
  source 1..71: mol_type = other DNA
caggcggtagctcaatgggcgcccaccaccctcagattttcatttaattgttttttttttttgacgcatat

SEQ ID NO: 795 - DNA, 63 bp, synthetic construct
  source 1..63: mol_type = other DNA
tagtttggtaggtaggctatcaggtcatattaagtaatagcgttttttttttttagtcctgtt

SEQ ID NO: 796 - DNA, 49 bp, synthetic construct
  source 1..49: mol_type = other DNA
atcagctatcgtaagcaattcatcaatattttttttttttagtaaccgt

SEQ ID NO: 797 - DNA, 63 bp, synthetic construct
  source 1..63: mol_type = other DNA
gaggccacctgagagtcaatagtgaattccaagaaccgaacgttttttttttttcgtcaatct

SEQ ID NO: 798 - DNA, 71 bp, synthetic construct
  source 1..71: mol_type = other DNA
catttttaacggattggttgagaggagtgtactggtagggaggtgcgtagttttttttttttcgctcatat

SEQ ID NO: 799 - DNA, 56 bp, synthetic construct
  source 1..56: mol_type = other DNA
gagccttacaactaaaggaatgagtgagggagcacttttttttttttatctgcctt

SEQ ID NO: 800 - DNA, 49 bp, synthetic construct
  source 1..49: mol_type = other DNA
aacccacgccaccaaaagattaagaggattttttttttttcgtcaatct

SEQ ID NO: 801 - DNA, 70 bp, synthetic construct
  source 1..70: mol_type = other DNA
cccccagaatacacaccgaccgtgtgatggcatttatccccctcaaatgttttttttttttatctgcctt

SEQ ID NO: 802 - DNA, 71 bp, synthetic construct
  source 1..71: mol_type = other DNA
caggcggtagctcaatgggcgcccaccaccctcagattttcatttaattgttttttttttttagtaaccgt

SEQ ID NO: 803 - DNA, 63 bp, synthetic construct
  source 1..63: mol_type = other DNA
tagtttggtaggtaggctatcaggtcatattaagtaatagcgttttttttttttcgctcatat

SEQ ID NO: 804 - DNA, 35 bp, synthetic construct; identical to SEQ ID NO: 1
  source 1..35: mol_type = other DNA

SEQ ID NO: 805 - DNA, 40 bp, synthetic construct; identical to SEQ ID NO: 2
  source 1..40: mol_type = other DNA

SEQ ID NO: 806 - DNA, 42 bp, synthetic construct; identical to SEQ ID NO: 134
  source 1..42: mol_type = other DNA

SEQ ID NO: 807 - DNA, 42 bp, synthetic construct; identical to SEQ ID NO: 3
  source 1..42: mol_type = other DNA

SEQ ID NO: 808 - DNA, 35 bp, synthetic construct; identical to SEQ ID NO: 4
  source 1..35: mol_type = other DNA

SEQ ID NO: 809 - DNA, 35 bp, synthetic construct; identical to SEQ ID NO: 5
  source 1..35: mol_type = other DNA

SEQ ID NO: 810 - DNA, 35 bp, synthetic construct; identical to SEQ ID NO: 6
  source 1..35: mol_type = other DNA

SEQ ID NO: 811 - DNA, 42 bp, synthetic construct; identical to SEQ ID NO: 7
  source 1..42: mol_type = other DNA

SEQ ID NO: 812 - DNA, 42 bp, synthetic construct; identical to SEQ ID NO: 13
  source 1..42: mol_type = other DNA

SEQ ID NO: 813 - DNA, 42 bp, synthetic construct; identical to SEQ ID NO: 14
  source 1..42: mol_type = other DNA

SEQ ID NO: 814 - DNA, 35 bp, synthetic construct; identical to SEQ ID NO: 15
  source 1..35: mol_type = other DNA

SEQ ID NO: 815 - DNA, 42 bp, synthetic construct; identical to SEQ ID NO: 16
  source 1..42: mol_type = other DNA

SEQ ID NO: 816 - DNA, 35 bp, synthetic construct; identical to SEQ ID NO: 17
  source 1..35: mol_type = other DNA

SEQ ID NO: 817 - DNA, 42 bp, synthetic construct; identical to SEQ ID NO: 18
  source 1..42: mol_type = other DNA

SEQ ID NO: 818 - DNA, 42 bp, synthetic construct; identical to SEQ ID NO: 19
  source 1..42: mol_type = other DNA

SEQ ID NO: 819 - DNA, 35 bp, synthetic construct; identical to SEQ ID NO: 20
  source 1..35: mol_type = other DNA

SEQ ID NO: 820 - DNA, 42 bp, synthetic construct; identical to SEQ ID NO: 21
  source 1..42: mol_type = other DNA

SEQ ID NO: 821 - DNA, 42 bp, synthetic construct; identical to SEQ ID NO: 22
  source 1..42: mol_type = other DNA

SEQ ID NO: 822 - DNA, 35 bp, synthetic construct; identical to SEQ ID NO: 23
  source 1..35: mol_type = other DNA

SEQ ID NO: 823 - DNA, 42 bp, synthetic construct; identical to SEQ ID NO: 24
  source 1..42: mol_type = other DNA

SEQ ID NO: 824 - DNA, 42 bp, synthetic construct; identical to SEQ ID NO: 25
  source 1..42: mol_type = other DNA

SEQ ID NO: 825 - DNA, 35 bp, synthetic construct; identical to SEQ ID NO: 26
  source 1..35: mol_type = other DNA

SEQ ID NO: 826 - DNA, 42 bp, synthetic construct; identical to SEQ ID NO: 27
  source 1..42: mol_type = other DNA

SEQ ID NO: 827 - DNA, 42 bp, synthetic construct; identical to SEQ ID NO: 28
  source 1..42: mol_type = other DNA

SEQ ID NO: 828 - DNA, 50 bp, synthetic construct; identical to SEQ ID NO: 29
  source 1..50: mol_type = other DNA

SEQ ID NO: 829 - DNA, 42 bp, synthetic construct; identical to SEQ ID NO: 30
  source 1..42: mol_type = other DNA

SEQ ID NO: 830 - DNA, 50 bp, synthetic construct; identical to SEQ ID NO: 31
  source 1..50: mol_type = other DNA

SEQ ID NO: 831 - DNA, 42 bp, synthetic construct; identical to SEQ ID NO: 32
  source 1..42: mol_type = other DNA

SEQ ID NO: 832 - DNA, 41 bp, synthetic construct; identical to SEQ ID NO: 35
  source 1..41: mol_type = other DNA

SEQ ID NO: 833 - DNA, 42 bp, synthetic construct; identical to SEQ ID NO: 37
  source 1..42: mol_type = other DNA

SEQ ID NO: 834 - DNA, 42 bp, synthetic construct; identical to SEQ ID NO: 135
  source 1..42: mol_type = other DNA

SEQ ID NO: 835 - DNA, 45 bp, synthetic construct; identical to SEQ ID NO: 38
  source 1..45: mol_type = other DNA

SEQ ID NO: 836 - DNA, 55 bp, synthetic construct; identical to SEQ ID NO: 39
  source 1..55: mol_type = other DNA

SEQ ID NO: 837 - DNA, 56 bp, synthetic construct; identical to SEQ ID NO: 40
  source 1..56: mol_type = other DNA

SEQ ID NO: 838 - DNA, 42 bp, synthetic construct; identical to SEQ ID NO: 41
  source 1..42: mol_type = other DNA

SEQ ID NO: 839 - DNA, 42 bp, synthetic construct; identical to SEQ ID NO: 42
  source 1..42: mol_type = other DNA

SEQ ID NO: 840 - DNA, 45 bp, synthetic construct; identical to SEQ ID NO: 43
  source 1..45: mol_type = other DNA

SEQ ID NO: 841 - DNA, 55 bp, synthetic construct; identical to SEQ ID NO: 44
  source 1..55: mol_type = other DNA

SEQ ID NO: 842 - DNA, 42 bp, synthetic construct; identical to SEQ ID NO: 45
  source 1..42: mol_type = other DNA

SEQ ID NO: 843 - DNA, 42 bp, synthetic construct; identical to SEQ ID NO: 136
  source 1..42: mol_type = other DNA

SEQ ID NO: 844 - DNA, 45 bp, synthetic construct; identical to SEQ ID NO: 46
  source 1..45: mol_type = other DNA

SEQ ID NO: 845 - DNA, 41 bp, synthetic construct; identical to SEQ ID NO: 48
  source 1..41: mol_type = other DNA

SEQ ID NO: 846 - DNA, 54 bp, synthetic construct; identical to SEQ ID NO: 50
  source 1..54: mol_type = other DNA

SEQ ID NO: 847 - DNA, 42 bp, synthetic construct; identical to SEQ ID NO: 51
  source 1..42: mol_type = other DNA

SEQ ID NO: 848 - DNA, 55 bp, synthetic construct; identical to SEQ ID NO: 52
  source 1..55: mol_type = other DNA

SEQ ID NO: 849 - DNA, 46 bp, synthetic construct; identical to SEQ ID NO: 53
  source 1..46: mol_type = other DNA

SEQ ID NO: 850 - DNA, 54 bp, synthetic construct; identical to SEQ ID NO: 54
  source 1..54: mol_type = other DNA

SEQ ID NO: 851 - DNA, 42 bp, synthetic construct; identical to SEQ ID NO: 55
  source 1..42: mol_type = other DNA

SEQ ID NO: 852 - DNA, 55 bp, synthetic construct; identical to SEQ ID NO: 56
  source 1..55: mol_type = other DNA

SEQ ID NO: 853 - DNA, 46 bp, synthetic construct; identical to SEQ ID NO: 57
  source 1..46: mol_type = other DNA

SEQ ID NO: 854 - DNA, 54 bp, synthetic construct; identical to SEQ ID NO: 58
  source 1..54: mol_type = other DNA

SEQ ID NO: 855 - DNA, 22 bp, synthetic construct
  source 1..22: mol_type = other DNA
ctgtccaacaggaaatcatagg

SEQ ID NO: 856 - DNA, 55 bp, synthetic construct; identical to SEQ ID NO: 60
  source 1..55: mol_type = other DNA

SEQ ID NO: 857 - DNA, 54 bp, synthetic construct; identical to SEQ ID NO: 61
  source 1..54: mol_type = other DNA

SEQ ID NO: 858 - DNA, 44 bp, synthetic construct; identical to SEQ ID NO: 62
  source 1..44: mol_type = other DNA

SEQ ID NO: 859 - DNA, 54 bp, synthetic construct; identical to SEQ ID NO: 63
  source 1..54: mol_type = other DNA

SEQ ID NO: 860 - DNA, 54 bp, synthetic construct; identical to SEQ ID NO: 64
  source 1..54: mol_type = other DNA

SEQ ID NO: 861 - DNA, 54 bp, synthetic construct; identical to SEQ ID NO: 65
  source 1..54: mol_type = other DNA

SEQ ID NO: 862 - DNA, 48 bp, synthetic construct; identical to SEQ ID NO: 66
  source 1..48: mol_type = other DNA

SEQ ID NO: 863 - DNA, 55 bp, synthetic construct; identical to SEQ ID NO: 68
  source 1..55: mol_type = other DNA

SEQ ID NO: 864 - DNA, 42 bp, synthetic construct; identical to SEQ ID NO: 138
  source 1..42: mol_type = other DNA

SEQ ID NO: 865 - DNA, 55 bp, synthetic construct; identical to SEQ ID NO: 69
  source 1..55: mol_type = other DNA

SEQ ID NO: 866 - DNA, 42 bp, synthetic construct; identical to SEQ ID NO: 70
  source 1..42: mol_type = other DNA

SEQ ID NO: 867 - DNA, 55 bp, synthetic construct; identical to SEQ ID NO: 71
  source 1..55: mol_type = other DNA

SEQ ID NO: 868 - DNA, 42 bp, synthetic construct; identical to SEQ ID NO: 72
  source 1..42: mol_type = other DNA

SEQ ID NO: 869 - DNA, 55 bp, synthetic construct; identical to SEQ ID NO: 73
  source 1..55: mol_type = other DNA

SEQ ID NO: 870 - DNA, 42 bp, synthetic construct; identical to SEQ ID NO: 139
  source 1..42: mol_type = other DNA

SEQ ID NO: 871 - DNA, 42 bp, synthetic construct; identical to SEQ ID NO: 74
  source 1..42: mol_type = other DNA

SEQ ID NO: 872 - DNA, 55 bp, synthetic construct; identical to SEQ ID NO: 75
  source 1..55: mol_type = other DNA

SEQ ID NO: 873 - DNA, 42 bp, synthetic construct; identical to SEQ ID NO: 76
  source 1..42: mol_type = other DNA

SEQ ID NO: 874 - DNA, 42 bp, synthetic construct; identical to SEQ ID NO: 140
  source 1..42: mol_type = other DNA

SEQ ID NO: 875 - DNA, 55 bp, synthetic construct; identical to SEQ ID NO: 77
  source 1..55: mol_type = other DNA

SEQ ID NO: 876 - DNA, 46 bp, synthetic construct; identical to SEQ ID NO: 79
  source 1..46: mol_type = other DNA

SEQ ID NO: 877 - DNA, 21 bp, synthetic construct
  source 1..21: mol_type = other DNA
atgtttagacttttatagcgt

SEQ ID NO: 878 - DNA, 46 bp, synthetic construct; identical to SEQ ID NO: 81
  source 1..46: mol_type = other DNA

SEQ ID NO: 879 - DNA, 21 bp, synthetic construct
  source 1..21: mol_type = other DNA
cagtatcggcttttaggaaga

SEQ ID NO: 880 - DNA, 46 bp, synthetic construct; identical to SEQ ID NO: 83
  source 1..46: mol_type = other DNA

SEQ ID NO: 881 - DNA, 56 bp, synthetic construct; identical to SEQ ID NO: 87
  source 1..56: mol_type = other DNA

SEQ ID NO: 882 - DNA, 56 bp, synthetic construct; identical to SEQ ID NO: 88
  source 1..56: mol_type = other DNA

SEQ ID NO: 883 - DNA, 46 bp, synthetic construct; identical to SEQ ID NO: 89
  source 1..46: mol_type = other DNA

SEQ ID NO: 884 - DNA, 42 bp, synthetic construct; identical to SEQ ID NO: 90
  source 1..42: mol_type = other DNA

SEQ ID NO: 885 - DNA, 56 bp, synthetic construct; identical to SEQ ID NO: 91
  source 1..56: mol_type = other DNA

SEQ ID NO: 886 - DNA, 46 bp, synthetic construct; identical to SEQ ID NO: 92
  source 1..46: mol_type = other DNA

SEQ ID NO: 887 - DNA, 42 bp, synthetic construct; identical to SEQ ID NO: 143
  source 1..42: mol_type = other DNA

SEQ ID NO: 888 - DNA, 56 bp, synthetic construct; identical to SEQ ID NO: 93
  source 1..56: mol_type = other DNA

SEQ ID NO: 889 - DNA, 56 bp, synthetic construct; identical to SEQ ID NO: 94
  source 1..56: mol_type = other DNA

SEQ ID NO: 890 - DNA, 42 bp, synthetic construct; identical to SEQ ID NO: 98
  source 1..42: mol_type = other DNA

SEQ ID NO: 891 - DNA, 42 bp, synthetic construct; identical to SEQ ID NO: 99
  source 1..42: mol_type = other DNA

SEQ ID NO: 892 - DNA, 59 bp, synthetic construct; identical to SEQ ID NO: 100
  source 1..59: mol_type = other DNA

SEQ ID NO: 893 - DNA, 45 bp, synthetic construct; identical to SEQ ID NO: 106
  source 1..45: mol_type = other DNA

SEQ ID NO: 894 - DNA, 48 bp, synthetic construct; identical to SEQ ID NO: 107
  source 1..48: mol_type = other DNA

SEQ ID NO: 895 - DNA, 48 bp, synthetic construct; identical to SEQ ID NO: 108
  source 1..48: mol_type = other DNA

SEQ ID NO: 896 - DNA, 45 bp, synthetic construct; identical to SEQ ID NO: 109
  source 1..45: mol_type = other DNA

SEQ ID NO: 897 - DNA, 45 bp, synthetic construct; identical to SEQ ID NO: 110
  source 1..45: mol_type = other DNA

SEQ ID NO: 898 - DNA, 48 bp, synthetic construct; identical to SEQ ID NO: 111
  source 1..48: mol_type = other DNA

SEQ ID NO: 899 - DNA, 48 bp, synthetic construct; identical to SEQ ID NO: 112
  source 1..48: mol_type = other DNA

SEQ ID NO: 900 - DNA, 59 bp, synthetic construct; identical to SEQ ID NO: 113
  source 1..59: mol_type = other DNA

SEQ ID NO: 901 - DNA, 45 bp, synthetic construct; identical to SEQ ID NO: 114
  source 1..45: mol_type = other DNA

SEQ ID NO: 902 - DNA, 48 bp, synthetic construct; identical to SEQ ID NO: 115
  source 1..48: mol_type = other DNA

SEQ ID NO: 903 - DNA, 35 bp, synthetic construct; identical to SEQ ID NO: 118
  source 1..35: mol_type = other DNA

SEQ ID NO: 904 - DNA, 42 bp, synthetic construct; identical to SEQ ID NO: 119
  source 1..42: mol_type = other DNA

SEQ ID NO: 905 - DNA, 50 bp, synthetic construct; identical to SEQ ID NO: 123
  source 1..50: mol_type = other DNA

SEQ ID NO: 906 - DNA, 35 bp, synthetic construct; identical to SEQ ID NO: 124
  source 1..35: mol_type = other DNA

SEQ ID NO: 907 - DNA, 35 bp, synthetic construct; identical to SEQ ID NO: 125
  source 1..35: mol_type = other DNA

SEQ ID NO: 908 - DNA, 42 bp, synthetic construct; identical to SEQ ID NO: 145
  source 1..42: mol_type = other DNA

SEQ ID NO: 909 - DNA, 35 bp, synthetic construct; identical to SEQ ID NO: 126
  source 1..35: mol_type = other DNA

SEQ ID NO: 910 - DNA, 21 bp, synthetic construct
  source 1..21: mol_type = other DNA
ttattaacgaactgaaagagt

SEQ ID NO: 911 - DNA, 28 bp, synthetic construct
  source 1..28: mol_type = other DNA
ctaaaatagccagcatgatgaaacaaac

SEQ ID NO: 912 - DNA, 43 bp, synthetic construct; identical to SEQ ID NO: 130
  source 1..43: mol_type = other DNA

SEQ ID NO: 913 - DNA, 41 bp, synthetic construct; identical to SEQ ID NO: 131
  source 1..41: mol_type = other DNA

SEQ ID NO: 914 - DNA, 56 bp, synthetic construct; identical to SEQ ID NO: 132
  source 1..56: mol_type = other DNA

SEQ ID NO: 915 - DNA, 21 bp, synthetic construct
  source 1..21: mol_type = other DNA
aatcggtaataaaggcaaatc

SEQ ID NO: 916 - DNA, 46 bp, synthetic construct; identical to SEQ ID NO: 96
  source 1..46: mol_type = other DNA

SEQ ID NO: 917 - DNA, 20 bp, synthetic construct
  source 1..20: mol_type = other DNA
cgcagagtcggcaaagtaac

SEQ ID NO: 918 - DNA, 42 bp, synthetic construct; identical to SEQ ID NO: 144
  source 1..42: mol_type = other DNA

SEQ ID NO: 919 - DNA, 42 bp, synthetic construct; identical to SEQ ID NO: 141
  source 1..42: mol_type = other DNA

SEQ ID NO: 920 - DNA, 28 bp, synthetic construct
  source 1..28: mol_type = other DNA
taacagtggtttaacattcaaccgattg

SEQ ID NO: 921 - DNA, 21 bp, synthetic construct
  source 1..21: mol_type = other DNA
gagaaacaagaaatgaaggta

SEQ ID NO: 922 - DNA, 26 bp, synthetic construct
  source 1..26: mol_type = other DNA
acgctcatggaaaggaacaaatacat

SEQ ID NO: 923 - DNA, 46 bp, synthetic construct; identical to SEQ ID NO: 95
  source 1..46: mol_type = other DNA

SEQ ID NO: 924 - DNA, 25 bp, synthetic construct
  source 1..25: mol_type = other DNA
gaacaccctcgtcacgtataaagcc

SEQ ID NO: 925 - DNA, 42 bp, synthetic construct
  source 1..42: mol_type = other DNA
tgagtttgaacaaattttttgtcagaggaggcttttgaggac

SEQ ID NO: 926 - DNA, 63 bp, synthetic construct
  source 1..63: mol_type = other DNA
ccagacttttttagtaaatgaatttttttgtttaattttttcgtcaaaaatgccagttacaaa

SEQ ID NO: 927 - DNA, 42 bp, synthetic construct; identical to SEQ ID NO: 164
  source 1..42: mol_type = other DNA

SEQ ID NO: 928 - DNA, 28 bp, synthetic construct
  source 1..28: mol_type = other DNA
acaccgctaatagacaacagtttcagcg

SEQ ID NO: 929 - DNA, 28 bp, synthetic construct
  source 1..28: mol_type = other DNA
atcagctatcgtaagcaattcatcaata

SEQ ID NO: 930 - DNA, 42 bp, synthetic construct; identical to SEQ ID NO: 169
  source 1..42: mol_type = other DNA

SEQ ID NO: 931 - DNA, 50 bp, synthetic construct; identical to SEQ ID NO: 170
  source 1..50: mol_type = other DNA

SEQ ID NO: 932 - DNA, 42 bp, synthetic construct; identical to SEQ ID NO: 172
  source 1..42: mol_type = other DNA

SEQ ID NO: 933 - DNA, 41 bp, synthetic construct; identical to SEQ ID NO: 173
  source 1..41: mol_type = other DNA

SEQ ID NO: 934 - DNA, 42 bp, synthetic construct; identical to SEQ ID NO: 174
  source 1..42: mol_type = other DNA

SEQ ID NO: 935 - DNA, 49 bp, synthetic construct; identical to SEQ ID NO: 175
  source 1..49: mol_type = other DNA

SEQ ID NO: 936 - DNA, 28 bp, synthetic construct
  source 1..28: mol_type = other DNA
aacccacgccaccaaaagattaagagga

SEQ ID NO: 937 - DNA, 49 bp, synthetic construct; identical to SEQ ID NO: 177
  source 1..49: mol_type = other DNA

SEQ ID NO: 938 - DNA, 50 bp, synthetic construct; identical to SEQ ID NO: 178
  source 1..50: mol_type = other DNA

SEQ ID NO: 939 - DNA, 42 bp, synthetic construct; identical to SEQ ID NO: 179
  source 1..42: mol_type = other DNA

SEQ ID NO: 940 - DNA, 28 bp, synthetic construct
  source 1..28: mol_type = other DNA
gcttagatgataagcagactgtagcgcg

SEQ ID NO: 941 - DNA, 28 bp, synthetic construct
  source 1..28: mol_type = other DNA
gctgtttgctcgaaatcaccagtagcac

SEQ ID NO: 942 - DNA, 49 bp, synthetic construct; identical to SEQ ID NO: 182
  source 1..49: mol_type = other DNA

SEQ ID NO: 943 - DNA, 28 bp, synthetic construct
  source 1..28: mol_type = other DNA
taacagtggtttaacattcaaccgattg

SEQ ID NO: 944 - DNA, 28 bp, synthetic construct
  source 1..28: mol_type = other DNA
aactaactttgcaatgtgctgcaaggcg

SEQ ID NO: 945 - DNA, 28 bp, synthetic construct
  source 1..28: mol_type = other DNA
caatcgccctgtttgtgccggaaaccag

SEQ ID NO: 946 - DNA, 50 bp, synthetic construct; identical to SEQ ID NO: 186
  source 1..50: mol_type = other DNA

SEQ ID NO: 947 - DNA, 42 bp, synthetic construct; identical to SEQ ID NO: 187
  source 1..42: mol_type = other DNA

SEQ ID NO: 948 - DNA, 38 bp, synthetic construct
  source 1..38: mol_type = other DNA
ccgcctcaccggaaaataggtttttaagtggtaagtgg

SEQ ID NO: 949 - DNA, 52 bp, synthetic construct
  source 1..52: mol_type = other DNA
agacgttggcaacagctgattattcggttaccgaattttaagtggtaagtgg

SEQ ID NO: 950 - DNA, 59 bp, synthetic construct
  source 1..59: mol_type = other DNA
gccctttagcccggccagagccacaagagaaggatgttgatattttaagtggtaagtgg

SEQ ID NO: 951 - DNA, 56 bp, synthetic construct
  source 1..56: mol_type = other DNA
gagactcctccaccgcctggccctgtcgcccaaagtaagttttaagtggtaagtgg

SEQ ID NO: 952 - DNA, 46 bp, synthetic construct
  source 1..46: mol_type = other DNA
ccagaaggcaccaggcggataagtgccgtttttaagtggtaagtgg

SEQ ID NO: 953 - DNA, 37 bp, synthetic construct
  source 1..37: mol_type = other DNA
agcggtccgccgacaagttattttaagtggtaagtgg

SEQ ID NO: 954 - DNA, 64 bp, synthetic construct
  source 1..64: mol_type = other DNA
tgaatttcttaagcgaaaaccctgcctatttcgcggaataatataaattttaagtggtaagtgg

SEQ ID NO: 955 - DNA, 73 bp, synthetic construct
  source 1..73: mol_type = other DNA
ttttgctcagtaaagacaccagaacctagtttgccccagcagacagcttggaaacgttttaagtggtaagtgg

SEQ ID NO: 956 - DNA, 39 bp, synthetic construct
  source 1..39: mol_type = other DNA
aaacgtagaatttttatacatattttaagtggtaagtgg

SEQ ID NO: 957 - DNA, 38 bp, synthetic construct
  source 1..38: mol_type = other DNA
gaacccatgtttttgtaacacttttaagttggaagagg

SEQ ID NO: 958 - DNA, 38 bp, synthetic construct
  source 1..38: mol_type = other DNA
taaagactttttcgcttaactttttaagttggaagagg

SEQ ID NO: 959 - DNA, 59 bp, synthetic construct
  source 1..59: mol_type = other DNA
attagacacgcctggcttatccgaaattcttaccacagtacattttaagttggaagagg

SEQ ID NO: 960 - DNA, 59 bp, synthetic construct
  source 1..59: mol_type = other DNA
atcagataggcgttaagccttaaatcaattatcctgaagcgcttttaagttggaagagg

SEQ ID NO: 961 - DNA, 44 bp, synthetic construct
  source 1..44: mol_type = other DNA
gaacgcgatagaagtagcattccacagttttaagttggaagagg

SEQ ID NO: 962 - DNA, 38 bp, synthetic construct
  source 1..38: mol_type = other DNA
acctcccccaatagcatagttttttaagttggaagagg

SEQ ID NO: 963 - DNA, 72 bp, synthetic construct
  source 1..72: mol_type = other DNA
atagctttttgatagctataaccttgcttctgtaaatcgtccctaatttgaaaatttttaagttggaagagg

SEQ ID NO: 964 - DNA, 49 bp, synthetic construct
  source 1..49: mol_type = other DNA
gaatccttgtcgtaggaagttttgtcgtctttttttaagttggaagagg

SEQ ID NO: 965 - DNA, 63 bp, synthetic construct
  source 1..63: mol_type = other DNA
tgctaaagtatccaaataagaaacgatttctgtatacatttgaggattttaggtagtgtgttg

SEQ ID NO: 966 - DNA, 44 bp, synthetic construct
  source 1..44: mol_type = other DNA
ataaacagccataattaccttaaaacattttaggtagtgtgttg

SEQ ID NO: 967 - DNA, 39 bp, synthetic construct
  source 1..39: mol_type = other DNA
ttttcaccaactttttagagccttttaggtagtgtgttg

SEQ ID NO: 968 - DNA, 46 bp, synthetic construct
  source 1..46: mol_type = other DNA
atcccatgaaaatctacatttaagtattattttaggtagtgtgttg

SEQ ID NO: 969 - DNA, 52 bp, synthetic construct
  source 1..52: mol_type = other DNA
gagccttacaactaaaggaatgagtgagggagcacttttaggtagtgtgttg

SEQ ID NO: 970 - DNA, 52 bp, synthetic construct
  source 1..52: mol_type = other DNA
tgcgaataaaggctccaaaagatcaagaagtgccattttaggtagtgtgttg

SEQ ID NO: 971 - DNA, 59 bp, synthetic construct
  source 1..59: mol_type = other DNA
cgctgagatctttaaatagaaagccttattacgcaaggttatttttaggtagtgtgttg

SEQ ID NO: 972 - DNA, 38 bp, synthetic construct
  source 1..38: mol_type = other DNA
atcggttaaagaagagcaaatttttaggtagtgtgttg

SEQ ID NO: 973 - DNA, 38 bp, synthetic construct
  source 1..38: mol_type = other DNA
gttggcaaatttttcagttgattttaggtagtgtgttg

SEQ ID NO: 974 - DNA, 56 bp, synthetic construct
  source 1..56: mol_type = other DNA
gatgcgaggctattctgtgtgtgtgtgtgtgtgtgtgtgtgtgtgtgtgtgtgtgt

SEQ ID NO: 975 - DNA, 30 bp, synthetic construct
  source 1..30: mol_type = other DNA
agaatagcctcgcatcccacttaccactta

SEQ ID NO: 976 - DNA, 30 bp, synthetic construct
  source 1..30: mol_type = other DNA
agaatagcctcgcatccaacacactaccta

SEQ ID NO: 977 - DNA, 30 bp, synthetic construct
  source 1..30: mol_type = other DNA
agaatagcctcgcatccctcttccaactta

SEQ ID NO: 978 - DNA, 35 bp, synthetic construct; identical to SEQ ID NO: 1
  source 1..35: mol_type = other DNA

SEQ ID NO: 979 - DNA, 40 bp, synthetic construct; identical to SEQ ID NO: 2
  source 1..40: mol_type = other DNA

SEQ ID NO: 980 - DNA, 42 bp, synthetic construct; identical to SEQ ID NO: 134
  source 1..42: mol_type = other DNA

SEQ ID NO: 981 - DNA, 42 bp, synthetic construct; identical to SEQ ID NO: 3
  source 1..42: mol_type = other DNA

SEQ ID NO: 982 - DNA, 35 bp, synthetic construct; identical to SEQ ID NO: 4
  source 1..35: mol_type = other DNA

SEQ ID NO: 983 - DNA, 35 bp, synthetic construct; identical to SEQ ID NO: 5
  source 1..35: mol_type = other DNA

SEQ ID NO: 984 - DNA, 35 bp, synthetic construct; identical to SEQ ID NO: 6
  source 1..35: mol_type = other DNA

SEQ ID NO: 985 - DNA, 42 bp, synthetic construct; identical to SEQ ID NO: 7
  source 1..42: mol_type = other DNA

SEQ ID NO: 986 - DNA, 42 bp, synthetic construct; identical to SEQ ID NO: 13
  source 1..42: mol_type = other DNA

SEQ ID NO: 987 - DNA, 42 bp, synthetic construct; identical to SEQ ID NO: 14
  source 1..42: mol_type = other DNA

SEQ ID NO: 988 - DNA, 35 bp, synthetic construct; identical to SEQ ID NO: 15
  source 1..35: mol_type = other DNA

SEQ ID NO: 989 - DNA, 42 bp, synthetic construct; identical to SEQ ID NO: 16
  source 1..42: mol_type = other DNA

SEQ ID NO: 990 - DNA, 35 bp, synthetic construct; identical to SEQ ID NO: 17
  source 1..35: mol_type = other DNA

SEQ ID NO: 991 - DNA, 42 bp, synthetic construct; identical to SEQ ID NO: 18
  source 1..42: mol_type = other DNA

SEQ ID NO: 992 - DNA, 42 bp, synthetic construct; identical to SEQ ID NO: 19
  source 1..42: mol_type = other DNA

SEQ ID NO: 993 - DNA, 35 bp, synthetic construct; identical to SEQ ID NO: 20
  source 1..35: mol_type = other DNA

SEQ ID NO: 994 - DNA, 42 bp, synthetic construct; identical to SEQ ID NO: 21
  source 1..42: mol_type = other DNA

SEQ ID NO: 995 - DNA, 42 bp, synthetic construct; identical to SEQ ID NO: 22
  source 1..42: mol_type = other DNA

SEQ ID NO: 996 - DNA, 35 bp, synthetic construct; identical to SEQ ID NO: 23
  source 1..35: mol_type = other DNA

SEQ ID NO: 997 - DNA, 42 bp, synthetic construct; identical to SEQ ID NO: 24
  source 1..42: mol_type = other DNA

SEQ ID NO: 998 - DNA, 42 bp, synthetic construct; identical to SEQ ID NO: 25
  source 1..42: mol_type = other DNA

SEQ ID NO: 999 - DNA, 35 bp, synthetic construct; identical to SEQ ID NO: 26
  source 1..35: mol_type = other DNA

SEQ ID NO: 1000 - DNA, 42 bp, synthetic construct; identical to SEQ ID NO: 27
  source 1..42: mol_type = other DNA

SEQ ID NO: 1001 - DNA, 42 bp, synthetic construct; identical to SEQ ID NO: 28
  source 1..42: mol_type = other DNA

SEQ ID NO: 1002 - DNA, 50 bp, synthetic construct; identical to SEQ ID NO: 29
  source 1..50: mol_type = other DNA

SEQ ID NO: 1003 - DNA, 42 bp, synthetic construct; identical to SEQ ID NO: 30
  source 1..42: mol_type = other DNA

SEQ ID NO: 1004 - DNA, 50 bp, synthetic construct; identical to SEQ ID NO: 31
  source 1..50: mol_type = other DNA

SEQ ID NO: 1005 - DNA, 42 bp, synthetic construct; identical to SEQ ID NO: 32
  source 1..42: mol_type = other DNA

SEQ ID NO: 1006 - DNA, 45 bp, synthetic construct; identical to SEQ ID NO: 33
  source 1..45: mol_type = other DNA

SEQ ID NO: 1007 - DNA, 41 bp, synthetic construct; identical to SEQ ID NO: 35
  source 1..41: mol_type = other DNA

SEQ ID NO: 1008 - DNA, 42 bp, synthetic construct; identical to SEQ ID NO: 37
  source 1..42: mol_type = other DNA

SEQ ID NO: 1009 - DNA, 42 bp, synthetic construct; identical to SEQ ID NO: 135
  source 1..42: mol_type = other DNA

SEQ ID NO: 1010 - DNA, 45 bp, synthetic construct; identical to SEQ ID NO: 38
  source 1..45: mol_type = other DNA

SEQ ID NO: 1011 - DNA, 55 bp, synthetic construct; identical to SEQ ID NO: 39
  source 1..55: mol_type = other DNA

SEQ ID NO: 1012 - DNA, 56 bp, synthetic construct; identical to SEQ ID NO: 40
  source 1..56: mol_type = other DNA

SEQ ID NO: 1013 - DNA, 42 bp, synthetic construct; identical to SEQ ID NO: 41
  source 1..42: mol_type = other DNA

SEQ ID NO: 1014 - DNA, 42 bp, synthetic construct; identical to SEQ ID NO: 42
  source 1..42: mol_type = other DNA

SEQ ID NO: 1015 - DNA, 45 bp, synthetic construct; identical to SEQ ID NO: 43
  source 1..45: mol_type = other DNA

SEQ ID NO: 1016 - DNA, 55 bp, synthetic construct; identical to SEQ ID NO: 44
  source 1..55: mol_type = other DNA

SEQ ID NO: 1017 - DNA, 42 bp, synthetic construct; identical to SEQ ID NO: 45
  source 1..42: mol_type = other DNA

SEQ ID NO: 1018 - DNA, 42 bp, synthetic construct; identical to SEQ ID NO: 136
  source 1..42: mol_type = other DNA

SEQ ID NO: 1019 - DNA, 45 bp, synthetic construct; identical to SEQ ID NO: 46
  source 1..45: mol_type = other DNA

SEQ ID NO: 1020 - DNA, 41 bp, synthetic construct; identical to SEQ ID NO: 48
  source 1..41: mol_type = other DNA

SEQ ID NO: 1021 - DNA, 46 bp, synthetic construct; identical to SEQ ID NO: 49
  source 1..46: mol_type = other DNA

SEQ ID NO: 1022 - DNA, 54 bp, synthetic construct; identical to SEQ ID NO: 50
  source 1..54: mol_type = other DNA

SEQ ID NO: 1023 - DNA, 42 bp, synthetic construct; identical to SEQ ID NO: 51
  source 1..42: mol_type = other DNA

SEQ ID NO: 1024 - DNA, 55 bp, synthetic construct; identical to SEQ ID NO: 52
  source 1..55: mol_type = other DNA

SEQ ID NO: 1025 - DNA, 46 bp, synthetic construct; identical to SEQ ID NO: 53
  source 1..46: mol_type = other DNA

SEQ ID NO: 1026 - DNA, 54 bp, synthetic construct; identical to SEQ ID NO: 54
  source 1..54: mol_type = other DNA

SEQ ID NO: 1027 - DNA, 42 bp, synthetic construct; identical to SEQ ID NO: 55
  source 1..42: mol_type = other DNA

SEQ ID NO: 1028 - DNA, 55 bp, synthetic construct; identical to SEQ ID NO: 56
  source 1..55: mol_type = other DNA

SEQ ID NO: 1029 - DNA, 46 bp, synthetic construct; identical to SEQ ID NO: 57
  source 1..46: mol_type = other DNA

SEQ ID NO: 1030 - DNA, 54 bp, synthetic construct; identical to SEQ ID NO: 58
  source 1..54: mol_type = other DNA

SEQ ID NO: 1031 - DNA, 22 bp, synthetic construct
  source 1..22: mol_type = other DNA
ctgtccaacaggaaatcatagg

SEQ ID NO: 1032 - DNA, 55 bp, synthetic construct; identical to SEQ ID NO: 60
  source 1..55: mol_type = other DNA

SEQ ID NO: 1033 - DNA, 54 bp, synthetic construct; identical to SEQ ID NO: 61
  source 1..54: mol_type = other DNA

SEQ ID NO: 1034 - DNA, 44 bp, synthetic construct; identical to SEQ ID NO: 62
  source 1..44: mol_type = other DNA

SEQ ID NO: 1035 - DNA, 54 bp, synthetic construct; identical to SEQ ID NO: 63
  source 1..54: mol_type = other DNA

SEQ ID NO: 1036 - DNA, 54 bp, synthetic construct; identical to SEQ ID NO: 64
  source 1..54: mol_type = other DNA

SEQ ID NO: 1037 - DNA, 54 bp, synthetic construct; identical to SEQ ID NO: 65
  source 1..54: mol_type = other DNA

SEQ ID NO: 1038 - DNA, 48 bp, synthetic construct; identical to SEQ ID NO: 66
  source 1..48: mol_type = other DNA

SEQ ID NO: 1039 - DNA, 55 bp, synthetic construct; identical to SEQ ID NO: 67
  source 1..55: mol_type = other DNA

SEQ ID NO: 1040 - DNA, 42 bp, synthetic construct; identical to SEQ ID NO: 137
  source 1..42: mol_type = other DNA

SEQ ID NO: 1041 - DNA, 55 bp, synthetic construct; identical to SEQ ID NO: 68
  source 1..55: mol_type = other DNA

SEQ ID NO: 1042 - DNA, 42 bp, synthetic construct; identical to SEQ ID NO: 138
  source 1..42: mol_type = other DNA

SEQ ID NO: 1043 - DNA, 55 bp, synthetic construct; identical to SEQ ID NO: 69
  source 1..55: mol_type = other DNA

SEQ ID NO: 1044 - DNA, 42 bp, synthetic construct; identical to SEQ ID NO: 70
  source 1..42: mol_type = other DNA

SEQ ID NO: 1045 - DNA, 55 bp, synthetic construct; identical to SEQ ID NO: 71
  source 1..55: mol_type = other DNA

SEQ ID NO: 1046 - DNA, 42 bp, synthetic construct; identical to SEQ ID NO: 72
  source 1..42: mol_type = other DNA

SEQ ID NO: 1047 - DNA, 55 bp, synthetic construct; identical to SEQ ID NO: 73
  source 1..55: mol_type = other DNA

SEQ ID NO: 1048 - DNA, 42 bp, synthetic construct; identical to SEQ ID NO: 139
  source 1..42: mol_type = other DNA

SEQ ID NO: 1049 - DNA, 42 bp, synthetic construct; identical to SEQ ID NO: 74
  source 1..42: mol_type = other DNA

SEQ ID NO: 1050 - DNA, 55 bp, synthetic construct; identical to SEQ ID NO: 75
  source 1..55: mol_type = other DNA

SEQ ID NO: 1051 - DNA, 42 bp, synthetic construct; identical to SEQ ID NO: 76
  source 1..42: mol_type = other DNA

SEQ ID NO: 1052 - DNA, 42 bp, synthetic construct; identical to SEQ ID NO: 140
  source 1..42: mol_type = other DNA

SEQ ID NO: 1053 - DNA, 55 bp, synthetic construct; identical to SEQ ID NO: 77
  source 1..55: mol_type = other DNA

SEQ ID NO: 1054 - DNA, 42 bp, synthetic construct; identical to SEQ ID NO: 142
  source 1..42: mol_type = other DNA

SEQ ID NO: 1055 - DNA, 21 bp, synthetic construct
  source 1..21: mol_type = other DNA
gaacccatgtttttgtaacac

SEQ ID NO: 1056 - DNA, 46 bp, synthetic construct; identical to SEQ ID NO: 79
  source 1..46: mol_type = other DNA

SEQ ID NO: 1057 - DNA, 21 bp, synthetic construct
  source 1..21: mol_type = other DNA
atgtttagacttttatagcgt

SEQ ID NO: 1058 - DNA, 46 bp, synthetic construct; identical to SEQ ID NO: 81
  source 1..46: mol_type = other DNA

SEQ ID NO: 1059 - DNA, 21 bp, synthetic construct
  source 1..21: mol_type = other DNA
cagtatcggcttttaggaaga

SEQ ID NO: 1060 - DNA, 46 bp, synthetic construct; identical to SEQ ID NO: 83
  source 1..46: mol_type = other DNA

SEQ ID NO: 1061 - DNA, 56 bp, synthetic construct; identical to SEQ ID NO: 87
  source 1..56: mol_type = other DNA

SEQ ID NO: 1062 - DNA, 56 bp, synthetic construct; identical to SEQ ID NO: 88
  source 1..56: mol_type = other DNA

SEQ ID NO: 1063 - DNA, 46 bp, synthetic construct; identical to SEQ ID NO: 89
  source 1..46: mol_type = other DNA

SEQ ID NO: 1064 - DNA, 42 bp, synthetic construct; identical to SEQ ID NO: 90
  source 1..42: mol_type = other DNA

SEQ ID NO: 1065 - DNA, 56 bp, synthetic construct; identical to SEQ ID NO: 91
  source 1..56: mol_type = other DNA

SEQ ID NO: 1066 - DNA, 46 bp, synthetic construct; identical to SEQ ID NO: 92
  source 1..46: mol_type = other DNA

SEQ ID NO: 1067 - DNA, 42 bp, synthetic construct; identical to SEQ ID NO: 143
  source 1..42: mol_type = other DNA

SEQ ID NO: 1068 - DNA, 56 bp, synthetic construct; identical to SEQ ID NO: 93
  source 1..56: mol_type = other DNA

SEQ ID NO: 1069 - DNA, 56 bp, synthetic construct; identical to SEQ ID NO: 94
  source 1..56: mol_type = other DNA

SEQ ID NO: 1070 - DNA, 42 bp, synthetic construct; identical to SEQ ID NO: 97
  source 1..42: mol_type = other DNA

SEQ ID NO: 1071 - DNA, 42 bp, synthetic construct; identical to SEQ ID NO: 98
  source 1..42: mol_type = other DNA

SEQ ID NO: 1072 - DNA, 42 bp, synthetic construct; identical to SEQ ID NO: 99
  source 1..42: mol_type = other DNA

SEQ ID NO: 1073 - DNA, 59 bp, synthetic construct; identical to SEQ ID NO: 100
  source 1..59: mol_type = other DNA

SEQ ID NO: 1074 - DNA, 50 bp, synthetic construct; identical to SEQ ID NO: 101
  source 1..50: mol_type = other DNA

SEQ ID NO: 1075 - DNA, 45 bp, synthetic construct; identical to SEQ ID NO: 106
  source 1..45: mol_type = other DNA

SEQ ID NO: 1076 - DNA, 48 bp, synthetic construct; identical to SEQ ID NO: 107
  source 1..48: mol_type = other DNA

SEQ ID NO: 1077 - DNA, 48 bp, synthetic construct; identical to SEQ ID NO: 108
  source 1..48: mol_type = other DNA

SEQ ID NO: 1078 - DNA, 45 bp, synthetic construct; identical to SEQ ID NO: 109
  source 1..45: mol_type = other DNA

SEQ ID NO: 1079 - DNA, 45 bp, synthetic construct; identical to SEQ ID NO: 110
  source 1..45: mol_type = other DNA

SEQ ID NO: 1080 - DNA, 48 bp, synthetic construct; identical to SEQ ID NO: 111
  source 1..48: mol_type = other DNA

SEQ ID NO: 1081 - DNA, 48 bp, synthetic construct; identical to SEQ ID NO: 112
  source 1..48: mol_type = other DNA

SEQ ID NO: 1082 - DNA, 59 bp, synthetic construct; identical to SEQ ID NO: 113
  source 1..59: mol_type = other DNA

SEQ ID NO: 1083 - DNA, 45 bp, synthetic construct; identical to SEQ ID NO: 114
  source 1..45: mol_type = other DNA

SEQ ID NO: 1084 - DNA, 48 bp, synthetic construct; identical to SEQ ID NO: 115
  source 1..48: mol_type = other DNA

SEQ ID NO: 1085 - DNA, 35 bp, synthetic construct; identical to SEQ ID NO: 118
  source 1..35: mol_type = other DNA

SEQ ID NO: 1086 - DNA, 42 bp, synthetic construct; identical to SEQ ID NO: 119
  source 1..42: mol_type = other DNA

SEQ ID NO: 1087 - DNA, 50 bp, synthetic construct; identical to SEQ ID NO: 123
  source 1..50: mol_type = other DNA

SEQ ID NO: 1088 - DNA, 35 bp, synthetic construct; identical to SEQ ID NO: 124
  source 1..35: mol_type = other DNA

SEQ ID NO: 1089 - DNA, 35 bp, synthetic construct; identical to SEQ ID NO: 125
  source 1..35: mol_type = other DNA

SEQ ID NO: 1090 - DNA, 42 bp, synthetic construct; identical to SEQ ID NO: 145
  source 1..42: mol_type = other DNA

SEQ ID NO: 1091 - DNA, 35 bp, synthetic construct; identical to SEQ ID NO: 126
  source 1..35: mol_type = other DNA

SEQ ID NO: 1092 - DNA, 21 bp, synthetic construct
  source 1..21: mol_type = other DNA
ttattaacgaactgaaagagt

SEQ ID NO: 1093 - DNA, 28 bp, synthetic construct
  source 1..28: mol_type = other DNA
ctaaaatagccagcatgatgaaacaaac

SEQ ID NO: 1094 - DNA, 21 bp, synthetic construct
  source 1..21: mol_type = other DNA
acctcccccaatagcatagtt

SEQ ID NO: 1095 - DNA, 43 bp, synthetic construct; identical to SEQ ID NO: 130
  source 1..43: mol_type = other DNA

SEQ ID NO: 1096 - DNA, 41 bp, synthetic construct; identical to SEQ ID NO: 131
  source 1..41: mol_type = other DNA

SEQ ID NO: 1097 - DNA, 56 bp, synthetic construct; identical to SEQ ID NO: 132
  source 1..56: mol_type = other DNA

SEQ ID NO: 1098 - DNA, 21 bp, synthetic construct
  source 1..21: mol_type = other DNA
aatcggtaataaaggcaaatc

SEQ ID NO: 1099 - DNA, 21 bp, synthetic construct
  source 1..21: mol_type = other DNA
gaggtgaagataatgggattt

SEQ ID NO: 1100 - DNA, 22 bp, synthetic construct
  source 1..22: mol_type = other DNA
ggcttttgacggaaagtaaaac

SEQ ID NO: 1101 - DNA, 24 bp, synthetic construct
  source 1..24: mol_type = other DNA
gaaaaatttgaggagtatgttagc

SEQ ID NO: 1102 - DNA, 25 bp, synthetic construct
  source 1..25: mol_type = other DNA
agtgcccgtttaccatatctggtca

SEQ ID NO: 1103 - DNA, 33 bp, synthetic construct
  source 1..33: mol_type = other DNA
tactggtagggaggtgcgtagttttaggacaca

SEQ ID NO: 1104 - DNA, 33 bp, synthetic construct
  source 1..33: mol_type = other DNA
gtcaatggcggtcacaatttcatttgattattt

SEQ ID NO: 1105 - DNA, 34 bp, synthetic construct
  source 1..34: mol_type = other DNA
aatattgatgatactgttgttccagttttaccta

SEQ ID NO: 1106 - DNA, 38 bp, synthetic construct
  source 1..38: mol_type = other DNA
gtatcaccgtacttttttaggaggtttagtaccaatag

SEQ ID NO: 1107 - DNA, 38 bp, synthetic construct
  source 1..38: mol_type = other DNA
ccgaacaaatgacaacaaccaagagagtttaagaggct

SEQ ID NO: 1108 - DNA, 42 bp, synthetic construct
  source 1..42: mol_type = other DNA
cataaaggaacggaatactttttccaaaagttgctttcgagg

SEQ ID NO: 1109 - DNA, 42 bp, synthetic construct; identical to SEQ ID NO: 164
  source 1..42: mol_type = other DNA

SEQ ID NO: 1110 - DNA, 28 bp, synthetic construct
  source 1..28: mol_type = other DNA
gaacgcgatagaagtagcattccacaga

SEQ ID NO: 1111 - DNA, 28 bp, synthetic construct
  source 1..28: mol_type = other DNA
acaccgctaatagacaacagtttcagcg

SEQ ID NO: 1112 - DNA, 28 bp, synthetic construct
  source 1..28: mol_type = other DNA
atcagctatcgtaagcaattcatcaata

SEQ ID NO: 1113 - DNA, 42 bp, synthetic construct; identical to SEQ ID NO: 169
  source 1..42: mol_type = other DNA

SEQ ID NO: 1114 - DNA, 50 bp, synthetic construct; identical to SEQ ID NO: 170
  source 1..50: mol_type = other DNA

SEQ ID NO: 1115 - DNA, 42 bp, synthetic construct; identical to SEQ ID NO: 172
  source 1..42: mol_type = other DNA

SEQ ID NO: 1116 - DNA, 41 bp, synthetic construct; identical to SEQ ID NO: 173
  source 1..41: mol_type = other DNA

SEQ ID NO: 1117 - DNA, 42 bp, synthetic construct; identical to SEQ ID NO: 174
  source 1..42: mol_type = other DNA

SEQ ID NO: 1118 - DNA, 49 bp, synthetic construct; identical to SEQ ID NO: 175
  source 1..49: mol_type = other DNA

SEQ ID NO: 1119 - DNA, 28 bp, synthetic construct
  source 1..28: mol_type = other DNA
aacccacgccaccaaaagattaagagga

SEQ ID NO: 1120 - DNA, 49 bp, synthetic construct; identical to SEQ ID NO: 177
  source 1..49: mol_type = other DNA

SEQ ID NO: 1121 - DNA, 50 bp, synthetic construct; identical to SEQ ID NO: 178
  source 1..50: mol_type = other DNA

SEQ ID NO: 1122 - DNA, 42 bp, synthetic construct; identical to SEQ ID NO: 179
  source 1..42: mol_type = other DNA

SEQ ID NO: 1123 - DNA, 28 bp, synthetic construct
  source 1..28: mol_type = other DNA
gcttagatgataagcagactgtagcgcg

SEQ ID NO: 1124 - DNA, 28 bp, synthetic construct
  source 1..28: mol_type = other DNA
gctgtttgctcgaaatcaccagtagcac

SEQ ID NO: 1125 - DNA, 49 bp, synthetic construct; identical to SEQ ID NO: 182
  source 1..49: mol_type = other DNA

SEQ ID NO: 1126 - DNA, 28 bp, synthetic construct
  source 1..28: mol_type = other DNA
aactaactttgcaatgtgctgcaaggcg

SEQ ID NO: 1127 - DNA, 28 bp, synthetic construct
  source 1..28: mol_type = other DNA
caatcgccctgtttgtgccggaaaccag

SEQ ID NO: 1128 - DNA, 50 bp, synthetic construct; identical to SEQ ID NO: 186
  source 1..50: mol_type = other DNA

SEQ ID NO: 1129 - DNA, 42 bp, synthetic construct; identical to SEQ ID NO: 187
  source 1..42: mol_type = other DNA

SEQ ID NO: 1130 - DNA, 37 bp, synthetic construct; identical to SEQ ID NO: 953
  source 1..37: mol_type = other DNA

SEQ ID NO: 1131 - DNA, 37 bp, synthetic construct
  source 1..37: mol_type = other DNA
cgcagagtcggcaaagtaacttttaagtggtaagtgg

SEQ ID NO: 1132 - DNA, 38 bp, synthetic construct; identical to SEQ ID NO: 948
  source 1..38: mol_type = other DNA

SEQ ID NO: 1133 - DNA, 38 bp, synthetic construct
  source 1..38: mol_type = other DNA
atcggttaaagaagagcaaatttttaagtggtaagtgg

SEQ ID NO: 1134 - DNA, 38 bp, synthetic construct
  source 1..38: mol_type = other DNA
gttggcaaatttttcagttgattttaagtggtaagtgg

SEQ ID NO: 1135 - DNA, 38 bp, synthetic construct
  source 1..38: mol_type = other DNA
gagaaacaagaaatgaaggtattttaagtggtaagtgg

SEQ ID NO: 1136 - DNA, 39 bp, synthetic construct; identical to SEQ ID NO: 956
  source 1..39: mol_type = other DNA

SEQ ID NO: 1137 - DNA, 39 bp, synthetic construct
  source 1..39: mol_type = other DNA
ttttcaccaactttttagagccttttaagtggtaagtgg

SEQ ID NO: 1138 - DNA, 43 bp, synthetic construct
  source 1..43: mol_type = other DNA
acgctcatggaaaggaacaaatacatttttaagtggtaagtgg

SEQ ID NO: 1139 - DNA, 44 bp, synthetic construct
  source 1..44: mol_type = other DNA
ataaacagccataattaccttaaaacattttaagtggtaagtgg

SEQ ID NO: 1140 - DNA, 46 bp, synthetic construct
  source 1..46: mol_type = other DNA
catttttaacggattggttgagaggagtgttttaagtggtaagtgg

SEQ ID NO: 1141 - DNA, 46 bp, synthetic construct
  source 1..46: mol_type = other DNA
atcccatgaaaatctacatttaagtattattttaagtggtaagtgg

SEQ ID NO: 1142 - DNA, 46 bp, synthetic construct; identical to SEQ ID NO: 952
  source 1..46: mol_type = other DNA

SEQ ID NO: 1143 - DNA, 52 bp, synthetic construct
  source 1..52: mol_type = other DNA
tgcgaataaaggctccaaaagatcaagaagtgccattttaagtggtaagtgg

SEQ ID NO: 1144 - DNA, 52 bp, synthetic construct; identical to SEQ ID NO: 949
  source 1..52: mol_type = other DNA

SEQ ID NO: 1145 - DNA, 56 bp, synthetic construct; identical to SEQ ID NO: 951
  source 1..56: mol_type = other DNA

SEQ ID NO: 1146 - DNA, 59 bp, synthetic construct; identical to SEQ ID NO: 950
  source 1..59: mol_type = other DNA

SEQ ID NO: 1147 - DNA, 59 bp, synthetic construct
  source 1..59: mol_type = other DNA
gggtcagagggcgacgtcagatgaccctcaatcaagcgccaattttaagtggtaagtgg

SEQ ID NO: 1148 - DNA, 59 bp, synthetic construct
  source 1..59: mol_type = other DNA
attttcaaccttttttgctttgaatacccaaaagatttaacgttttaagtggtaagtgg

SEQ ID NO: 1149 - DNA, 59 bp, synthetic construct
  source 1..59: mol_type = other DNA
cgctgagatctttaaatagaaagccttattacgcaaggttatttttaagtggtaagtgg

SEQ ID NO: 1150 - DNA, 63 bp, synthetic construct
  source 1..63: mol_type = other DNA
agaaataaagacgctcaatcgtctgaaatttgcacttattcattaattttaagtggtaagtgg

SEQ ID NO: 1151 - DNA, 63 bp, synthetic construct
  source 1..63: mol_type = other DNA
caataattggcaacagtttattttgtcacaatcaattttaaaattcttttaagtggtaagtgg

SEQ ID NO: 1152 - DNA, 63 bp, synthetic construct
  source 1..63: mol_type = other DNA
tgctaaagtatccaaataagaaacgatttctgtatacatttgaggattttaagtggtaagtgg

SEQ ID NO: 1153 - DNA, 64 bp, synthetic construct; identical to SEQ ID NO: 954
  source 1..64: mol_type = other DNA

SEQ ID NO: 1154 - DNA, 73 bp, synthetic construct; identical to SEQ ID NO: 955
  source 1..73: mol_type = other DNA

SEQ ID NO: 1155 - DNA, 38 bp, synthetic construct; identical to SEQ ID NO: 948
  source 1..38: mol_type = other DNA

SEQ ID NO: 1156 - DNA, 52 bp, synthetic construct; identical to SEQ ID NO: 949
  source 1..52: mol_type = other DNA

SEQ ID NO: 1157 - DNA, 59 bp, synthetic construct; identical to SEQ ID NO: 950
  source 1..59: mol_type = other DNA

SEQ ID NO: 1158 - DNA, 56 bp, synthetic construct; identical to SEQ ID NO: 951
  source 1..56: mol_type = other DNA

SEQ ID NO: 1159 - DNA, 46 bp, synthetic construct; identical to SEQ ID NO: 952
  source 1..46: mol_type = other DNA

SEQ ID NO: 1160 - DNA, 37 bp, synthetic construct; identical to SEQ ID NO: 953
  source 1..37: mol_type = other DNA

SEQ ID NO: 1161 - DNA, 64 bp, synthetic construct; identical to SEQ ID NO: 954
  source 1..64: mol_type = other DNA

SEQ ID NO: 1162 - DNA, 73 bp, synthetic construct; identical to SEQ ID NO: 955
  source 1..73: mol_type = other DNA

SEQ ID NO: 1163 - DNA, 39 bp, synthetic construct; identical to SEQ ID NO: 956
  source 1..39: mol_type = other DNA

SEQ ID NO: 1164 - DNA, 63 bp, synthetic construct; identical to SEQ ID NO: 965
  source 1..63: mol_type = other DNA

SEQ ID NO: 1165 - DNA, 44 bp, synthetic construct; identical to SEQ ID NO: 966
  source 1..44: mol_type = other DNA

SEQ ID NO: 1166 - DNA, 39 bp, synthetic construct; identical to SEQ ID NO: 967
  source 1..39: mol_type = other DNA

SEQ ID NO: 1167 - DNA, 46 bp, synthetic construct; identical to SEQ ID NO: 968
  source 1..46: mol_type = other DNA

SEQ ID NO: 1168 - DNA, 52 bp, synthetic construct; identical to SEQ ID NO: 969
  source 1..52: mol_type = other DNA

SEQ ID NO: 1169 - DNA, 52 bp, synthetic construct; identical to SEQ ID NO: 970
  source 1..52: mol_type = other DNA

SEQ ID NO: 1170 - DNA, 59 bp, synthetic construct; identical to SEQ ID NO: 971
  source 1..59: mol_type = other DNA

SEQ ID NO: 1171 - DNA, 38 bp, synthetic construct; identical to SEQ ID NO: 972
  source 1..38: mol_type = other DNA

SEQ ID NO: 1172 - DNA, 38 bp, synthetic construct; identical to SEQ ID NO: 973
  source 1..38: mol_type = other DNA

SEQ ID NO: 1173 - DNA, 63 bp, synthetic construct
  source 1..63: mol_type = other DNA
caataattggcaacagtttattttgtcacaatcaattttaaaattcttttaagttggaagagg

SEQ ID NO: 1174 - DNA, 37 bp, synthetic construct
  source 1..37: mol_type = other DNA
cgcagagtcggcaaagtaacttttaagttggaagagg

SEQ ID NO: 1175 - DNA, 59 bp, synthetic construct
  source 1..59: mol_type = other DNA
gggtcagagggcgacgtcagatgaccctcaatcaagcgccaattttaagttggaagagg

SEQ ID NO: 1176 - DNA, 59 bp, synthetic construct
  source 1..59: mol_type = other DNA
attttcaaccttttttgctttgaatacccaaaagatttaacgttttaagttggaagagg

SEQ ID NO: 1177 - DNA, 45 bp, synthetic construct
  source 1..45: mol_type = other DNA
taacagtggtttaacattcaaccgattgttttaagttggaagagg

SEQ ID NO: 1178 - DNA, 46 bp, synthetic construct
  source 1..46: mol_type = other DNA
catttttaacggattggttgagaggagtgttttaagttggaagagg

SEQ ID NO: 1179 - DNA, 38 bp, synthetic construct
  source 1..38: mol_type = other DNA
gagaaacaagaaatgaaggtattttaagttggaagagg

SEQ ID NO: 1180 - DNA, 43 bp, synthetic construct
  source 1..43: mol_type = other DNA
acgctcatggaaaggaacaaatacatttttaagttggaagagg

SEQ ID NO: 1181 - DNA, 63 bp, synthetic construct
  source 1..63: mol_type = other DNA
agaaataaagacgctcaatcgtctgaaatttgcacttattcattaattttaagttggaagagg

(19) United States
(12) Patent Application Publication
(10) Pub. No.: US 2026/0226546 A1
(43) Pub. Date: Aug. 6, 2026

(54) UNIVERSAL 3D NANOSCALE ASSEMBLY WITH DNA POLYCUBES

(71) Applicants: Petr SULC, Scottsdale, AZ (US); Hao LIU, Tempe, AZ (US); Thong DIEP, Tempe, AZ (US); Matthew SAMPLE, Tempe, AZ (US); Michael MATTHIES, Mesa, AZ (US); Joshua EVANS, Tempe, AZ (US)

(72) Inventors: Petr SULC, Scottsdale, AZ (US); Hao LIU, Tempe, AZ (US); Thong DIEP, Tempe, AZ (US); Matthew SAMPLE, Tempe, AZ (US); Michael MATTHIES, Mesa, AZ (US); Joshua EVANS, Tempe, AZ (US)

(21) Appl. No.: 19/468,195

(22) Filed: Feb. 3, 2026

Related U.S. Application Data
(60) Provisional application No. 63/753,685, filed on Feb. 4, 2025.
(60) Provisional application No. 63/914,758, filed on Nov. 10, 2025.

(51) Int. Cl. C12Q 1/6876; B82Y 30/00; B82Y 40/00

(52) U.S. Cl. CPC C12Q 1/6876; B82Y 30/00; B82Y 40/00

(57) ABSTRACT

Described herein is a 3D nanoscale building platform. The platform is based on cube-shaped DNA nanostructures that are rigid enough to preserve cubic shape. Different cubes are connected together through complementary overhangs. We have developed a computational design algorithm that finds the smallest number of different cube types required to self-assemble target 3D shape. Each cube can be functionalized by attaching a cargo inside of it or adjacent to it (e.g., gold nanoparticles, carbon nanotubes, proteins, etc.) and can thus be used as a “scaffold” to construct arbitrary shapes in 3D from individual nanoscale building blocks. The final assembly can be a 3D periodic lattice, or a finite-sized object. From our simulation-designed cubes, we export to an experimental blueprint, where the strands and interactions between them are designed to self-assemble into a target shape.

### CROSS-REFERENCE TO RELATED APPLICATIONS

This claims priority to U.S. Provisional Patent Application No. 63/753,685, filed on Feb. 4, 2025, and U.S. Provisional Patent Application No. 63/914,758, filed on Nov. 10, 2025, each of which is incorporated by reference herein in its entirety.

### FEDERALLY SPONSORED RESEARCH

This invention was made with government support under grant DE-SC0025265 awarded by the Department of Energy. The government has certain rights in this invention.

### REFERENCE TO SEQUENCE LISTING

This application was filed with a Sequence Listing XML in ST.26 XML format in accordance with 37 C.F.R. § 1.831. The Sequence Listing XML file submitted in the USPTO Patent Center, “208192-0033-US03_sequence_listing_xml_28-JAN-2026.xml,” was created on Jan. 28, 2026, contains 1181 sequences, has a file size of 1.02 megabytes (1,077,613 bytes), and is incorporated by reference in its entirety into the specification.

### BACKGROUND

One of the main issues in nanoscale manufacturing is the difficulty to precisely position objects in 3D. The most popular design to position objects at nanoscale is DNA origami, where each DNA strand can be functionalized with an attached material. However, single DNA origami can only span directions of about 20-30 nanometers, and is usually quite flexible, especially if it is a 3D shape.

What is needed are methods, systems, and platforms for designing larger and rigid multicomponent 3D objects, built from multiple individual DNA origami cubes.

### SUMMARY

One embodiment described herein is a cube DNA origami nanostructure, comprising: a ssDNA scaffold folded into a cubic frame, comprising: six helical bundles (HB) defining six edges of the cubic frame; and a plurality of staple strands, each staple strand being positioned proximate to a center of an edge of the cubic frame. In one aspect, the plurality of staple strands comprises pairs of complementary sequences configured to promote binding between two edges of two cube DNA origami nanostructures. In another aspect, the HBs comprise 6-12 helical turns. In another aspect, the cube DNA origami nanostructure comprises mitered vertices. In another aspect, the plurality of staple strands comprises a plurality of types of orthogonal staple strands. In another aspect, the cubic frame comprises six faces, each face comprising four edges; the plurality of staple strands comprises four types of orthogonal staple strands; and each one of the four edges of each face comprises one of the four types of orthogonal staple strands such that the four edges of each face comprise all four types of orthogonal staple strands. In another aspect, the melting temperature of the plurality of staple strands is below 55° C. In another aspect, the nanostructure further comprises carbon nanotubes, gold nanoparticles, proteins, or other molecules. In another aspect, the nanostructure further comprises carbon nanotubes. In another aspect, the nanostructure further comprises a positioning DNA strand, wherein: the positioning DNA strand is located on an exterior surface of the carbon nanotubes; and the positioning DNA strand is complementary to another sequence within the nanostructure.

Another embodiment described herein is a polycube assembly, comprising: a plurality of the cube DNA origami nanostructures described herein, wherein: each of the plurality of the cube DNA origami nanostructures belongs to one of a plurality of cube types; the plurality of staple strands of the cube DNA origami nanostructures of each of the plurality of cube types independently comprise a set of staple strands; each of the cube types has a unique set of staple strands; and the plurality of the staple strands of the plurality of cube types are designed to promote binding between the plurality of cube types, whereby the cube DNA origami nanostructures assemble into a specific 3D configuration. In one aspect, each cube DNA origami nanostructure comprises a set of six faces comprising a top, a bottom, a left, a right, a front, and a back; each face comprises four edges; and each edge of each face of each of the plurality of cube types has a unique sequence. In another aspect, each edge of each face of each of the plurality of cube types has a sequence selected from SEQ ID NO: 1-1181. In another aspect, the polycube assembly further comprises one or more blocker strands configured to prevent aggregation of the cube DNA origami nanostructures. In another aspect, the plurality of cube types comprises 2-19 cube types. In another aspect, each of the cube DNA origami nanostructures of each of the plurality of cube types comprises a unique set of sequences selected from SEQ ID NO: 1-1181. In another aspect, the specific 3D configuration is selected from the group consisting of: a 3D cross, a hollow 3×3×3 cube, a human only polycube, a wolf only polycube, a multifarious werewolf polycube, fractal Menger cube M₂, a simple cubic crystal lattice, and a solid 4×4×4 assembly. In another aspect, the polycube assembly further comprises carbon nanotubes, gold nanoparticles, proteins, or other molecules. In another aspect, the polycube assembly further comprises carbon nanotubes. In another aspect, the polycube assembly further comprises a positioning DNA strand, wherein: the positioning DNA strand is located on an exterior surface of the carbon nanotubes; and the positioning DNA strand is complementary to another sequence within the polycube assembly.

Another embodiment described herein is a method of making a three-dimensional (3D) DNA nanocube capable of self-assembly into larger 3D multicomponent structures, the method comprising: obtaining one or more DNA origami monomers comprising staple strands; and incubating the DNA origami monomers for in a solution a period of time and at a temperature sufficient for self-assembly into the DNA nanocube. In one aspect, the one or more DNA origami monomers comprise a ssDNA scaffold. In another aspect, the one or more DNA origami monomers comprise an M13mp18 scaffold. In another aspect, the incubating step is performed in a buffer. In another aspect, the incubating step is performed in TAE Mg²⁺ buffer.

In another aspect, the method further comprises purifying the DNA nanocubes via gel electrophoresis and extracting a monomer band. In another aspect, one or more of the staple strands is hybridized to a DNA-CNT conjugate, the DNA-CNT conjugate comprising a carbon nanotube and a positioning sequence, the positioning sequence being complementary to the one or more staple strands.

Another embodiment described herein is a method of making a polycube assembly, the method comprising: providing a solution comprising a plurality of DNA origami nanostructures, each of the plurality of the DNA origami nanostructures comprising: a ssDNA scaffold folded into a cubic frame, comprising: six helical bundles (HB) defining six edges of the cubic frame; and a plurality of staple strands, each staple strand being positioned proximate to a center of an edge of the cubic frame; heating the solution to a temperature for a period of time such that plurality of DNA origami nanostructures assembles into a polycube assembly. In another aspect, each of the plurality of the cube DNA origami nanostructures belongs to one of a plurality of cube types; the plurality of staple strands of the cube DNA origami nanostructures of each of the plurality of cube types independently comprise a set of staple strands; each of the cube types has a unique set of staple strands; and the plurality of the staple strands of the plurality of cube types are designed to promote assembly of the plurality of DNA origami nanostructures assembles into the polycube assembly. In another aspect, each cube DNA origami nanostructure comprises a set of six faces comprising a top, a bottom, a left, a right, a front, and a back; each face comprises four edges; and each edge of each face of each of the plurality of cube types has a unique sequence. In another aspect, each edge of each face of each of the plurality of cube types has a unique sequence selected from SEQ ID NO: 1-1181. In another aspect, the method further comprises adding one or more blocker strands configured to prevent aggregation of the cube DNA origami nanostructures. In another aspect, the plurality of cube types comprises 2-19 cube types. In another aspect, each of the cube DNA origami nanostructures of each of the plurality of cube types comprises a unique set of sequences selected from SEQ ID NO: 1-1181. In another aspect, the polycube assembly is selected from the group consisting of: a 3D cross, a hollow 3×3×3 cube, a human only polycube, a wolf only polycube, a multifarious werewolf polycube, fractal Menger cube M₂, a simple cubic crystal lattice, and a solid 4×4×4 assembly. In another aspect, the plurality of DNA origami nanostructures further comprises carbon nanotubes, gold nanoparticles, proteins, or other molecules. In another aspect, the plurality of DNA origami nanostructures further comprises carbon nanotubes, the method further comprising: positioning the carbon nanotubes in one or more 2D or 3D junctions, thereby forming one or more carbon nanotube circuits.

Another embodiment described herein is a system for designing a three-dimensional structure assembled from multiple DNA nanocubes, the system comprising: a processor configured to execute instructions to: conduct one or more computer simulations to identify a minimal number of cube types required to assemble a target three-dimensional structure; assign complementary DNA sequences to faces of the cube types to define binding interactions between the cube types; and generate an output specifying DNA origami monomers for assembly into the target three-dimensional structure. In one aspect, the computer simulations comprise a satisfiability solver to identify solutions that minimize misassembly states. In another aspect, the processor is further configured to execute instructions to conduct patchy particle simulations to evaluate kinetics and success of assembly for the target three-dimensional structure. In another aspect, the processor is further configured to execute instructions to identify aggregated structures during the patchy particle simulations and feed the aggregated structures back into the satisfiability solver to refine the design process. In another aspect, the target three-dimensional structure comprises a 3D cross, a hollow 3×3×3 cube, a human only polycube, a wolf only polycube, a multifarious werewolf polycube, fractal Menger cube M₂, a simple cubic crystal lattice, or a solid 4×4×4 assembly, and wherein the satisfiability solver determines a minimal number of cube types and a ratio of each cube type required for assembly. In another aspect, the target three-dimensional structure comprises a carbon nanotube circuit.

Another embodiment described herein is a method of designing a three-dimensional structure assembled from multiple DNA nanocubes, the method comprising: conducting one or more computer simulations to identify a minimal number of cube types required to assemble a target three-dimensional structure; assigning complementary DNA sequences to faces of the cube types to define binding interactions between the cube types; and generating an output specifying DNA origami monomers for assembly into the target three-dimensional structure. In one aspect, the target three-dimensional structure comprises a carbon nanotube circuit. In another aspect, the target three-dimensional structure comprises DNA nanocubes attached to gold nanoparticles, proteins, or other molecules.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-B show a framework of polycube self-assemblies. FIG. 1A shows an algorithm implementation of SAT to identify minimal solutions for producing an exemplary 4×4×4 solid cube constructed using minimal solutions with six CDO subunits and Patchy particle simulations for the kinetics and success of the 4×4×4 solid cube assembly. Aggregated structures identified during simulations were fed back into the SAT solver to refine the design process. FIG. 1B shows an oxView visualization of the CDO design with its sticky-ends, an oxView mean structure of the solid 4×4×4 DNA origami on the assembled lattice, verifying mechanical stability; the thermal annealing process of DNA origami using a scaffold and excess staple strands; an agarose gel electrophoresis to characterize the formation of each design and to quantify yields based on band intensity; and the application of various electron microscopy techniques to characterize the formation of structures.

FIG. 2 shows experimental evidence of reconfigurability.

FIG. 3 shows experimental characterization of cubic building unit.

FIG. 4 shows experimental characterization of larger multi-cube assemblies.

FIG. 5 shows experimental evidence: assembly of largest 3D DNA nanostructure to date (Nov. 8, 2025) using the design pipeline and cubic blocks.

FIG. 6 shows schematics: additive 3D manufacturing on surface.

FIG. 7 shows a flowchart illustrating the of Patchy→Origami algorithm.

FIG. 8 shows a flowchart illustrating the algorithm used to associate each patchy particle type to a DNA and assign patches to 3′-ends of specific staples.

FIG. 9 shows a schematic illustration of strand displacement mechanism for blocking and releasing patches involved in self-binding interactions during cube DNA origami (CDO) folding.

FIG. 10 shows Cadnano design of the CDO with mitered joints created using Athena software. The CDO is composed of 6 HB along each edge, with an average edge length of 94 nucleotides, corresponding to 9 helical turns. The blue strand is the scaffold, and staple strands are shown in various colors. Overhang extensions for torsional controls are positioned at the midpoint of each edge and designed using oxView to define binding interactions between CDOs.

FIG. 11A-C shows structural representation of CDO and design for polycube self-assembly. FIG. 11A shows a cylindrical model of the CDO with a mitered edge design constructed from 6 HBs. FIG. 11B shows a CDO design in ChimeraX illustrating the blue strand as the scaffold and the white strands as staples. The edge length of the CDO design is approximately 43 nm. FIG. 11C shows a schematic illustrating the single-facet DNA handle design of the CDO using oxView. To enable torsional binding control, four orthogonal sequences were positioned at the middle of each edge on the CDO and validated their design using oxDNA MD simulations. These sequences are depicted as smaller spheres with binding limited to spheres of the same color. For each DNA handle strand, poly-T spacers (yellow strands) were added before the handle strands (multicolor strands) to provide flexibility and expand the configurational space for patch binding during polycube assembly.

FIG. 12A-C show AGE characterization of the folding and purification of CDO. FIG. 12A shows unpurified CDO from 20 hours of annealing results in a high yield of CDO monomers across different magnesium concentrations. The standard condition of 12.5 mM magnesium was used for further assembly. FIG. 12B shows a comparison between annealed unpurified and purified CDO. Unpurified CDO shows some contamination in the upper band, whereas purified CDO from gel extraction displays a clean monomer band with no contamination. FIG. 12C shows a typical yield profile of unpurified CDO with more than 81% monomer yield, contains some aggregated dimers and multimers, which can affect the assembly of polycubes. The red arrow indicates the target monomeric CDO.

FIG. 13A-B show folded CDO structural characterization. FIG. 13A shows AFM imaging of folded CDOs, showing structural rigidity maintained under the AFM tip due to the mitered edge design. FIG. 13B shows an nsTEM micrograph of folded CDOs.

FIG. 14A-C show cryo-TEM characterization of CDO. FIG. 14A shows Cryo-TEM imaging shows folded CDOs with well-defined structures. Scale bars: 40 nm. FIG. 14B shows representative 2D class averages highlighting the uniformity of the particles. FIG. 14C shows single-particle 3D reconstruction from 1,805 CDO particles presents both a face-on view (left) and a rotated perspective (right), emphasizing the miter edge design that enhances structural rigidity and is critical for hierarchical assembly. Scale bars: 10 nm.

FIG. 15A-C show polycube structure designs with minimal complexity building blocks shown as polycube models and CDO representations in oxView. FIG. 15A shows a cross cube design that uses 2 CTs to assemble a structure of 7 cubes. FIG. 15B shows a 3×3×3 hollow cube design that is achieved by modifying the placement of binding patches, assembling 20 cubes from 2 CTs. FIG. 15C shows a 4×4×4 solid cube design that is constructed with 6 CTs and self-complementary patches, forming a total of 64 cubes.

FIG. 16 shows DLS profile for self-assembly of static polycube structures at 12.5 mM Mg²⁺. The DLS data illustrates the Z-average (Z-ave) hydrodynamic size of the ensemble as a function of temperature. As the temperature decreases, the initially monodisperse CDOs begin to aggregate, resulting in an increase in particle size. This broad annealing window is optimized and adapted for assembling finite-size polycubes. Before the DLS measurement, the cuvette is equilibrated at the starting temperature for 5 minutes to dissociate any binding events of the CDOs. The program then decreases the temperature at a ramping rate of −0.1° C. per 5 minutes. Since the cross has the smallest structural size and the 4×4×4 solid cube has the largest, the Z-ave is expected to follow a similar trend.

FIG. 17A-B show comparison of cross cube assembly methods using only amicon and gel extraction for CDO purification. FIG. 17A shows AGE characterization of cross cube assembly. Amicon purification removes excess staples but does not eliminate aggregated or misfolded CDOs, resulting in a lower yield of the target cross cube. In contrast, gel purification isolates monomeric CDOs and significantly improves assembly fidelity. The red arrow highlights the target cross cube band. FIG. 17B shows yield quantification of cross cube assembly. The yield demonstrates over a 50% improvement with gel extraction compared to Amicon purification.

FIG. 18A-B show nsTEM characterization of cross cube assembly. FIG. 18A shows cross cube assembly from Amicon-purified CDOs shows a low yield of the target structure due to the presence of misfolded CDOs, which lead to aggregation during hierarchical assembly. Red circles highlight misfolded CDOs affecting the assembly. FIG. 18B shows cross cube assembly using gel-extracted CDOs demonstrates a high yield of the target structure, as the purification process ensures clean, monomeric CDOs. Based on these results, gel extraction is adopted for further polycube assemblies.

FIG. 19A-C show cryo-TEM characterization of the 3D cross cube assembly. FIG. 19A shows Cryo-TEM imaging reveals the well-defined structure of the 3D cross cube. FIG. 19B shows representative 2D class averages highlight the symmetrical DNA handle design, which facilitates face-to-face interactions and ensures the success of the assembly. FIG. 19C shows single-particle 3D reconstruction of the cross cube presents a face-on view (left) and a rotated perspective (right). The outer cubes exhibit lower resolution due to the higher flexibility and variance of their single-face binding design, whereas the inner cube achieves higher resolution with six-face binding interactions. Scale bar: 40 nm.

FIG. 20A-B show experimental characterization of 3×3×3 hollow cube assembly. FIG. 21A shows AGE characterization of the annealed 3×3×3 hollow cube demonstrating a 90% yield of the assembly. A 0.4% agarose gel is used due to the large size of the structure, and the gel is run at 100 V for 1 hour. FIG. 20B shows nsTEM images of the silicated 3×3×3 hollow cube assembly further confirm the high yield of the assembled structure.

FIG. 21A-C show AGE characterization of CDO encapsulated with AuNPs. FIG. 21A shows purified CDOs with and without AuNPs imaged under regular light. Modified-DNA AuNPs are visible, and only the CDO sample containing AuNPs appears under regular light due to their distinct optical properties. FIG. 21B shows the same gel imaged under UV revealing slower migration of CDOs with AuNPs, attributed to the increased molecular weight from the incorporated gold, while modified-DNA AuNPs alone were not visible under UV light. FIG. 21C shows high yields of CDOs encapsulating AuNPs were achieved using our design. Red arrows mark the target CDOs encapsulated with AuNPs, and the green arrow indicates free modified-DNA AuNPs.

FIG. 22 shows nsTEM characterization of purified CDO encapsulated with AuNPs. TEM images reveal a high density of monomeric CDOs with AuNPs encapsulated inside, showing minimal empty cubes and no detectable aggregates. This high encapsulation efficiency is critical for subsequent hierarchical assembly, as it ensures a precise 1:1 ratio of CDO to AuNPs, which is essential for maintaining structural integrity and functional design.

FIG. 23A-C show AGE characterization of 3×3×3 hollow cube encapsulated with AuNPs. FIG. 23A shows an annealed 3×3×3 hollow cubes with and without AuNPs imaged under regular light. Only the hollow cube sample containing AuNPs is visible under regular light due to the optical properties of gold. FIG. 23B shows the same gel imaged under UV reveals identical migration speeds for hollow cubes with and without AuNPs, indicating consistent structural integrity. FIG. 23C shows the yield quantification illustrating a 20% reduction for hollow cubes with AuNPs. Some aggregates were observed, likely due to kinetic traps caused by DNA-modified AuNPs bridging two or more CDOs together, instead of the intended DNA handle binding. Red arrows indicate the target 3×3×3 hollow cube encapsulated with AuNPs.

FIG. 24 shows nsTEM characterization of silicated 3×3×3 hollow cube encapsulated with AuNPs. High yield formation of the 3×3×3 hollow cube encapsulated with AuNPs is confirmed by TEM images. Each hollow cube, constructed from 20 individual CDOs, encapsulates 20 AuNPs, demonstrating precise spatial arrangement and high encapsulation efficiency. The uniform distribution of AuNPs within the hollow cube underscores the robustness of the assembly strategy, showcasing its potential as a delivery vehicle or a scaffold for organizing inorganic materials.

FIG. 25 shows nsTEM characterization of silicated 4×4×4 solid cube assembly. The structure, composed of 64 cubes, is too large to migrate out of the gel, preventing yield quantification by AGE. An estimated yield of approximately 65% is obtained through manual particle counting in TEM images. This value may underestimate the actual yield due to structural collapse during sample preparation and drying for nsTEM. Nonetheless, TEM images clearly demonstrate a high yield of the designed assembly.

FIG. 26A-C show SEM characterization of silicated 4×4×4 solid cube assembly at different tilt angles. Consistent with TEM images, a high yield of the target assembly is observed. The 3D structure of the cubes is clearly visible under various tilt angles. FIG. 26A shows zero-degree tilt. Scale bars: left, 20 nm; right, 1 μm. FIG. 26B shows 25-degree tilt. Scale bars: 200 nm. FIG. 26C shows 40-degree tilt. Scale bars: left, 100 nm; right, 200 nm.

FIG. 27 shows design schematic for multifarious werewolf self-assembly. A shared set of components is combined to assemble two distinct structures. The human-only structure requires 6 CTs with a total of 13 cubes, while the wolf-only structure requires 6 CTs with a total of 14 cubes. For multifarious assembly, 10 CTs were mixed in a one-pot annealing process to produce both the human and wolf structures. Inset: CDO representations of the human and wolf structures visualized in oxView.

FIG. 28 shows DLS profile of multifarious polycube self-assembly at 12.5 mM Mg²⁺. We showcase here the thermal profiles of three polycube designs as an example of our simulation design method: human-only, wolf-only, and werewolf. The multifarious werewolf forms either human or wolf structures within the same test tube. For each design, the cuvette is first incubated at a high temperature for 5 minutes, followed by a temperature decrease at a ramping rate of −0.1° C. every 5 minutes. The human design is composed of 13 cubes and exhibits a slightly smaller Z-ave compared to the wolf design, which contains 14 cubes. For the werewolf design, the Z-ave reflects the average particle size derived from both human and wolf structures, resulting in a distribution that lies between the sizes of these two individual designs.

FIG. 29A-B show experimental characterization of multifarious werewolf assembly. FIG. 29A shows AGE characterization of annealed werewolf assembly showing three structures: human only, wolf only, and multifarious werewolf. The human structure consists of 13 cubes, while the wolf structure requires 14 cubes. Consequently, the human band exhibits faster gel mobility compared to the wolf band. When all shared components were mixed for the multifarious werewolf assembly, two distinct bands appear in the gel, confirming successful multifarious assembly. The green arrow indicates the human structure, and the red arrow indicates the wolf structure. FIG. 29B shows the yield quantification from AGE shows a high yield for the multifarious werewolf assembly.

FIG. 30A-C show nsTEM characterization of silicated multifarious werewolf assembly. FIG. 30A shows human-only structure. FIG. 30B shows wolf-only structure. FIG. 30C shows multifarious werewolf assembly, where both human and wolf structures can be identified. It is worth noting that some wolf structures tend to collapse under TEM, likely due to their more pronounced 3D nature compared to the human design.

FIG. 31 shows DLS measurement for SC crystal lattice at 25 mM Mg²⁺ using different lengths of polyT spacers. The cuvette is equilibrated at the starting temperature for 5 minutes, followed by a temperature decrease at a ramping rate of −0.1° C. every 2 minutes. We observe a narrower annealing window and larger Z-ave size for the SC system compared to finite-size polycube systems. Despite using the same DNA handle sequences for both designs, shorter 5T spacers result in a higher annealing temperature range because they reduce the entropic freedom of the overhangs, leading to stronger hybridization compared to 15T spacers. Conversely, longer 15T spacers provide greater flexibility and increase entropy but weaken hybridization strength. This highlights the critical role of spacer length in modulating the assembly process and the annealing temperature window.

FIG. 32A-B show SEM imaging of SC lattice design with face-to-face interactions and polyT spacers of varying lengths after one annealing cycle. FIG. 32A shows the 5T spacer design results in smaller crystals due to the reduced conformational space of the handles, creating more constraints for CDO hybridization and limiting the formation of larger crystals. Scale bars: left, 300 nm, right, 1 μm. FIG. 32B shows the 15T spacer design yields larger crystals due to the increased flexibility of the handles, which promotes long-range order in the crystalline structures. Based on these observations, the 15T spacer design is selected for the re-annealing strategy. Scale bars: 1 μm.

FIG. 33 shows nsTEM characterization of 15T spacer unsilicated SC lattice design. A clear CDO binding pattern in an SC arrangement is observed in the zoomed-in image, while the zoomed-out images reveal a high density of SC lattices. Due to the structural depth, electrons are unable to penetrate the crystal lattice for higher resolution imaging. A 25 mM Mg²⁺ concentration is used during grid preparation to stabilize the lattice under high vacuum conditions.

FIG. 34A-B show SEM imaging of the SC lattice design with 15 polyT spacers using a re-annealing strategy. Crystal orderliness improves as small seeds were removed by heating the sample slightly above its melting point. FIG. 34A shows zoomed-in images. Scale bars: 1 μm. FIG. 34B shows zoomed-out images. Scale bars: left, 1 μm, right, 10 μm.

FIG. 35 shows a schematic illustration for the dimer cubes displacement reaction. The target cube (CT0) contains a toehold (TH) region positioned between the polyT spacers and the complementary sticky end (SE*) strand, allowing the invader cubes to recognize and displace the incumbent cube (CT1). An invader strand is added in front of the sticky end (SE) strand of the invader cube (CT2) to invade the substrate cubes. CT3 serves as a labeled cube on CT2 to indicate the success of the displacement reaction. Both invader cubes and substrate cubes were assembled individually and then mixed in 1:1 ratio. During the intermediate state, the invader cubes recognize the substrate cubes via the toehold region and bind to CT0. They gradually orient themselves to achieve face-to-face alignment, which represents the lowest free energy state. Because the (invader+SE)-(toehold+SE*) interaction has a lower free energy than the SE-SE* interaction, the invader cubes successfully displace CT1. The final products were trimer cubes (CT0+CT2+CT3) and monomer CT1 as a by-product.

FIG. 36A-B show experimental characterization of the dimer cubes displacement reaction at different temperatures overnight. The displacement is performed by mixing the two annealed dimers:substrate cubes with invader cubes at a 1:1 ratio. The reaction is conducted with two conditions. The substrate cubes without the TH region act as a negative control, which does not give a displacement signal for the invading cubes to interact and displace the substrate cubes. In contrast, the displacement occurs when the substrate cubes have the TH region which enable binding and result in the formation of trimers and monomer CDOs. FIG. 36A shows dimer cubes displacement at RT. The experiment is performed at RT to ensure that temperature does not influence the displacement reaction or produce false positive results due to the melting temperature of the DNA handles. No significant band of the trimer product is observed in the “Displacement—No TH” lane, demonstrating that displacement does not occur without the TH region. However, the “Displacement—With TH” lane appears to have significant bands corresponding to trimers and tetramers, representing the target product and intermediate state, respectively. Yield quantification in the “Displacement—With TH” lane confirms successful displacement, albeit with a low yield of trimers. The high yield of tetramers likely results from slow particle diffusion kinetics and leads to a trapped state. Aggregates form due to multiple invading cubes interacting with substrate cubes simultaneously. FIG. 36B shows dimer cubes displacement at 35° C. Increasing the temperature to 35° C. enhances the kinetic energy of particle diffusion. Both “Displacement—No TH” and “Displacement—With TH” samples were incubated at this elevated temperature for comparison. The “Displacement—No TH” sample shows a very faint trimer band due to random diffusion and nonspecific binding events. In contrast, the “Displacement—With TH” sample results in nearly three times the displacement yield and successfully eliminates the intermediate state. The higher yield of target trimers and monomers indicates the success of the cube displacement mechanism. The final gel lane represents the incubation of the substrate cubes at 35° C. to ensure that the dimers remain bound. The heated sample is quenched with glycerol and promptly loaded into the gel pocket. The yield comparison for the substrate cubes at 35° C. shows only a 5% reduction compared to substrate cubes at RT. The red arrows mark the target trimer cube product, and the green arrows mark the intermediate state during the displacement event.

FIG. 37 shows AFM structural characterization of the dimer cube displacement reaction incubated at RT overnight. Despite the low diffusion rate, invading cubes successfully displace substrate cubes, resulting in the formation of trimer and monomer cubes, as observed in the AFM images. However, numerous intermediate states were also visible, where invading cubes partially bind to substrate cubes, preventing complete trimer formation. Additionally, low-occupancy aggregates, such as 5mers formed by multiple invading cubes interacting with a single substrate cube, were detected, consistent with the displacement mechanism and gel analysis.

FIG. 38A-B show structural characterization of the dimer cube displacement reaction incubated at 35° C. overnight. FIG. 38A shows AFM images that reveal successful displacement with the formation of trimers and monomers, alongside some dimers, which likely represent unreacted substrate cubes. These unreacted cubes limit the reaction as multiple invading cubes can bind to reactive substrate cubes, depleting the available substrate for further displacement. Aggregates were reduced due to heating, which facilitates the favorable face-to-face alignment of cubes. FIG. 38B shows nsTEM analyses that corroborates these findings, aligning with results from AGE and AFM, and confirms the success of the cube displacement reaction.

FIG. 39A-C show illustrative design of the fractal Menger cube using the polycube model up to M₂, the second iteration. FIG. 39A shows M₀, which represents the base CDO unit. FIG. 39B shows M₁, the first iteration, that is a 3×3×3 hollow cube comprising 20 cubes assembled from 2 CTs as determined by the SAT solver. FIG. 39C shows the final structure, M₂, that comprises 400 cubes utilizing 192 orthogonal DNA sequences and is assembled in a one-pot annealing using 19 CTs. Each M₁ unit consists of 20 cubes, and M₂ is constructed using 20 such M₁ units. This M₂ design utilizes 8 CTs to define the M₁ corners and 11 CTs to construct the M₁ edges, ensuring a minimal solution for CTs.

FIG. 40 shows DLS spectrum for the self-assembly of a fractal Menger cube M₂ at 12.5 mM Mg²⁺. The fractal Menger cube is assembled in a one-pot reaction, constructing 400 cubes from 19 CTs. Given the complexity and large number of CTs for the assembly, a slow cooling rate is implemented to obtain the thermal profile of this fractal structure. The cuvette is equilibrated at the starting temperature for 15 minutes, followed by a temperature decrease at a ramping rate of −0.1° C. every 20 minutes. Based on the DLS profile, the optimized annealing protocol for the Menger cube is set from 44 to 20° C. at a slow annealing rate to ensure structural integrity and high assembly fidelity.

FIG. 41A-B show experimental characterization of fractal Menger cube assembly after 4 days of annealing. FIG. 41A shows AGE analysis comparing the different CTs required to assemble the structure. The M₁ control design, referred to as the 3×3×3 hollow cube, is assembled using 2 CTs determined by the SAT solver. The M₁ corner and M₁ edge constructs exhibit identical migration speeds compared to the M₁ control. M₂, composed of 400 cubes in a one-pot assembly, is too large to migrate out of the gel pocket. The red arrow highlights the M₁ target structure. FIG. 41B shows yield quantification for the M₁ design using different CTs demonstrates consistent yields regardless of the number of CTs used in the assembly. This result highlights the robustness of our computational design pipeline for hierarchical assemblies, minimizing kinetic traps and metastable states.

FIG. 42 shows nsTEM characterization of the silicated M₁ corner after 4 days of annealing. Well-defined M₁ Menger cube structures were observed, demonstrating the success of the assembly.

FIG. 43 shows nsTEM characterization of the one-pot self-assembly of the silicated fractal Menger cube M₂ after 4 days of annealing. TEM images reveal amorphous aggregates and partially formed Menger cubes, indicating that the annealing time was insufficient for the successful assembly of the design.

FIG. 44 shows nsTEM characterization of the one-pot self-assembly of the silicated fractal Menger cube M₂ after 12 days of annealing. The alignment of nine cubes across both vertical and horizontal directions, along with fractal holes, demonstrates successful assembly. Notably, the Menger cube M₂ features an empty center with large and small holes intrinsic to its design, leading to structural collapse during sample preparation for EM characterization. Consequently, a 12-day annealing period was selected for further EM characterizations of the fractal Menger cube M₂.

FIG. 45 shows SEM characterization of the one-pot self-assembly of the silicated fractal Menger cube M₂. Fractal holes and the alignment of nine cubes were observed across horizontal and vertical directions, as well as through the depth of the structure, within the resolution limits of SEM. Structural collapse is attributed to insufficient silica growth and the intrinsic fractal holes of the M₂ design.

FIG. 46 shows the application of the platform to position carbon nanotubes in 2D and 3D using DNA nanocube. The carbon nanotubes are wrapped in DNA strands. The DNA strands on the DNA nanocube are extended to be complementary to the DNA strands wrapped around the carbon nanotube. This allows precise placement in 2D and 3D junctions, as illustrated in the schematic figure and experimentally verified by TEM.

FIG. 47A-D show AFM characterization and schematic illustration of DNA-CNT dispersions after PEG-induced length sorting and subsequent DNA hybridization. FIG. 47A shows representative AFM image of DNA-CNTs sorted with 8 kDa PEG. FIG. 57B shows a representative AFM image of DNA-CNTs sorted with 6 kDa PEG. FIG. 47C shows a length distribution histogram of 8 kDa PEG-sorted DNA-CNTs fitted with a Gaussian function. FIG. 47D shows a schematic illustration of the hybridization between L1-wrapped CNTs (L1: yellow; SEQ ID NO: 974) and the DNA anchor strands (L2: green, L3: blue, L4: red; SEQ ID NO: 975-944).

FIG. 48A-D show AGE characterization for CNT-functionalized CDOs. FIG. 48A shows the same sequences on three sides for the three-way junction (Same seq—3WJ). FIG. 48B shows different sequences on one to three sides for the three-way junction (Diff seq—3WJ). FIG. 48C shows different sequences on one to three sides for the U-shape (Diff seq—U). Arrows indicate the target product with three CNTs bound to CDO. Band shifting can be observed between CDO only and 1-3 CNT-bound structures. FIG. 48D shows yield estimation for CDOs bound with three CNTs in the 3WJ and U-shape designs; note that aggregation in the wells can cause overestimation of the yield. Designs with different sequences show higher yield compared to same-sequence design.

FIG. 49A-C show nsTEM characterization of CNT-functionalized CDOs showing CNT junction formation. FIG. 49A shows 3WJ with same sequences and longer CNTs. FIG. 49B shows 3WJ with different sequences using shorter CNTs. FIG. 49C shows U-shape with different sequences using shorter CNTs. Arrows highlight CNTs bound to CDOs. Scale bars: 50 nm.

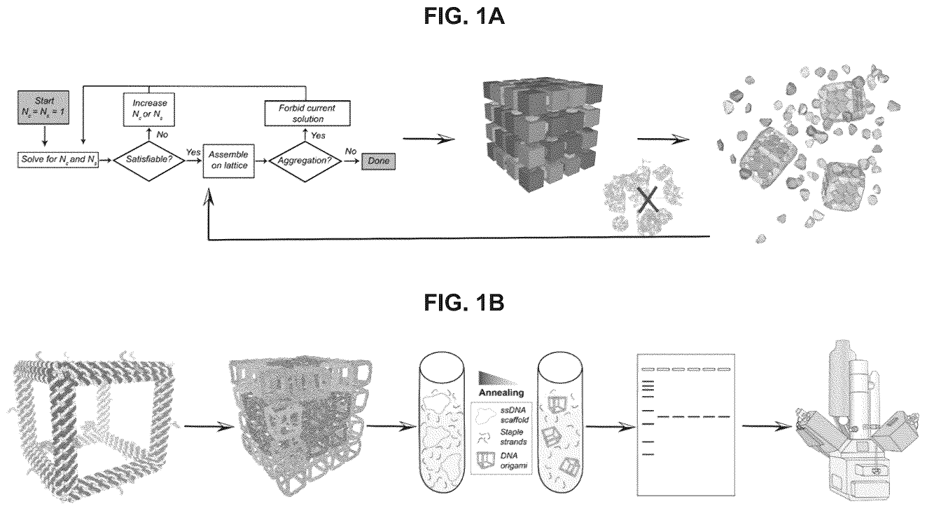

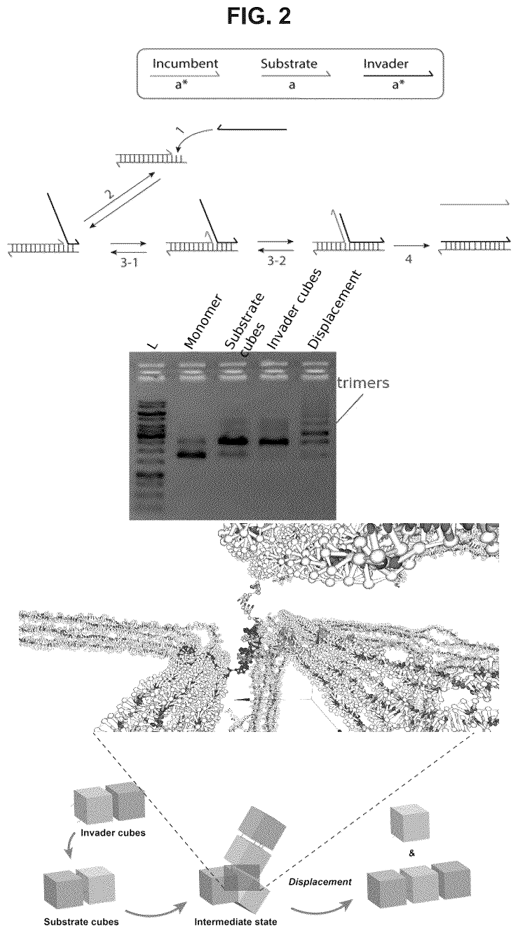

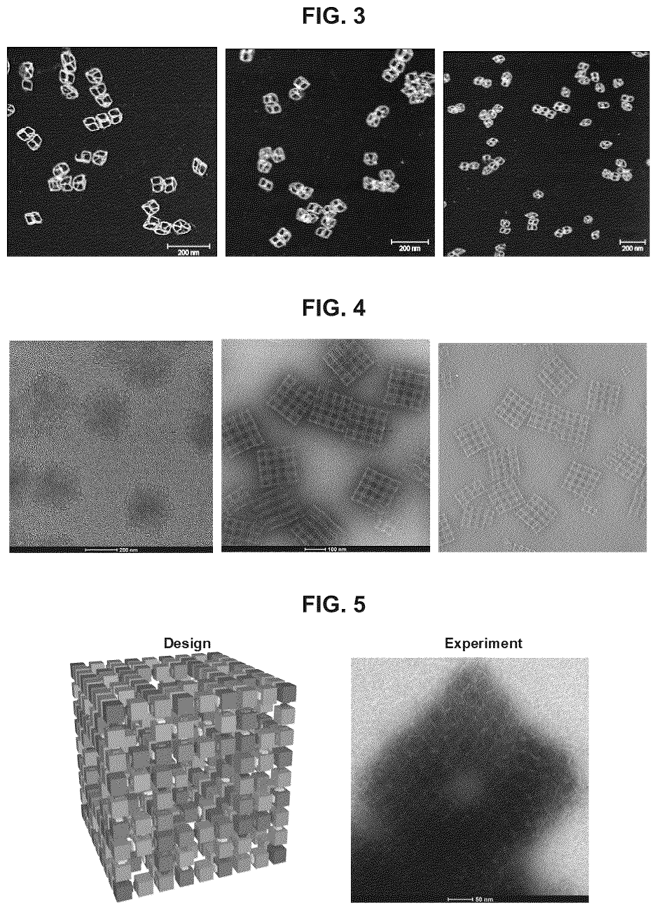

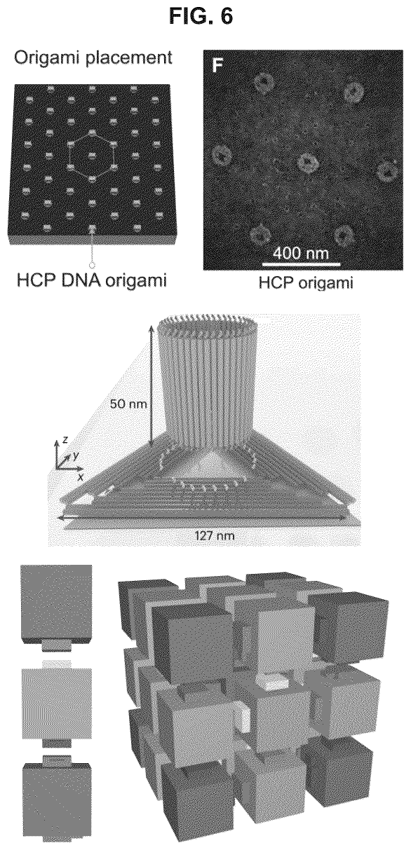

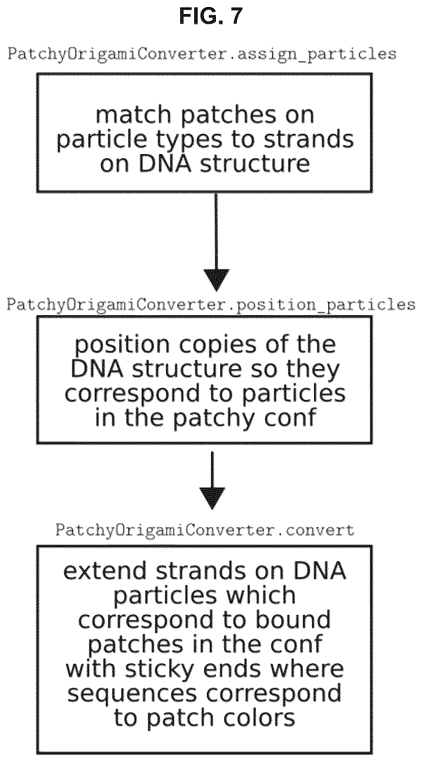

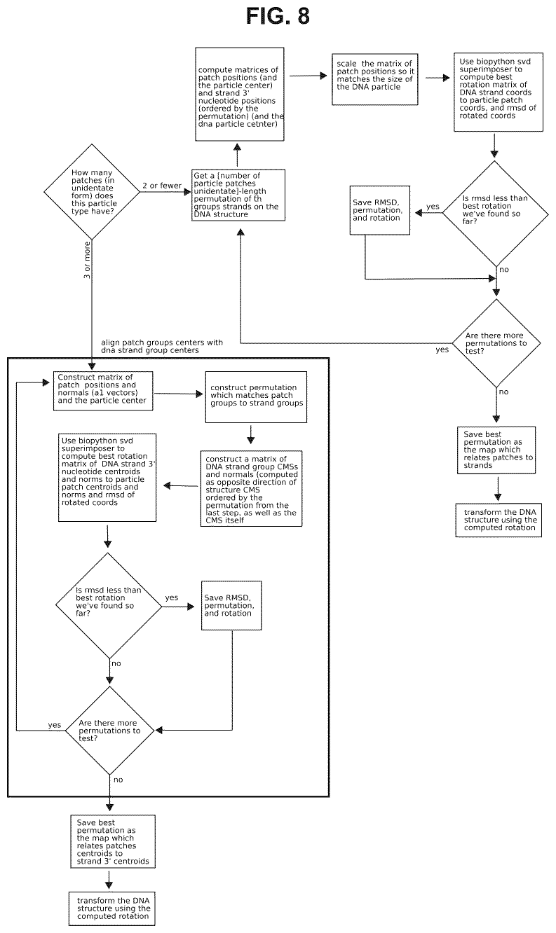

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points, or as described above in the definition of “about.”

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers a temperature of about 15-30° C., including all integers and endpoints within the specified range. In another aspect, “room temperature” refers a temperature of about 15-30° C.; about 20-30° C.; about 22-30° C.; about 25-30° C.; about 27-30° C.; about 15-22° C.; about 15-25° C.; about 15-27° C.; about 20-22° C.; about 20-25° C.; about 20-27° C.; about 22-25° C.; about 22-27° C.; about 25-27° C.; about 15° C.±10%; about 20° C.±10%; about 22° C.±10%; about 25° C.±10%; about 27° C.±10%; ~20° C., ~22° C., ~25° C., or ~27° C., at standard atmospheric pressure.

The terms “control,” or “reference” are used herein interchangeably. A “reference” or “control” level may be a predetermined value or range, which is employed as a baseline or benchmark against which to assess a measured result. “Control” also refers to control experiments.

As used herein, the terms “polycube” and “polycube assembly” refer to a three-dimensional assembly comprising two or more cube DNA origami nanostructures.

As used herein, the term “helical bundle” refers to an aggregate comprising a plurality of parallel helical molecules.

As used herein, the terms “staple strand,” “positioning strand,” and “overhang” are interchangeably used to refer to a DNA strand attached to a scaffold, wherein the DNA strand is designed to be complementary to another staple strand on another scaffold, thereby promoting the association of the two scaffolds via the binding of the two staple strands.

As used herein, the term “blocker strand” refers to a DNA strand designed to be complementary to a specific staple strand, thereby preventing the association of that staple strand to its complementary strand on another DNA nanostructure.

As used herein, the term “cube type” refers to a set of cube DNA origami nanostructures which share a specific set of sequences.

As used herein, the term “carbon nanotube circuit” refers to a 3D integrated circuit with at least one component comprising carbon nanotubes, e.g., carbon nanotube field-effect transistors (CNTFETs).

One of the key challenges of nanoscale manufacturing is precise positioning of different functional materials in 3D. To achieve this, a 3D nanomanufacturing platform was developed based on cubic-shaped small building blocks (30 nm per side length) that can be connected to each other through complementary interfaces. The building blocks are manufactured out of a DNA scaffold which can be further functionalized, e.g., by attaching proteins, metals, or other molecules and materials that can be cross-linked with DNA. The platform also includes computational optimization tool to prevent misassembly into undesired structures. Thus, for an arbitrary provided 3D shape, our platform designs cube shaped building blocks and interactions between them that self-assemble into target shape, with applications for e.g., manufacturing of photonic devices, enzymatic cascades, and nanoscale scaffolding for precise positioning of material/molecules in 3D.

Described herein are methods for designing and preparing cube-shaped DNA nanostructure, where each face of the cube has single-stranded DNA overhangs. Two cubes with complementary strands on the cube face can connect. Thus, by designing different cubes with different single-stranded regions on their face, we can connect multiple cubes together by self-assembly to build larger 3D structure. One of the main obstacles of construction by self-assembly is misassembled states (interactions between cubes that are not supposed to be together in the final larger structure). To avoid misassembly, this pipeline involves a computational tool that runs simulations and optimizes the design of complementary regions to avoid this misassembled state. For input of an arbitrary shape of a nanostructure, our pipeline finds interactions that lead to the assembly of a target structure in high yield. The cubes can be functionalized by attaching to them carbon nanotubes, gold nanoparticles, proteins, drugs, other molecules, or any other materials that can be crosslinked with DNA handles. Thus, the pipeline provides a general 3D precise positioning platform at nanoscale, that can bridge distances of hundreds of nanometers or larger.

(1) Currently, most 3D biomolecular shapes are very floppy. The DNA nanocube is very rigid, allowing us to scale the self-assembly into large nanostructures, as evidenced by assembling the larges finite-sized 3D DNA nanostructure to date.

(2) The pipeline described herein features computer simulations that can simulate assembly of 3D nanostructures in order to obtain high yields of the specific target assembly.

The described assembly methods can achieve larger 3D multicomponent structures from monomers than currently available by other approaches thanks to the individual building block design as well as the computational pipeline support.

The polynucleotides described herein include variants that have substitutions, deletions, and/or additions that can involve one or more nucleotides. The variants can be altered in coding regions, non-coding regions, or both. Alterations in the coding regions can produce conservative or non-conservative amino acid substitutions, deletions, or additions. Especially preferred among these are silent substitutions, additions, and deletions, which do not alter the properties and activities of the binding.

Further embodiments described herein include nucleic acid molecules comprising polynucleotides having nucleotide sequences about 50%, 55%, 60%, 65%, 70%, 75%, 80%, 85%, 90%, 91%, 92%, 93%, 94%, 95%, 96%, 97%, 98%, or 99% identical, and more preferably at least about 90-99% or 100% identical to (a) nucleotide sequences, or degenerate, homologous, or codon-optimized variants thereof, encoding polypeptides having the amino acid sequences in SEQ ID NO: 1-1181; (b) nucleotide sequences, or degenerate, homologous, or codon-optimized variants thereof, encoding polypeptides having the amino acid sequences in SEQ ID NO: 1-1181; and (c) nucleotide sequences capable of hybridizing to the complement of any of the nucleotide sequences in (a) or (b) above and capable of expressing functional polypeptides of amino acid sequences in SEQ ID NO: 1-1181.

By a polynucleotide having a nucleotide sequence at least, for example, 90-99% “identical” to a reference nucleotide sequence of SEQ ID NO: 1-1181 is intended that the nucleotide sequence of the polynucleotide be identical to the reference sequence except that the polynucleotide sequence can include up to about 10 to 1 point mutations, additions, or deletions per each 100 nucleotides of the reference nucleotide sequence of SEQ ID NO: 1-1181.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at the 5′- or 3′-terminal positions of the reference nucleotide sequence or anywhere between those terminal positions, interspersed either individually among nucleotides in the reference sequence or in one or more contiguous groups within the reference sequence. The same is applicable to polypeptide sequences about at least 90-99% identical to a reference polypeptide sequence.

As noted above, two or more polynucleotide sequences can be compared by determining their percent identity. Two or more amino acid sequences likewise can be compared by determining their percent identity. The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2: 4 82-489 (1981). This algorithm can be extended to use with peptide sequences using the scoring matrix developed by Dayhoff, Atlas of Protein Sequences and Structure, Dayhoff ed., 5 Suppl. 3: 353-358, National Biomedical Research Foundation, Washington, D.C., USA, and normalized by Gribskov, Nucl. Acids Res. 14(6): 6745-6763 (1986).

For example, due to the degeneracy of the genetic code, one having ordinary skill in the art will recognize that a large number of the nucleic acid molecules having a sequence at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequence shown in SEQ ID NO: 1-1181, or degenerate, homologous, or codon-optimized variants thereof.

The polynucleotides described herein include those encoding mutations, variations, substitutions, additions, deletions, and particular examples of the polypeptides described herein. For example, guidance concerning how to make phenotypically silent amino acid substitutions is provided in Bowie et al., Science 247: 1306-1310 (1990), wherein the authors indicate that proteins are surprisingly tolerant of amino acid substitutions.

Another embodiment described herein is a polynucleotide vector comprising one or more nucleotide sequences described herein.

Another embodiment described herein is a cell comprising one or more nucleotide sequences described herein or a polynucleotide vector described herein.

Another embodiment described herein is a process for manufacturing one or more of the nucleotide sequence described herein or a polypeptide encoded by the nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a means for manufacturing one or more of the nucleotide sequences described herein or a polypeptide encoded by a nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a nucleotide sequence or a polypeptide encoded by the nucleotide sequence produced by the method or the means described herein

Another embodiment described herein is the use of an effective amount of a polypeptide encoded by one or more of the nucleotide sequences described herein in 1-1181.

Another embodiment described herein is a research tool comprising a polypeptide encoded by a nucleotide sequence described herein.

Another embodiment described herein is a reagent comprising a polypeptide encoded by a nucleotide sequence described herein.

One embodiment described herein is a cube DNA origami nanostructure, comprising: a ssDNA scaffold folded into a cubic frame, comprising: six helical bundles (HB) defining six edges of the cubic frame; and a plurality of staple strands, each staple strand being positioned proximate to a center of an edge of the cubic frame. In one aspect, the plurality of staple strands comprises pairs of complementary sequences configured to promote binding between two edges of two cube DNA origami nanostructures. In another aspect, the HBs comprise 6-12 helical turns. In another aspect, the cube DNA origami nanostructure comprises mitered vertices. In another aspect, the plurality of staple strands comprises a plurality of types of orthogonal staple strands. In another aspect, the cubic frame comprises six faces, each face comprising four edges; the plurality of staple strands comprises four types of orthogonal staple strands; and each one of the four edges of each face comprises one of the four types of orthogonal staple strands such that the four edges of each face comprise all four types of orthogonal staple strands. In another aspect, the melting temperature of the plurality of staple strands is below 55° C. In another aspect, the nanostructure further comprises carbon nanotubes, gold nanoparticles, proteins, or other molecules. In another aspect, the nanostructure further comprises carbon nanotubes. In another aspect, the nanostructure further comprises a positioning DNA strand, wherein: the positioning DNA strand is located on an exterior surface of the carbon nanotubes; and the positioning DNA strand is complementary to another sequence within the nanostructure.

Another embodiment described herein is a polycube assembly, comprising: a plurality of the cube DNA origami nanostructures described herein, wherein: each of the plurality of the cube DNA origami nanostructures belongs to one of a plurality of cube types; the plurality of staple strands of the cube DNA origami nanostructures of each of the plurality of cube types independently comprise a set of staple strands; each of the cube types has a unique set of staple strands; and the plurality of the staple strands of the plurality of cube types are designed to promote binding between the plurality of cube types, whereby the cube DNA origami nanostructures assemble into a specific 3D configuration. In one aspect, each cube DNA origami nanostructure comprises a set of six faces comprising a top, a bottom, a left, a right, a front, and a back; each face comprises four edges; and each edge of each face of each of the plurality of cube types has a unique sequence. In another aspect, each edge of each face of each of the plurality of cube types has a sequence selected from SEQ ID NO: 1-1181. In another aspect, the polycube assembly further comprises one or more blocker strands configured to prevent aggregation of the cube DNA origami nanostructures. In another aspect, the plurality of cube types comprises 2-19 cube types. In another aspect, each of the cube DNA origami nanostructures of each of the plurality of cube types comprises a unique set of sequences selected from SEQ ID NO: 1-1181. In another aspect, the specific 3D configuration is selected from the group consisting of: a 3D cross, a hollow 3×3×3 cube, a human only polycube, a wolf only polycube, a multifarious werewolf polycube, fractal Menger cube M₂, a simple cubic crystal lattice, and a solid 4×4×4 assembly. In another aspect, the polycube assembly further comprises carbon nanotubes, gold nanoparticles, proteins, or other molecules. In another aspect, the polycube assembly further comprises carbon nanotubes. In another aspect, the polycube assembly further comprises a positioning DNA strand, wherein: the positioning DNA strand is located on an exterior surface of the carbon nanotubes; and the positioning DNA strand is complementary to another sequence within the polycube assembly.

Another embodiment described herein is a method of making a three-dimensional (3D) DNA nanocube capable of self-assembly into larger 3D multicomponent structures, the method comprising: obtaining one or more DNA origami monomers comprising staple strands; and incubating the DNA origami monomers for in a solution a period of time and at a temperature sufficient for self-assembly into the DNA nanocube. In one aspect, the one or more DNA origami monomers comprise a ssDNA scaffold. In another aspect, the one or more DNA origami monomers comprise an M13mp18 scaffold. In another aspect, the incubating step is performed in a buffer. In another aspect, the incubating step is performed in TAE Mg²⁺ buffer.

In another aspect, the method further comprises purifying the DNA nanocubes via gel electrophoresis and extracting a monomer band. In another aspect, one or more of the staple strands is hybridized to a DNA-CNT conjugate, the DNA-CNT conjugate comprising a carbon nanotube and a positioning sequence, the positioning sequence being complementary to the one or more staple strands.

Another embodiment described herein is a method of making a polycube assembly, the method comprising: providing a solution comprising a plurality of DNA origami nanostructures, each of the plurality of the DNA origami nanostructures comprising: a ssDNA scaffold folded into a cubic frame, comprising: six helical bundles (HB) defining six edges of the cubic frame; and a plurality of staple strands, each staple strand being positioned proximate to a center of an edge of the cubic frame; heating the solution to a temperature for a period of time such that plurality of DNA origami nanostructures assembles into a polycube assembly. In another aspect, each of the plurality of the cube DNA origami nanostructures belongs to one of a plurality of cube types; the plurality of staple strands of the cube DNA origami nanostructures of each of the plurality of cube types independently comprise a set of staple strands; each of the cube types has a unique set of staple strands; and the plurality of the staple strands of the plurality of cube types are designed to promote assembly of the plurality of DNA origami nanostructures assembles into the polycube assembly. In another aspect, each cube DNA origami nanostructure comprises a set of six faces comprising a top, a bottom, a left, a right, a front, and a back; each face comprises four edges; and each edge of each face of each of the plurality of cube types has a unique sequence. In another aspect, each edge of each face of each of the plurality of cube types has a unique sequence selected from SEQ ID NO: 1-1181. In another aspect, the method further comprises adding one or more blocker strands configured to prevent aggregation of the cube DNA origami nanostructures. In another aspect, the plurality of cube types comprises 2-19 cube types. In another aspect, each of the cube DNA origami nanostructures of each of the plurality of cube types comprises a unique set of sequences selected from SEQ ID NO: 1-1181. In another aspect, the polycube assembly is selected from the group consisting of: a 3D cross, a hollow 3×3×3 cube, a human only polycube, a wolf only polycube, a multifarious werewolf polycube, fractal Menger cube M₂, a simple cubic crystal lattice, and a solid 4×4×4 assembly. In another aspect, the plurality of DNA origami nanostructures further comprises carbon nanotubes, gold nanoparticles, proteins, or other molecules. In another aspect, the plurality of DNA origami nanostructures further comprises carbon nanotubes, the method further comprising: positioning the carbon nanotubes in one or more 2D or 3D junctions, thereby forming one or more carbon nanotube circuits.

Another embodiment described herein is a system for designing a three-dimensional structure assembled from multiple DNA nanocubes, the system comprising: a processor configured to execute instructions to: conduct one or more computer simulations to identify a minimal number of cube types required to assemble a target three-dimensional structure; assign complementary DNA sequences to faces of the cube types to define binding interactions between the cube types; and generate an output specifying DNA origami monomers for assembly into the target three-dimensional structure. In one aspect, the computer simulations comprise a satisfiability solver to identify solutions that minimize misassembly states. In another aspect, the processor is further configured to execute instructions to conduct patchy particle simulations to evaluate kinetics and success of assembly for the target three-dimensional structure. In another aspect, the processor is further configured to execute instructions to identify aggregated structures during the patchy particle simulations and feed the aggregated structures back into the satisfiability solver to refine the design process. In another aspect, the target three-dimensional structure comprises a 3D cross, a hollow 3×3×3 cube, a human only polycube, a wolf only polycube, a multifarious werewolf polycube, fractal Menger cube M₂, a simple cubic crystal lattice, or a solid 4×4×4 assembly, and wherein the satisfiability solver determines a minimal number of cube types and a ratio of each cube type required for assembly. In another aspect, the target three-dimensional structure comprises a carbon nanotube circuit.

Another embodiment described herein is a method of designing a three-dimensional structure assembled from multiple DNA nanocubes, the method comprising: conducting one or more computer simulations to identify a minimal number of cube types required to assemble a target three-dimensional structure; assigning complementary DNA sequences to faces of the cube types to define binding interactions between the cube types; and generating an output specifying DNA origami monomers for assembly into the target three-dimensional structure. In one aspect, the target three-dimensional structure comprises a carbon nanotube circuit. In another aspect, the target three-dimensional structure comprises DNA nanocubes attached to gold nanoparticles, proteins, or other molecules.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A cube DNA origami nanostructure, comprising:
   a ssDNA scaffold folded into a cubic frame, comprising:
      six helical bundles (HB) defining six edges of the cubic frame; and
      a plurality of staple strands, each staple strand being positioned proximate to a center of an edge of the cubic frame.
Clause 2. The nanostructure of clause 1, wherein the plurality of staple strands comprises pairs of complementary sequences configured to promote binding between two edges of two cube DNA origami nanostructures.
Clause 3. The nanostructure of clause 1 or 2, wherein the HBs comprise 6-12 helical turns.
Clause 4. The nanostructure of any one of clauses 1-3, wherein the cube DNA origami nanostructure comprises mitered vertices.
Clause 5. The nanostructure of any one of clauses 1-4, wherein the plurality of staple strands comprises a plurality of types of orthogonal staple strands.
Clause 6. The nanostructure of any one of clauses 1-5, wherein:
   the cubic frame comprises six faces, each face comprising four edges;
   the plurality of staple strands comprises four types of orthogonal staple strands; and
   each one of the four edges of each face comprises one of the four types of orthogonal staple strands such that the four edges of each face comprise all four types of orthogonal staple strands.
Clause 7. The nanostructure of any one of clauses 1-6, wherein the melting temperature of the plurality of staple strands is below 55° C.
Clause 8. The nanostructure of any one of clauses 1-7, further comprising carbon nanotubes, gold nanoparticles, proteins, or other molecules.
Clause 9. The nanostructure of any one of clauses 1-8, further comprising carbon nanotubes.
Clause 10. The nanostructure of any one of clauses 1-9, further comprising a positioning DNA strand, wherein:
   the positioning DNA strand is located on an exterior surface of the carbon nanotubes; and
   the positioning DNA strand is complementary to another sequence within the nanostructure.
Clause 11. A polycube assembly, comprising:
   a plurality of the cube DNA origami nanostructures of any one of clauses 1-10, wherein:
   each of the plurality of the cube DNA origami nanostructures belongs to one of a plurality of cube types;
   the plurality of staple strands of the cube DNA origami nanostructures of each of the plurality of cube types independently comprise a set of staple strands;
   each of the cube types has a unique set of staple strands; and
   the plurality of the staple strands of the plurality of cube types are designed to promote binding between the plurality of cube types, whereby the cube DNA origami nanostructures assemble into a specific 3D configuration.
Clause 12. The polycube assembly of clause 11, wherein:
   each cube DNA origami nanostructure comprises a set of six faces comprising a top, a bottom, a left, a right, a front, and a back;
   each face comprises four edges; and
   each edge of each face of each of the plurality of cube types has a unique sequence.
Clause 13. The polycube assembly of clause 11 or 12, wherein each edge of each face of each of the plurality of cube types has a sequence selected from SEQ ID NO: 1-1181.
Clause 14. The polycube assembly of any one of clauses 11-13, further comprising one or more blocker strands configured to prevent aggregation of the cube DNA origami nanostructures.
Clause 15. The polycube assembly of any one of clauses 11-14, wherein the plurality of cube types comprises 2-19 cube types.
Clause 16. The polycube assembly of any one of clauses 11-15, wherein each of the cube DNA origami nanostructures of each of the plurality of cube types comprises a unique set of sequences selected from SEQ ID NO: 1-1181.
Clause 17. The polycube assembly of any one of clauses 11-16, wherein the specific 3D configuration is selected from the group consisting of: a 3D cross, a hollow 3×3×3 cube, a human only polycube, a wolf only polycube, a multifarious werewolf polycube, fractal Menger cube M₂, a simple cubic crystal lattice, and a solid 4×4×4 assembly.
Clause 18. The polycube assembly of any one of clauses 11-17, further comprising carbon nanotubes, gold nanoparticles, proteins, or other molecules.
Clause 19. The polycube assembly of any one of clauses 11-18, further comprising carbon nanotubes.
Clause 20. The polycube assembly of clause 19, further comprising a positioning DNA strand, wherein:
   the positioning DNA strand is located on an exterior surface of the carbon nanotubes; and
   the positioning DNA strand is complementary to another sequence within the polycube assembly.
Clause 21. A method of making a three-dimensional (3D) DNA nanocube capable of self-assembly into larger 3D multicomponent structures, the method comprising:
   obtaining one or more DNA origami monomers comprising staple strands; and
   incubating the DNA origami monomers for in a solution a period of time and at a temperature sufficient for self-assembly into the DNA nanocube.
Clause 22. The method of clause 21, wherein the one or more DNA origami monomers comprise a ssDNA scaffold.
Clause 23. The method of clause 21 or 22, wherein the one or more DNA origami monomers comprise an M13mp18 scaffold.
Clause 24. The method of any one of clauses 21-23, wherein the incubating step is performed in a buffer.
Clause 25. The method of any one of clauses 21-24, wherein the incubating step is performed in TAE Mg²⁺ buffer.
Clause 26. The method of any one of clauses 21-25, further comprising purifying the DNA nanocubes via gel electrophoresis and extracting a monomer band.
Clause 27. The method of any one of clauses 21-26, wherein one or more of the staple strands is hybridized to a DNA-CNT conjugate, the DNA-CNT conjugate comprising a carbon nanotube and a positioning sequence, the positioning sequence being complementary to the one or more staple strands.
Clause 28. A method of making a polycube assembly, the method comprising:
   providing a solution comprising a plurality of DNA origami nanostructures, each of the plurality of the DNA origami nanostructures comprising:
   a ssDNA scaffold folded into a cubic frame, comprising:
   six helical bundles (HB) defining six edges of the cubic frame; and
   a plurality of staple strands, each staple strand being positioned proximate to a center of an edge of the cubic frame;
   heating the solution to a temperature for a period of time such that plurality of DNA origami nanostructures assembles into a polycube assembly.
Clause 29. The method of clause 28, wherein:
   each of the plurality of the cube DNA origami nanostructures belongs to one of a plurality of cube types;
   the plurality of staple strands of the cube DNA origami nanostructures of each of the plurality of cube types independently comprise a set of staple strands;
   each of the cube types has a unique set of staple strands; and
   the plurality of the staple strands of the plurality of cube types are designed to promote assembly of the plurality of DNA origami nanostructures assembles into the polycube assembly.
Clause 30. The method of clause 28 or 29, wherein
   each cube DNA origami nanostructure comprises a set of six faces comprising a top, a bottom, a left, a right, a front, and a back;
   each face comprises four edges; and
   each edge of each face of each of the plurality of cube types has a unique sequence.
Clause 31. The method of clause 30, wherein each edge of each face of each of the plurality of cube types has a unique sequence selected from SEQ ID NO: 1-1181.
Clause 32. The method of any one of clauses 28-31, further comprising adding one or more blocker strands configured to prevent aggregation of the cube DNA origami nanostructures.
Clause 33. The method of any one of clauses 28-32, wherein the plurality of cube types comprises 2-19 cube types.
Clause 34. The method of any one of clauses 28-33, wherein each of the cube DNA origami nanostructures of each of the plurality of cube types comprises a unique set of sequences selected from SEQ ID NO: 1-1181.
Clause 35. The method of any one of clauses 28-34, wherein the polycube assembly is selected from the group consisting of: a 3D cross, a hollow 3×3×3 cube, a human only polycube, a wolf only polycube, a multifarious werewolf polycube, fractal Menger cube M₂, a simple cubic crystal lattice, and a solid 4×4×4 assembly.
Clause 36. The method of any one of clauses 28-35, wherein the plurality of DNA origami nanostructures further comprises carbon nanotubes, gold nanoparticles, proteins, or other molecules.
Clause 37. The method of any one of clauses 28-36, wherein the plurality of DNA origami nanostructures further comprises carbon nanotubes, the method further comprising:
   positioning the carbon nanotubes in one or more 2D or 3D junctions, thereby forming one or more carbon nanotube circuits.
Clause 38. A system for designing a three-dimensional structure assembled from multiple DNA nanocubes, the system comprising:
   a processor configured to execute instructions to:
   conduct one or more computer simulations to identify a minimal number of cube types required to assemble a target three-dimensional structure;
   assign complementary DNA sequences to faces of the cube types to define binding interactions between the cube types; and
   generate an output specifying DNA origami monomers for assembly into the target three-dimensional structure.
Clause 39. The system of clause 38, wherein the computer simulations comprise a satisfiability solver to identify solutions that minimize misassembly states.
Clause 40. The system of clause 38 or 29, wherein the processor is further configured to execute instructions to conduct patchy particle simulations to evaluate kinetics and success of assembly for the target three-dimensional structure.
Clause 41. The system of clause 40, wherein the processor is further configured to execute instructions to identify aggregated structures during the patchy particle simulations and feed the aggregated structures back into the satisfiability solver to refine the design process.
Clause 42. The system of any one of clauses 38-41, wherein the target three-dimensional structure comprises a 3D cross, a hollow 3×3×3 cube, a human only polycube, a wolf only polycube, a multifarious werewolf polycube, fractal Menger cube M₂, a simple cubic crystal lattice, or a solid 4×4×4 assembly, and wherein the satisfiability solver determines a minimal number of cube types and a ratio of each cube type required for assembly.
Clause 43. The system of any one of clauses 38-42, wherein the target three-dimensional structure comprises a carbon nanotube circuit.
Clause 44. A method of designing a three-dimensional structure assembled from multiple DNA nanocubes, the method comprising:
   conducting one or more computer simulations to identify a minimal number of cube types required to assemble a target three-dimensional structure;
   assigning complementary DNA sequences to faces of the cube types to define binding interactions between the cube types; and
   generating an output specifying DNA origami monomers for assembly into the target three-dimensional structure.
Clause 45. The method of clause 44, wherein the target three-dimensional structure comprises a carbon nanotube circuit.
Clause 46. The method of clause 44 or 45, wherein the target three-dimensional structure comprises DNA nanocubes attached to gold nanoparticles, proteins, or other molecules.

### Examples

### Design of Cube DNA Origami (CDO) Subunits

The CDO was designed using ATHENA. Jun et al., Nucleic Acids Res. 49(18):10265-10274 (2021). CDO was constructed using an M13mp18 scaffold, with six helix bundles (HB) along each edge, and each consisting of approximately 9 helical turns. To enhance structural rigidity and maintain binding symmetry for hierarchical assemblies, a mitered vertex was employed. DNA handles (sticky end strands) were precisely positioned at the midpoint of each edge to facilitate the assembly of CDOs. Four orthogonal DNA handles were placed on each face of CDO to allow torsional control of face-binding interactions according to the polycube model. The structural rigidity and handles placement of CDO were validated through oxDNA molecular dynamics (MD) simulations. Poppleton et al., J. Open Source Software 8(81): 4693 (2023).

### Folding and Purification of CDO

CDO was assembled by mixing M13mp18 scaffold (50 nM) with a mixture of staples (500 nM per staple) and handles (1 μM per handle) in 1×TAE Mg²⁺ buffer (40 mM Tris, 20 mM acetic acid, 1 mM EDTA, 12.5 mM Mg²⁺ pH 8.0). The folding samples were annealed over 20 hours with the following protocol: from 75° C. to 65° C. at a rate of 1° C. per 5 minutes, from 65° C. to 40° C. at a rate of 0.1° C. per 4 minutes, from 40° C. to 20° C. at 0.1° C. per 2 minutes, then incubated at 20° C. in a thermal cycling device (ProFlex PCR System, Applied Biosystems™).

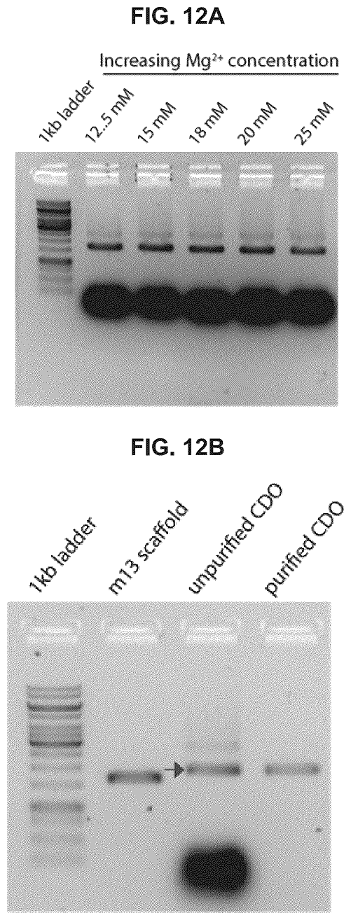

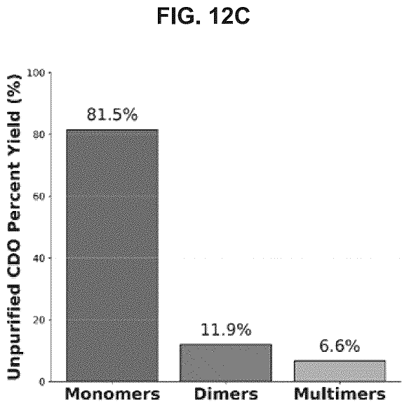

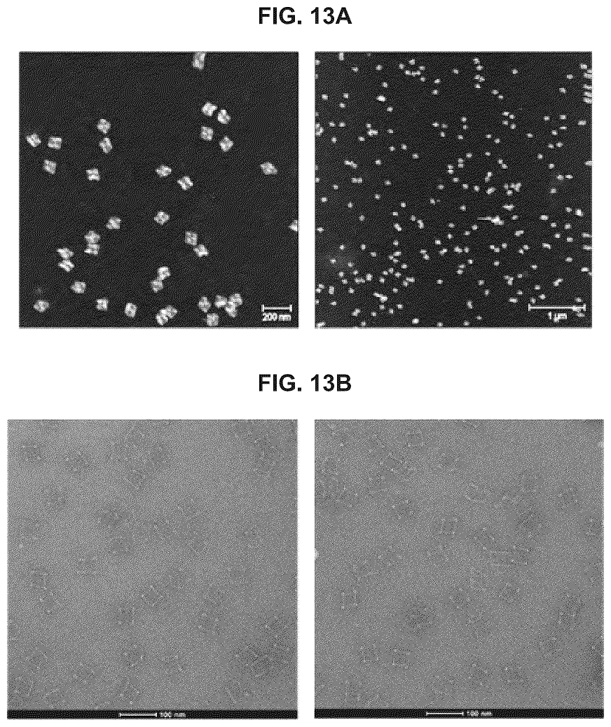

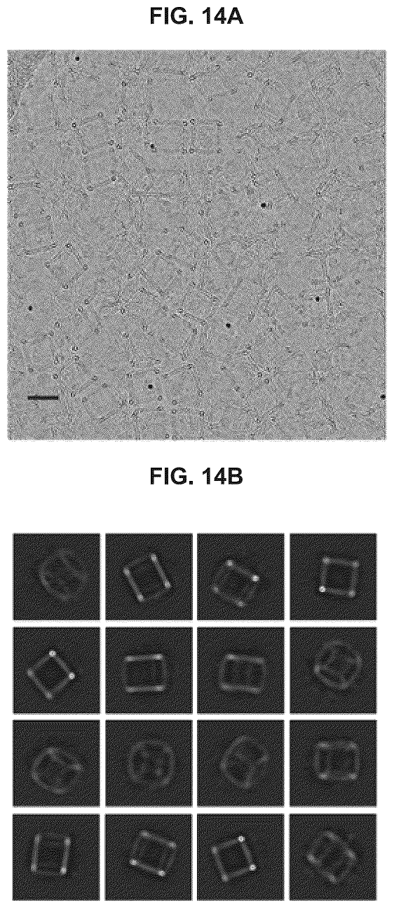

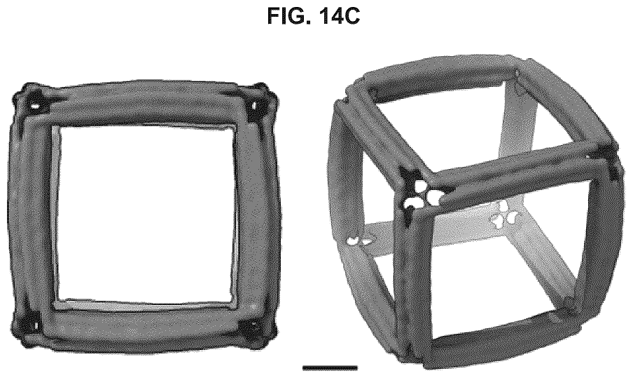

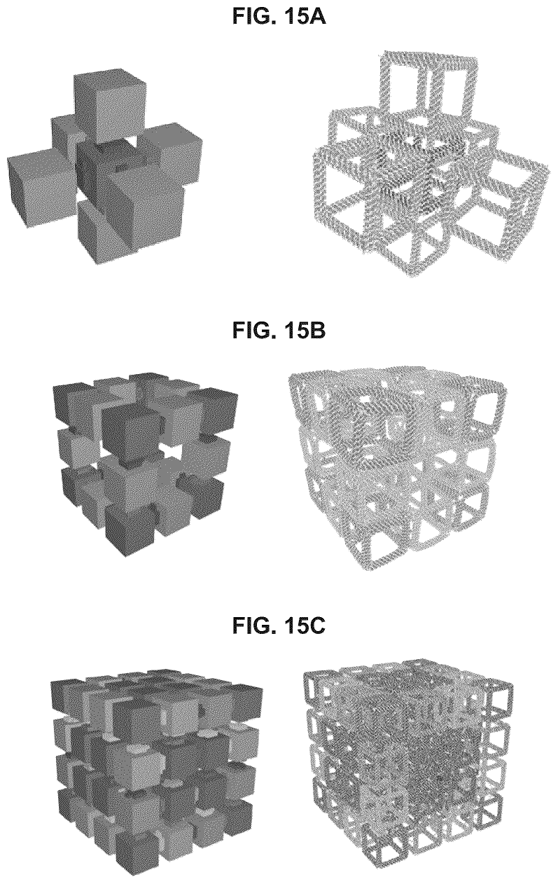

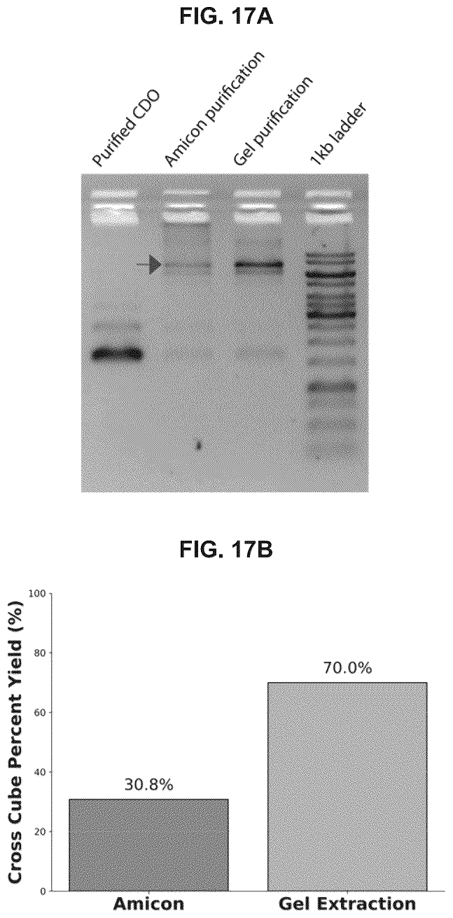

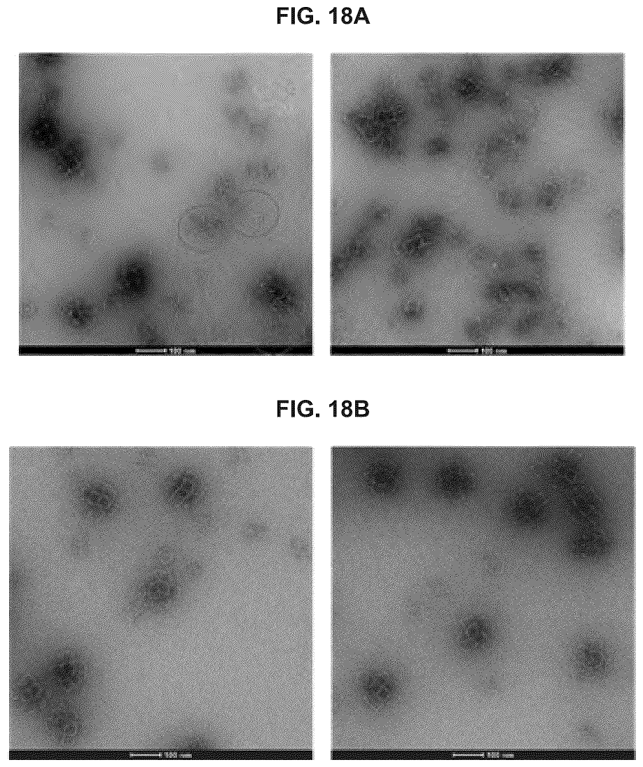

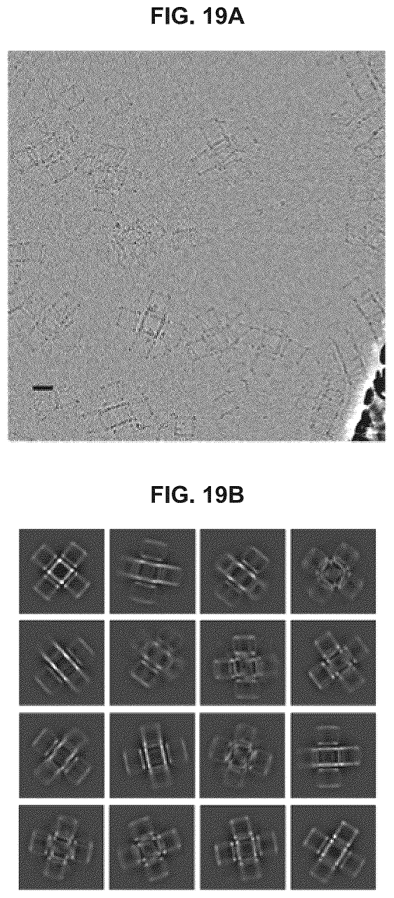

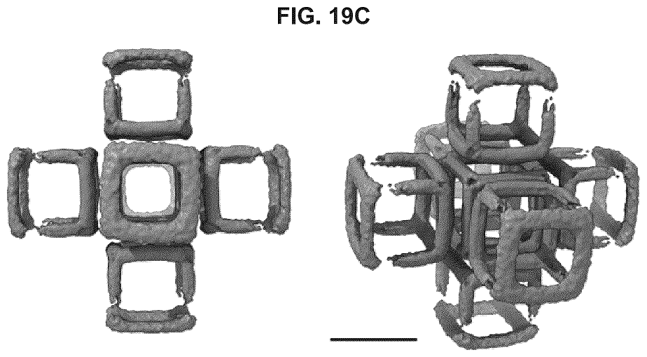

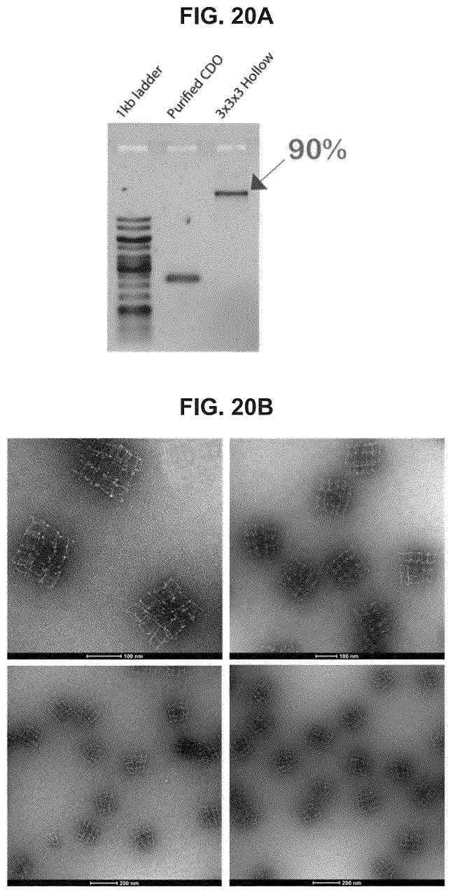

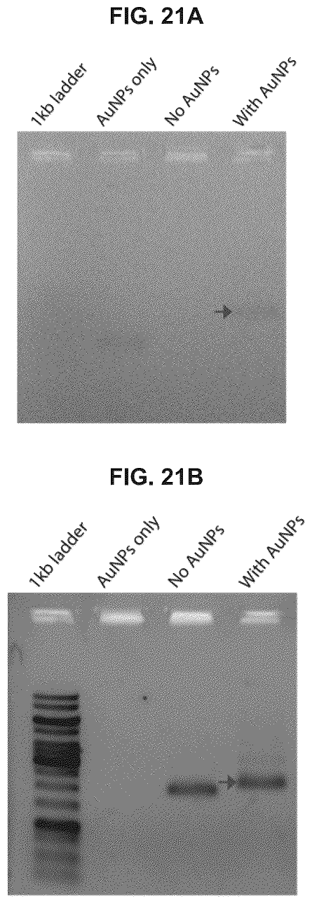

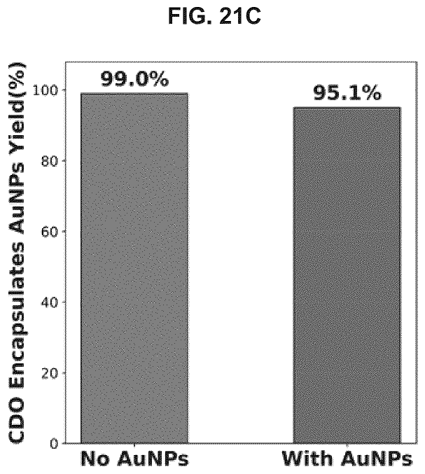

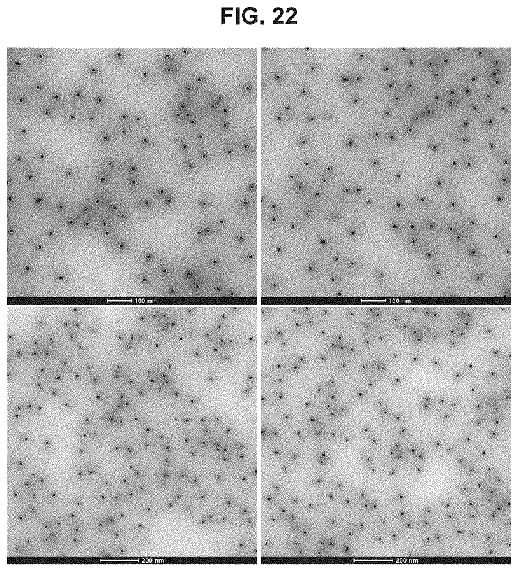

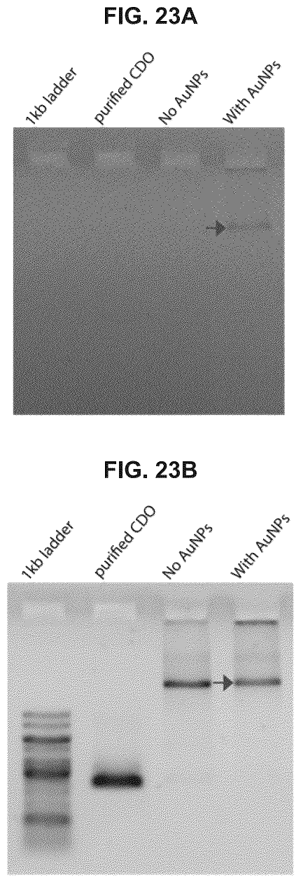

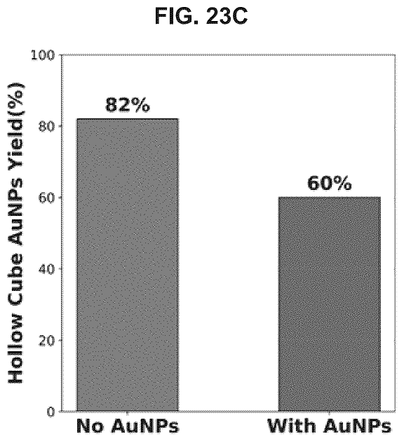

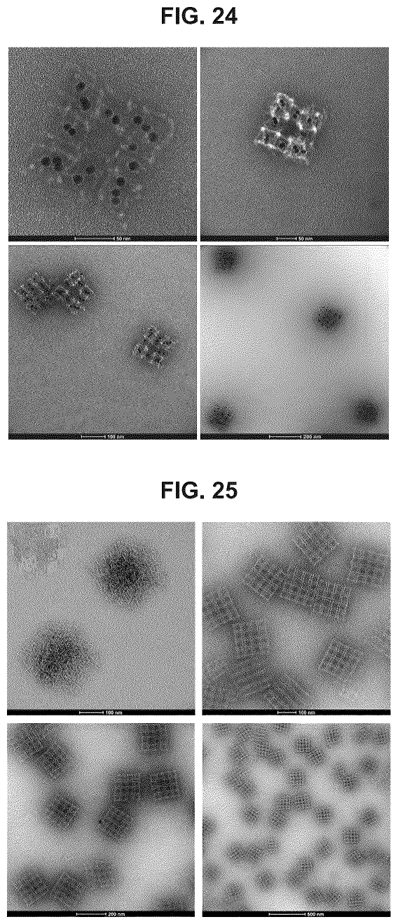

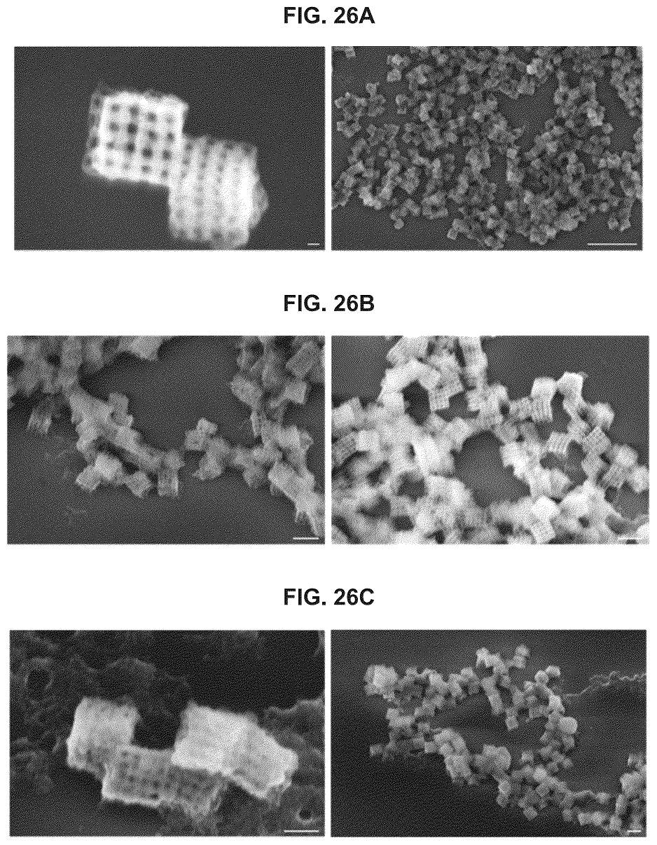

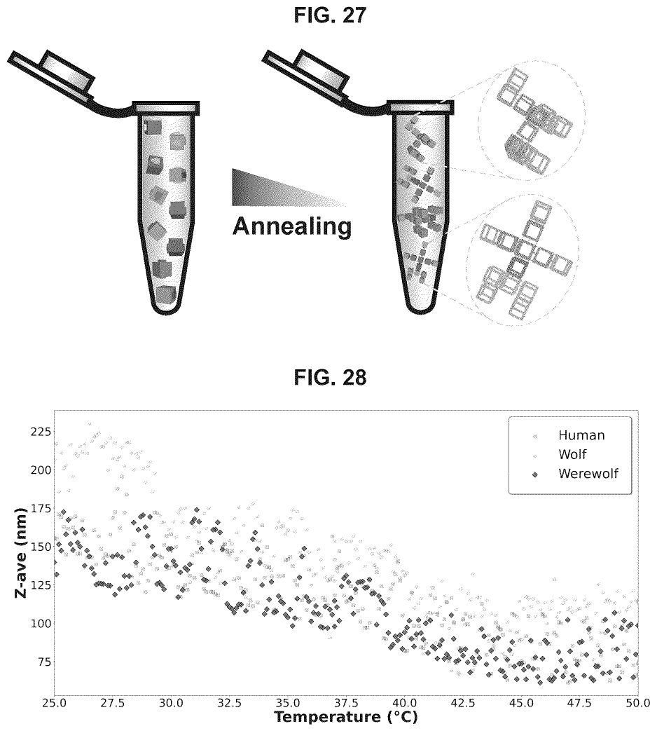

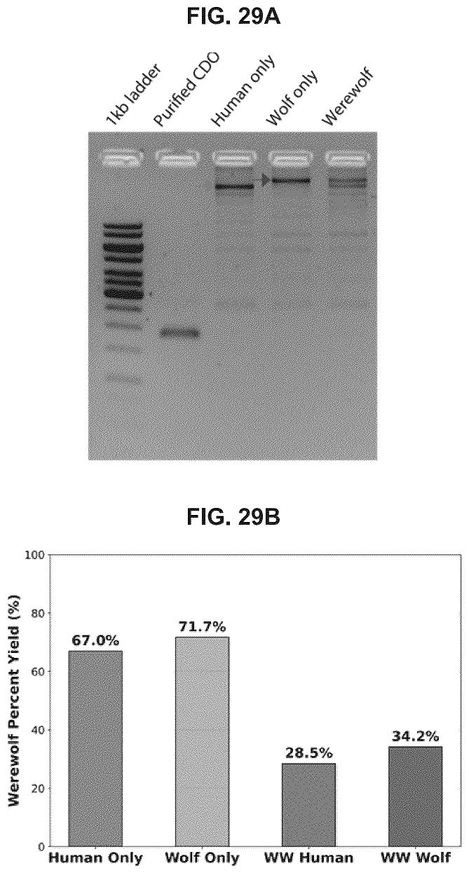

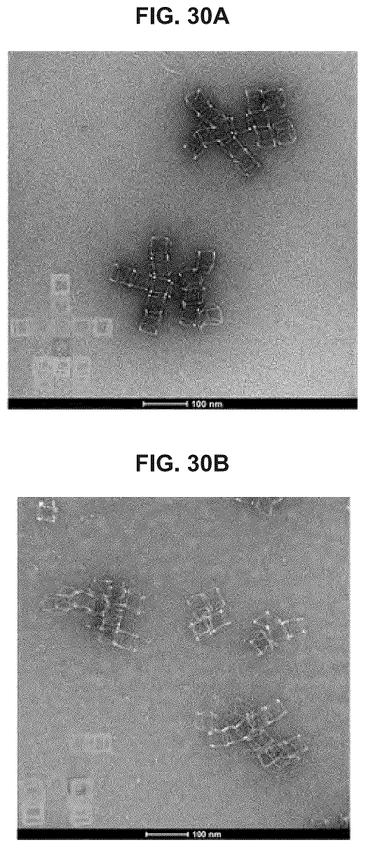

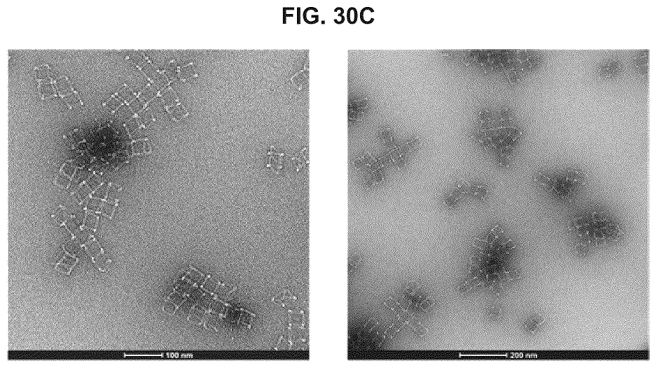

The folded samples were then purified using agarose gel electrophoresis (AGE) by excising the monomer band. In doing so, the excess staples, multimers, and misfolded structures were removed. The aggregate and multimer structures will significantly deteriorate the hierarchical assembly of cube structures (polycube) (FIG. 17). Briefly, the samples were loaded in 1% agarose with 1×TAE Mg²⁺ buffer and run at 100 V for 1.5 hours in an ice bath. The CDO monomer band was excised, cut into small pieces, and collected into Freeze'N Squeeze DNA gel extraction spin columns (Biorad) with 500 μL of 1×TAE Mg²⁺ buffer. The columns were centrifuged at 3000 rcf for 45 minutes at 4° C. to extract the monomers from the gel matrix. The CDO monomers were then concentrated using 100 kDa amicon ultrafiltration (Millipore Sigma). Amicon columns were first passivated with 1×TAE Mg²⁺ buffer, with the extracted solution from gel loaded into the column later. Centrifugation was performed at 2600 rcf for 5 mins, with 5 rounds of buffer exchange. An exemplary result of CDO folding and purification is shown in FIG. 12. The concentration of CDO was measured using NanoDrop at 260 nm absorbance. The samples were stored in a −20° C. freezer until further assembly.

### Agarose Gel Electrophoresis and Yield Quantification

Agarose gel electrophoresis (AGE) was used to characterize and quantify yields for polycube assemblies. Polycube structures were loaded in 0.4-1% agarose gels with 1×TAE Mg²⁺ buffer and 1×SYBR Safe (Thermo Fisher Scientific). The gel was run at 100 V for 1 to 1.5 hours in an ice bath. Gel percentage and run time were adjusted based on the size of the polycube assemblies.

ImageJ was used to quantify the yield of correctly assembled polycubes through gel densitometry. The band intensity of the target structure was divided by the total lane intensity to calculate the yield. For structures that are too large to migrate into the gel, such as the 4×4×4 solid cubes and the Menger cube, TEM image analysis was employed by capturing a set of zoomed-out images, and correctly formed structures were identified to quantify the yield. It is important to note that while imaging provides more structural details, there is a potential for bias and underestimation of yields, since native structures may undergo structural collapse during sample preparation and can dry out while being negatively-stained (ns) for TEM characterization.

### Determination of the Annealing Protocol for Polycubes

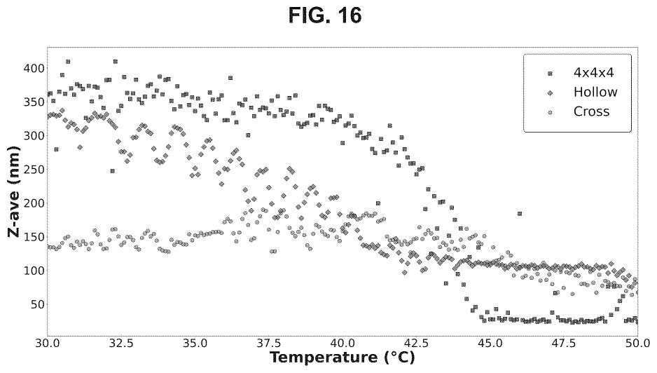

The annealing protocol was determined by monitoring the transition of CDO monomers to the aggregate stage across a range of temperatures, as indicated by an increase in particle size measured using Dynamic Light Scattering (DLS, Malvern Zetasizer Nano). The temperature protocol for DLS ranges from 53° C. to 20° C., with a cooling rate of 0.1° C. per 300 seconds, which identifies the optimal annealing window for each polycube. Since each structure can have between 1 and 6 face-binding interactions with different handle sets, the melting temperature for each design varies slightly. The purified CDO particle-type structures (7 nM, 80 μL) were loaded into a clean, disposable 40 μL cuvette with a cap (Malvern Panalytical) for measurement. After mixing to a final volume of 80 μL, 30 μL of mineral oil (CVS) was slowly deposited on top of the sample to minimize water evaporation caused by prolonged incubation at high temperatures during DLS measurements. The thermal profile was used to establish the annealing protocol for polycubes, starting just above the transition from the monomer stage to room temperature (RT). For finite-size polycube assemblies, the DLS measurement identifies a temperature range where dispersed monomer CDO particles begin to aggregate, as indicated by an increase in particle size, until a plateau was reached where no further growth was observed. This plateau marks the critical temperature for polycube self-assembly within this range. By monitoring size changes at decreasing temperatures, we observed that most polycubes begin to assemble at 45° C. and complete the assembly at 30° C. (FIG. 16, FIG. 28). The annealing protocol targets this broad temperature range. To ensure a monodisperse CDO solution prior to assembly, the sample was first incubated at 53° C. for 1 hour to account for thermodynamic variations such as sample preparation and buffer conditions. The protocol then proceeds, allowing the CDOs to aggregate as the temperature was gradually reduced to 25° C. This broader annealing range ensures a more reliable and consistent assembly process.

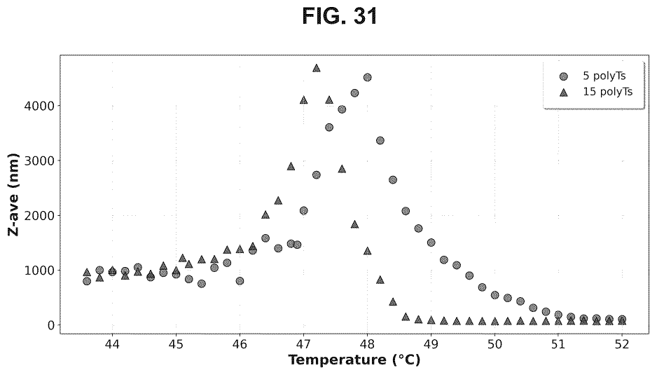

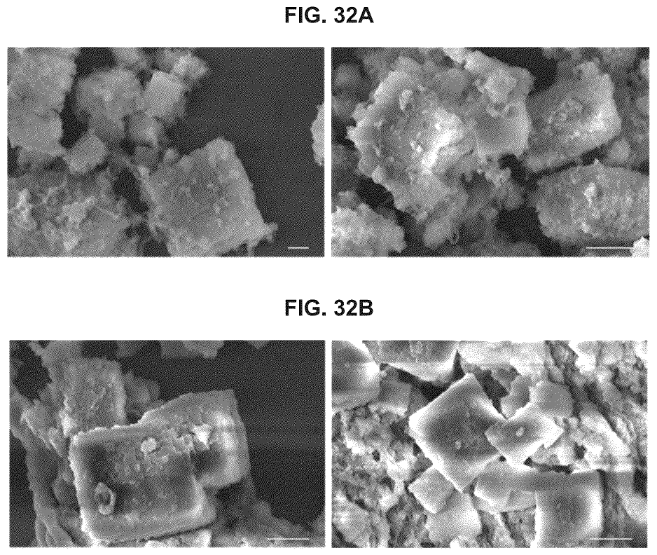

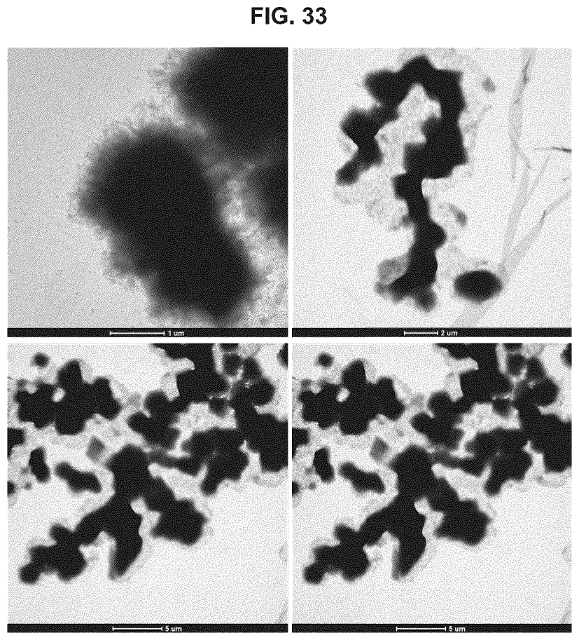

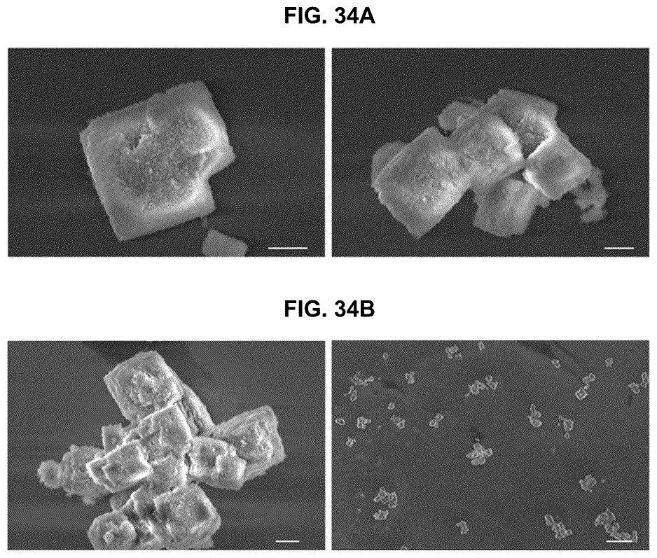

For simple cubic (SC) crystal lattice assembly, the same finite-size DLS protocol was applied. The DLS measurement identifies a narrower annealing window for the lattice assembly, as no further aggregation can be observed (FIG. 31). The annealing protocol begins with incubation at 53° C. for 1 hour to ensure a monodisperse CDO solution, followed by targeting this narrow temperature range to seed crystal growth and achieve precise formation.

### Polycube Assemblies

We computationally design orthogonal sequence sets and utilize DLS to verify that the binding strength of the handle sets for each structure are not too strong. This ensures that the melting temperature remains below 55° C., preserving the structural integrity of CDO prior to assembly. For polycube assemblies, the number and ratio of individual cube types (CTs) were optimized using a SAT solver that meets the requirements of each targeted structure while minimizing the building block complexity. For all polycube assemblies, purified CDO monomers were combined in the correct ratios, with the final monomer concentration ranging from 7 to 10 nM in 1×TAE buffer containing 12.5 mM Mg²⁺. The mixed samples were annealed with a customized protocol based on their respective DLS profiles.

A summary of assembly protocols for polycube structures is provided in Table 1.

For the 3D cross, which requires 7 cubes, the two purified CTs were mixed in a ratio of 1 CT0:6 CT1. The annealing protocol was: 53° C. for 1 hour, 50 to 30° C. with 0.1° C. per 15 minutes, and then incubated at RT indefinitely.

For the hollow 3×3×3 cube, which constructed from 20 cubes, the two purified CTs were mixed in a ratio of 8 CT0:12 CT1. The annealing protocol was as follows: 53° C. for 1 hour, 45 to 32° C. with 0.1° C. per 20 minutes, 32 to 25° C. over 10 minutes, and then incubated at RT indefinitely.

For the multifarious werewolf assembly, a set of particles was used to assemble two distinct targets: human and wolf structures. Three polycube assemblies were prepared: human only, wolf only, and multifarious werewolf. For the human-only assembly, consisting of 13 cubes, six purified CTs were mixed in the ratio 1 CT0:1 CT3:2 CT4:3 CT5:1 CT7:5 CT9. For the wolf-only assembly, consisting of 14 cubes, the 6 purified CTs were mixed in the ratio 1 CT1:1 CT2:5 CT4:1 CT6:1 CT8:5 CT9. For the one-pot multifarious werewolf assembly, combining 27 cubes for both structures, the 10 purified CTs were mixed in the ratio 1 CT0:1 CT1:1 CT2:1 CT3:7 CT4:3 CT5:1 CT6:1 CT7:1 CT8:10 CT9. The annealing protocol was as follows: 53° C. for 1 hour, followed by cooling from 50 to 30° C. at a rate of 0.1° C. per 45 minutes, then 30 to 25° C. over 6 minutes, and then incubated at RT indefinitely.

For the fractal Menger cube M₂, which consists of 400 cubes, the 19 purified CTs were mixed in a ratio of 8 CT0:24 CT1:24 CT2:24 CT3:24 CT4:24 CT5:24 CT6:8 CT7:24 CT8:24 CT9:24 CT10:24 CT11:24 CT12:24 CT13:24 CT14:24 CT15:24 CT16:12 CT17:12 CT18. The annealing protocol was as follows: 53° C. for 1 hour, 44 to 28.5° C. with 0.1° C. per 100 minutes, 28.5 to 20° C. for 6 minutes, and then incubated at 20° C. indefinitely.

For the SC crystal lattice, the 2 CTs were mixed together in an equal ratio of 1:1. For SC with 5T spacers, the annealing protocol was: 53° C. for 1.5 hours, 51.5 to 47.8° C. with 0.1° C. per 6 hours, 47.7 to 25° C. for 10 minutes, and then incubation at RT indefinitely. For SC with 15T spacers, the annealing protocol was: 53° C. for 1.5 hours, 49.5 to 47° C. with 0.1° C. per 6 hours, 46.9 to 25° C. for 10 minutes, and then incubation at RT indefinitely.

The SC lattice crystal was further subjected to a re-annealing strategy, adapted from Huang et al., Chemical Sci. 14(41): 11507-11514 (2023) and confirmed in Liu et al., Science 384(6697): 776-781 (2024) to improve the quality of the crystal assembly. Briefly, 70 μL of supernatant was carefully removed without disturbing the SC crystal sedimented at the bottom of the PCR tube, and a second annealing protocol was applied. This protocol includes adjustments to achieve lower dissociation temperatures and faster ramping rates: 52° C. for the 5T system and 50° C. for the 15T system, with ramping rates of 51.5 to 47.8° C. (0.1° C. per 4 hours for the 5T system) and 49.5 to 47° C. (0.1° C. per 4 hours for the 15T system). These adjustments aim to dissociate small seeds and improve the orderliness of assembly. The annealed SC lattices were stored at 4° C. for subsequent EM characterization.

### Assembly Protocol for Solid 4×4×4 Using CDO Self-Complementary Patches

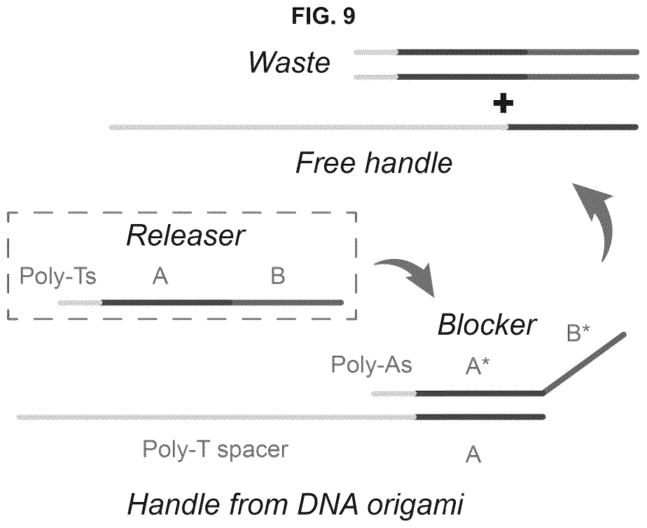

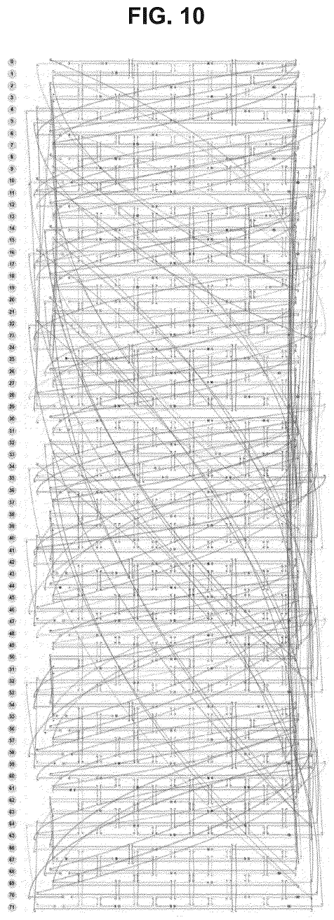

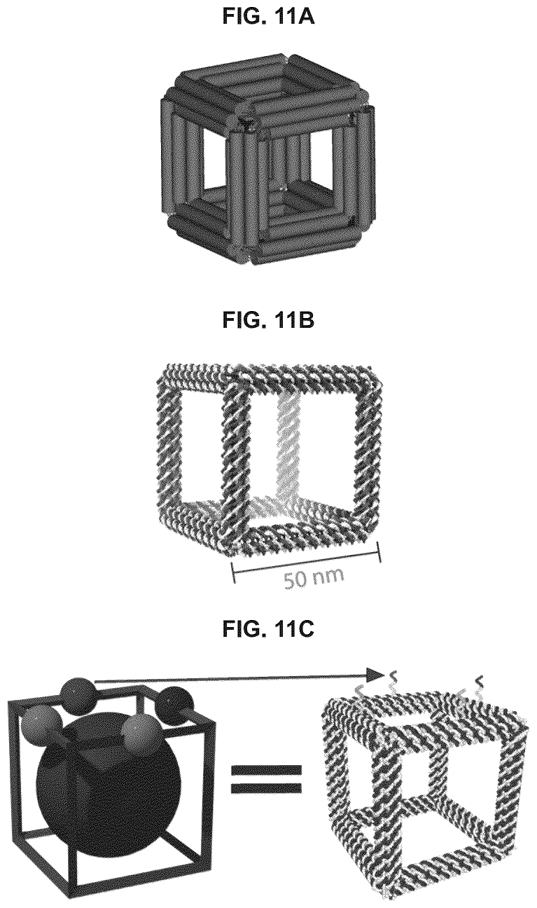

For the solid 4×4×4, composed of 64 cubes, the SAT solver output indicated that 6 CTs were required to assemble the target structure. However, 3 CTs have self-complementary patches that can bind to the same CTs, leading to aggregation during CDO folding and posing challenges during purification. To address this, we adapted our blocking and releasing mechanism, previously developed for the pyrochlore lattice, to isolate monomeric COs, as shown in FIG. 9. A blocker strand was used to inhibit self-binding interactions of the DNA handles. This blocker strand contains regions that hybridize with both the poly-T spacer and the DNA handles, resulting in a lower free energy than the handle-handle interactions.

A 50× excess of blocker strands was added during CDO folding, followed by gel purification to obtain monomeric CDOs. For solid 4×4×4 assembly, the 6 purified CTs were mixed in the ratio of 4 CT0:4 CT1:24 CT2:12 CT3:12 CT4:8 CT5. A hox excess of releasing strand with orthogonal toehold sequences was added to ensure the removal of the blocker strands and exposure of self-complementary DNA handles for polycube assemblies. The annealing protocol was set as the following: 53° C. for 1 hour, 45 to 35° C. with 0.1° C. per 70 minutes, 35 to 25° C. for 15 minutes, and then incubated at RT indefinitely.

TABLE 1
Summary of Assembly Protocols for Polycube Structures

| | Total | | |
|---|---|---|---|
| Structure | Cubes | CT Ratio | Annealing Protocol |
| 3D Cross | 7 | 1 CT0:6 CT1 | 53° C. for 1 hour, 50 to 30° C. at |
| | | | 0.1° C. per 15 min, RT indefinitely |
| Hollow | 20 | 8 CT0:12 CT1 | 53° C. for 1 hour, 45 to 32° C. at |
| 3 × 3 × 3 Cube | | | 0.1° C. per 20 min, 32 to 25° C. for |
| | | | 10 min, RT indefinitely |
| Human-Only | 13 | 1 CT0:1 CT3:2 CT4:3 | 53° C. for 1 hour, 50 to 30° C. at |
| | | CT5:1 CT7:5 CT9 | 0.1° C. per 45 min, 30 to 25° C. for |
| | | | 6 min, RT indefinitely |
| Wolf-Only | 14 | 1 CT1:1 CT2:5 CT4:1 | 53° C. for 1 hour, 50 to 30° C. at |
| | | CT6:1 CT8:5 CT9 | 0.1° C. per 45 min, 30 to 25° C. for |
| | | | 6 min, RT indefinitely |
| Multifarious | 27 | 1 CT0:1 CT1:1 CT2:1 | 53° C. for 1 hour, 50 to 30° C. at |
| Werewolf | | CT3:7 CT4:3 CT5:1 | 0.1° C. per 45 min, 30 to 25° C. for |
| | | CT6:1 CT7:1 CT8:10 | 6 min, RT indefinitely |
| | | CT9 | |
| Solid 4 × 4 × 4 | 64 | 4 CT0:4 CT1:24 CT2:12 | 53° C. for 1 hour, 45 to 35° C. at |
| Cube | | CT3:12 CT4:8 CT5 | 0.1° C. per 70 min, 35 to 25° C. for |
| | | | 15 min, RT indefinitely |
| Menger Cube | 400 | 8 CT0:24 CT1:24 CT2:24 | 53° C. for 1 hour, 44 to 28.5° C. at |
| M₂ | | CT3:24 CT4:24 CT5:24 | 0.1° C. per 100 min, 28.5 to 20° C. |
| | | CT6:8 CT7:24 CT8:24 | for 6 min, then 20° C. indefinitely |
| | | CT9:24 CT10:24 CT11:24 | |
| | | CT12:24 CT13:24 | |
| | | CT14:24 CT15:24 | |
| | | CT16:12 CT17:12 CT18 | |
| SC Lattice (5T | — | 1 CT0:1 CT1 | 53° C. for 1.5 hours, 51.5 to 47.8° |
| Spacer) | | | C. at 0.1° C. per 6 hours, 47.7 to |
| | | | 25° C. for 10 min, RT indefinitely |
| SC Lattice | — | 1 CT0:1 CT1 | 53° C. for 1.5 hours, 49.5 to 47° C. |
| (15T Spacer) | | | at 0.1° C. per 6 hours, 46.9 to 25° |
| | | | C. for 10 min, RT indefinitely |

### Cubes Displacement Reaction

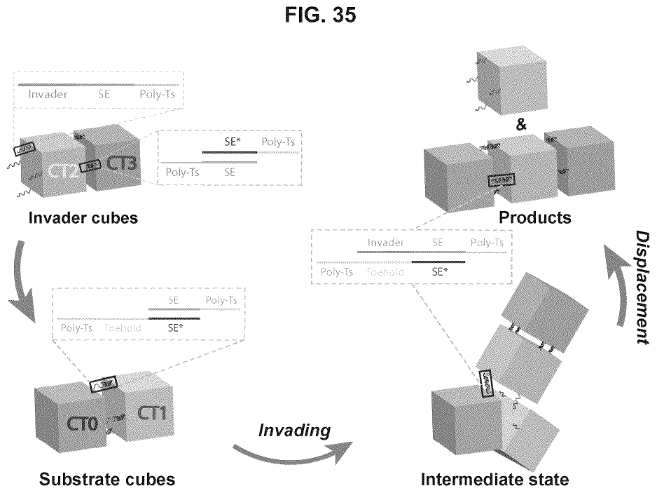

For the dimers experiment, the displacement reaction requires 4 CTs to assemble substrate cubes (CT0 and CT1) and invader cubes (CT2 and CT3) as shown in FIG. 35. The expected products were trimers and monomer CDOs as a by-product. Substrate dimers and invader dimers were first assembled individually from purified CDOs. Both dimers were assembled in a 1:1 ratio, with the final monomers concentration of 7 nM. The annealing protocol was as follows: 53° C. for 15 minutes, 45 to 30° C. with 0.1° C. per 5 minutes, and then incubated at RT indefinitely.

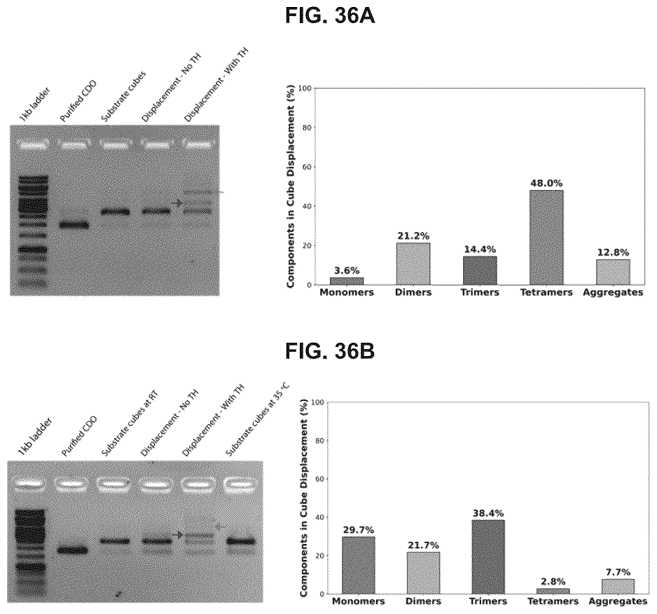

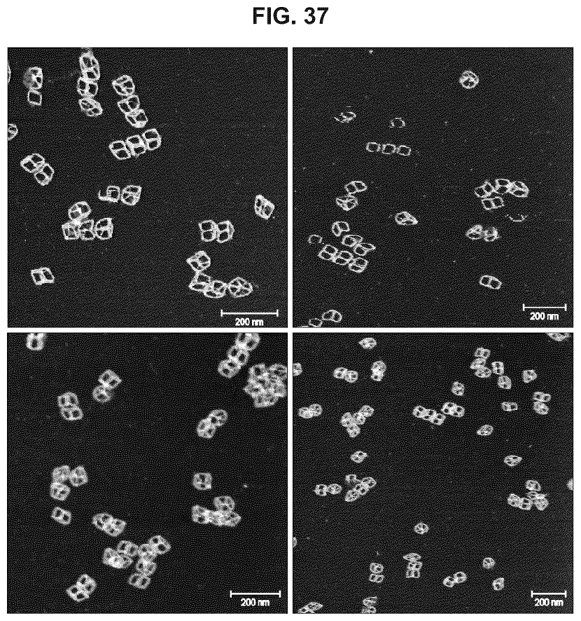

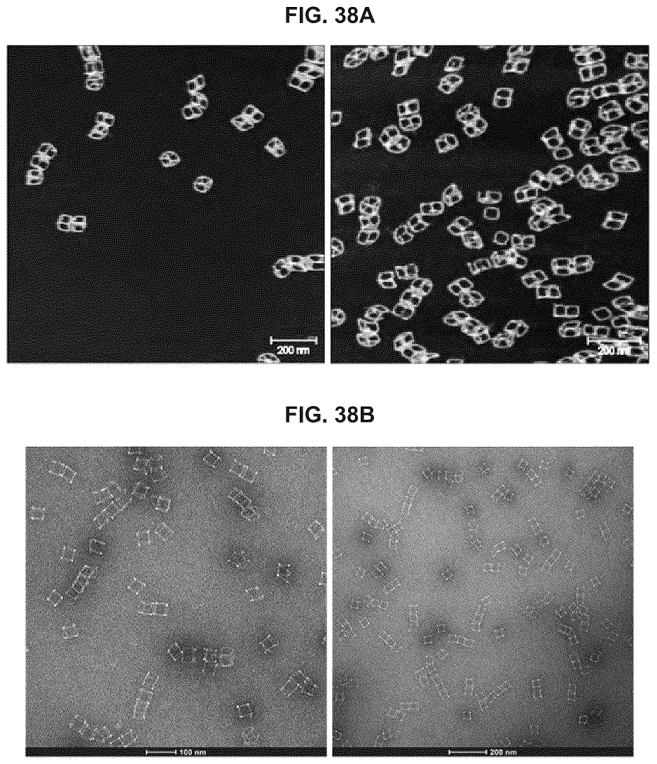

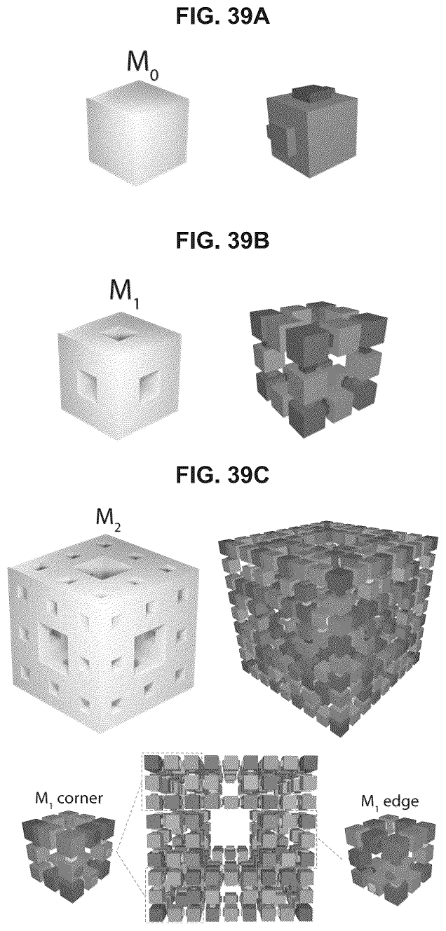

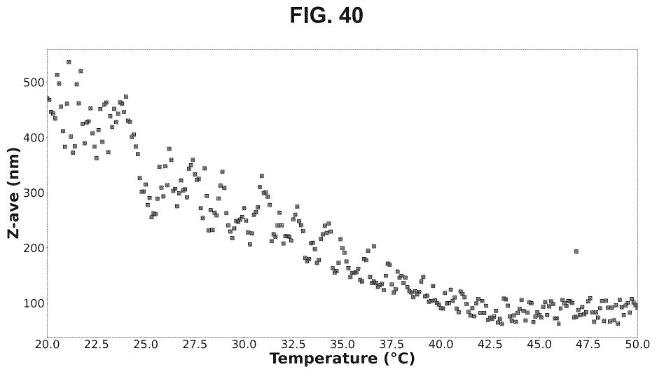

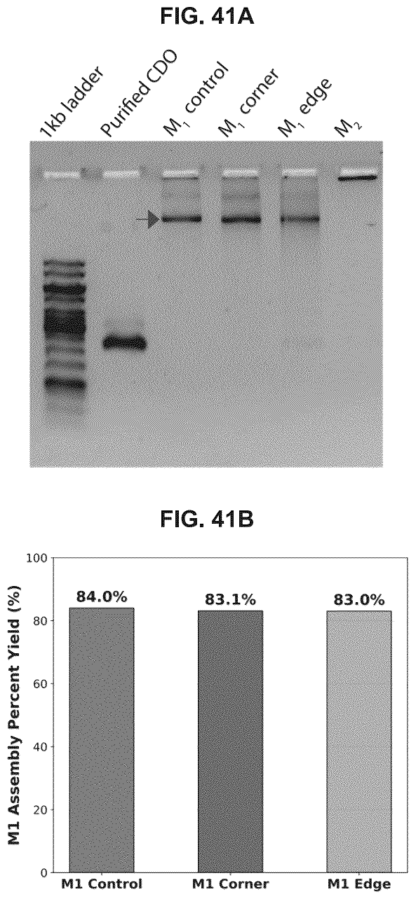

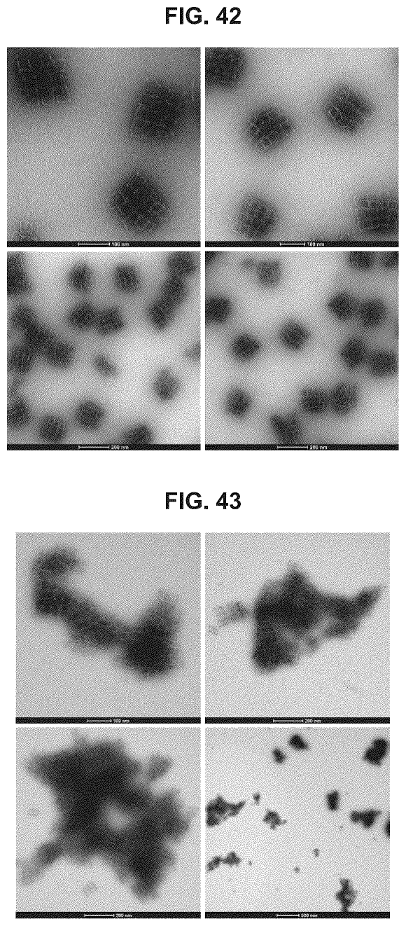

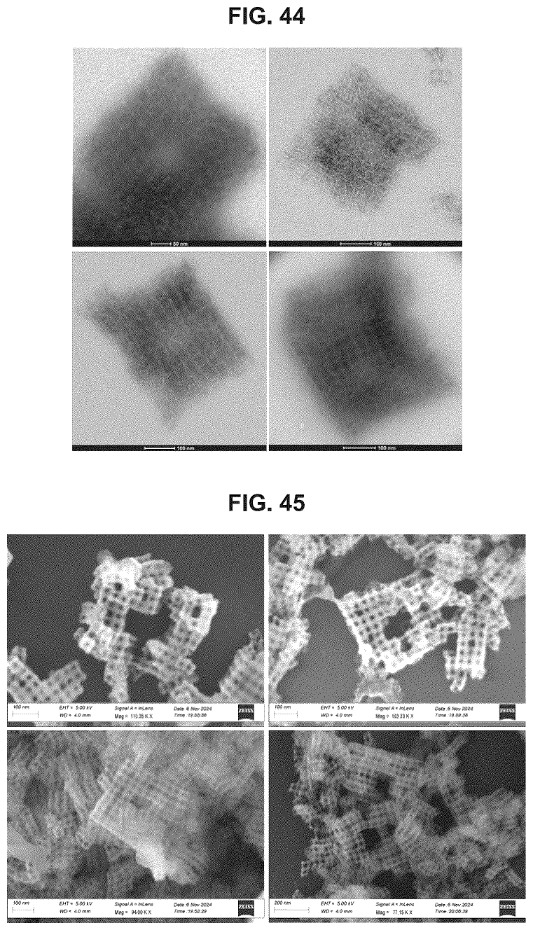

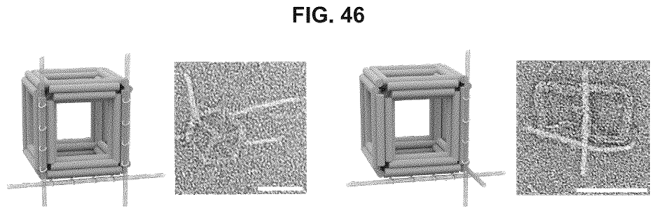

The melting temperature for the dimers was determined by heating the dimers to a target temperature. After 1 hour of incubation, glycerol was added to the dimers to slow the diffusion rates of the CDOs, ensuring that dissociated dimers do not reform. This step prevents false positive results by confirming that a specific temperature does not dissociate the dimers. The heated dimers with glycerol were then quickly transferred to AGE to analyze their yield. Based on this analysis, the displacement reaction was performed at 35° C., where the dimer yield decreases by only 5% (FIG. 36).

For the dimer displacement reaction, invader cubes were added to the substrate cubes at a 1:1 ratio. The sample was incubated at 35° C. overnight and prepared for structural characterization.

### DNA-AuNPs Incorporation

We used an adapted salt-aging method to functionalize AuNPs with targeted DNA. Briefly, 10 nm AuNPs (Nanopartz) were functionalized with single-stranded DNA (ssDNA) through the salt-aging method. The single-stranded thiolated DNA (IDT) was treated with tris[2-carboxyethyl]phosphine (TCEP) in an ice bath for 1 hour to break disulfide bonds prior to conjugation with the AuNPs. TCEP was added to thiolated ssDNA at a molar ratio of 100:1. A desalting column (G-25, GE Healthcare) was used to purify the monothiol ssDNA from excess TCEP along with the reduced by-product.

The monothiol ssDNA was then mixed with AuNPs with a molar ratio of 1:300 and incubated at RT for another 1 hour. Phosphate-buffered saline (PBS) was added to adjust the solution to a concentration of approximately 10 mM, followed by an additional 1 hour incubation. Afterward, 2 M NaCl was added in 5 rounds, with a 1-hour incubation after each addition, and the solution was sonicated for 30 seconds to gradually reach a final concentration of 300 mM. The sample was incubated at RT for at least 18 hours overnight.

The mixed sample was centrifuged at 15,000 rcf and washed four times with fresh buffer (0.1 M 1×PBS) to remove excess ssDNA from the functionalized AuNPs. The final concentration of DNA-functionalized AuNPs was determined using UV-Vis spectroscopy by measuring absorbance at 520 nm.

For polycube AuNP encapsulation, 1.2× excess AuNPs were added to each purified CDO individually using the following protocol: 50 to 30° C. with a ramping rate of 0.1° C. per 20 minutes. The AuNP-encapsulated CDOs were then mixed together with the required ratios of CTs to assemble polycubes, and the same annealing protocol mentioned in the polycube assemblies was applied.

### Silicification of Polycubes

We adapted the silicification protocol from our previously reported pyrochlore lattice assembly. See Liu et al., Science 384(6697): 776-781 (2024). Briefly, a thin layer of silica was coated on the finite-size and crystal lattice polycubes to support the structures for EM characterization. For finite-size assembly, polycubes were loaded into 10 kDa dialysis tubing (Thermo Fisher) and buffer-exchanged three times in 1.5 L of 1×TAE 3 mM Mg²⁺ to reduce the Mg²⁺ concentration. This step allows TMAPS to bind more effectively to the DNA phosphate backbone with a reduced screening effect of cations to the phosphate backbone, thus enhancing the co-condensation sites needed for the silica precursor. The sample concentration was measured using NanoDrop to ensure the correct ratio before the silicification process. TMAPS (50% in methanol, TCI), diluted in methanol, was carefully added to the sample using a microsyringe and gently mixed with a pipette. The mixture was then shaken at 1000 rpm for an hour at 4° C. to promote diffusion and facilitate the binding of TMAPS to the DNA phosphate backbone. Subsequently, TEOS (Sigma-Aldrich), diluted in methanol, was added to the sample and gently mixed, followed by shaking for another hour at 1000 rpm at 4° C. The molar ratio of nucleotide:TMAPS:TEOS was 1:10:20. After the process, the sample was incubated at RT for several days undisturbed prior to EM imaging. Note the prolonged incubation time compared to the procedure applied to superlattices, presumably due to the less accumulated negative charges to efficiently attract TMAPS through electrostatic interactions.

For crystal lattice, the annealed sample was washed three times at RT through buffer exchange by carefully removing the supernatant and replacing it with new buffer of 1×TAE 12.5 mM Mg²⁺. This step helps to remove the monomers and multimers, ensuring a cleaner sample. The same silicification protocol used for finite-size polycubes was applied. The molar ratio of nucleotide:TMAPS:TEOS was 1:10:20. After silicification, the sample was incubated undisturbed at RT for 14 hours. MilliQ water was then added to quench silica growth and remove any residual silanol groups from the lattice structures. The sample was washed four times with 200 μL of MilliQ water, followed by two additional washes with isopropanol, and 180 μL of the supernatant was removed before EM imaging.

### AFM Characterization

AFM imaging was operated with Peakforce tapping mode (Scanasyst) in fluid with a multimode 8 AFM (Vecco 8 from Veeco, Inc.). 6 μL of a purified CDO sample at a concentration of 4.5 nM was deposited on a freshly cleaved mica surface (Ted Pella, Inc.) along with 60 μL of buffer (1×TAE Mg²⁺). The CDO sample was allowed to adsorb to the mica surface for 2 minutes, after which 3 μL of 100 mM NiCl₂ was deposited to the surface before scanning. Scanning parameters were adjusted individually according to scale, size, and CDO conditions.

### Grid Preparation and TEM Characterization

For bare origami samples, formvar carbon-coated copper grids (Ted Pella, Inc.) were glow discharged for 30 seconds. 5 μL of annealed samples were absorbed onto the plasma-charged grid and incubated for 5 minutes. Excess liquid was blotted with filter paper and then washed once with 5 μL of 1×TAE Mg²⁺. For visualization, the samples were negatively stained with 2% uranyl acetate twice, with each incubation time for 30 seconds, followed by blotting with filter paper. The grids were allowed to air dry for at least 30 minutes.

For silicified origami samples, formvar carbon-coated copper grids (Ted Pella, Inc.) were glow discharged for a minute. 7 μL of silicified samples were absorbed onto the plasma-charged grid and incubated for 15 minutes. Excess liquid was blotted with filter paper and then washed once with 5 μL of 1×TAE Mg²⁺. The silicified samples were prepared under both stained and unstained conditions. For the unstained condition, which provides lower resolution, the silicified samples were simply washed once with 1×TAE Mg²⁺, blotted with filter paper, and allowed to air dry for 30 minutes. For the stained condition, the silicified samples were washed once with 1×TAE Mg²⁺ and treated with two rounds of 2% uranyl acetate, with each incubation lasting 30 seconds, followed by blotting with filter paper. The grids were allowed to air dry for at least 30 minutes.

TEM imaging was conducted using a Talos L120C (Thermo Scientific) at an accelerating voltage of 120 kV.

### Cryo-TEM Characterization

For tomography data collection, individual assemblies at 30-50 nm concentrations were absorbed for 2 minutes to the graphene oxide support surfaces on lacy carbon, 200 mesh copper grids that were prepared with 6 nm Au fiducials preabsorbed. Grids were then manually face blotted for 3-6 seconds to form a thin vicinal film prior to vitrification in liquid nitrogen cool liquid ethane. For single particle data collection, 3-6 μL of above concentrations of DNA origami assemblies were applied to Au ultrafoil grids (Quantifoil) and blotted 1.5 seconds prior to vitrification in liquid nitrogen cool liquid ethane in a Thermofisher Scientific Vitrobot MarkIV at 25° C. and 100% relative humidity. Data was collected at 300 KeV on an TFS Titan Krios G2 on a Gatan K2 summit camera operating in counting mode.

Tilt series fractionated 100 electron per °A² dose across 89 tilts using a dose symmetric scheme with a 1.5° angular step resulting in +/−65° tilt ranges using custom scripting in SerialEM. Each tilt angle consisted of 10 frames collected over 2 seconds with movies aligned and summed using UCSF MotionCorr ver. 2.1.4.5. Summed images were restacked from −65 to +65 degrees and tomographic reconstruction was performed with ETOMO (CU Bould 3D lab IMOD package). Tilts were aligned utilizing the 6 nm fiducials to solve for rotation and magnification, then CTF correction and dose weighting were applied prior to tomographic reconstruction using weighted back-projection. Six tomograms for CDO and twelve tomograms for the 3D cross were reconstructed for modeling and volume averaging. Origami assemblies were modeled as scatter points with care to select centers in X, Y, and Z as carefully as possible, selected volumes were then averaged in PEET.

Based on the success of volume averaging from tomograms, we are encouraged to attempt single particle reconstruction methods to improve the resolution of the origami assemblies. 600 movies with 40 frames at 18,000× magnification with a total dose of 36 electron/Å² were acquired for both the CDO assemblies. Movies were full frame motion corrected in cryosparc v 4.3.1, CTF corrections were performed in CTFIND4 and particles were then manually picked. Initial 2D classes were generated and then used to template pick 50,333 cube particles. 2D classification rounds eliminated particles and refined centers resulting in 1,805 cube particles in final reconstructions with/symmetry applied. Resolutions for each complex appear limited by variation in DNA origami structure to 18 Å as neither C₁ or/symmetry refinements altered the resulting resolutions. 3D cross structures were also reconstructed, with initial picks of 12,152 particles. Final reconstructions of the 3D cross structure reach 18.9 Å resolution with 5,432 particles.

### SEM Characterization

For finite-size assembly, silicified samples were loaded into 10 kDa SnakeSkin™ Dialysis Tubing (ThermoFisher Scientific) and buffer-exchanged three times in 1.5 L of MilliQ water to remove the buffering components in the solution. These components, if the liquid was allowed to dry, can precipitate out and create a dirty background, further reducing resolution during imaging. The water-exchanged samples were drop-cast onto a silicon wafer cleaned with isopropanol and air-dried at RT until no visible liquid remains.

For crystal lattice assembly, 20 μL of silicified samples were taken from the bottom of the tube and deposited onto a silicon wafer cleaned with isopropanol. The samples were allowed to air-dry at RT until no visible liquid remains.

SEM imaging was conducted using either Auriga (Zeiss) or Helios 5 UX (Thermo Scientific). Imaging parameters, including accelerating voltages and currents were carefully optimized individually to minimize charging effects.

TABLE 2
DNA SEQUENCES
Staple Sequences for Cube DNA Origami (CDO)

| | | SEQ |
|---|---|---|
| Label | DNA Sequence (5′→3′) | ID NO |
| core strand 0 | GAACAAGCAATTTTTCCGTTTTTATTTTCAAAAAC | 1 |
| core strand 1 | AGCAGCCCGATCTAAATCATTACCGCGCGATTTTCCCTTA | 2 |
| core strand 2 | TGCGTTATACGTTGCTATTTTATGAGGAAGTTTAGTATCATA | 3 |
| core strand 3 | AACGCTCAACTTTTTGTAGGGCTTAATTGACTAGA | 4 |
| core strand 4 | GACAAAAGGTTTTTTAAGTAATTCTGTCCATAATT | 5 |
| core strand 5 | ATAATATCCCTTTTTTCCTAATTTACGAGCGCTTA | 6 |
| core strand 6 | TAACAACCTGCAACAAACAAAATTAATTCCAAAAAAATAATT | 7 |
| core strand 7 | AAACGTAGAATTTTTATACATACATAAAGGAACGG | 8 |
| core strand 8 | TTTTGCTCAGTAAAGACACCAGAACCTAGTTTGCCCCAGCAGACAGCTTGGAAAC | 9 |
| | G | |
| core strand 9 | GAGACTCCTCCACCGCCTGGCCCTGTCGCCCAAAGTAAG | 10 |
| core strand 10 | AGACGTTGGCAACAGCTGATTATTCGGTTACCGAA | 11 |
| core strand 11 | CCGCCTCACCGGAAAATAGGTGTATCACCGTACTTTTTTAGGAGGTTTAGTACCA | 12 |
| | ATAG | |
| core strand 12 | CAAATATATTACCCAAGGGAACCGAACTGGCCGCTGCAAGAA | 13 |
| core strand 13 | GCGGATTAGGCTTGTCCATGTTACTTAGCATCGGAGAGAGAT | 14 |
| core strand 14 | GCCGCCAGGCGGTTTGCGTATCATGTTTTGCTGAA | 15 |
| core strand 15 | TTTGCCTAGAGCCGATCGGCCAACGCGCAAGTACGGCGGATG | 16 |
| core strand 16 | TAGTAAGAGCTTTTTACACTATCATAACCCTTAGG | 17 |
| core strand 17 | CGCCAGGCTACAAAAAGATTCAAAAGGGCGAACGATCATCAG | 18 |
| core strand 18 | TGGCGAACAAGAGACCTCATATATTTTAATGGTCATAAAACG | 19 |
| core strand 19 | TCCTCATACTCACATTAATTGACACAACGAAATTG | 20 |
| core strand 20 | TTAGAGCAAAGCGCGGGTGCCTAATGAGCATAAAGGGTCATA | 21 |
| core strand 21 | CAACATTAAAATTGTAAAGCCACGTCAAAGGGCAGCTTTCAT | 22 |
| core strand 22 | GGATTCTCCGTTTTTGGGAACAAACGGCGGCTGGC | 23 |
| core strand 23 | TGGATTAACGACCAAACAATATTACCGCTAGCGGTAAAATAA | 24 |
| core strand 24 | ATTATCAACCCTTCAGTAGAAGAACTCAATGCGCCTTGTTAA | 25 |
| core strand 25 | AATAAGTATAGCCCGAGATAGCGCCTGAACATCGG | 26 |
| core strand 26 | AGACAAATGCCTTGAATCCCTTATAAATAAGTTACTATACAG | 27 |
| core strand 27 | TTGAGATAGAATGACATATTTAACAACGTAAACACACGAAGG | 28 |
| core strand 28 | TATGCGATCATTGATCGAGCCAGTAATATTGAAATTAAAACACTGCATCA | 29 |
| core strand 29 | TATGTACAGGCTGCACAATAAACAACATCTTTTTCCATACAG | 30 |
| core strand 30 | AGATTGTGCTTCTGATCAACAATAGATATGCTGATCCTCAGAGCTTTCCT | 31 |
| core strand 31 | TTTTGAATATCATTAACCAATCAATAATAGACTACTAAAGGG | 32 |
| core strand 32 | TGAATTTTTTTGTTTAATTTTTTCGTCAAAAATGCCAGTTACAAA | 33 |
| core strand 33 | TTTTCACCAACTTTTTAGAGCCGTCAATGGCGGTCACAATTTCATTTGATTATTT | 34 |
| core strand 34 | TGAGCATATCAGCAACTGGCATGATTAAGACTGATAATTGT | 35 |
| core strand 35 | AATACTTTTTCCAAAAGTTGCTTTCGAGG | 36 |
| core strand 36 | GATATTCCGCGTTTTCAGAACCGCCACCAATAAGATTTGCGG | 37 |
| core strand 37 | AGCAAACATCAAGAGTATTTTTTATCTTGACATTTGAAAGAGGAC | 38 |
| core strand 38 | TATAATGAGTACCTCGGCATTTTCGGTCCTCAGAGCAGGGTGGTTTTTCACAGAC | 39 |
| core strand 39 | ACCTGTTTCGTGCCAGCTGCACATTCCAAACTAATGCAGATACATAATTGTGTCT | 40 |
| | G | |
| core strand 40 | GAGAGATGTTTTCCCCAGACGACGATAAGAAAGATGTAGATT | 41 |
| core strand 41 | GAGCAAAAGGGGGAAAGAAGTTTTGCCAACGTTAAATAACCT | 42 |
| core strand 42 | CCAAGCTTAATGCCGGATTTTTTGAGGGTAGCGGCCGGAGACAGT | 43 |
| core strand 43 | TTATCCGTAGAGGAATTAGCAAGGCCGGTGATATTCTCACTGCCCGCTTATATGA | 44 |
| core strand 44 | CAGTCACTACTTCTGTAATGGGATAGGTGCCATCACACGCTG | 45 |
| core strand 45 | AAAATTATGGATTATTTTTTTTTACATTGGCATGCAACAGGAAAA | 46 |
| core strand 46 | GAGAAACAAGAAATGAAGGTAAATATTGATGATACTGTTGTTCCAGTTTTACCTA | 47 |
| core strand 47 | CCGAAAGCGAATTGCTGAACCTCAAATATCAAAAAAAATCG | 48 |
| core strand 48 | TGAGTTTGAACAAATTTTTTGTCAGAGGAGGCTTTTGAGGACTAAA | 49 |
| core strand 49 | AAATCAGGTCTTTTACCCTGACTATTAACCAGAACTTTTTGAGTAGTAACTGAT | 50 |
| core strand 50 | ATAAGAACCAACATCAGTTCAGAAAACGGGTTTAAAACGGAG | 51 |
| core strand 51 | ATTCTACTAAATTTAATGGTAGAGAATATCGTCATAAATATTTTTAAGTCTTTGA | 52 |
| core strand 52 | CCAATAACCAGTCATTTTTTGGACGTTTCATTTGGTGGCGCGAGCT | 53 |
| core strand 53 | GAAGGGCGATCTTTTGCGGGCCTCTTCATCGTAAATTTTTACTAGCATGATTTC | 54 |
| core strand 54 | GAGAAAAGTTCAGCGCCATTCGCCATTCCCCGGTTACTTTTG | 55 |
| core strand 55 | CGTTAGTAACTATATGTAAAAGTCCTGAGCTTTCCGGCACCATAAGCAAAAGCTA | 56 |
| core strand 56 | TCGCACAAACGTTATTTTTTATATTTTTTGCTTTGTACGAGCACGT | 57 |
| core strand 57 | AGAAACCACCATTTAGGAGCGGAATTAAAGAATACTTTTTGTGGCACAGTTCTT | 58 |
| core strand 58 | CTGTCCAACAGGAAATCATAGG | 59 |
| core strand 59 | GAGTGATAGATTAAGACGCTAGTACCGAACTCGTATTAAATTCGCCATAATCAGT | 60 |
| core strand 60 | TTTAGAAGTATTTTGACTTTACAAACAGAACCACCTTTTTAGCAGAAGATTTTT | 61 |
| core strand 61 | AAGGAACTAAAGCATTTTTTTCACCTTATTCATTTTCAATTACC | 62 |
| core strand 62 | TTTGCCATCTTTTTCATAATCAAAATCCCTCAGAGTTTTTCCGCCACCCCAGTG | 63 |
| core strand 63 | CATCGATAGCATTTACCGTAATCAGTAAGGAGGTTTTTTTGAGGCAGGTGGGAA | 64 |
| core strand 64 | AGGTGAATTATTTTCCGTCACCGACTTCCGTTCCATTTTTGTAAGCGTCGAGTC | 65 |
| core strand 65 | ATATGGTATAAACATTTTTGTTAATGCCTCCTGTTTGATTTGGTGGTT | 66 |
| core strand 66 | ATAGCTTTTTGATAGCTATAACCTTGCTTCTGTAAATCGTCCCTAATTTGAAAAT | 67 |
| core strand 67 | AAAAGTTTTTCCTGTTTCCATTAAACGGGTAAAATACGTAATCATCGCATTGGGC | 68 |
| core strand 68 | TCATCTTTTTTTCTGACCTAATAGTAGTAGCATTAACATCCAGCCTTTTCAATCA | 69 |
| core strand 69 | GAACGCGAAGACAATAGCAAAATTAAGCTGTACCAAACAGGA | 70 |
| core strand 70 | GGTTGTTTTTGGTTATAAATCAGAGCGGGAGCTAAACAGGAGCAATACACAATAT | 71 |
| core strand 71 | TAAGTATTTAAGAACGCATAACCGATATGCCCTTCACCGGAA | 72 |
| core strand 72 | CAATATTTTTGCTATCTCGCTGAGGCTTGCAGGGAGTTAAAGACCAACAGAACCG | 73 |
| core strand 73 | CTCCTTTGCTTAATTAAATATGCAACTAGGGGAGACCAGAAC | 74 |
| core strand 74 | AATACTTTTTCACATTCTATAACAGTTGATTCCCAATTCTGTGAGAAATATTTTT | 75 |
| core strand 75 | AGAGGCTGGAACAAGATACATTTCGCAAAATGCAAGAGTCTG | 76 |
| core strand 76 | CTTCCTTTTTTGTAGCCGAAAAACCGTCTATCAGGCAAGTGCAGCCATGATTCAC | 77 |
| core strand 77 | GAACCCATGTTTTTGTAACAC | 78 |
| core strand 78 | ATTTGTATGCCACTCGGAATCATAATTAGAATCGCCCATAAATCAA | 79 |
| core strand 79 | ATGTTTAGACTTTTATAGCGT | 80 |
| core strand 80 | CGGGAGAAATAAATAAATATATTTTAGTGACGACGGCAACTGTTGG | 81 |
| core strand 81 | CAGTATCGGCTTTTAGGAAGA | 82 |
| core strand 82 | CGTTGTAGGCCGATCTTTTTAACCTCCGATGTAGATTGCGGAACAA | 83 |
| core strand 83 | TGCTAAAGTATCCAAATAAGAAACGATTTCTGTATACATTTGAGGA | 84 |
| core strand 84 | CGCTGAGATCTTTAAATAGAAAGCCTTATTACGCAAGGTTAT | 85 |
| core strand 85 | GTTGGCAAATTTTTCAGTTGA | 86 |
| core strand 86 | AAGCTGCGAGGCGCAGACGGTACCCTCAGAGTTAAGCCCAATCTCAGAAAAGACT | 87 |
| | T | |
| core strand 87 | AAATTTTGTGTCGAAATCCGCGGCTACAGGTAATTGAGCGCTGATAGCAAAGCAA | 88 |
| | A | |
| core strand 88 | ATTAGAGCTCATAGGCTGGCTGACCTTCTCCAACACCTTATTAGCG | 89 |
| core strand 89 | CACCACCTTAGCGTAGGTCATTTCGCCAAAAGGAAAATCAAG | 90 |
| core strand 90 | AACGCTTAAGGATAAAAATTTTATATTTGGGAAGAAAAATCTGAGGGGGCGCCAG | 91 |
| | C | |
| core strand 91 | TCGACTCCTACCGTTCTAGCTGATAAATTGCATGCACCAATGAAAC | 92 |
| core strand 92 | ATTCTGGGGCCTTGCTGGTAACCACCACTAACCAATAGGAACCACGTTGGATTGT | 93 |
| | T | |
| core strand 93 | TGATTTTAGTAATAACATCACACTATGGGTTAAAATTCGCATGTTTGAGATTCCT | 94 |
| | G | |
| core strand 94 | AGAAATAAAGACGCTCAATCGTCTGAAATTTGCACTTATTCATTAA | 95 |
| core strand 95 | CAATAATTGGCAACAGTTTATTTTGTCACAATCAATTTTAAAATTC | 96 |
| core strand 96 | GACTTTTTCGCTTAACTGAACACCCTCGTCACGTATAAAGCC | 97 |
| core strand 97 | GAAAAGGTGCAAACTGGCTCATTATCTGCGGAATAAAGTACC | 98 |
| core strand 98 | ATAACGTGCATAATATTTAAATTGTTCCAGCCAACAAGAAAA | 99 |
| core strand 99 | AATGGTTAAACAGTACATAAATCAATATTAAAAATACCGAACATTCGACCACTCA | 100 |
| | TCGA | |
| core strand 100 | GAATCCTTGTCGTAGGAAGTTTTGTCGTCTTTCCAGACTTTTTTAGTAAA | 101 |
| core strand 101 | ATAAACAGCCATAATTACCTTAAAACAGAGGTGAAGATAATGGGATTT | 102 |
| core strand 102 | ATCGGTTAAAGAAGAGCAAATGAAAAATTTGAGGAGTATGTTAGC | 103 |
| core strand 103 | TGAATTTCTTAAGCGAAAACCCTGCCTATTTCGCGGAATAATATAAA | 104 |
| core strand 104 | GCCCTTTAGCCCGGCCAGAGCCACAAGAGAAGGATGTTGATA | 105 |
| core strand 105 | TAGCAACGACCTGCCCCTGACGAGAAACTAGTCAGAGCCCAATAG | 106 |
| core strand 106 | ACAATGAGCCACCCTAATTCGAGCTTCAAAGCGAACTTTTAGACCGGA | 107 |
| core strand 107 | AGATGAACGGTGTTTTTCACTCAGAACCGCCACCATAGCCCGGTCAGG | 108 |
| core strand 108 | GAAGTTTTTAATGACCGCCAGCATTGACGCGACAGTTACGAGGCA | 109 |
| core strand 109 | GTTTAGCTTAGAACATCGATGAACGGTAGCTATTATAATAGTAAA | 110 |
| core strand 110 | TTGAGATTCGTTTACAGTCACGACGTTGTAAAACGATTTTGGCCAGTG | 111 |
| core strand 111 | CAAATCACCATCATCCAGTCCAGACGATTGGCCTAAACGTCCTGCAGG | 112 |
| core strand 112 | CACTATTTTAAAGAACGTGGACTCCATGAGTCTCTGAATTTAGAGCCATACAACC | 113 |
| | CGTC | |
| core strand 113 | GGCGCGTTTGCCTGTGACCTGAAAGCGTTCATCATGGGACGACGA | 114 |
| core strand 114 | TTCGCGTATTGACCGAATAATGGAAGGGTTAGAACCTTTTACCATATC | 115 |
| core strand 115 | ACGCTCATGGAAAGGAACAAATACATGGCTTTTGACGGAAAGTAAAAC | 116 |
| core strand 116 | CGCAGAGTCGGCAAAGTAACAGTGCCCGTTTACCATATCTGGTCA | 117 |
| core strand 117 | AAACAGGGAATCTTACCAACGGGTTTTGTTAGCGA | 118 |
| core strand 118 | CAGATAGCGAGAGGTAGGATTAGCGGGGGAAAGTATGCAGCA | 119 |
| core strand 119 | GTGCCGTCCGAACAAATGACAACAACCAAGAGAGTTTAAGAGGCT | 120 |
| core strand 120 | TGCGAATAAAGGCTCCAAAAGATCAAGAAGTGCCA | 121 |
| core strand 121 | ATCCCATGAAAATCTACATTTAAGTATTA | 122 |
| core strand 122 | TAAAACACCTTTGCCGGGTATTAAACCAGAGAAGAAGTGTTTTTATATGT | 123 |
| core strand 123 | CCCCAAAAAAACATTATGACCAAAGAATAGAACGC | 124 |
| core strand 124 | ATTGTGAACCAAGCGCGAAACCTAAAACGCGTTAA | 125 |
| core strand 125 | TATCAAACGGTACGCCAGAATCCGAGTAATAGCCC | 126 |
| core strand 126 | TTATTAACGAACTGAAAGAGT | 127 |
| core strand 127 | CTAAAATAGCCAGCATGATGAAACAAAC | 128 |
| core strand 128 | ACCTCCCCCAATAGCATAGTT | 129 |
| core strand 129 | AGCGTAATTTACAGGCGTCTTTCCAGAGGCTATTAATTAACTT | 130 |
| core strand 130 | TCTGAGCGGCTGTAGTTTGAGTAACATTGGCTATAATTAAC | 131 |
| core strand 131 | TGCCTGATGCCTGAGTAATGTACCATTACATTATTACAGGTAAAACCAATGGGTA | 132 |
| | A | |
| core strand 132 | AATCGGTAATAAAGGCAAATC | 133 |
| core strand 133 | AACTACAGGGAGAAACCCAGCTACAATTGATTAGTATTCTAA | 134 |
| core strand 134 | GTGAATAGCATCAACCCTCATTTTCAGGAATATCAACGAGGG | 135 |
| core strand 135 | AGATAGAGATGATGCCGTGCATCTGCCATAAATTTGCTACAG | 136 |
| core strand 136 | ATCAGATAGGCGTTAAGCCTTAAATCAATTATCCTGAAGCGC | 137 |
| core strand 137 | AGGCAGAAAATAAGGAAAGAGGCAAAAGCGATTATATTACCT | 138 |
| core strand 138 | CCTCAGAAAGAATTGCAGCGAAAGACAGCCGGAACTCATTCA | 139 |
| core strand 139 | GTACCGACCTGTGTATACGAGCCGGAAGTGAGCTATAAAGCC | 140 |
| core strand 140 | ATTTTCAACCTTTTTTGCTTTGAATACCCAAAAGATTTAACG | 141 |
| core strand 141 | ATTAGACACGCCTGGCTTATCCGAAATTCTTACCACAGTACA | 142 |
| core strand 142 | AGAATGGCAGCAAATTCGTAATCTGTGAGCGAGTATTGGGAA | 143 |
| core strand 143 | GGGTCAGAGGGCGACGTCAGATGACCCTCAATCAAGCGCCAA | 144 |
| core strand 144 | TGGGCGCCATTTTTACCCGCCGCGCTTAAACTATCCCAACAG | 145 |

Table 3 shows staple sequences from the CDO that were modified to enable encapsulation of AuNPs. “Core strand 133” to “Core strand 144” were removed and replaced with a new set of staple sequences labeled as “AuNP core strand”. The rest of the “Core strand” remained the same. The sequences labeled “AuNP handle” were used to capture DNA-modified AuNPs, while “AuNP functionalize DNA” is the sequence designed for conjugation with AuNPs.

TABLE 3
Staple and Functionalization Sequences

| | | |
|---|---|---|
| | | SEQ ID |
| Label | DNA Sequence (5′→3′) | NO: |

AuNP Core Strands

| | | |
|---|---|---|
| AuNP core strand 0 | GTACCGACCTGTGTATACGAG | 146 |
| AuNP core strand 1 | AGGCAGAAAATAAGGAAAGAG | 147 |
| AuNP core strand 2 | TCTGTGAGCGAGTATTGGGAA | 148 |
| AuNP core strand 3 | ATTTTCAACCTTTTTTGCTTT | 149 |
| AuNP core strand 4 | ATCAGATAGGCGTTAAGCCTT | 150 |
| AuNP core strand 5 | TGACCCTCAATCAAGCGCCAA | 151 |
| AuNP core strand 6 | TGGGCGCCATTTTTACCCGCC | 152 |
| AuNP core strand 7 | TCTGCCATAAATTTGCTACAG | 153 |
| AuNP core strand 8 | TTTCAGGAATATCAACGAGGG | 154 |
| AuNP core strand 9 | CCTCAGAAAGAATTGCAGCGAA | 155 |
| AuNP core strand 10 | GAAATTCTTACCACAGTACAAACTACAGGGAGAAACCCAGCTACAATTGA | 156 |
| | TTAGTATTCTAA | |

AuNP Handles

| | | |
|---|---|---|
| AuNP handle 0 | TCACTTCATACTCTACGTTGTTGTTGTTGTTGTTGTTGTTGTTGTTGCAA | 157 |
| | AAGCGATTATATTACCT | |
| AuNP handle 1 | TCACTTCATACTCTACGTTGTTGTTGTTGTTGTTGTTGTTGTTGTTAGAC | 158 |
| | AGCCGGAACTCATTCAGTGAATAGCATCAACCCTCAT | |
| AuNP handle 2 | TCACTTCATACTCTACGTTGTTGTTGTTGTTGTTGTTGTTGTTGTTGAAT | 159 |
| | ACCCAAAAGATTTAACGGGGTCAGAGGGCGACGTCAGA | |
| AuNP handle 3 | TCACTTCATACTCTACGTTGTTGTTGTTGTTGTTGTTGTTGTTGTTCCGG | 160 |
| | AAGTGAGCTATAAAGCCAGAATGGCAGCAAATTCGTAA | |
| AuNP handle 4 | TCACTTCATACTCTACGTTGTTGTTGTTGTTGTTGTTGTTGTTGTTGCGC | 161 |
| | TTAAACTATCCCAACAGAGATAGAGATGATGCCGTGCA | |
| AuNP handle 5 | TCACTTCATACTCTACGTTGTTGTTGTTGTTGTTGTTGTTGTTGTTAAAT | 162 |
| | CAATTATCCTGAAGCGCATTAGACACGCCTGGCTTATCC | |

DNA-functionalized AuNPs

| | | |
|---|---|---|
| AuNP conjugation | TATGAAGTGATGGATGATGATGATGATGATGAT-SH | 163 |

Table 4 shows handle sequences without sticky ends (SEs) that were used to cover the faces of CDOs where DNA handles are not required for binding. For each design, if a cube facet is not listed, handle sequences without SEs should be used to cover the empty handle. Each face requires four DNA handles for torsional control. The cube facets are classified as front, bottom, left, top, right, and back.

TABLE 4
Handle Sequences without Sticky Ends (SEs)

| | | SEQ ID |
|---|---|---|
| Label | DNA Sequence (5′→3′) | NO: |
| no SEs FRONT 0 | GATCGTCCAATCATAAATCAACGTAACAAGCCCGACCGCCAC | 164 |
| no SEs FRONT 1 | AGCGGTCCGCCGACAAGTTACCAGAAGGCACCAGGCGGATAA | 165 |
| no SEs FRONT 2 | GAACGCGATAGAAGTAGCATTCCACAGA | 166 |
| no SEs FRONT 3 | ACACCGCTAATAGACAACAGTTTCAGCG | 167 |
| no SEs BOT 0 | ATCAGCTATCGTAAGCAATTCATCAATA | 168 |
| no SEs BOT 1 | GAGGCCACCTGAGAGTCAATAGTGAATTCCAAGAACCGAACG | 169 |
| no SEs BOT 2 | CATTTTTAACGGATTGGTTGAGAGGAGTGTACTGGTAGGGAGGTGCGTAG | 170 |
| no SEs BOT 3 | GAGCCTTACAACTAAAGGAATGAGTGAGGGAGCAC | 171 |
| no SEs LEFT 0 | CACCAACAAAGTACTTTCAACTTTAATCCTTTAAAGTAATTT | 172 |
| no SEs LEFT 1 | GCGGGACTAACGAAGAGAATAACATAACAGCCCTCAAGCAA | 173 |
| no SEs LEFT 2 | GCAAGGCCTGTAATGATAATCAGAAAAGGCAAAGCTAATGCA | 174 |
| no SEs LEFT 3 | ATTTTAGTCACGCATAGTCTTTAATGCGTTTTAAACTTTCCTTATCATT | 175 |
| no SEs TOP 0 | AACCCACGCCACCAAAAGATTAAGAGGA | 176 |
| no SEs TOP 1 | CCCCCAGAATACACACCGACCGTGTGATGGCATTTATCCCCCTCAAATG | 177 |
| no SEs TOP 2 | CAGGCGGTAGCTCAATGGGCGCCCACCACCCTCAGATTTTCATTTAATTG | 178 |
| no SEs TOP 3 | TAGTTTGGTAGGTAGGCTATCAGGTCATATTAAGTAATAGCG | 179 |
| no SEs RIGHT 0 | GCTTAGATGATAAGCAGACTGTAGCGCG | 180 |
| no SEs RIGHT 1 | GCTGTTTGCTCGAAATCACCAGTAGCAC | 181 |
| no SEs RIGHT 2 | AGAAACGAAACCGAGATACCGATAGTTGCACGCTGTTATTCTGAAACAT | 182 |
| no SEs RIGHT 3 | TAACAGTGGTTTAACATTCAACCGATTG | 183 |
| no SEs BACK 0 | AACTAACTTTGCAATGTGCTGCAAGGCG | 184 |
| no SEs BACK 1 | CAATCGCCCTGTTTGTGCCGGAAACCAG | 185 |
| no SEs BACK 2 | TATTCACACAATTCCCGTTGCGCACAAACAAATAAACATTACCTCCCCGG | 186 |
| no SEs BACK 3 | CGCGTAATATCCAGGTAATAAAAGGGACTAATCCTGTGTAGA | 187 |

TABLE 5
Handle Sequences for the 3D Cross Cube

| | | SEQ ID |
|---|---|---|
| Label | DNA Sequence (5′→3′) | NO |
| CTO FRONT 0 | GATCGTCCAATCATAAATCAACGTAACAAGCCCGACCGCCACTTTTTTACGCAT | 188 |
| | CT | |
| CTO FRONT 1 | AGCGGTCCGCCGACAAGTTACCAGAAGGCACCAGGCGGATAATTTTTTGAGTGT | 189 |
| | GT | |
| CTO FRONT 2 | GAACGCGATAGAAGTAGCATTCCACAGATTTTTTACAAGGCA | 190 |
| CTO FRONT 3 | ACACCGCTAATAGACAACAGTTTCAGCGTTTTTTGTTCTGGT | 191 |
| CTO BOT 0 | ATCAGCTATCGTAAGCAATTCATCAATATTTTTTACGCATCT | 192 |
| CTO BOT 1 | GAGGCCACCTGAGAGTCAATAGTGAATTCCAAGAACCGAACGTTTTTTGAGTGT | 193 |
| | GT | |
| CTO BOT 2 | CATTTTTAACGGATTGGTTGAGAGGAGTGTACTGGTAGGGAGGTGCGTAGTTTT | 194 |
| | TTACAAGGCA | |
| CTO BOT 3 | GAGCCTTACAACTAAAGGAATGAGTGAGGGAGCACTTTTTTGTTCTGGT | 195 |
| CTO LEFT 0 | CACCAACAAAGTACTTTCAACTTTAATCCTTTAAAGTAATTUTACGCATCT | 196 |
| CTO LEFT 1 | GCGGGACTAACGAAGAGAATAACATAACAGCCCTCAAGCAATTTTTTGAGTGTG | 197 |
| | T | |
| CTO LEFT 2 | GCAAGGCCTGTAATGATAATCAGAAAAGGCAAAGCTAATGCATTTTTTACAAGG | 198 |
| | CA | |
| CTO LEFT 3 | ATTTTAGTCACGCATAGTCTTTAATGCGTTTTAAACTTTCCTTATCATTTGTTC | 199 |
| | TGGT | |
| CTO TOP 0 | AACCCACGCCACCAAAAGATTAAGAGGATTTTTTACGCATCT | 200 |
| CTO TOP 1 | CCCCCAGAATACACACCGACCGTGTGATGGCATTTATCCCCCTCAAATGTTTTT | 201 |
| | TGAGTGTGT | |
| CTO TOP 2 | CAGGCGGTAGCTCAATGGGCGCCCACCACCCTCAGATTTTCATTTAATTGTTTT | 202 |
| | TTACAAGGCA | |
| CTO TOP 3 | TAGTTTGGTAGGTAGGCTATCAGGTCATATTAAGTAATAGCGTTTTTTGTTCTG | 203 |
| | GT | |
| CTO RIGHT 0 | GCTTAGATGATAAGCAGACTGTAGCGCGTTTTTTACGCATCT | 204 |
| CTO RIGHT 1 | GCTGTTTGCTCGAAATCACCAGTAGCACTTTTTTGAGTGTGT | 205 |
| CTO RIGHT 2 | AGAAACGAAACCGAGATACCGATAGTTGCACGCTGTTATTCTGAAACATTACAA | 206 |
| | GGCA | |
| CTO RIGHT 3 | TAACAGTGGTTTAACATTCAACCGATTGTTTTTTGTTCTGGT | 207 |
| CTO BACK 0 | AACTAACTTTGCAATGTGCTGCAAGGCGTTTTTTACGCATCT | 208 |
| CTO BACK 1 | CAATCGCCCTGTTTGTGCCGGAAACCAGTTTTTTGAGTGTGT | 209 |
| CTO BACK 2 | TATTCACACAATTCCCGTTGCGCACAAACAAATAAACATTACCTCCCCGGTTTT | 210 |
| | TTACAAGGCA | |
| CTO BACK 3 | CGCGTAATATCCAGGTAATAAAAGGGACTAATCCTGTGTAGATTTTTTGTTCTG | 211 |
| | GT | |
| CT1 FRONT 0 | GATCGTCCAATCATAAATCAACGTAACAAGCCCGACCGCCACTTTTTAGATGCG | 212 |
| | TA | |
| CT1 FRONT 1 | GAACGCGATAGAAGTAGCATTCCACAGATTTTTACACACTCA | 213 |
| CT1 FRONT 2 | AGCGGTCCGCCGACAAGTTACCAGAAGGCACCAGGCGGATAATTTTTTGCCTTG | 214 |
| | TA | |
| CT1 FRONT 3 | ACACCGCTAATAGACAACAGTTTCAGCGTTTTTACCAGAACA | 215 |

TABLE 6
Handle sequences for the 3 × 3 × 3 Hollow Cube

| | | SEQ ID |
|---|---|---|
| Label | DNA Sequence (5′→3′) | NO |
| CTO FRONT 0 | GATCGTCCAATCATAAATCAACGTAACAAGCCCGACCGCCACTTTTTTTTTTTT | 216 |
| | TACAAGGCA | |
| CTO FRONT 1 | AGCGGTCCGCCGACAAGTTACCAGAAGGCACCAGGCGGATAATTTTTTTTTTTT | 217 |
| | TGTTCTGGT | |
| CTO FRONT 2 | GAACGCGATAGAAGTAGCATTCCACAGATTTTTTTTTTTTTGAGTGTGT | 218 |
| CTO FRONT 3 | ACACCGCTAATAGACAACAGTTTCAGCGTTTTTTTTTTTTTACGCATCT | 219 |
| CTO BOT 0 | ATCAGCTATCGTAAGCAATTCATCAATATTTTTTTTTTTTTGTTCTGGT | 220 |
| CTO BOT 1 | GAGGCCACCTGAGAGTCAATAGTGAATTCCAAGAACCGAACGTTTTTTTTTTTT | 221 |
| | TACGCATCT | |
| CTO BOT 2 | CATTTTTAACGGATTGGTTGAGAGGAGTGTACTGGTAGGGAGGTGCGTAGTTTT | 222 |
| | TTTTTTTTTACAAGGCA | |
| CTO BOT 3 | GAGCCTTACAACTAAAGGAATGAGTGAGGGAGCACTTTTTTTTTTTTTGAGTGT | 223 |
| | GT | |
| CTO LEFT 0 | CACCAACAAAGTACTTTCAACTTTAATCCTTTAAAGTAATTTTTTTTTTTTTTT | 224 |
| | TGTTCTGGT | |
| CTO LEFT 1 | GCGGGACTAACGAAGAGAATAACATAACAGCCCTCAAGCAATTTTTTTTTTTTT | 225 |
| | ACGCATCT | |
| CTO LEFT 2 | GCAAGGCCTGTAATGATAATCAGAAAAGGCAAAGCTAATGCATTTTTTTTTTTT | 226 |
| | TACAAGGCA | |
| CTO LEFT 3 | ATTTTAGTCACGCATAGTCTTTAATGCGTTTTAAACTTTCCTTATCATTTTTTT | 227 |
| | TTTTTTTTGAGTGTGT | |
| CT1 FRONT 0 | GATCGTCCAATCATAAATCAACGTAACAAGCCCGACCGCCACTTTTTTTTTTTT | 228 |
| | ACCAGAACA | |
| CT1 FRONT 1 | AGCGGTCCGCCGACAAGTTACCAGAAGGCACCAGGCGGATAATTTTTTTTTTTT | 229 |
| | TGCCTTGTA | |
| CT1 FRONT 2 | GAACGCGATAGAAGTAGCATTCCACAGATTTTTTTTTTTTAGATGCGTA | 230 |
| CT1 FRONT 3 | ACACCGCTAATAGACAACAGTTTCAGCGTTTTTTTTTTTTACACACTCA | 231 |
| CT1 BACK 0 | AACTAACTTTGCAATGTGCTGCAAGGCGTTTTTTTTTTTTTGCCTTGTA | 232 |
| CT1 BACK 1 | CAATCGCCCTGTTTGTGCCGGAAACCAGTTTTTTTTTTTTACACACTCA | 233 |
| CT1 BACK 2 | TATTCACACAATTCCCGTTGCGCACAAACAAATAAACATTACCTCCCCGGTTTT | 234 |
| | TTTTTTTTACCAGAACA | |
| CT1 BACK 3 | CGCGTAATATCCAGGTAATAAAAGGGACTAATCCTGTGTAGATTTTTTTTTTTT | 235 |
| | AGATGCGTA | |

TABLE 7
Handle Sequences for the 4 × 4 × 4 Solid Cube with Blocking and Releasing Strands

| | | SEQ ID |
|---|---|---|
| Label | DNA Sequence (5′→3′) | NO |
| CTO FRONT 0 | GATCGTCCAATCATAAATCAACGTAACAAGCCCGACCGCCACTTTTTTTTTTTT | 236 |
| | TAGGCATCA | |
| CTO FRONT 1 | AGCGGTCCGCCGACAAGTTACCAGAAGGCACCAGGCGGATAATTTTTTTTTTTT | 237 |
| | TATGAGGCA | |
| CTO FRONT 2 | GAACGCGATAGAAGTAGCATTCCACAGATTTTTTTTTTTTTGTTGTTCG | 238 |
| CTO FRONT 3 | ACACCGCTAATAGACAACAGTTTCAGCGTTTTTTTTTTTTTAATTCGCG | 239 |
| CTO BOT 0 | ATCAGCTATCGTAAGCAATTCATCAATATTTTTTTTTTTTTACACCACA | 240 |
| CTO BOT 1 | GAGGCCACCTGAGAGTCAATAGTGAATTCCAAGAACCGAACGTTTTTTTTTTTT | 241 |
| | TAGCCGTTA | |
| CTO BOT 2 | CATTTTTAACGGATTGGTTGAGAGGAGTGTACTGGTAGGGAGGTGCGTAGTTTT | 242 |
| | TTTTTTTTTGCTCGATA | |
| CTO BOT 3 | GAGCCTTACAACTAAAGGAATGAGTGAGGGAGCACTTTTTTTTTTTTTATCGAC | 243 |
| | GA | |
| CTO LEFT 0 | CACCAACAAAGTACTTTCAACTTTAATCCTTTAAAGTAATTTTTTTTTTTTTTT | 244 |
| | TGATGCCTA | |
| CTO LEFT 1 | GCGGGACTAACGAAGAGAATAACATAACAGCCCTCAAGCAATTTTTTTTTTTTC | 245 |
| | GAACAACA | |
| CTO LEFT 2 | GCAAGGCCTGTAATGATAATCAGAAAAGGCAAAGCTAATGCATTTTTTTTTTTT | 246 |
| | TGCCTCATA | |
| CTO LEFT 3 | ATTTTAGTCACGCATAGTCTTTAATGCGTTTTAAACTTTCCTTATCATTTTTTT | 247 |
| | TTTTTTTCGCGAATTA | |
| CTO TOP 0 | AACCCACGCCACCAAAAGATTAAGAGGATTTTTTTTTTTTACATTCACG | 248 |
| CTO TOP 1 | CCCCCAGAATACACACCGACCGTGTGATGGCATTTATCCCCCTCAAATGTTTTT | 249 |
| | TTTTTTTGCAAGAACA | |
| CTO TOP 2 | CAGGCGGTAGCTCAATGGGCGCCCACCACCCTCAGATTTTCATTTAATTGTTTT | 250 |
| | TTTTTTTTTCAACTCCA | |
| CTO TOP 3 | TAGTTTGGTAGGTAGGCTATCAGGTCATATTAAGTAATAGCGTTTTTTTTTTTT | 251 |
| | TCCAAGTCA | |
| CTO RIGHT 0 | GCTTAGATGATAAGCAGACTGTAGCGCGTTTTTTTTTTTTTCCAAGTCA | 252 |
| CTO RIGHT 1 | GCTGTTTGCTCGAAATCACCAGTAGCACTTTTTTTTTTTTTCAACTCCA | 253 |
| CTO RIGHT 2 | AGAAACGAAACCGAGATACCGATAGTTGCACGCTGTTATTCTGAAACATTTTTT | 254 |
| | TTTTTTTGCAAGAACA | |
| CTO RIGHT 3 | TAACAGTGGTTTAACATTCAACCGATTGTTTTTTTTTTTTACATTCACG | 255 |
| CTO BACK 0 | AACTAACTTTGCAATGTGCTGCAAGGCGTTTTTTTTTTTTTCAACTCCA | 256 |
| CTO BACK 1 | CAATCGCCCTGTTTGTGCCGGAAACCAGTTTTTTTTTTTTACATTCACG | 257 |
| CTO BACK 2 | TATTCACACAATTCCCGTTGCGCACAAACAAATAAACATTACCTCCCCGGTTTT | 258 |
| | TTTTTTTTTCCAAGTCA | |
| CTO BACK 3 | CGCGTAATATCCAGGTAATAAAAGGGACTAATCCTGTGTAGATTTTTTTTTTTT | 259 |
| | GCAAGAACA | |
| CT1 FRONT 0 | GATCGTCCAATCATAAATCAACGTAACAAGCCCGACCGCCACTTTTTTTTTTTT | 260 |
| | TAGTGAGCA | |
| CT1 FRONT 1 | AGCGGTCCGCCGACAAGTTACCAGAAGGCACCAGGCGGATAATTTTTTTTTTTT | 261 |
| | TCCTTGTCA | |
| CT1 FRONT 2 | GAACGCGATAGAAGTAGCATTCCACAGATTTTTTTTTTTTGCAATCACA | 262 |
| CT1 FRONT 3 | ACACCGCTAATAGACAACAGTTTCAGCGTTTTTTTTTTTTTAACAAGCG | 263 |
| CT1 BOT 0 | ATCAGCTATCGTAAGCAATTCATCAATATTTTTTTTTTTTTCAACTCCA | 264 |
| CT1 BOT 1 | GAGGCCACCTGAGAGTCAATAGTGAATTCCAAGAACCGAACGTTTTTTTTTTTT | 265 |
| | ACATTCACG | |
| CT1 BOT 2 | CATTTTTAACGGATTGGTTGAGAGGAGTGTACTGGTAGGGAGGTGCGTAGTTTT | 266 |
| | TTTTTTTTTCCAAGTCA | |
| CT1 BOT 3 | GAGCCTTACAACTAAAGGAATGAGTGAGGGAGCACTTTTTTTTTTTTGCAAGAA | 267 |
| | CA | |
| CT1 LEFT 0 | CACCAACAAAGTACTTTCAACTTTAATCCTTTAAAGTAATTTTTTTTTTTTTTT | 268 |
| | TGCTCACTA | |
| CT1 LEFT 1 | GCGGGACTAACGAAGAGAATAACATAACAGCCCTCAAGCAATTTTTTTTTTTTT | 269 |
| | GTGATTGC | |
| CT1 LEFT 2 | GCAAGGCCTGTAATGATAATCAGAAAAGGCAAAGCTAATGCATTTTTTTTTTTT | 270 |
| | TGACAAGGA | |
| CT1 LEFT 3 | ATTTTAGTCACGCATAGTCTTTAATGCGTTTTAAACTTTCCTTATCATTTTTTT | 271 |
| | TTTTTTTCGCTTGTTA | |
| CT1 TOP 0 | AACCCACGCCACCAAAAGATTAAGAGGATTTTTTTTTTTTTCGTCGATA | 272 |
| CT1 TOP 1 | CCCCCAGAATACACACCGACCGTGTGATGGCATTTATCCCCCTCAAATGTTTTT | 273 |
| | TTTTTTTTAACGGCTA | |
| CT1 TOP 2 | CAGGCGGTAGCTCAATGGGCGCCCACCACCCTCAGATTTTCATTTAATTGTTTT | 274 |
| | TTTTTTTTTATCGAGCA | |
| CT1 TOP 3 | TAGTTTGGTAGGTAGGCTATCAGGTCATATTAAGTAATAGCGTTTTTTTTTTTT | 275 |
| | TGTGGTGTA | |
| CT1 RIGHT 0 | GCTTAGATGATAAGCAGACTGTAGCGCGTTTTTTTTTTTTGCAAGAACA | 276 |
| CT1 RIGHT 1 | GCTGTTTGCTCGAAATCACCAGTAGCACTTTTTTTTTTTTTCCAAGTCA | 277 |
| CT1 RIGHT 2 | AGAAACGAAACCGAGATACCGATAGTTGCACGCTGTTATTCTGAAACATTTTTT | 278 |
| | TTTTTTTACATTCACG | |
| CT1 RIGHT 3 | TAACAGTGGTTTAACATTCAACCGATTGTTTTTTTTTTTTTCAACTCCA | 279 |
| CT1 BACK 0 | AACTAACTTTGCAATGTGCTGCAAGGCGTTTTTTTTTTTTACATTCACG | 280 |
| CT1 BACK 1 | CAATCGCCCTGTTTGTGCCGGAAACCAGTTTTTTTTTTTTGCAAGAACA | 281 |
| CT1 BACK 2 | TATTCACACAATTCCCGTTGCGCACAAACAAATAAACATTACCTCCCCGGTTTT | 282 |
| | TTTTTTTTTCAACTCCA | |
| CT1 BACK 3 | CGCGTAATATCCAGGTAATAAAAGGGACTAATCCTGTGTAGATTTTTTTTTTTT | 283 |
| | TCCAAGTCA | |
| CT2 FRONT 0 | GATCGTCCAATCATAAATCAACGTAACAAGCCCGACCGCCACTTTTTTTTTTTT | 284 |
| | TATCTCGCA | |
| CT2 FRONT 1 | AGCGGTCCGCCGACAAGTTACCAGAAGGCACCAGGCGGATAATTTTTTTTTTTT | 285 |
| | AGTTCCGTA | |
| CT2 FRONT 2 | GAACGCGATAGAAGTAGCATTCCACAGATTTTTTTTTTTTTATCGCTCA | 286 |
| CT2 FRONT 3 | ACACCGCTAATAGACAACAGTTTCAGCGTTTTTTTTTTTTTACGTTCCT | 287 |
| CT2 BOT 0 | ATCAGCTATCGTAAGCAATTCATCAATATTTTTTTTTTTTTACCGCATA | 288 |
| CT2 BOT 1 | GAGGCCACCTGAGAGTCAATAGTGAATTCCAAGAACCGAACGTTTTTTTTTTTT | 289 |
| | TATGGCGTA | |
| CT2 BOT 2 | CATTTTTAACGGATTGGTTGAGAGGAGTGTACTGGTAGGGAGGTGCGTAGTTTT | 290 |
| | TTTTTTTTTGTCTGTGA | |
| CT2 BOT 3 | GAGCCTTACAACTAAAGGAATGAGTGAGGGAGCACTTTTTTTTTTTTACGATCT | 291 |
| | CA | |
| CT2 LEFT 0 | CACCAACAAAGTACTTTCAACTTTAATCCTTTAAAGTAATTTTTTTTTTTTTTT | 292 |
| | TGGAGTTGA | |
| CT2 LEFT 1 | GCGGGACTAACGAAGAGAATAACATAACAGCCCTCAAGCAATTTTTTTTTTTTT | 293 |
| | GACTTGGA | |
| CT2 LEFT 2 | GCAAGGCCTGTAATGATAATCAGAAAAGGCAAAGCTAATGCATTTTTTTTTTTT | 294 |
| | CGTGAATGT | |
| CT2 LEFT 3 | ATTTTAGTCACGCATAGTCTTTAATGCGTTTTAAACTTTCCTTATCATTTTTTT | 295 |
| | TTTTTTTTGTTCTTGC | |
| CT2 TOP 0 | AACCCACGCCACCAAAAGATTAAGAGGATTTTTTTTTTTTTGGTCAAGA | 296 |
| CT2 TOP 1 | CCCCCAGAATACACACCGACCGTGTGATGGCATTTATCCCCCTCAAATGTTTTT | 297 |
| | TTTTTTTTGAACTGGA | |
| CT2 TOP 2 | CAGGCGGTAGCTCAATGGGCGCCCACCACCCTCAGATTTTCATTTAATTGTTTT | 298 |
| | TTTTTTTTGCTGCATTA | |
| CT2 TOP 3 | TAGTTTGGTAGGTAGGCTATCAGGTCATATTAAGTAATAGCGTTTTTTTTTTTT | 299 |
| | TGCTTATGC | |
| CT2 BACK 0 | AACTAACTTTGCAATGTGCTGCAAGGCGTTTTTTTTTTTTTGAGATCGT | 300 |
| CT2 BACK 1 | CAATCGCCCTGTTTGTGCCGGAAACCAGTTTTTTTTTTTTTACGCCATA | 301 |
| CT2 BACK 2 | TATTCACACAATTCCCGTTGCGCACAAACAAATAAACATTACCTCCCCGGTTTT | 302 |
| | TTTTTTTTTCACAGACA | |
| CT2 BACK 3 | CGCGTAATATCCAGGTAATAAAAGGGACTAATCCTGTGTAGATTTTTTTTTTTT | 303 |
| | TATGCGGTA | |
| CT3 BOT 0 | ATCAGCTATCGTAAGCAATTCATCAATATTTTTTTTTTTTTGAGCGATA | 304 |
| CT3 BOT 1 | GAGGCCACCTGAGAGTCAATAGTGAATTCCAAGAACCGAACGTTTTTTTTTTTT | 305 |
| | AGGAACGTA | |
| CT3 BOT 2 | CATTTTTAACGGATTGGTTGAGAGGAGTGTACTGGTAGGGAGGTGCGTAGTTTT | 306 |
| | TTTTTTTTTGCGAGATA | |
| CT3 BOT 3 | GAGCCTTACAACTAAAGGAATGAGTGAGGGAGCACTTTTTTTTTTTTTACGGAA | 307 |
| | CT | |
| CT3 LEFT 0 | CACCAACAAAGTACTTTCAACTTTAATCCTTTAAAGTAATTTTTTTTTTTTTTT | 308 |
| | AGAATCGGA | |
| CT3 LEFT 1 | GCGGGACTAACGAAGAGAATAACATAACAGCCCTCAAGCAATTTTTTTTTTTTT | 309 |
| | AGCACGTA | |
| CT3 LEFT 2 | GCAAGGCCTGTAATGATAATCAGAAAAGGCAAAGCTAATGCATTTTTTTTTTTT | 310 |
| | TACGAACCT | |
| CT3 LEFT 3 | ATTTTAGTCACGCATAGTCTTTAATGCGTTTTAAACTTTCCTTATCATTTTTTT | 311 |
| | TTTTTTTTACGCACTA | |
| CT3 RIGHT 0 | GCTTAGATGATAAGCAGACTGTAGCGCGTTTTTTTTTTTTCGCAACATA | 312 |
| CT3 RIGHT 1 | GCTGTTTGCTCGAAATCACCAGTAGCACTTTTTTTTTTTTTACAGAGCA | 313 |
| CT3 RIGHT 2 | AGAAACGAAACCGAGATACCGATAGTTGCACGCTGTTATTCTGAAACATTTTTT | 314 |
| | TTTTTTTCGCAATGTA | |
| CT3 RIGHT 3 | TAACAGTGGTTTAACATTCAACCGATTGTTTTTTTTTTTTTACTCAGCA | 315 |
| CT3 BACK 0 | AACTAACTTTGCAATGTGCTGCAAGGCGTTTTTTTTTTTTTAATGCAGC | 316 |
| CT3 BACK 1 | CAATCGCCCTGTTTGTGCCGGAAACCAGTTTTTTTTTTTTGCATAAGCA | 317 |
| CT3 BACK 2 | TATTCACACAATTCCCGTTGCGCACAAACAAATAAACATTACCTCCCCGGTTTT | 318 |
| | TTTTTTTTTCTTGACCA | |
| CT3 BACK 3 | CGCGTAATATCCAGGTAATAAAAGGGACTAATCCTGTGTAGATTTTTTTTTTTT | 319 |
| | TCCAGTTCA | |
| CT4 BOT 0 | ATCAGCTATCGTAAGCAATTCATCAATATTTTTTTTTTTTTGAGCGATA | 320 |
| CT4 BOT 1 | GAGGCCACCTGAGAGTCAATAGTGAATTCCAAGAACCGAACGTTTTTTTTTTTT | 321 |
| | AGGAACGTA | |
| CT4 BOT 2 | CATTTTTAACGGATTGGTTGAGAGGAGTGTACTGGTAGGGAGGTGCGTAGTTTT | 322 |
| | TTTTTTTTTGCGAGATA | |
| CT4 BOT 3 | GAGCCTTACAACTAAAGGAATGAGTGAGGGAGCACTTTTTTTTTTTTTACGGAA | 323 |
| | CT | |
| CT4 LEFT 0 | CACCAACAAAGTACTTTCAACTTTAATCCTTTAAAGTAATTTTTTTTTTTTTTT | 324 |
| | TACGTGCTA | |
| CT4 LEFT 1 | GCGGGACTAACGAAGAGAATAACATAACAGCCCTCAAGCAATTTTTTTTTTTTT | 325 |
| | CCGATTCT | |
| CT4 LEFT 2 | GCAAGGCCTGTAATGATAATCAGAAAAGGCAAAGCTAATGCATTTTTTTTTTTT | 326 |
| | TAGTGCGTA | |
| CT4 LEFT 3 | ATTTTAGTCACGCATAGTCTTTAATGCGTTTTAAACTTTCCTTATCATTTTTTT | 327 |
| | TTTTTTTAGGTTCGTA | |
| CT4 RIGHT 0 | GCTTAGATGATAAGCAGACTGTAGCGCGTTTTTTTTTTTTCGCAACATA | 328 |
| CT4 RIGHT 1 | GCTGTTTGCTCGAAATCACCAGTAGCACTTTTTTTTTTTTTACAGAGCA | 329 |
| CT4 RIGHT 2 | AGAAACGAAACCGAGATACCGATAGTTGCACGCTGTTATTCTGAAACATTTTTT | 330 |
| | TTTTTTTCGCAATGTA | |
| CT4 RIGHT 3 | TAACAGTGGTTTAACATTCAACCGATTGTTTTTTTTTTTTTACTCAGCA | 331 |
| CT4 BACK 0 | AACTAACTTTGCAATGTGCTGCAAGGCGTTTTTTTTTTTTTAATGCAGC | 332 |
| CT4 BACK 1 | CAATCGCCCTGTTTGTGCCGGAAACCAGTTTTTTTTTTTTGCATAAGCA | 333 |
| CT4 BACK 2 | TATTCACACAATTCCCGTTGCGCACAAACAAATAAACATTACCTCCCCGGTTTT | 334 |
| | TTTTTTTTTCTTGACCA | |
| CT4 BACK 3 | CGCGTAATATCCAGGTAATAAAAGGGACTAATCCTGTGTAGATTTTTTTTTTTT | 335 |
| | TCCAGTTCA | |
| CT5 BOT 0 | ATCAGCTATCGTAAGCAATTCATCAATATTTTTTTTTTTTTGCTCTGTA | 336 |
| CT5 BOT 1 | GAGGCCACCTGAGAGTCAATAGTGAATTCCAAGAACCGAACGTTTTTTTTTTTT | 337 |
| | TATGTTGCG | |
| CT5 BOT 2 | CATTTTTAACGGATTGGTTGAGAGGAGTGTACTGGTAGGGAGGTGCGTAGTTTT | 338 |
| | TTTTTTTTTGCTGAGTA | |
| CT5 BOT 3 | GAGCCTTACAACTAAAGGAATGAGTGAGGGAGCACTTTTTTTTTTTTTACATTG | 339 |
| | CG | |
| CT5 RIGHT 0 | GCTTAGATGATAAGCAGACTGTAGCGCGTTTTTTTTTTTTTACATTGCG | 340 |
| CT5 RIGHT 1 | GCTGTTTGCTCGAAATCACCAGTAGCACTTTTTTTTTTTTTGCTGAGTA | 341 |
| CT5 RIGHT 2 | AGAAACGAAACCGAGATACCGATAGTTGCACGCTGTTATTCTGAAACATTTTTT | 342 |
| | TTTTTTTTATGTTGCG | |
| CT5 RIGHT 3 | TAACAGTGGTTTAACATTCAACCGATTGTTTTTTTTTTTTTGCTCTGTA | 343 |
| CT5 BACK 0 | AACTAACTTTGCAATGTGCTGCAAGGCGTTTTTTTTTTTTTATGTTGCG | 344 |
| CT5 BACK 1 | CAATCGCCCTGTTTGTGCCGGAAACCAGTTTTTTTTTTTTTACATTGCG | 345 |
| CT5 BACK 2 | TATTCACACAATTCCCGTTGCGCACAAACAAATAAACATTACCTCCCCGGTTTT | 346 |
| | TTTTTTTTTGCTCTGTA | |
| CT5 BACK 3 | CGCGTAATATCCAGGTAATAAAAGGGACTAATCCTGTGTAGATTTTTTTTTTTT | 347 |
| | TGCTGAGTA | |

Blocking Strands

| | | |
|---|---|---|
| CTO blocking 0 | TGATGCCTAAAAAAAAAACGTATGGA | 348 |
| CTO blocking 1 | CGAACAACAAAAAAAAATACAGCGTA | 349 |
| CTO blocking 2 | TGCCTCATAAAAAAAAATATCTGCGA | 350 |
| CTO blocking 3 | CGCGAATTAAAAAAAAAGCATGTGAA | 351 |
| CT1 blocking 0 | TGCTCACTAAAAAAAAATGAAGGACA | 352 |
| CT1 blocking 1 | TGTGATTGCAAAAAAAATACTGACGT | 353 |
| CT1 blocking 2 | TGACAAGGAAAAAAAAATACGAGTGT | 354 |
| CT1 blocking 3 | CGCTTGTTAAAAAAAAATCCACTTGA | 355 |
| CT2 blocking 0 | TGAGATCGTAAAAAAAATGAAGTCCA | 356 |
| CT2 blocking 1 | TACGCCATAAAAAAAAATGTCTTCCA | 357 |
| CT2 blocking 2 | TCACAGACAAAAAAAAATGTTCTCCA | 358 |
| CT2 blocking 3 | TATGCGGTAAAAAAAAATGTCTAGCA | 359 |

Releasing Strands

| | | |
|---|---|---|
| CTO releasing 0 | TCCATACGTTTTTTTTTTAGGCATCA | 360 |
| CTO releasing 1 | TACGCTGTATTTTTTTTTGTTGTTCG | 361 |
| CTO releasing 2 | TCGCAGATATTTTTTTTTATGAGGCA | 362 |
| CTO releasing 3 | TTCACATGCTTTTTTTTTAATTCGCG | 363 |
| CT1 releasing 0 | TGTCCTTCATTTTTTTTTAGTGAGCA | 364 |
| CT1 releasing 1 | ACGTCAGTATTTTTTTTGCAATCACA | 365 |
| CT1 releasing 2 | ACACTCGTATTTTTTTTTCCTTGTCA | 366 |
| CT1 releasing 3 | TCAAGTGGATTTTTTTTTAACAAGCG | 367 |
| CT2 releasing 0 | TGGACTTCATTTTTTTTACGATCTCA | 368 |
| CT2 releasing 1 | TGGAAGACATTTTTTTTTATGGCGTA | 369 |
| CT2 releasing 2 | TGGAGAACATTTTTTTTTGTCTGTGA | 370 |
| CT2 releasing 3 | TGCTAGACATTTTTTTTTACCGCATA | 371 |

TABLE 8
Handle Sequences for the Multifarious Werewolf

| | | SEQ ID |
|---|---|---|
| Label | DNA Sequence (5′→3′) | NO |
| CTO FRONT 0 | GATCGTCCAATCATAAATCAACGTAACAAGCCCGACCGCCACTTTTTTACGGAA | 372 |
| | CT | |
| CTO FRONT 1 | AGCGGTCCGCCGACAAGTTACCAGAAGGCACCAGGCGGATAATTTTTTACGGAA | 373 |
| | CT | |
| CTO FRONT 2 | GAACGCGATAGAAGTAGCATTCCACAGATTTTTTACGGAACT | 374 |
| CTO FRONT 3 | ACACCGCTAATAGACAACAGTTTCAGCGTTTTTTACGGAACT | 375 |
| CTO BACK 0 | AACTAACTTTGCAATGTGCTGCAAGGCGTTTTTTACGGAACT | 376 |
| CTO BACK 1 | CAATCGCCCTGTTTGTGCCGGAAACCAGTTTTTTACGGAACT | 377 |
| CTO BACK 2 | TATTCACACAATTCCCGTTGCGCACAAACAAATAAACATTACCTCCCCGGTTTT | 378 |
| | TTACGGAACT | |
| CTO BACK 3 | CGCGTAATATCCAGGTAATAAAAGGGACTAATCCTGTGTAGATTTTTTACGGAA | 379 |
| | CT | |
| CTO TOP 0 | AACCCACGCCACCAAAAGATTAAGAGGATTTTTTACGGAACT | 380 |
| CTO TOP 1 | CCCCCAGAATACACACCGACCGTGTGATGGCATTTATCCCCCTCAAATGTTTTT | 381 |
| | TACGGAACT | |
| CTO TOP 2 | CAGGCGGTAGCTCAATGGGCGCCCACCACCCTCAGATTTTCATTTAATTGTTTT | 382 |
| | TTACGGAACT | |
| CTO TOP 3 | TAGTTTGGTAGGTAGGCTATCAGGTCATATTAAGTAATAGCGTTTTTTACGGAA | 383 |
| | CT | |
| CTO BOT 0 | ATCAGCTATCGTAAGCAATTCATCAATATTTTTTACACCACA | 384 |
| CTO BOT 1 | GAGGCCACCTGAGAGTCAATAGTGAATTCCAAGAACCGAACGTTTTTTAACGGC | 385 |
| | TA | |
| CTO BOT 2 | CATTTTTAACGGATTGGTTGAGAGGAGTGTACTGGTAGGGAGGTGCGTAGTTTT | 386 |
| | TTACAGAGCA | |
| CTO BOT 3 | GAGCCTTACAACTAAAGGAATGAGTGAGGGAGCACTTTTTGCTGCATTA | 387 |
| CT1 FRONT 0 | GATCGTCCAATCATAAATCAACGTAACAAGCCCGACCGCCACTTTTTTATCGCT | 388 |
| | CA | |
| CT1 FRONT 1 | AGCGGTCCGCCGACAAGTTACCAGAAGGCACCAGGCGGATAATTTTTTATCGAG | 389 |
| | CA | |
| CT1 FRONT 2 | GAACGCGATAGAAGTAGCATTCCACAGATTTTTTATCTCGCA | 390 |
| CT1 FRONT 3 | ACACCGCTAATAGACAACAGTTTCAGCGTTTTTTATCGACGA | 391 |
| CT1 BOT 0 | ATCAGCTATCGTAAGCAATTCATCAATATTTTTTAGGCATCA | 392 |
| CT1 BOT 1 | GAGGCCACCTGAGAGTCAATAGTGAATTCCAAGAACCGAACGTTTTTTAGTGAG | 393 |
| | CA | |
| CT1 BOT 2 | CATTTTTAACGGATTGGTTGAGAGGAGTGTACTGGTAGGGAGGTGCGTAGTTTT | 394 |
| | TTACTCAGCA | |
| CT1 BOT 3 | GAGCCTTACAACTAAAGGAATGAGTGAGGGAGCACTTTTTTACGTGCTA | 395 |
| CT1 TOP 0 | AACCCACGCCACCAAAAGATTAAGAGGATTTTTTACGTGCTA | 396 |
| CT1 TOP 1 | CCCCCAGAATACACACCGACCGTGTGATGGCATTTATCCCCCTCAAATGTTTTT | 397 |
| | TACTCAGCA | |
| CT1 TOP 2 | CAGGCGGTAGCTCAATGGGCGCCCACCACCCTCAGATTTTCATTTAATTGTTTT | 398 |
| | TTAGTGAGCA | |
| CT1 TOP 3 | TAGTTTGGTAGGTAGGCTATCAGGTCATATTAAGTAATAGCGTTTTTTAGGCAT | 399 |
| | CA | |
| CT1 RIGHT 0 | GCTTAGATGATAAGCAGACTGTAGCGCGTTTTTTATGAGGCA | 400 |
| CT1 RIGHT 1 | GCTGTTTGCTCGAAATCACCAGTAGCACTTTTTTCAACTCCA | 401 |
| CT1 RIGHT 2 | AGAAACGAAACCGAGATACCGATAGTTGCACGCTGTTATTCTGAAACATTTTTT | 402 |
| | TATCTGCGA | |
| CT1 RIGHT 3 | TAACAGTGGTTTAACATTCAACCGATTGTTTTTTCACAGACA | 403 |
| CT2 FRONT 0 | GATCGTCCAATCATAAATCAACGTAACAAGCCCGACCGCCACTTTTTTCCAGTT | 404 |
| | CA | |
| CT2 FRONT 1 | AGCGGTCCGCCGACAAGTTACCAGAAGGCACCAGGCGGATAATTTTTTCCAAGT | 405 |
| | CA | |
| CT2 FRONT 2 | GAACGCGATAGAAGTAGCATTCCACAGATTTTTTCCTTGTCA | 406 |
| CT2 FRONT 3 | ACACCGCTAATAGACAACAGTTTCAGCGTTTTTTCTTGACCA | 407 |
| CT2 BOT 0 | ATCAGCTATCGTAAGCAATTCATCAATATTTTTTACTCAGCA | 408 |
| CT2 BOT 1 | GAGGCCACCTGAGAGTCAATAGTGAATTCCAAGAACCGAACGTTTTTTAGGCAT | 409 |
| | CA | |
| CT2 BOT 2 | CATTTTTAACGGATTGGTTGAGAGGAGTGTACTGGTAGGGAGGTGCGTAGTTTT | 410 |
| | TTACGTGCTA | |
| CT2 BOT 3 | GAGCCTTACAACTAAAGGAATGAGTGAGGGAGCACTTTTTTAGTGAGCA | 411 |
| CT2 TOP 0 | AACCCACGCCACCAAAAGATTAAGAGGATTTTTTACTCAGCA | 412 |
| CT2 TOP 1 | CCCCCAGAATACACACCGACCGTGTGATGGCATTTATCCCCCTCAAATGTTTTT | 413 |
| | TAGGCATCA | |
| CT2 TOP 2 | CAGGCGGTAGCTCAATGGGCGCCCACCACCCTCAGATTTTCATTTAATTGTTTT | 414 |
| | TTACGTGCTA | |
| CT2 TOP 3 | TAGTTTGGTAGGTAGGCTATCAGGTCATATTAAGTAATAGCGTTTTTTAGTGAG | 415 |
| | CA | |
| CT3 FRONT 0 | GATCGTCCAATCATAAATCAACGTAACAAGCCCGACCGCCACTTTTTTAGGCAT | 416 |
| | CA | |
| CT3 FRONT 1 | AGCGGTCCGCCGACAAGTTACCAGAAGGCACCAGGCGGATAATTTTTTAGTGAG | 417 |
| | CA | |
| CT3 FRONT 2 | GAACGCGATAGAAGTAGCATTCCACAGATTTTTTACTCAGCA | 418 |
| CT3 FRONT 3 | ACACCGCTAATAGACAACAGTTTCAGCGTTTTTTACGTGCTA | 419 |
| CT3 BACK 0 | AACTAACTTTGCAATGTGCTGCAAGGCGTTTTTTAGGCATCA | 420 |
| CT3 BACK 1 | CAATCGCCCTGTTTGTGCCGGAAACCAGTTTTTTAGTGAGCA | 421 |
| CT3 BACK 2 | TATTCACACAATTCCCGTTGCGCACAAACAAATAAACATTACCTCCCCGGTTTT | 422 |
| | TTACTCAGCA | |
| CT3 BACK 3 | CGCGTAATATCCAGGTAATAAAAGGGACTAATCCTGTGTAGATTTTTTACGTGC | 423 |
| | TA | |
| CT3 BOT 0 | ATCAGCTATCGTAAGCAATTCATCAATATTTTTTACGCTGTA | 424 |
| CT3 BOT 1 | GAGGCCACCTGAGAGTCAATAGTGAATTCCAAGAACCGAACGTTTTTTATGCGG | 425 |
| | TA | |
| CT3 BOT 2 | CATTTTTAACGGATTGGTTGAGAGGAGTGTACTGGTAGGGAGGTGCGTAGTTTT | 426 |
| | TTATGGCGTA | |
| CT3 BOT 3 | GAGCCTTACAACTAAAGGAATGAGTGAGGGAGCACTTTTTTAGTGCGTA | 427 |
| CT4 FRONT 0 | GATCGTCCAATCATAAATCAACGTAACAAGCCCGACCGCCACTTTTTTAGCACG | 428 |
| | TA | |
| CT4 FRONT 1 | AGCGGTCCGCCGACAAGTTACCAGAAGGCACCAGGCGGATAATTTTTTGCTCAC | 429 |
| | TA | |
| CT4 FRONT 2 | GAACGCGATAGAAGTAGCATTCCACAGATTTTTTGCTGAGTA | 430 |
| CT4 FRONT 3 | ACACCGCTAATAGACAACAGTTTCAGCGTTTTTTGATGCCTA | 431 |
| CT4 BOT 0 | ATCAGCTATCGTAAGCAATTCATCAATATTTTTTCCGATTCT | 432 |
| CT4 BOT 1 | GAGGCCACCTGAGAGTCAATAGTGAATTCCAAGAACCGAACGTTTTTTCCGATT | 433 |
| | CT | |
| CT4 BOT 2 | CATTTTTAACGGATTGGTTGAGAGGAGTGTACTGGTAGGGAGGTGCGTAGTTTT | 434 |
| | TTCCGATTCT | |
| CT4 BOT 3 | GAGCCTTACAACTAAAGGAATGAGTGAGGGAGCACTTTTTTCCGATTCT | 435 |
| CT5 FRONT 0 | GATCGTCCAATCATAAATCAACGTAACAAGCCCGACCGCCACTTTTTAGTTCCG | 436 |
| | TA | |
| CT5 FRONT 1 | AGCGGTCCGCCGACAAGTTACCAGAAGGCACCAGGCGGATAATTTTTAGTTCCG | 437 |
| | TA | |
| CT5 FRONT 2 | GAACGCGATAGAAGTAGCATTCCACAGATTTTTAGTTCCGTA | 438 |
| CT5 FRONT 3 | ACACCGCTAATAGACAACAGTTTCAGCGTTTTTAGTTCCGTA | 439 |
| CT5 BACK 0 | AACTAACTTTGCAATGTGCTGCAAGGCGTTTTTTCCGATTCT | 440 |
| CT5 BACK 1 | CAATCGCCCTGTTTGTGCCGGAAACCAGTTTTTTCCGATTCT | 441 |
| CT5 BACK 2 | TATTCACACAATTCCCGTTGCGCACAAACAAATAAACATTACCTCCCCGGTTTT | 442 |
| | TTCCGATTCT | |
| CT5 BACK 3 | CGCGTAATATCCAGGTAATAAAAGGGACTAATCCTGTGTAGATTTTTTCCGATT | 443 |
| | CT | |
| CT6 FRONT 0 | GATCGTCCAATCATAAATCAACGTAACAAGCCCGACCGCCACTTTTTTACGTGC | 444 |
| | TA | |
| CT6 FRONT 1 | AGCGGTCCGCCGACAAGTTACCAGAAGGCACCAGGCGGATAATTTTTTACTCAG | 445 |
| | CA | |
| CT6 FRONT 2 | GAACGCGATAGAAGTAGCATTCCACAGATTTTTTAGTGAGCA | 446 |
| CT6 FRONT 3 | ACACCGCTAATAGACAACAGTTTCAGCGTTTTTTAGGCATCA | 447 |
| CT6 BACK 0 | AACTAACTTTGCAATGTGCTGCAAGGCGTTTTTTGCTCGATA | 448 |
| CT6 BACK 1 | CAATCGCCCTGTTTGTGCCGGAAACCAGTTTTTTGAGCGATA | 449 |
| CT6 BACK 2 | TATTCACACAATTCCCGTTGCGCACAAACAAATAAACATTACCTCCCCGGTTTT | 450 |
| | TTCGTCGATA | |
| CT6 BACK 3 | CGCGTAATATCCAGGTAATAAAAGGGACTAATCCTGTGTAGATTTTTTGCGAGA | 451 |
| | TA | |
| CT7 FRONT 0 | GATCGTCCAATCATAAATCAACGTAACAAGCCCGACCGCCACtttttTGTGGTG | 452 |
| | TA | |
| CT7 FRONT 1 | AGCGGTCCGCCGACAAGTTACCAGAAGGCACCAGGCGGATAAtttttTGCTCTG | 453 |
| | TA | |
| CT7 FRONT 2 | GAACGCGATAGAAGTAGCATTCCACAGAtttttTAGCCGTTA | 454 |
| CT7 FRONT 3 | ACACCGCTAATAGACAACAGTTTCAGCGtttttTAATGCAGC | 455 |
| CT7 BACK 0 | AACTAACTTTGCAATGTGCTGCAAGGCGtttttTACGCACTA | 456 |
| CT7 BACK 1 | CAATCGCCCTGTTTGTGCCGGAAACCAGtttttTACCGCATA | 457 |
| CT7 BACK 2 | TATTCACACAATTCCCGTTGCGCACAAACAAATAAACATTACCTCCCCGGtttt | 458 |
| | tTACGCCATA | |
| CT7 BACK 3 | CGCGTAATATCCAGGTAATAAAAGGGACTAATCCTGTGTAGAtttttTACAGCG | 459 |
| | TA | |
| CT8 FRONT 0 | GATCGTCCAATCATAAATCAACGTAACAAGCCCGACCGCCACTTTTTTCGCAGA | 460 |
| | TA | |
| CT8 FRONT 1 | AGCGGTCCGCCGACAAGTTACCAGAAGGCACCAGGCGGATAATTTTTTGTCTGT | 461 |
| | GA | |
| CT8 FRONT 2 | GAACGCGATAGAAGTAGCATTCCACAGATTTTTTGCCTCATA | 462 |
| CT8 FRONT 3 | ACACCGCTAATAGACAACAGTTTCAGCGTTTTTTGGAGTTGA | 463 |
| CT8 BACK 0 | AACTAACTTTGCAATGTGCTGCAAGGCGTTTTTTGACTTGGA | 464 |
| CT8 BACK 1 | CAATCGCCCTGTTTGTGCCGGAAACCAGTTTTTTGAACTGGA | 465 |
| CT8 BACK 2 | TATTCACACAATTCCCGTTGCGCACAAACAAATAAACATTACCTCCCCGGTTTT | 466 |
| | TTGGTCAAGA | |
| CT8 BACK 3 | CGCGTAATATCCAGGTAATAAAAGGGACTAATCCTGTGTAGATTTTTTGACAAG | 467 |
| | GA | |
| CT9 FRONT 0 | GATCGTCCAATCATAAATCAACGTAACAAGCCCGACCGCCACTTTTTAGAATCG | 468 |
| | GA | |
| CT9 FRONT 1 | AGCGGTCCGCCGACAAGTTACCAGAAGGCACCAGGCGGATAATTTTTAGAATCG | 469 |
| | GA | |
| CT9 FRONT 2 | GAACGCGATAGAAGTAGCATTCCACAGATTTTTAGAATCGGA | 470 |
| CT9 FRONT 3 | ACACCGCTAATAGACAACAGTTTCAGCGTTTTTAGAATCGGA | 471 |

TABLE 9
Handle Sequences for 5- and 15-polyT Spacers in the Simple Cubic Lattice

| | | |
|---|---|---|
| | | SEQ ID |
| Label | DNA Sequence (5′→3′) | NO |

SC 5 polyT spacers

| | | |
|---|---|---|
| CT0 FRONT 0 | GATCGTCCAATCATAAATCAACGTAACAAGCCCGACCGCCACTTTTTACTTGAC | 472 |
| | G | |
| CT0 FRONT 1 | AGCGGTCCGCCGACAAGTTACCAGAAGGCACCAGGCGGATAATTTTTACTTGAC | 473 |
| | G | |
| CT0 FRONT 2 | GAACGCGATAGAAGTAGCATTCCACAGATTTTTACTTGACG | 474 |
| CT0 FRONT 3 | ACACCGCTAATAGACAACAGTTTCAGCGTTTTTACTTGACG | 475 |
| CT0 BOT 0 | ATCAGCTATCGTAAGCAATTCATCAATATTTTTACTTGACG | 476 |
| CT0 BOT 1 | GAGGCCACCTGAGAGTCAATAGTGAATTCCAAGAACCGAACGTTTTTACTTGAC | 477 |
| | G | |
| CT0 BOT 2 | CATTTTTAACGGATTGGTTGAGAGGAGTGTACTGGTAGGGAGGTGCGTAGTTTT | 478 |
| | TACTTGACG | |
| CT0 BOT 3 | GAGCCTTACAACTAAAGGAATGAGTGAGGGAGCACTTTTTACTTGACG | 479 |
| CT0 LEFT 0 | CACCAACAAAGTACTTTCAACTTTAATCCTTTAAAGTAATTTTTTTTACTTGAC | 480 |
| | G | |
| CT0 LEFT 1 | GCGGGACTAACGAAGAGAATAACATAACAGCCCTCAAGCAATTTTTACTTGACG | 481 |
| CT0 LEFT 2 | GCAAGGCCTGTAATGATAATCAGAAAAGGCAAAGCTAATGCATTTTTACTTGAC | 482 |
| | G | |
| CT0 LEFT 3 | ATTTTAGTCACGCATAGTCTTTAATGCGTTTTAAACTTTCCTTATCATTTTTTT | 483 |
| | ACTTGACG | |
| CT0 TOP 0 | AACCCACGCCACCAAAAGATTAAGAGGATTTTTACTTGACG | 484 |
| CT0 TOP 1 | CCCCCAGAATACACACCGACCGTGTGATGGCATTTATCCCCCTCAAATGTTTTT | 485 |
| | ACTTGACG | |
| CT0 TOP 2 | CAGGCGGTAGCTCAATGGGCGCCCACCACCCTCAGATTTTCATTTAATTGTTTT | 486 |
| | TACTTGACG | |
| CT0 TOP 3 | TAGTTTGGTAGGTAGGCTATCAGGTCATATTAAGTAATAGCGTTTTTACTTGAC | 487 |
| | G | |
| CT0 RIGHT 0 | GCTTAGATGATAAGCAGACTGTAGCGCGTTTTTACTTGACG | 488 |
| CT0 RIGHT 1 | GCTGTTTGCTCGAAATCACCAGTAGCACTTTTTACTTGACG | 489 |
| CT0 RIGHT 2 | AGAAACGAAACCGAGATACCGATAGTTGCACGCTGTTATTCTGAAACATTTTTT | 490 |
| | ACTTGACG | |
| CT0 RIGHT 3 | TAACAGTGGTTTAACATTCAACCGATTGTTTTTACTTGACG | 491 |
| CT0 BACK 0 | AACTAACTTTGCAATGTGCTGCAAGGCGTTTTTACTTGACG | 492 |
| CT0 BACK 1 | CAATCGCCCTGTTTGTGCCGGAAACCAGTTTTTACTTGACG | 493 |
| CT0 BACK 2 | TATTCACACAATTCCCGTTGCGCACAAACAAATAAACATTACCTCCCCGGTTTT | 494 |
| | TACTTGACG | |
| CT0 BACK 3 | CGCGTAATATCCAGGTAATAAAAGGGACTAATCCTGTGTAGATTTTTACTTGAC | 495 |
| | G | |
| CT1 FRONT 0 | GATCGTCCAATCATAAATCAACGTAACAAGCCCGACCGCCACTTTTTCGTCAAG | 496 |
| | T | |
| CT1 FRONT 1 | AGCGGTCCGCCGACAAGTTACCAGAAGGCACCAGGCGGATAATTTTTCGTCAAG | 497 |
| | T | |
| CT1 FRONT 2 | GAACGCGATAGAAGTAGCATTCCACAGATTTTTCGTCAAGT | 498 |
| CT1 FRONT 3 | ACACCGCTAATAGACAACAGTTTCAGCGTTTTTCGTCAAGT | 499 |
| CT1 BOT 0 | ATCAGCTATCGTAAGCAATTCATCAATATTTTTCGTCAAGT | 500 |
| CT1 BOT 1 | GAGGCCACCTGAGAGTCAATAGTGAATTCCAAGAACCGAACGTTTTTCGTCAAG | 501 |
| | T | |
| CT1 BOT 2 | CATTTTTAACGGATTGGTTGAGAGGAGTGTACTGGTAGGGAGGTGCGTAGTTTT | 502 |
| | TCGTCAAGT | |
| CT1 BOT 3 | GAGCCTTACAACTAAAGGAATGAGTGAGGGAGCACTTTTTCGTCAAGT | 503 |
| CT1 LEFT 0 | CACCAACAAAGTACTTTCAACTTTAATCCTTTAAAGTAATTTTTTTTCGTCAAG | 504 |
| | T | |
| CT1 LEFT 1 | GCGGGACTAACGAAGAGAATAACATAACAGCCCTCAAGCAATTTTTCGTCAAGT | 505 |
| CT1 LEFT 2 | GCAAGGCCTGTAATGATAATCAGAAAAGGCAAAGCTAATGCATTTTTCGTCAAG | 506 |
| | T | |
| CT1 LEFT 3 | ATTTTAGTCACGCATAGTCTTTAATGCGTTTTAAACTTTCCTTATCATTTTTTT | 507 |
| | CGTCAAGT | |
| CT1 TOP 0 | AACCCACGCCACCAAAAGATTAAGAGGATTTTTCGTCAAGT | 508 |
| CT1 TOP 1 | CCCCCAGAATACACACCGACCGTGTGATGGCATTTATCCCCCTCAAATGTTTTT | 509 |
| | CGTCAAGT | |
| CT1 TOP 2 | CAGGCGGTAGCTCAATGGGCGCCCACCACCCTCAGATTTTCATTTAATTGTTTT | 510 |
| | TCGTCAAGT | |
| CT1 TOP 3 | TAGTTTGGTAGGTAGGCTATCAGGTCATATTAAGTAATAGCGTTTTTCGTCAAG | 511 |
| | T | |
| CT1 RIGHT 0 | GCTTAGATGATAAGCAGACTGTAGCGCGTTTTTCGTCAAGT | 512 |
| CT1 RIGHT 1 | GCTGTTTGCTCGAAATCACCAGTAGCACTTTTTCGTCAAGT | 513 |
| CT1 RIGHT 2 | AGAAACGAAACCGAGATACCGATAGTTGCACGCTGTTATTCTGAAACATTTTTT | 514 |
| | CGTCAAGT | |
| CT1 RIGHT 3 | TAACAGTGGTTTAACATTCAACCGATTGTTTTTCGTCAAGT | 515 |
| CT1 BACK 0 | AACTAACTTTGCAATGTGCTGCAAGGCGTTTTTCGTCAAGT | 516 |
| CT1 BACK 1 | CAATCGCCCTGTTTGTGCCGGAAACCAGTTTTTCGTCAAGT | 517 |
| CT1 BACK 2 | TATTCACACAATTCCCGTTGCGCACAAACAAATAAACATTACCTCCCCGGTTTT | 518 |
| | TCGTCAAGT | |
| CT1 BACK 3 | CGCGTAATATCCAGGTAATAAAAGGGACTAATCCTGTGTAGATTTTTCGTCAAG | 519 |
| | T | |

SC 15 polyT spacers

| | | |
|---|---|---|
| CT0 FRONT 0 | GATCGTCCAATCATAAATCAACGTAACAAGCCCGACCGCCACTTTTTTTTTTTT | 520 |
| | TTTACTTGACG | |
| CT0 FRONT 1 | AGCGGTCCGCCGACAAGTTACCAGAAGGCACCAGGCGGATAATTTTTTTTTTTT | 521 |
| | TTTACTTGACG | |
| CT0 FRONT 2 | GAACGCGATAGAAGTAGCATTCCACAGATTTTTTTTTTTTTTTACTTGACG | 522 |
| CT0 FRONT 3 | ACACCGCTAATAGACAACAGTTTCAGCGTTTTTTTTTTTTTTTACTTGACG | 523 |
| CT0 BOT 0 | ATCAGCTATCGTAAGCAATTCATCAATATTTTTTTTTTTTTTTACTTGACG | 524 |
| CT0 BOT 1 | GAGGCCACCTGAGAGTCAATAGTGAATTCCAAGAACCGAACGTTTTTTTTTTTT | 525 |
| | TTTACTTGACG | |
| CT0 BOT 2 | CATTTTTAACGGATTGGTTGAGAGGAGTGTACTGGTAGGGAGGTGCGTAGTTTT | 526 |
| | TTTTTTTTTTTACTTGACG | |
| CT0 BOT 3 | GAGCCTTACAACTAAAGGAATGAGTGAGGGAGCACTTTTTTTTTTTTTTTACTT | 527 |
| | GACG | |
| CT0 LEFT 0 | CACCAACAAAGTACTTTCAACTTTAATCCTTTAAAGTAATTTTTTTTTTTTTTT | 528 |
| | TTTACTTGACG | |
| CT0 LEFT 1 | GCGGGACTAACGAAGAGAATAACATAACAGCCCTCAAGCAATTTTTTTTTTTTT | 529 |
| | TTACTTGACG | |
| CT0 LEFT 2 | GCAAGGCCTGTAATGATAATCAGAAAAGGCAAAGCTAATGCATTTTTTTTTTTT | 530 |
| | TTTACTTGACG | |
| CT0 LEFT 3 | ATTTTAGTCACGCATAGTCTTTAATGCGTTTTAAACTTTCCTTATCATTTTTTT | 531 |
| | TTTTTTTTttACTTGACG | |
| CT0 TOP 0 | AACCCACGCCACCAAAAGATTAAGAGGATTTTTTTTTTTTTTTACTTGACG | 532 |
| CT0 TOP 1 | CCCCCAGAATACACACCGACCGTGTGATGGCATTTATCCCCCTCAAATGTTTTT | 533 |
| | TTTTTTTTTTACTTGACG | |
| CT0 TOP 2 | CAGGCGGTAGCTCAATGGGCGCCCACCACCCTCAGATTTTCATTTAATTGTTTT | 534 |
| | TTTTTTTTTTTACTTGACG | |
| CT0 TOP 3 | TAGTTTGGTAGGTAGGCTATCAGGTCATATTAAGTAATAGCGTTTTTTTTTTTT | 535 |
| | TTTACTTGACG | |
| CT0 RIGHT 0 | GCTTAGATGATAAGCAGACTGTAGCGCGTTTTTTTTTTTTTTTACTTGACG | 536 |
| CT0 RIGHT 1 | GCTGTTTGCTCGAAATCACCAGTAGCACTTTTTTTTTTTTTTTACTTGACG | 537 |
| CT0 RIGHT 2 | AGAAACGAAACCGAGATACCGATAGTTGCACGCTGTTATTCTGAAACATTTTTT | 538 |
| | TTTTTTTTTtACTTGACG | |
| CT0 RIGHT 3 | TAACAGTGGTTTAACATTCAACCGATTGTTTTTTTTTTTTTTTACTTGACG | 539 |
| CT0 BACK 0 | AACTAACTTTGCAATGTGCTGCAAGGCGTTTTTTTTTTTTTTTACTTGACG | 540 |
| CT0 BACK 1 | CAATCGCCCTGTTTGTGCCGGAAACCAGTTTTTTTTTTTTTTTACTTGACG | 541 |
| CT0 BACK 2 | TATTCACACAATTCCCGTTGCGCACAAACAAATAAACATTACCTCCCCGGTTTT | 542 |
| | TTTTTTTTTTTACTTGACG | |
| CT0 BACK 3 | CGCGTAATATCCAGGTAATAAAAGGGACTAATCCTGTGTAGATTTTTTTTTTTT | 543 |
| | TTTACTTGACG | |
| CT1 FRONT 0 | GATCGTCCAATCATAAATCAACGTAACAAGCCCGACCGCCACTTTTTTTTTTTT | 544 |
| | TTTCGTCAAGT | |
| CT1 FRONT 1 | AGCGGTCCGCCGACAAGTTACCAGAAGGCACCAGGCGGATAATTTTTTTTTTTT | 545 |
| | TTTCGTCAAGT | |
| CT1 FRONT 2 | GAACGCGATAGAAGTAGCATTCCACAGATTTTTTTTTTTTTTTCGTCAAGT | 546 |
| CT1 FRONT 3 | ACACCGCTAATAGACAACAGTTTCAGCGTTTTTTTTTTTTTTTCGTCAAGT | 547 |
| CT1 BOT 0 | ATCAGCTATCGTAAGCAATTCATCAATATTTTTTTTTTTTTTTCGTCAAGT | 548 |
| CT1 BOT 1 | GAGGCCACCTGAGAGTCAATAGTGAATTCCAAGAACCGAACGTTTTTTTTTTTT | 549 |
| | TTTCGTCAAGT | |
| CT1 BOT 2 | CATTTTTAACGGATTGGTTGAGAGGAGTGTACTGGTAGGGAGGTGCGTAGTTTT | 550 |
| | TTTTTTTTTTTCGTCAAGT | |
| CT1 BOT 3 | GAGCCTTACAACTAAAGGAATGAGTGAGGGAGCACTTTTTTTTTTTTTTTCGTC | 551 |
| | AAGT | |
| CT1 LEFT 0 | CACCAACAAAGTACTTTCAACTTTAATCCTTTAAAGTAATTTTTTTTTTTTTTT | 552 |
| | tttCGTCAAGT | |
| CT1 LEFT 1 | GCGGGACTAACGAAGAGAATAACATAACAGCCCTCAAGCAATTTTTTTTTTTTT | 553 |
| | TTCGTCAAGT | |
| CT1 LEFT 2 | GCAAGGCCTGTAATGATAATCAGAAAAGGCAAAGCTAATGCATTTTTTTTTTTT | 554 |
| | TTTCGTCAAGT | |
| CT1 LEFT 3 | ATTTTAGTCACGCATAGTCTTTAATGCGTTTTAAACTTTCCTTATCATTTTTTT | 555 |
| | TTTTTTTTtt CGTCAAGT | |
| CT1 TOP 0 | AACCCACGCCACCAAAAGATTAAGAGGATTTTTTTTTTTTTTTCGTCAAGT | 556 |
| CT1 TOP 1 | CCCCCAGAATACACACCGACCGTGTGATGGCATTTATCCCCCTCAAATGTTTTT | 557 |
| | TTTTTTTTTTCGTCAAGT | |
| CT1 TOP 2 | CAGGCGGTAGCTCAATGGGCGCCCACCACCCTCAGATTTTCATTTAATTGTTTT | 558 |
| | TTTTTTTTTTTCGTCAAGT | |
| CT1 TOP 3 | TAGTTTGGTAGGTAGGCTATCAGGTCATATTAAGTAATAGCGTTTTTTTTTTTT | 559 |
| | TTTCGTCAAGT | |
| CT1 RIGHT 0 | GCTTAGATGATAAGCAGACTGTAGCGCGTTTTTTTTTTTTTTTCGTCAAGT | 560 |
| CT1 RIGHT 1 | GCTGTTTGCTCGAAATCACCAGTAGCACTTTTTCGTCAAGT | 561 |
| CT1 RIGHT 2 | AGAAACGAAACCGAGATACCGATAGTTGCACGCTGTTATTCTGAAACATTTTTT | 562 |
| | TTTTTTTTTt CGTCAAGT | |
| CT1 RIGHT 3 | TAACAGTGGTTTAACATTCAACCGATTGTTTTTTTTTTTTTTTCGTCAAGT | 563 |
| CT1 BACK 0 | AACTAACTTTGCAATGTGCTGCAAGGCGTTTTTTTTTTTTTTTCGTCAAGT | 564 |
| CT1 BACK 1 | CAATCGCCCTGTTTGTGCCGGAAACCAGTTTTTTTTTTTTTTTCGTCAAGT | 565 |
| CT1 BACK 2 | TATTCACACAATTCCCGTTGCGCACAAACAAATAAACATTACCTCCCCGGTTTT | 566 |
| | TTTTTTTTTTTCGTCAAGT | |
| CT1 BACK 3 | CGCGTAATATCCAGGTAATAAAAGGGACTAATCCTGTGTAGATTTTTTTTTTTT | 567 |
| | TTTCGTCAAGT | |

TABLE 10
Handle Sequences for the Dimer Cubes Displacement Reaction

| | | SEQ ID |
|---|---|---|
| Label | DNA Sequence (5′→3′) | NO |
| CT0 toehold | GATCGTCCAATCATAAATCAACGTAACAAGCCCGACCGCCACTTTTTTTTTTTT | 568 |
| FRONT 0 | TACAAGGCAACCTCAACA | |
| CT0 toehold | AGCGGTCCGCCGACAAGTTACCAGAAGGCACCAGGCGGATAATTTTTTTTTTTT | 569 |
| FRONT 1 | TGTTCTGGTAGCGATGTA | |
| CT0 toehold | GAACGCGATAGAAGTAGCATTCCACAGATTTTTTTTTTTTTGAGTGTGTATCGT | 570 |
| FRONT 2 | GCTA | |
| CT0 toehold | ACACCGCTAATAGACAACAGTTTCAGCGTTTTTTTTTTTTTACGCATCTATTCG | 571 |
| FRONT 3 | CCTA | |
| CT1 FRONT 0 | GATCGTCCAATCATAAATCAACGTAACAAGCCCGACCGCCACTTTTTTTTTTTT | 572 |
| | ACCAGAACA | |
| CT1 FRONT 1 | AGCGGTCCGCCGACAAGTTACCAGAAGGCACCAGGCGGATAATTTTTTTTTTTT | 573 |
| | TGCCTTGTA | |
| CT1 FRONT 2 | GAACGCGATAGAAGTAGCATTCCACAGATTTTTTTTTTTTAGATGCGTA | 574 |
| CT1 FRONT 3 | ACACCGCTAATAGACAACAGTTTCAGCGTTTTTTTTTTTTACACACTCA | 575 |
| CT2 invader | GATCGTCCAATCATAAATCAACGTAACAAGCCCGACCGCCACTTTTTTTTTTTT | 576 |
| FRONT 0 | TACATCGCTACCAGAACA | |
| CT2 invader | AGCGGTCCGCCGACAAGTTACCAGAAGGCACCAGGCGGATAATTTTTTTTTTTT | 577 |
| FRONT 1 | TGTTGAGGTTGCCTTGTA | |
| CT2 invader | GAACGCGATAGAAGTAGCATTCCACAGATTTTTTTTTTTTTAGGCGAATAGATG | 578 |
| FRONT 2 | CGTA | |
| CT2 invader | ACACCGCTAATAGACAACAGTTTCAGCGTTTTTTTTTTTTTAGCACGATACACA | 579 |
| FRONT 3 | CTCA | |
| CT2 BACK 0 | AACTAACTTTGCAATGTGCTGCAAGGCGTTTTTTTTTTTTTAACAGCCA | 580 |
| CT2 BACK 1 | CAATCGCCCTGTTTGTGCCGGAAACCAGTTTTTTTTTTTTTAAGCCACA | 581 |
| CT2 BACK 2 | TATTCACACAATTCCCGTTGCGCACAAACAAATAAACATTACCTCCCCGGTTTT | 582 |
| | TTTTTTTTTACCAAGCA | |
| CT2 BACK 3 | CGCGTAATATCCAGGTAATAAAAGGGACTAATCCTGTGTAGATTTTTTTTTTTT | 583 |
| | TAGGTTGCA | |
| CT3 label LEFT | CACCAACAAAGTACTTTCAACTTTAATCCTTTAAAGTAATTTTTTTTTTTTTTT | 584 |
| 0 | TGGCTGTTA | |
| CT3 label LEFT | GCGGGACTAACGAAGAGAATAACATAACAGCCCTCAAGCAATTTTTTTTTTTTT | 585 |
| 1 | GCTTGGTA | |
| CT3 label LEFT | GCAAGGCCTGTAATGATAATCAGAAAAGGCAAAGCTAATGCATTTTTTTTTTTT | 586 |
| 2 | TGTGGCTTA | |
| CT3 label LEFT | ATTTTAGTCACGCATAGTCTTTAATGCGTTTTAAACTTTCCTTATCATTTTTTT | 587 |
| 3 | TTTTTTTTGCAACCTA | |

TABLE 11
Handle Sequences for the Fractal Menger Cube M²

| | | SEQ ID |
|---|---|---|
| Label | DNA Sequence (5′→3′) | NO |
| CT0 TOP 0 | AACCCACGCCACCAAAAGATTAAGAGGATTTTTTTTTTTTACCAGACAT | 588 |
| CT0 TOP 1 | CCCCCAGAATACACACCGACCGTGTGATGGCATTTATCCCCCTCAAATGTTTT | 589 |
| | TTTTTTTTATACAGCAC | |
| CT0 TOP 2 | CAGGCGGTAGCTCAATGGGCGCCCACCACCCTCAGATTTTCATTTAATTGTTT | 590 |
| | TTTTTTTTTACTGTCCAT | |
| CT0 TOP 3 | TAGTTTGGTAGGTAGGCTATCAGGTCATATTAAGTAATAGCGTTTTTTTTTTT | 591 |
| | TACAGGTCAT | |
| CT0 RIGHT 0 | GCTTAGATGATAAGCAGACTGTAGCGCGTTTTTTTTTTTTACAGGTCAT | 592 |
| CT0 RIGHT 1 | GCTGTTTGCTCGAAATCACCAGTAGCACTTTTTTTTTTTTACTGTCCAT | 593 |
| CT0 RIGHT 2 | AGAAACGAAACCGAGATACCGATAGTTGCACGCTGTTATTCTGAAACATTTTT | 594 |
| | TTTTTTTTATACAGCAC | |
| CT0 RIGHT 3 | TAACAGTGGTTTAACATTCAACCGATTGTTTTTTTTTTTTACCAGACAT | 595 |
| CT0 BACK 0 | AACTAACTTTGCAATGTGCTGCAAGGCGTTTTTTTTTTTTACTGTCCAT | 596 |
| CT0 BACK 1 | CAATCGCCCTGTTTGTGCCGGAAACCAGTTTTTTTTTTTTACCAGACAT | 597 |
| CT0 BACK 2 | TATTCACACAATTCCCGTTGCGCACAAACAAATAAACATTACCTCCCCGGTTT | 598 |
| | TTTTTTTTTACAGGTCAT | |
| CT0 BACK 3 | CGCGTAATATCCAGGTAATAAAAGGGACTAATCCTGTGTAGATTTTTTTTTTT | 599 |
| | TATACAGCAC | |
| CT1 FRONT 0 | GATCGTCCAATCATAAATCAACGTAACAAGCCCGACCGCCACTTTTTTTTTTT | 600 |
| | TGTGCTGTAT | |
| CT1 FRONT 1 | AGCGGTCCGCCGACAAGTTACCAGAAGGCACCAGGCGGATAATTTTTTTTTTT | 601 |
| | TATGTCTGGT | |
| CT1 FRONT 2 | GAACGCGATAGAAGTAGCATTCCACAGATTTTTTTTTTTTATGACCTGT | 602 |
| CT1 FRONT 3 | ACACCGCTAATAGACAACAGTTTCAGCGTTTTTTTTTTTTATGGACAGT | 603 |
| CT1 TOP 0 | AACCCACGCCACCAAAAGATTAAGAGGATTTTTTTTTTTTAGATGAACG | 604 |
| CT1 TOP 1 | CCCCCAGAATACACACCGACCGTGTGATGGCATTTATCCCCCTCAAATGTTTT | 605 |
| | TTTTTTTTACTAATGCG | |
| CT1 TOP 2 | CAGGCGGTAGCTCAATGGGCGCCCACCACCCTCAGATTTTCATTTAATTGTTT | 606 |
| | TTTTTTTTTAAGGTCTGT | |
| CT1 TOP 3 | TAGTTTGGTAGGTAGGCTATCAGGTCATATTAAGTAATAGCGTTTTTTTTTTT | 607 |
| | TACTTGATCG | |
| CT1 RIGHT 0 | GCTTAGATGATAAGCAGACTGTAGCGCGTTTTTTTTTTTTATATTGCCG | 608 |
| CT1 RIGHT 1 | GCTGTTTGCTCGAAATCACCAGTAGCACTTTTTTTTTTTTACTGGAACT | 609 |
| CT1 RIGHT 2 | AGAAACGAAACCGAGATACCGATAGTTGCACGCTGTTATTCTGAAACATTTTT | 610 |
| | TTTTTTTTTATGTCCGA | |
| CT1 RIGHT 3 | TAACAGTGGTTTAACATTCAACCGATTGTTTTTTTTTTTTACTAACACG | 611 |
| CT1 BACK 0 | AACTAACTTTGCAATGTGCTGCAAGGCGTTTTTTTTTTTTAGCTCTCAT | 612 |
| CT1 BACK 1 | CAATCGCCCTGTTTGTGCCGGAAACCAGTTTTTTTTTTTTATAAGTGCG | 613 |
| CT1 BACK 2 | TATTCACACAATTCCCGTTGCGCACAAACAAATAAACATTACCTCCCCGGTTT | 614 |
| | TTTTTTTTTACGTTACAG | |
| CT1 BACK 3 | CGCGTAATATCCAGGTAATAAAAGGGACTAATCCTGTGTAGATTTTTTTTTTT | 615 |
| | TAGCAGGTTA | |
| CT2 TOP 0 | AACCCACGCCACCAAAAGATTAAGAGGATTTTTTTTTTTTAACTAGGCA | 616 |
| CT2 TOP 1 | CCCCCAGAATACACACCGACCGTGTGATGGCATTTATCCCCCTCAAATGTTTT | 617 |
| | TTTTTTTTATTGTCTCG | |
| CT2 TOP 2 | CAGGCGGTAGCTCAATGGGCGCCCACCACCCTCAGATTTTCATTTAATTGTTT | 618 |
| | TTTTTTTTTATTGGATCG | |
| CT2 TOP 3 | TAGTTTGGTAGGTAGGCTATCAGGTCATATTAAGTAATAGCGTTTTTTTTTTT | 619 |
| | TAGTTAACGC | |
| CT2 RIGHT 0 | GCTTAGATGATAAGCAGACTGTAGCGCGTTTTTTTTTTTTTAACCTGCT | 620 |
| CT2 RIGHT 1 | GCTGTTTGCTCGAAATCACCAGTAGCACTTTTTTTTTTTTCGCACTTAT | 621 |
| CT2 RIGHT 2 | AGAAACGAAACCGAGATACCGATAGTTGCACGCTGTTATTCTGAAACATTTTT | 622 |
| | TTTTTTTTCTGTAACGT | |
| CT2 RIGHT 3 | TAACAGTGGTTTAACATTCAACCGATTGTTTTTTTTTTTTATGAGAGCT | 623 |
| CT2 BACK 0 | AACTAACTTTGCAATGTGCTGCAAGGCGTTTTTTTTTTTTAGGTCGTTA | 624 |
| CT2 BACK 1 | CAATCGCCCTGTTTGTGCCGGAAACCAGTTTTTTTTTTTTATACGATGC | 625 |
| CT2 BACK 2 | TATTCACACAATTCCCGTTGCGCACAAACAAATAAACATTACCTCCCCGGTTT | 626 |
| | TTTTTTTTTAGGCCAATA | |
| CT2 BACK 3 | CGCGTAATATCCAGGTAATAAAAGGGACTAATCCTGTGTAGATTTTTTTTTTT | 627 |
| | TATAGTTGCG | |
| CT3 FRONT 0 | GATCGTCCAATCATAAATCAACGTAACAAGCCCGACCGCCACTTTTTTTTTTT | 628 |
| | TCGAGACAAT | |
| CT3 FRONT 1 | AGCGGTCCGCCGACAAGTTACCAGAAGGCACCAGGCGGATAATTTTTTTTTTT | 629 |
| | TTGCCTAGTT | |
| CT3 FRONT 2 | GAACGCGATAGAAGTAGCATTCCACAGATTTTTTTTTTTTGCGTTAACT | 630 |
| CT3 FRONT 3 | ACACCGCTAATAGACAACAGTTTCAGCGTTTTTTTTTTTTCGATCCAAT | 631 |
| CT3 RIGHT 0 | GCTTAGATGATAAGCAGACTGTAGCGCGTTTTTTTTTTTTCAGATTCGT | 632 |
| CT3 RIGHT 1 | GCTGTTTGCTCGAAATCACCAGTAGCACTTTTTTTTTTTTCGCCATAAT | 633 |
| CT3 RIGHT 2 | AGAAACGAAACCGAGATACCGATAGTTGCACGCTGTTATTCTGAAACATTTTT | 634 |
| | TTTTTTTTCGTTGTAGT | |
| CT3 RIGHT 3 | TAACAGTGGTTTAACATTCAACCGATTGTTTTTTTTTTTTTAAGACGGT | 635 |
| CT3 BACK 0 | AACTAACTTTGCAATGTGCTGCAAGGCGTTTTTTTTTTTTACGTACCAT | 636 |
| CT3 BACK 1 | CAATCGCCCTGTTTGTGCCGGAAACCAGTTTTTTTTTTTTAGAGTGCAT | 637 |
| CT3 BACK 2 | TATTCACACAATTCCCGTTGCGCACAAACAAATAAACATTACCTCCCCGGTTT | 638 |
| | TTTTTTTTTACCAGAACT | |
| CT3 BACK 3 | CGCGTAATATCCAGGTAATAAAAGGGACTAATCCTGTGTAGATTTTTTTTTTT | 639 |
| | TACTGTTCCT | |
| CT4 TOP 0 | AACCCACGCCACCAAAAGATTAAGAGGATTTTTTTTTTTTATGGTACGT | 640 |
| CT4 TOP 1 | CCCCCAGAATACACACCGACCGTGTGATGGCATTTATCCCCCTCAAATGTTTT | 641 |
| | TTTTTTTTAGTTCTGGT | |
| CT4 TOP 2 | CAGGCGGTAGCTCAATGGGCGCCCACCACCCTCAGATTTTCATTTAATTGTTT | 642 |
| | TTTTTTTTTATGCACTCT | |
| CT4 TOP 3 | TAGTTTGGTAGGTAGGCTATCAGGTCATATTAAGTAATAGCGTTTTTTTTTTT | 643 |
| | TAGGAACAGT | |
| CT4 RIGHT 0 | GCTTAGATGATAAGCAGACTGTAGCGCGTTTTTTTTTTTTAGCATGAGT | 644 |
| CT4 RIGHT 1 | GCTGTTTGCTCGAAATCACCAGTAGCACTTTTTTTTTTTTATTCCTCGT | 645 |
| CT4 RIGHT 2 | AGAAACGAAACCGAGATACCGATAGTTGCACGCTGTTATTCTGAAACATTTTT | 646 |
| | TTTTTTTTTAAGGACGT | |
| CT4 RIGHT 3 | TAACAGTGGTTTAACATTCAACCGATTGTTTTTTTTTTTTTAACGGTCT | 647 |
| CT4 BACK 0 | AACTAACTTTGCAATGTGCTGCAAGGCGTTTTTTTTTTTTGCAACATCT | 648 |
| CT4 BACK 1 | CAATCGCCCTGTTTGTGCCGGAAACCAGTTTTTTTTTTTTGCAGTGAAT | 649 |
| CT4 BACK 2 | TATTCACACAATTCCCGTTGCGCACAAACAAATAAACATTACCTCCCCGGTTT | 650 |
| | TTTTTTTTTGCGACTTAT | |
| CT4 BACK 3 | CGCGTAATATCCAGGTAATAAAAGGGACTAATCCTGTGTAGATTTTTTTTTTT | 651 |
| | TTATTCCGGT | |
| CT5 FRONT 0 | GATCGTCCAATCATAAATCAACGTAACAAGCCCGACCGCCACTTTTTTTTTTT | 652 |
| | TACCGGAATA | |
| CT5 FRONT 1 | AGCGGTCCGCCGACAAGTTACCAGAAGGCACCAGGCGGATAATTTTTTTTTTT | 653 |
| | TATTCACTGC | |
| CT5 FRONT 2 | GAACGCGATAGAAGTAGCATTCCACAGATTTTTTTTTTTTATAAGTCGC | 654 |
| CT5 FRONT 3 | ACACCGCTAATAGACAACAGTTTCAGCGTTTTTTTTTTTTAGATGTTGC | 655 |
| CT5 RIGHT 0 | GCTTAGATGATAAGCAGACTGTAGCGCGTTTTTTTTTTTTGCCATGAAT | 656 |
| CT5 RIGHT 1 | GCTGTTTGCTCGAAATCACCAGTAGCACTTTTTTTTTTTTCACACGTAT | 657 |
| CT5 RIGHT 2 | AGAAACGAAACCGAGATACCGATAGTTGCACGCTGTTATTCTGAAACATTTTT | 658 |
| | TTTTTTTTCGATGACAT | |
| CT5 RIGHT 3 | TAACAGTGGTTTAACATTCAACCGATTGTTTTTTTTTTTTCGAACATGT | 659 |
| CT5 BACK 0 | AACTAACTTTGCAATGTGCTGCAAGGCGTTTTTTTTTTTTGCATCGTAT | 660 |
| CT5 BACK 1 | CAATCGCCCTGTTTGTGCCGGAAACCAGTTTTTTTTTTTTTAACGACCT | 661 |
| CT5 BACK 2 | TATTCACACAATTCCCGTTGCGCACAAACAAATAAACATTACCTCCCCGGTTT | 662 |
| | TTTTTTTTTCGCAACTAT | |
| CT5 BACK 3 | CGCGTAATATCCAGGTAATAAAAGGGACTAATCCTGTGTAGATTTTTTTTTTT | 663 |
| | TTATTGGCCT | |
| CT6 BOT 0 | ATCAGCTATCGTAAGCAATTCATCAATATTTTTTTTTTTTACGAGGAAT | 664 |
| CT6 BOT 1 | GAGGCCACCTGAGAGTCAATAGTGAATTCCAAGAACCGAACGTTTTTTTTTTT | 665 |
| | TACTCATGCT | |
| CT6 BOT 2 | CATTTTTAACGGATTGGTTGAGAGGAGTGTACTGGTAGGGAGGTGCGTAGTTT | 666 |
| | TTTTTTTTTAGACCGTTA | |
| CT6 BOT 3 | GAGCCTTACAACTAAAGGAATGAGTGAGGGAGCACTTTTTTTTTTTTACGTCC | 667 |
| | TTA | |
| CT6 TOP 0 | AACCCACGCCACCAAAAGATTAAGAGGATTTTTTTTTTTTATAGAACGC | 668 |
| CT6 TOP 1 | CCCCCAGAATACACACCGACCGTGTGATGGCATTTATCCCCCTCAAATGTTTT | 669 |
| | TTTTTTTTATAACCACG | |
| CT6 TOP 2 | CAGGCGGTAGCTCAATGGGCGCCCACCACCCTCAGATTTTCATTTAATTGTTT | 670 |
| | TTTTTTTTTATTGAACCG | |
| CT6 TOP 3 | TAGTTTGGTAGGTAGGCTATCAGGTCATATTAAGTAATAGCGTTTTTTTTTTT | 671 |
| | TACCATGTCT | |
| CT7 TOP 0 | AACCCACGCCACCAAAAGATTAAGAGGATTTTTTTTTTTTGCGTTCTAT | 672 |
| CT7 TOP 1 | CCCCCAGAATACACACCGACCGTGTGATGGCATTTATCCCCCTCAAATGTTTT | 673 |
| | TTTTTTTTCGGTTCAAT | |
| CT7 TOP 2 | CAGGCGGTAGCTCAATGGGCGCCCACCACCCTCAGATTTTCATTTAATTGTTT | 674 |
| | TTTTTTTTTCGTGGTTAT | |
| CT7 TOP 3 | TAGTTTGGTAGGTAGGCTATCAGGTCATATTAAGTAATAGCGTTTTTTTTTTT | 675 |
| | TAGACATGGT | |
| CT7 RIGHT 0 | GCTTAGATGATAAGCAGACTGTAGCGCGTTTTTTTTTTTTAGACATGGT | 676 |
| CT7 RIGHT 1 | GCTGTTTGCTCGAAATCACCAGTAGCACTTTTTTTTTTTTCGTGGTTAT | 677 |
| CT7 RIGHT 2 | AGAAACGAAACCGAGATACCGATAGTTGCACGCTGTTATTCTGAAACATTTTT | 678 |
| | TTTTTTTTCGGTTCAAT | |
| CT7 RIGHT 3 | TAACAGTGGTTTAACATTCAACCGATTGTTTTTTTTTTTTGCGTTCTAT | 679 |
| CT7 BACK 0 | AACTAACTTTGCAATGTGCTGCAAGGCGTTTTTTTTTTTTCGTGGTTAT | 680 |
| CT7 BACK 1 | CAATCGCCCTGTTTGTGCCGGAAACCAGTTTTTTTTTTTTGCGTTCTAT | 681 |
| CT7 BACK 2 | TATTCACACAATTCCCGTTGCGCACAAACAAATAAACATTACCTCCCCGGTTT | 682 |
| | TTTTTTTTTAGACATGGT | |
| CT7 BACK 3 | CGCGTAATATCCAGGTAATAAAAGGGACTAATCCTGTGTAGATTTTTTTTTTT | 683 |
| | TCGGTTCAAT | |
| CT8 TOP 0 | AACCCACGCCACCAAAAGATTAAGAGGATTTTTTTTTTTTATTCTCCGT | 684 |
| CT8 TOP 1 | CCCCCAGAATACACACCGACCGTGTGATGGCATTTATCCCCCTCAAATGTTTT | 685 |
| | TTTTTTTTTGCGGATAT | |
| CT8 TOP 2 | CAGGCGGTAGCTCAATGGGCGCCCACCACCCTCAGATTTTCATTTAATTGTTT | 686 |
| | TTTTTTTTTTAACTGTGC | |
| CT8 TOP 3 | TAGTTTGGTAGGTAGGCTATCAGGTCATATTAAGTAATAGCGTTTTTTTTTTT | 687 |
| | TATCGTCTCT | |
| CT8 RIGHT 0 | GCTTAGATGATAAGCAGACTGTAGCGCGTTTTTTTTTTTTATATGCAGC | 688 |
| CT8 RIGHT 1 | GCTGTTTGCTCGAAATCACCAGTAGCACTTTTTTTTTTTTATCATACGC | 689 |
| CT8 RIGHT 2 | AGAAACGAAACCGAGATACCGATAGTTGCACGCTGTTATTCTGAAACATTTTT | 690 |
| | TTTTTTTTAGGTGTCAT | |
| CT8 RIGHT 3 | TAACAGTGGTTTAACATTCAACCGATTGTTTTTTTTTTTTACCACTGAT | 691 |
| CT8 BACK 0 | AACTAACTTTGCAATGTGCTGCAAGGCGTTTTTTTTTTTTGCAGTTGAT | 692 |
| CT8 BACK 1 | CAATCGCCCTGTTTGTGCCGGAAACCAGTTTTTTTTTTTTGCAAGGAAT | 693 |
| CT8 BACK 2 | TATTCACACAATTCCCGTTGCGCACAAACAAATAAACATTACCTCCCCGGTTT | 694 |
| | TTTTTTTTTGCATGGATT | |
| CT8 BACK 3 | CGCGTAATATCCAGGTAATAAAAGGGACTAATCCTGTGTAGATTTTTTTTTTT | 695 |
| | TAGCCTGATT | |
| CT9 FRONT 0 | GATCGTCCAATCATAAATCAACGTAACAAGCCCGACCGCCACTTTTTTTTTTT | 696 |
| | TAGAGACGAT | |
| CT9 FRONT 1 | AGCGGTCCGCCGACAAGTTACCAGAAGGCACCAGGCGGATAATTTTTTTTTTT | 697 |
| | TATATCCGCA | |
| CT9 FRONT 2 | GAACGCGATAGAAGTAGCATTCCACAGATTTTTTTTTTTTGCACAGTTA | 698 |
| CT9 FRONT 3 | ACACCGCTAATAGACAACAGTTTCAGCGTTTTTTTTTTTTACGGAGAAT | 699 |
| CT9 RIGHT 0 | GCTTAGATGATAAGCAGACTGTAGCGCGTTTTTTTTTTTTACCGTCTTA | 700 |
| CT9 RIGHT 1 | GCTGTTTGCTCGAAATCACCAGTAGCACTTTTTTTTTTTTATTATGGCG | 701 |
| CT9 RIGHT 2 | AGAAACGAAACCGAGATACCGATAGTTGCACGCTGTTATTCTGAAACATTTTT | 702 |
| | TTTTTTTTACTACAACG | |
| CT9 RIGHT 3 | TAACAGTGGTTTAACATTCAACCGATTGTTTTTTTTTTTTACGAATCTG | 703 |
| CT9 BACK 0 | AACTAACTTTGCAATGTGCTGCAAGGCGTTTTTTTTTTTTATTGTAGCG | 704 |
| CT9 BACK 1 | CAATCGCCCTGTTTGTGCCGGAAACCAGTTTTTTTTTTTTAGCAGAGAT | 705 |
| CT9 BACK 2 | TATTCACACAATTCCCGTTGCGCACAAACAAATAAACATTACCTCCCCGGTTT | 706 |
| | TTTTTTTTTAGGACACAT | |
| CT9 BACK 3 | CGCGTAATATCCAGGTAATAAAAGGGACTAATCCTGTGTAGATTTTTTTTTTT | 707 |
| | TAGTTCTTGC | |
| CT10 TOP 0 | AACCCACGCCACCAAAAGATTAAGAGGATTTTTTTTTTTTAGGCTGTTA | 708 |
| CT10 TOP 1 | CCCCCAGAATACACACCGACCGTGTGATGGCATTTATCCCCCTCAAATGTTTT | 709 |
| | TTTTTTTTAGTGACCAT | |
| CT10 TOP 2 | CAGGCGGTAGCTCAATGGGCGCCCACCACCCTCAGATTTTCATTTAATTGTTT | 710 |
| | TTTTTTTTTAATACGTCG | |
| CT10 TOP 3 | TAGTTTGGTAGGTAGGCTATCAGGTCATATTAAGTAATAGCGTTTTTTTTTTT | 711 |
| | TAAGATCCGT | |
| CT10 RIGHT 0 | GCTTAGATGATAAGCAGACTGTAGCGCGTTTTTTTTTTTTATATGCGTC | 712 |
| CT10 RIGHT 1 | GCTGTTTGCTCGAAATCACCAGTAGCACTTTTTTTTTTTTAACAGGACT | 713 |
| CT10 RIGHT 2 | AGAAACGAAACCGAGATACCGATAGTTGCACGCTGTTATTCTGAAACATTTTT | 714 |
| | TTTTTTTTACTTGGACT | |
| CT10 RIGHT 3 | TAACAGTGGTTTAACATTCAACCGATTGTTTTTTTTTTTTACCAGCTTA | 715 |
| CT10 BACK 0 | AACTAACTTTGCAATGTGCTGCAAGGCGTTTTTTTTTTTTCGCTACAAT | 716 |
| CT10 BACK 1 | CAATCGCCCTGTTTGTGCCGGAAACCAGTTTTTTTTTTTTATGTGTCCT | 717 |
| CT10 BACK 2 | TATTCACACAATTCCCGTTGCGCACAAACAAATAAACATTACCTCCCCGGTTT | 718 |
| | TTTTTTTTTATCTCTGCT | |
| CT10 BACK 3 | CGCGTAATATCCAGGTAATAAAAGGGACTAATCCTGTGTAGATTTTTTTTTTT | 719 |
| | TGCAAGAACT | |
| CT11 FRONT 0 | GATCGTCCAATCATAAATCAACGTAACAAGCCCGACCGCCACTTTTTTTTTTT | 720 |
| | TATGATAGCG | |
| CT11 FRONT 1 | AGCGGTCCGCCGACAAGTTACCAGAAGGCACCAGGCGGATAATTTTTTTTTTT | 721 |
| | TGCATGTACA | |
| CT11 FRONT 2 | GAACGCGATAGAAGTAGCATTCCACAGATTTTTTTTTTTTACTGGTTCT | 722 |
| CT11 FRONT 3 | ACACCGCTAATAGACAACAGTTTCAGCGTTTTTTTTTTTTATACAACCG | 723 |
| CT11 RIGHT 0 | GCTTAGATGATAAGCAGACTGTAGCGCGTTTTTTTTTTTTATTCATGGC | 724 |
| CT11 RIGHT 1 | GCTGTTTGCTCGAAATCACCAGTAGCACTTTTTTTTTTTTATGTCATCG | 725 |
| CT11 RIGHT 2 | AGAAACGAAACCGAGATACCGATAGTTGCACGCTGTTATTCTGAAACATTTTT | 726 |
| | TTTTTTTTATACGTGTG | |
| CT11 RIGHT 3 | TAACAGTGGTTTAACATTCAACCGATTGTTTTTTTTTTTTACATGTTCG | 727 |
| CT11 BACK 0 | AACTAACTTTGCAATGTGCTGCAAGGCGTTTTTTTTTTTTTAACAGCCT | 728 |
| CT11 BACK 1 | CAATCGCCCTGTTTGTGCCGGAAACCAGTTTTTTTTTTTTCGACGTATT | 729 |
| CT11 BACK 2 | TATTCACACAATTCCCGTTGCGCACAAACAAATAAACATTACCTCCCCGGTTT | 730 |
| | TTTTTTTTTATGGTCACT | |
| CT11 BACK 3 | CGCGTAATATCCAGGTAATAAAAGGGACTAATCCTGTGTAGATTTTTTTTTTT | 731 |
| | TACGGATCTT | |
| CT12 TOP 0 | AACCCACGCCACCAAAAGATTAAGAGGATTTTTTTTTTTTAGGTTCGTA | 732 |
| CT12 TOP 1 | CCCCCAGAATACACACCGACCGTGTGATGGCATTTATCCCCCTCAAATGTTTT | 733 |
| | TTTTTTTTATAGCTCGA | |
| CT12 TOP 2 | CAGGCGGTAGCTCAATGGGCGCCCACCACCCTCAGATTTTCATTTAATTGTTT | 734 |
| | TTTTTTTTTTATCAGCGA | |
| CT12 TOP 3 | TAGTTTGGTAGGTAGGCTATCAGGTCATATTAAGTAATAGCGTTTTTTTTTTT | 735 |
| | TATACCAACG | |
| CT12 RIGHT 0 | GCTTAGATGATAAGCAGACTGTAGCGCGTTTTTTTTTTTTATGTGATCG | 736 |
| CT12 RIGHT 1 | GCTGTTTGCTCGAAATCACCAGTAGCACTTTTTTTTTTTTATACGGCTA | 737 |
| CT12 RIGHT 2 | AGAAACGAAACCGAGATACCGATAGTTGCACGCTGTTATTCTGAAACATTTTT | 738 |
| | TTTTTTTTACATTGTCG | |
| CT12 RIGHT 3 | TAACAGTGGTTTAACATTCAACCGATTGTTTTTTTTTTTTAGCATATCG | 739 |
| CT12 BACK 0 | AACTAACTTTGCAATGTGCTGCAAGGCGTTTTTTTTTTTTCGGTTGTAT | 740 |
| CT12 BACK 1 | CAATCGCCCTGTTTGTGCCGGAAACCAGTTTTTTTTTTTTTGTACATGC | 741 |
| CT12 BACK 2 | TATTCACACAATTCCCGTTGCGCACAAACAAATAAACATTACCTCCCCGGTTT | 742 |
| | TTTTTTTTTAGAACCAGT | |
| CT12 BACK 3 | CGCGTAATATCCAGGTAATAAAAGGGACTAATCCTGTGTAGATTTTTTTTTTT | 743 |
| | TCGCTATCAT | |
| CT13 FRONT 0 | GATCGTCCAATCATAAATCAACGTAACAAGCCCGACCGCCACTTTTTTTTTTT | 744 |
| | TCGTTGGTAT | |
| CT13 FRONT 1 | AGCGGTCCGCCGACAAGTTACCAGAAGGCACCAGGCGGATAATTTTTTTTTTT | 745 |
| | TTCGAGCTAT | |
| CT13 FRONT 2 | GAACGCGATAGAAGTAGCATTCCACAGATTTTTTTTTTTTTCGCTGATA | 746 |
| CT13 FRONT 3 | ACACCGCTAATAGACAACAGTTTCAGCGTTTTTTTTTTTTTACGAACCT | 747 |
| CT13 RIGHT 0 | GCTTAGATGATAAGCAGACTGTAGCGCGTTTTTTTTTTTTCGCATTAGT | 748 |
| CT13 RIGHT 1 | GCTGTTTGCTCGAAATCACCAGTAGCACTTTTTTTTTTTTCGTTCATCT | 749 |
| CT13 RIGHT 2 | AGAAACGAAACCGAGATACCGATAGTTGCACGCTGTTATTCTGAAACATTTTT | 750 |
| | TTTTTTTTCGATCAAGT | |
| CT13 RIGHT 3 | TAACAGTGGTTTAACATTCAACCGATTGTTTTTTTTTTTTACAGACCTT | 751 |
| CT13 BACK 0 | AACTAACTTTGCAATGTGCTGCAAGGCGTTTTTTTTTTTTACCATTACG | 752 |
| CT13 BACK 1 | CAATCGCCCTGTTTGTGCCGGAAACCAGTTTTTTTTTTTTACAAGATCG | 753 |
| CT13 BACK 2 | TATTCACACAATTCCCGTTGCGCACAAACAAATAAACATTACCTCCCCGGTTT | 754 |
| | TTTTTTTTTAGTGGCATA | |
| CT13 BACK 3 | CGCGTAATATCCAGGTAATAAAAGGGACTAATCCTGTGTAGATTTTTTTTTTT | 755 |
| | TATATCGTGC | |
| CT14 TOP 0 | AACCCACGCCACCAAAAGATTAAGAGGATTTTTTTTTTTTAACACATCG | 756 |
| CT14 TOP 1 | CCCCCAGAATACACACCGACCGTGTGATGGCATTTATCCCCCTCAAATGTTTT | 757 |
| | TTTTTTTTATGATGTCG | |
| CT14 TOP 2 | CAGGCGGTAGCTCAATGGGCGCCCACCACCCTCAGATTTTCATTTAATTGTTT | 758 |
| | TTTTTTTTTACATCAAGC | |
| CT14 TOP 3 | TAGTTTGGTAGGTAGGCTATCAGGTCATATTAAGTAATAGCGTTTTTTTTTTT | 759 |
| | TATAACACCG | |
| CT14 RIGHT 0 | GCTTAGATGATAAGCAGACTGTAGCGCGTTTTTTTTTTTTAAGGCAGAT | 760 |
| CT14 RIGHT 1 | GCTGTTTGCTCGAAATCACCAGTAGCACTTTTTTTTTTTTAGATTGACG | 761 |
| CT14 RIGHT 2 | AGAAACGAAACCGAGATACCGATAGTTGCACGCTGTTATTCTGAAACATTTTT | 762 |
| | TTTTTTTTATATGAGCG | |
| CT14 RIGHT 3 | TAACAGTGGTTTAACATTCAACCGATTGTTTTTTTTTTTTACGGTTACT | 763 |
| CT14 BACK 0 | AACTAACTTTGCAATGTGCTGCAAGGCGTTTTTTTTTTTTCGTAATGGT | 764 |
| CT14 BACK 1 | CAATCGCCCTGTTTGTGCCGGAAACCAGTTTTTTTTTTTTTATGCCACT | 765 |
| CT14 BACK 2 | TATTCACACAATTCCCGTTGCGCACAAACAAATAAACATTACCTCCCCGGTTT | 766 |
| | TTTTTTTTTCGATCTTGT | |
| CT14 BACK 3 | CGCGTAATATCCAGGTAATAAAAGGGACTAATCCTGTGTAGATTTTTTTTTTT | 767 |
| | TGCACGATAT | |
| CT15 FRONT 0 | GATCGTCCAATCATAAATCAACGTAACAAGCCCGACCGCCACTTTTTTTTTTT | 768 |
| | TAATCAGGCT | |
| CT15 FRONT 1 | AGCGGTCCGCCGACAAGTTACCAGAAGGCACCAGGCGGATAATTTTTTTTTTT | 769 |
| | TATTCCTTGC | |
| CT15 FRONT 2 | GAACGCGATAGAAGTAGCATTCCACAGATTTTTTTTTTTTAATCCATGC | 770 |
| CT15 FRONT 3 | ACACCGCTAATAGACAACAGTTTCAGCGTTTTTTTTTTTTATCAACTGC | 771 |
| CT15 RIGHT 0 | GCTTAGATGATAAGCAGACTGTAGCGCGTTTTTTTTTTTTCGGCAATAT | 772 |
| CT15 RIGHT 1 | GCTGTTTGCTCGAAATCACCAGTAGCACTTTTTTTTTTTTTCGGACATA | 773 |
| CT15 RIGHT 2 | AGAAACGAAACCGAGATACCGATAGTTGCACGCTGTTATTCTGAAACATTTTT | 774 |
| | TTTTTTTTAGTTCCAGT | |
| CT15 RIGHT 3 | TAACAGTGGTTTAACATTCAACCGATTGTTTTTTTTTTTTCGTGTTAGT | 775 |
| CT15 BACK 0 | AACTAACTTTGCAATGTGCTGCAAGGCGTTTTTTTTTTTTCGATGTGTT | 776 |
| CT15 BACK 1 | CAATCGCCCTGTTTGTGCCGGAAACCAGTTTTTTTTTTTTGCTTGATGT | 777 |
| CT15 BACK 2 | TATTCACACAATTCCCGTTGCGCACAAACAAATAAACATTACCTCCCCGGTTT | 778 |
| | TTTTTTTTTCGACATCAT | |
| CT15 BACK 3 | CGCGTAATATCCAGGTAATAAAAGGGACTAATCCTGTGTAGATTTTTTTTTTT | 779 |
| | TCGGTGTTAT | |
| CT16 BOT 0 | ATCAGCTATCGTAAGCAATTCATCAATATTTTTTTTTTTTCGATCACAT | 780 |
| CT16 BOT 1 | GAGGCCACCTGAGAGTCAATAGTGAATTCCAAGAACCGAACGTTTTTTTTTTT | 781 |
| | TCGACAATGT | |
| CT16 BOT 2 | CATTTTTAACGGATTGGTTGAGAGGAGTGTACTGGTAGGGAGGTGCGTAGTTT | 782 |
| | TTTTTTTTTTAGCCGTAT | |
| CT16 BOT 3 | GAGCCTTACAACTAAAGGAATGAGTGAGGGAGCACTTTTTTTTTTTTCGATAT | 783 |
| | GCT | |
| CT16 TOP 0 | AACCCACGCCACCAAAAGATTAAGAGGATTTTTTTTTTTTATGACACCT | 784 |
| CT16 TOP 1 | CCCCCAGAATACACACCGACCGTGTGATGGCATTTATCCCCCTCAAATGTTTT | 785 |
| | TTTTTTTTATCAGTGGT | |
| CT16 TOP 2 | CAGGCGGTAGCTCAATGGGCGCCCACCACCCTCAGATTTTCATTTAATTGTTT | 786 |
| | TTTTTTTTTGCTGCATAT | |
| CT16 TOP 3 | TAGTTTGGTAGGTAGGCTATCAGGTCATATTAAGTAATAGCGTTTTTTTTTTT | 787 |
| | TGCGTATGAT | |
| CT17 BOT 0 | ATCAGCTATCGTAAGCAATTCATCAATATTTTTTTTTTTTGACGCATAT | 788 |
| CT17 BOT 1 | GAGGCCACCTGAGAGTCAATAGTGAATTCCAAGAACCGAACGTTTTTTTTTTT | 789 |
| | TAGTCCAAGT | |
| CT17 BOT 2 | CATTTTTAACGGATTGGTTGAGAGGAGTGTACTGGTAGGGAGGTGCGTAGTTT | 790 |
| | TTTTTTTTTAGTCCTGTT | |
| CT17 BOT 3 | GAGCCTTACAACTAAAGGAATGAGTGAGGGAGCACTTTTTTTTTTTTTAAGCT | 791 |
| | GGT | |
| CT17 TOP 0 | AACCCACGCCACCAAAAGATTAAGAGGATTTTTTTTTTTTAGTCCAAGT | 792 |
| CT17 TOP 1 | CCCCCAGAATACACACCGACCGTGTGATGGCATTTATCCCCCTCAAATGTTTT | 793 |
| | TTTTTTTTTAAGCTGGT | |
| CT17 TOP 2 | CAGGCGGTAGCTCAATGGGCGCCCACCACCCTCAGATTTTCATTTAATTGTTT | 794 |
| | TTTTTTTTTGACGCATAT | |
| CT17 TOP 3 | TAGTTTGGTAGGTAGGCTATCAGGTCATATTAAGTAATAGCGTTTTTTTTTTT | 795 |
| | TAGTCCTGTT | |
| CT18 BOT 0 | ATCAGCTATCGTAAGCAATTCATCAATATTTTTTTTTTTTAGTAACCGT | 796 |
| CT18 BOT 1 | GAGGCCACCTGAGAGTCAATAGTGAATTCCAAGAACCGAACGTTTTTTTTTTT | 797 |
| | TCGTCAATCT | |
| CT18 BOT 2 | CATTTTTAACGGATTGGTTGAGAGGAGTGTACTGGTAGGGAGGTGCGTAGTTT | 798 |
| | TTTTTTTTTCGCTCATAT | |
| CT18 BOT 3 | GAGCCTTACAACTAAAGGAATGAGTGAGGGAGCACTTTTTTTTTTTTATCTGC | 799 |
| | CTT | |
| CT18 TOP 0 | AACCCACGCCACCAAAAGATTAAGAGGATTTTTTTTTTTTCGTCAATCT | 800 |
| CT18 TOP 1 | CCCCCAGAATACACACCGACCGTGTGATGGCATTTATCCCCCTCAAATGTTTT | 801 |
| | TTTTTTTTATCTGCCTT | |
| CT18 TOP 2 | CAGGCGGTAGCTCAATGGGCGCCCACCACCCTCAGATTTTCATTTAATTGTTT | 802 |
| | TTTTTTTTTAGTAACCGT | |
| CT18 TOP 3 | TAGTTTGGTAGGTAGGCTATCAGGTCATATTAAGTAATAGCGTTTTTTTTTTT | 803 |
| | TCGCTCATAT | |

### Converting Patchy Files to Origamis

The Patchy→Origami library in PyPatchy is designed to enable conversion of patchy particle data to DNA origamis. Since the Patchy→Origami library is part of PyPatchy.

The conversion library is highly modular and extendable, allowing for great flexibility when converting patchy particles to DNA structures.

### Setup

Conversion of patchy particles into DNA origamis requires: a patchy particles structure, a DNA origami monomer, and the IDs of the strands for extension with sticky ends.

### The Scene

The conversion script requires the patchy particle structure to be in multidentate PL Patchy Particle form. If the structure is in a different format, the PyPatchy conversion library can be used to convert it.

Converting a Polycube to a Patchy Scene

| | |
|---|---|
| 1 | # nessecary imports |
| 2 | from pypatchy.patchy.pl.plpatchylib import polycube_to_pl |
| 3 | from pypatchy.polycubeutil.polycube_structure import load_polycube |
| 4 | from pypatchy.patchy.pl.plparticleset import MultidentateConvertSettings |
| 5 | |
| 6 | # extra imports for MyPy annotations, to aid readability |
| 7 | from pypatchy.patchy.pl.plscene import PLPSimulation |
| 8 | from pypatchy.polycubeutil.polycube_structure import PolycubeStructure |
| 9 | |
| 10 | # load a polycube from a file at ~/.pypatchy/input/mypolycube.json |
| 11 | polycube: PolycubeStructure = load_polycube(“mypolycube.json”) |
| 12 | # specify settings that the program will use to convert the polycube to a patchy |
| | particle set |
| 13 | convert_settings = MultidentateConvertSettings( |
| 14 | n_teeth=4, # each patch on the particle corresponds to 4 “multidentate” patches |
| | on the particles, |
| 15 | # which will be mapped to 4 strands on the DNA particle monomer(we'll load that |
| | later) |
| 16 | dental_radius=0.5 # the distance in simulation units from the center of the |
| | patch to the “teeth” |
| 17 | # Here, I assume that the patches are on the edges of the face of a square particle |
| | 18 |
| 18 | # with radius 0.5, so set my dental radius to 0.5 |
| 19 | ) |
| 20 | # convert the polycube to a single patchy conf |
| 21 | # (disregard class name “PLPSimulation”, we are not simulating anything right |
| | now) |
| 22 | scene: PLPSimulation = polycube_to_pl(polycube, convert_settings) |

Importing a Patchy Scene - Lorenzian

| | |
|---|---|
| 1 | # nessecary imports |
| 2 | from pypatchy.patchy.pl.patchyio import get_writer |
| 3 | from pypatchy.patchy.pl.plparticleset import MultidentateConvertSettings |
| 4 | |
| 5 | # lookup patchy writer object |
| 6 | # our patchy files are in Lorenzo's format so use the build in Lorenzo-Writer |
| 7 | # (it can read or write particle data but today we are using it as a reader) |
| 8 | # honestly this should be a context manager, srry |
| 9 | reader = get_writer(“lorenzo”) |
| 10 | |
| 11 | # set the writer's working directory to the location of our files |
| 12 | my_patchy_dir = “/path/to/my/patchy/directory” |
| 13 | reader.set_directory(my_patchy_dir) |
| 14 | |
| 15 | # first load patchy particle set |
| 16 | # this varies a lot between patchy particle formats, this is only the correct |
| | method for Lorenzo-format |
| 17 | patchy_particle_set = reader.read particle types(topology=“init.top”, |
| 18 | DPS_interaction_matrix_file=“interactions.txt”) |
| 19 | # specify settings that the program will use to convert the polycube to a patchy |
| | particle set |
| 20 | convert_settings = MultidentateConvertSettings( |
| 21 | n_teeth=4, # each patch on the particle corresponds to 4 “multidentate” patches |
| | on the particles, |
| 22 | # which will be mapped to 4 strands on the DNA particle monomer(we'll load that |
| | later) |
| 23 | dental_radius=0.5, # the distance in simulation units from the center of the patch |
| | to the “teeth” |
| 24 | # Here, I assume that the patches are on the edges of the face of a square particle |
| 25 | # with radius 0.5, so set my dental radius to 0.5 |
| 26 | torsion=false |
| 27 | ) |
| 28 | # convert particles to multidentate |
| 29 | patchy_particle_set = patchy_particle_set.to_multidentate(convert_settings) |
| 30 | |
| 31 | scene = reader.read_scene(top_file=“init.top”, # topology file |
| 32 | traj_file=“last_cont.dat”, # here we will give it a single conf |
| 33 | # but if we wanted we could pass a trajectory and a timestep |
| 34 | particle_types=patchy_particle_set |
| 35 | ) |
| 36 | |
| 37 | # if we wanted to convert this scene to multidentate we would do it here, |
| 38 | # but the scene is already multidentate |

Define Particle Types Pythonically

| | |
|---|---|
| 1 | patchy_particle_set = PLParticleSet([ |
| 2 | PLPatchyParticle( |
| 3 | type_id=0, # each particle should have a unique type ID |
| 4 | radius=0.5, # optional(default is 0.5) |
| 5 | # can specify custom radius, but since the particle will be auto-scaled to match |
| | the DNA monomer it doesn't matter |
| 6 | patches=[ |
| 7 | PLPatch( |
| 8 | type_id=0, # patch type IDs must be unique within the entire particle set |
| 9 | color=21, # colors start at 21, and −21 |
| 10 | relposition=np.array([0.5, 0, 0]), # patch position relative to particle |
| 11 | a1=np.array([1, 0, 0]), # patch direction vector, should point “outwards” |
| 12 | a2=np.array([0, 1, 0]) # patch orientation vector. in torsional models, patch |
| | orientations must be parallel for patches to bind2 3 4 5 6 7 8 9 10 11 12 |
| 13 | # even w/o torsion, a2 vector is required as starting pt for multidentate |
| | conversion |
| 14 | ), PLPatch( |
| 15 | type_id=1, |
| 16 | color=22 |
| 18 | # |
| 19 | ) |
| 20 | ] |
| 21 | ), |
| 22 | PLPatchyParticle( |
| 23 | type_id=1, |
| 24 | radius=0.5, |
| 25 | patches= [ |
| 26 | PLPatch( |
| 27 | type_id=3, |
| 28 | color =−21, |
| 29 | # etc |
| 30 | ) |
| 31 | ] |
| 32 | ) |
| 33 | ]) |

### The Monomers

The conversion script requires at least one monomer to be used to produce the structure. Monomers should be provided as .top and .dat files

### Strand IDs

Identify which staple strands on the origami have 3′-ends which can be used to extend sticky ends. Strands should be grouped together as patches which correspond to the patches in your particle set. The program will automatically handle it from there.

| | |
|---|---|
| 1 | patch_positions = [ |
| 2 | [134, 139, 131, 133], # group of strands that form a patch |
| 3 | [153, 156, 157, 154], # group of strands that form another patch |
| 4 | [149, 160, 151, 148], |
| 5 | [159, 167, 162, 158], |
| 6 | [146, 147, 145, 144], |
| 7 | [143, 141, 150, 163] |
| 8 | ] |

The grouping of the patch IDs matters, but the order of the two lists does not matter because the converter script will automatically assign the patches to ideally align the patchy particle with the DNA strands.

Importing a DNA Structure

| | |
|---|---|
| 1 | # imports |
| 2 | from ipy_oxdna.dna_structure import load_dna_structure |
| 3 | from pypatchy.util import get_input_dir |
| 4 | from pypatchy.patchy.dna_particle import DNAParticle |
| 5 | |
| 6 | # load a DNA structure to use as our monomer. |
| 7 | # here we will load the files at ~/pypatchy/input/scenes/cube_monomer.top and |
| | ~/pypatchy/input/scenes/cube_monomer. |
| 8 | # unlike the aforementioned load_polycube function this will NOT automatically |
| | prepent ~/.pypatchy/input |
| 9 | dna = load_dna_structure(get_input_dir( ) / “scenes/cube_monomer.top”, |
| 10 | get_input_dir( ) / “scenes/cube_monomer.dat”) |
| 11 | # create DNA monomer from DNA structure and patch positions |
| 12 | dna_particle = DNAParticle(dna, patch_positions) |

Converter Setup

| | |
|---|---|
| 1 | # imports |
| 2 | from pypatchy.patchy.patchy_origami_convert import PatchyOrigamiConverter |
| 3 | |
| 4 | converter = PatchyOrigamiConverter(scene, |
| 5 | sticky_length=10, # length of the binding portion of single-stranded overhangs |
| 6 | # if we set this to a number, the program will generate a random |
| 7 | # sequence (not guaranteed to be orthogonal!!!!) |
| 8 | # if we set this to None you will have to manually enter all sequences |
| 9 | # below |
| 10 | spacer_length=12, # poly-T spacer which will seperate the binding portion of the |
| 11 | # overhang from the rest of the staple strand |
| 12 | # there/ are a lot more optional args |
| 13 | ) |
| 14 | # patchy color sequences start with 21 and −21 and proceed from there |
| 15 | converter.assign_color_sequence(21, # color in patchy particle |
| 16 | “ATGCATCGAT”, # sequence |
| 17 | update_rc=True # the converter will automatically assign −21 to the |
| 18 | # reverse compliment ATCGATGCAT(True is default value) |
| 19 | ) |
| 20 | |
| 21 | # set the converter to use our DNA particle from before |
| 22 | # if you want to have multiple distinct DNA monomers for different particle types, |
| 23 | # you can assign them here |
| 24 | converter.assign_particles(dna_particle) |

Converting

| | |
|---|---|
| 1 | # position DNA particles |
| 2 | converter.position_particles( ) |
| 3 | |
| 4 | # join them together |
| 5 | # can take a while |
| 6 | converter.convert( ) |

Exporting .top and .conf

| | |
|---|---|
| 1 | # if you provide a relative path, pypatchy will place in the directory |
| | ~/ .pypathy/output |
| 2 | # alternatively, if you provide an absolute path (beginning with “/”) the program |
| | will use that directly |
| 3 | converter.save_top_dat(write_top_path=“mystructure_dna.top”, |
| 4 | write_conf_path=“mystructure_dna.conf”) |

Exporting as .oxview

| | |
|---|---|
| 1 | converter.save_oxview(write_oxview_path=“mystructure_dna.oxview”, |
| | color_by_type=True) |

### Exporting Sequences

The converter has a built in function to export sequences for easy ordering. Continuing from the previous example, call:
1 converter.export_stickys_staples(“/path/to/folder/example.xlsx”)
   This feature is designed to format the spreadsheet according to IDT specifications. The resulting notebook will have one sheet for each 96-well plate. Sheets each have three columns labeled Wells, Name, and Sequences.
by_row
Options: True, False. If set to True, the excel spreadsheet will export so as to make each row on the 96-well plate (each pair of letters) correspond to a specific particle. If each DNA particle doesn't have exactly 24 sticky candidates, the program will throw an error. Default is currently True.
incl_no_sticky
Options: True, False. If set to True, the spreadsheet will only include strands which have sticky-ends (the strands that facilitate interparticle interaction). Default is True.
incl_absent
Options: True, False. If set to True, the converted will include all staples required for the origami, not just ones which have been modified to attach sticky ends. Default is True.
incl_original_patch_nums
Options: True, False. If set to True, the converter will include the patch numbers or (for polycuebes) direction names. This currently only works with polycubes. Default is False.

### Example Design Script

The following example converts a Polycube to patchy and then converts the patchy particle scene.

| | |
|---|---|
| 1 | from pypatchy.patchy.pl.plpatchylib import polycube_to_pl |
| 2 | |
| 3 | from pypatchy.dna_structure import load_dna_structure |
| 4 | from pypatchy.patchy.dna_particle import DNAParticle |
| 5 | from pypatchy.patchy.patchy_origami_convert import PatchyOrigamiConverter |
| 6 | from pypatchy.patchyio import get_writer |
| 7 | |
| 8 | from pypatchy.polycubeutil.polycube_structure import load_polycube |
| 9 | from pypatchy.util import get_output_dir |
| 10 | |
| 11 | name=“cube 4x4x4” |
| 12 | structure_file_name = f“scenes/ {name}.json” |
| 13 | |
| 14 | strand_ids = [ |
| 15 | [134, 139, 131, 133], |
| 16 | [153, 156, 157, 154], |
| 17 | [149, 160, 151, 148], |
| 18 | [159, 167, 162, 158], |
| 19 | [146, 147, 145, 144], |
| 20 | [143, 141, 150, 163] |
| 21 | ] |
| 22 | |
| 23 | # load dna particles |
| 24 | dna = load_dna_structure(“scenes/cube_monomer.top”, “scenes/cube_monomer.dat”) |
| 25 | # load polycube from file |
| 26 | polycube = load_polycube(structure_file_name) |
| 27 | # convert polycube to a multidentate patchy particle conf |
| 28 | scene = polycube_to_pl(polycube, nteeth=4, dental_radius=0.25) |
| 29 | # construct dna particle object |
| 30 | dna_particle = DNAParticle(dna, patch_positions) |
| 31 | |
| 32 | # construct converter object |
| 33 | converter = PatchyOrigamiConverter(scene, |
| 34 | padding=1, |
| 35 | spacer_length=8, |
| 36 | sticky_length=8, |
| 37 | expected_num_edges=4 * polycube.num_connections( ) |
| 38 | # flexable_patch_distances=True |
| 39 | |
| 40 | ) |
| 41 | |
| 42 | # the converter uses patchy-particle nomenclature, so color 1 is 21, color −1 is |
| | −21, color 2 is 22, etc. |
| 43 | converter.assign_color_sequence(21, “ATGCATGC”) # put example sequences here |
| 44 | # the converter will automatically assign reverse-compliments to negative colors |
| 45 | |
| 46 | # add more converter.assign_color_sequence calls to assign 22, 23, 24, etc. |
| 47 | |
| 48 | # assigns DNA particles and strands to patchy particles |
| 49 | converter.assign_particles(dna_particle) |
| 50 | |
| 51 | # this function saves the DNA particle monomers of the sequence as individual |
| | oxDNA .top and .dat files |
| 52 | converter.dump_monomers(name) |
| 53 | |
| 54 | # the next two lines convert the patch particles to DNA |
| 55 | converter.position particles( ) |
| 56 | converter.convert( ) |
| 57 | |
| 58 | # the next line saves the full patchy particle scene / polycube structure as a |
| | single oxDNA .top and .dat |
| 59 | converter.save_top_dat(write_top_path=f” {name} /dna.top”, |
| | write_conf_path=f” {name} /dna.conf”) |
| 60 | |
| 61 | |

Conjugation of Single Walled Carbon Nanotubes (CNT) with DNA Nanocube for Scaffolding of Carbon Nanotube Circuits

### Fabrication of DNA-CNTs Conjugates

Carbon nanotubes (CNTs) enriched in (6,5) chirality (>95% as carbon nanotube basis; average diameter 0.78 nm, Sigma-Aldrich) are used in this study. The L1 DNA strand used for CNT functionalization is: L1 (SEQ ID NO: 974). L1-CNT complexes are prepared by mixing (6,5) CNTs and L1 ssDNA at a mass ratio of 2.5:1 (DNA:CNT) in 30 mM NaCl in Milli-Q water (18.2 MΩ·cm). The mixture is subjected to tip sonication using a VC50 sonicator (Sonics & Materials Inc.; power output 50 W maximum, frequency 20 kHz, amplitude 90%) in an ice bath for 2 h.

The resulting L1-CNT dispersion is diluted to a final concentration of 80 μg/mL CNTs (using the published value of 13 μg/mL for OD=1 at 990 nm for (6,5)-enriched CoMoCAT). NaCl is added to reach a final concentration of 500 mM, and a 50% (w/v) polyethylene glycol (PEG, 8 kDa) solution is introduced to achieve the desired PEG concentration for the first precipitation step.

PEG (8 kDa) is added stepwise from 2% to 14% (w/v) in 2% increments. The CNT/PEG mixture is incubated at 4° C. for 24 h for PEG concentrations up to 8%, for 48 h at 10-12%, and for 72 h at 14%. After incubation, each sample is centrifuged at 11,300 rcf for 10 min at 4° C.; the vial is rotated 180° and centrifuged again to ensure complete pelleting. The pellet is removed, and only the supernatant is collected for further steps. For the 14% PEG step, the resulting pellet is redispersed by gentle pipetting in 100 mM NaCl solution to prevent DNA unwrapping and preserve the π-π stacking interaction between the DNA and CNT surface.

Unlike the original protocol, no additional free ssDNA is added after redispersion. The L1-wrapped CNTs remain stable for more than one week at 4° C. For long-term storage, shorter oligonucleotides such as GT10 or GT20 are added to preserve solution stability without interfering with subsequent L2, L3, and L4 hybridizations.

Hybridization of L1-CNTs with DNA Anchors

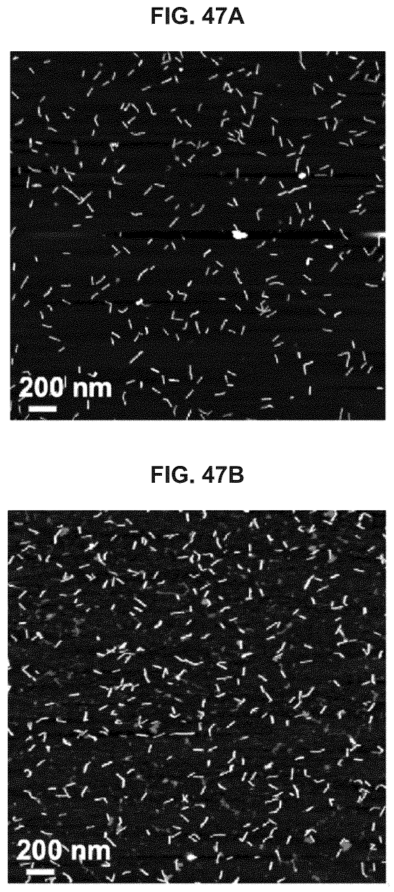

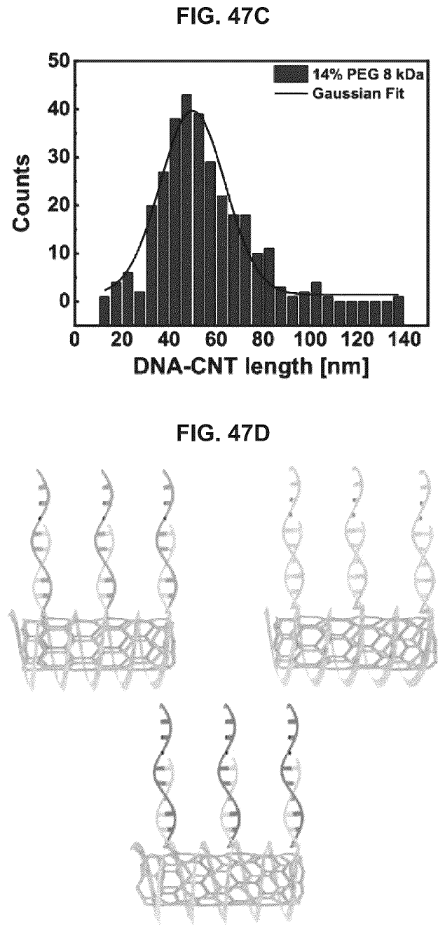

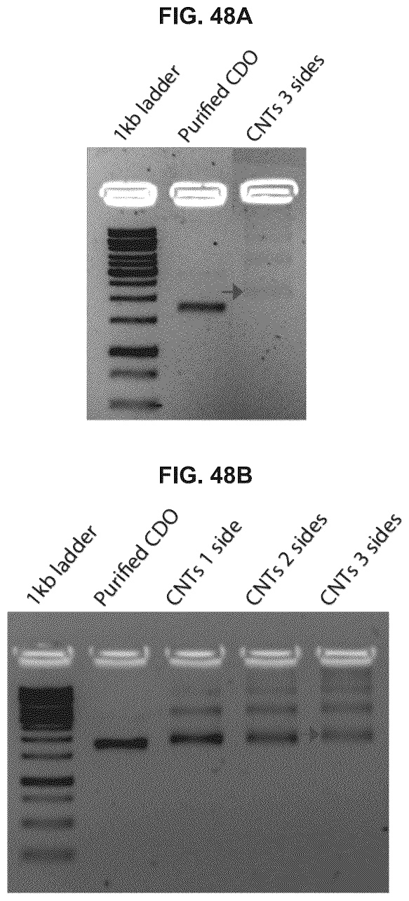

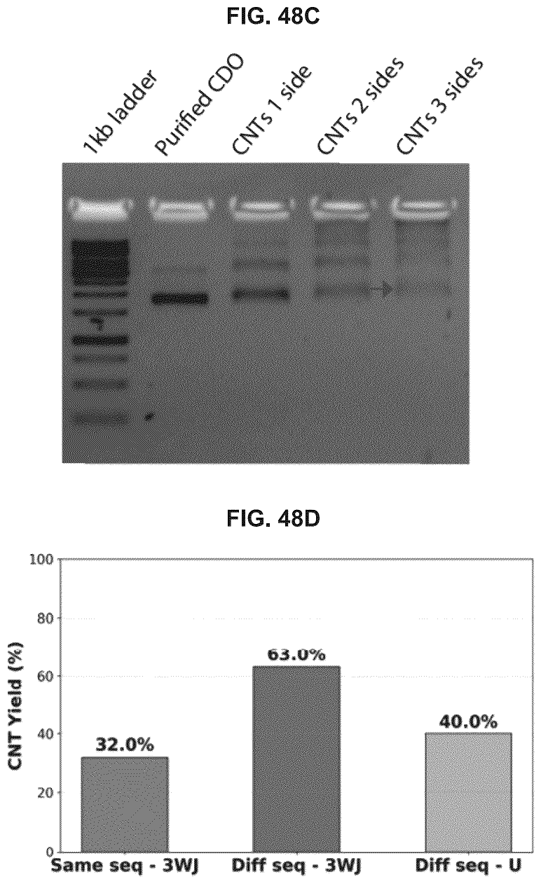

Three distinct DNA anchor strands (L2, L3, and L4; SEQ ID NO: 975-977) are designed to be partially complementary to the L1 sequence on the CNTs and to contain a binding domain complementary to the binding sites on the DNA origami cube. A schematic illustration of the hybridization process between L1-wrapped CNTs and the three DNA anchor strands is shown in FIG. 47A-B.

Hybridization between L1-wrapped CNTs and each corresponding DNA anchor strand (L2, L3, or L4) is carried out following an adapted protocol from Chen et al. Specifically, mixtures of L1-CNT dispersions and each anchor strand are incubated in 100 mM NaCl in Milli-Q water adjusted to pH 5.8. The concentration of each anchor strand is set equal to that of the L1 strand used for CNT functionalization to ensure stoichiometric hybridization. The hybridization temperature is gradually decreased from 37° C. to 25° C. over 2 h, with two repeated cycles of 1° C. cooling every 5 min using a programmable thermal cycler.

After hybridization, the resulting DNA-CNT conjugates carrying the L2, L3, and L4 overhangs are stored at 4° C. until further assembly with the 3D DNA origami cubes. Throughout this process, the hybridization buffer contains only NaCl (100 mM); MgCl₂ is deliberately excluded to prevent CNT aggregation. Mg²⁺ ions are introduced only after the conjugation step with the DNA origami structures.

The fabrication of DNA-CNT conjugates is achieved through sequential PEG-induced precipitation, AFM-based characterization, and DNA hybridization with designed anchor strands. CNTs (Signis SG65i, Sigma-Aldrich) were functionalized with single-stranded DNA (L1) in 30 mM NaCl using controlled sonication to ensure stable π-π stacking between DNA and CNT surfaces. PEG-induced precipitation was then applied stepwise at 10° C. to fractionate nanotubes by length. AFM characterization confirmed uniform DNA coverage and revealed an average nanotube length of approximately 50 nm for PEG (8 kDa) sorted samples. Subsequently, each batch of L1-CNT were hybridized separately with one of three DNA anchor strands (L2, L3, or L4), which were partially complementary to L1 and contained toehold domains for DNA origami binding.

The hybridization process proceeded in 100 mM NaCl, pH~5.8, with gradual cooling from 37° C. to 25° C. with a ramping rate of 1° C. per 5 min. The resulting DNA-CNT conjugates carrying L2, L3, and L4 overhangs were stored at 4° C. for later assembly with DNA cubes.

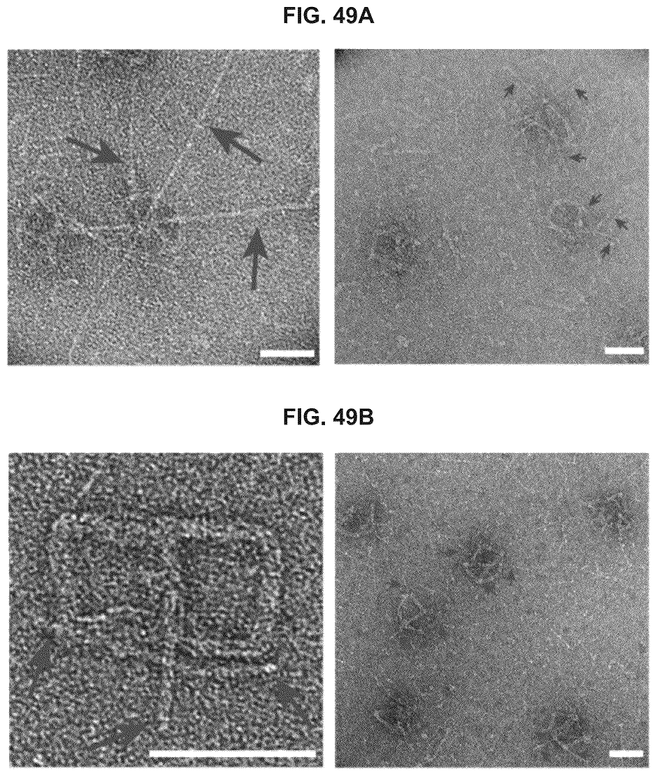

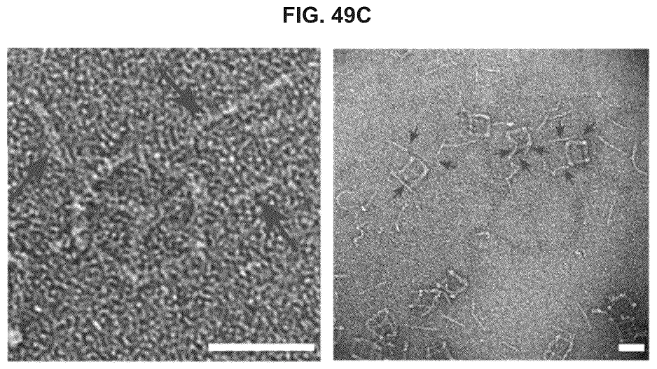

For DNA cube-CNT conjugation, a 1.5× excess of single-walled CNTs was added to the purified cube at final concentrations of 200 mM NaCl and 5 mM Mg²⁺. The strands were annealed with gradual cooling from 47° C. to 33° C. with a ramping rate of 0.1° C. per 15 min. The resulting 2D and 3D CNT junctions, scaffolded by DNA nanocubes are shown in FIG. 49A-C.

TABLE 12
Handle sequences for CNT U-shape design. The core cube sequences were
rearranged to create anchor sites for CNT docking in both U-shape and 3WJ designs. L1
wraps around the CNTs, while L2-L4 hybridize with L1 to attach the CNTs to the CDOs.
Three polyT spacers are used for the sticky ends on the CDOs to bind with the CNTs, and
14-nucleotide hybridization is used for CNT-CDO binding.

| | | |
|---|---|---|
| | | SEQ ID |
| Name | DNA Sequence (5′→3′) | NO |

CDO cores

| | | |
|---|---|---|
| core CNT strand 0 | GAACAAGCAATTTTTCCGTTTTTATTTTCAAAAAC | 804 |
| core CNT strand 1 | AGCAGCCCGATCTAAATCATTACCGCGCGATTTTCCCTTA | 805 |
| core CNT strand 2 | AACTACAGGGAGAAACCCAGCTACAATTGATTAGTATTCTAA | 806 |
| core CNT strand 3 | TGCGTTATACGTTGCTATTTTATGAGGAAGTTTAGTATCATA | 807 |
| core CNT strand 4 | AACGCTCAACTTTTTGTAGGGCTTAATTGACTAGA | 808 |
| core CNT strand 5 | GACAAAAGGTTTTTTAAGTAATTCTGTCCATAATT | 809 |
| core CNT strand 6 | ATAATATCCCTTTTTTCCTAATTTACGAGCGCTTA | 810 |
| core CNT strand 7 | TAACAACCTGCAACAAACAAAATTAATTCCAAAAAAATAATT | 811 |
| core CNT strand 8 | CAAATATATTACCCAAGGGAACCGAACTGGCCGCTGCAAGAA | 812 |
| core CNT strand 9 | GCGGATTAGGCTTGTCCATGTTACTTAGCATCGGAGAGAGAT | 813 |
| core CNT strand 10 | GCCGCCAGGCGGTTTGCGTATCATGTTTTGCTGAA | 814 |
| core CNT strand 11 | TTTGCCTAGAGCCGATCGGCCAACGCGCAAGTACGGCGGATG | 815 |
| core CNT strand 12 | TAGTAAGAGCTTTTTACACTATCATAACCCTTAGG | 816 |
| core CNT strand 13 | CGCCAGGCTACAAAAAGATTCAAAAGGGCGAACGATCATCAG | 817 |
| core CNT strand 14 | TGGCGAACAAGAGACCTCATATATTTTAATGGTCATAAAACG | 818 |
| core CNT strand 15 | TCCTCATACTCACATTAATTGACACAACGAAATTG | 819 |
| core CNT strand 16 | TTAGAGCAAAGCGCGGGTGCCTAATGAGCATAAAGGGTCATA | 820 |
| core CNT strand 17 | CAACATTAAAATTGTAAAGCCACGTCAAAGGGCAGCTTTCAT | 821 |
| core CNT strand 18 | GGATTCTCCGTTTTTGGGAACAAACGGCGGCTGGC | 822 |
| core CNT strand 19 | TGGATTAACGACCAAACAATATTACCGCTAGCGGTAAAATAA | 823 |
| core CNT strand 20 | ATTATCAACCCTTCAGTAGAAGAACTCAATGCGCCTTGTTAA | 824 |
| core CNT strand 21 | AATAAGTATAGCCCGAGATAGCGCCTGAACATCGG | 825 |
| core CNT strand 22 | AGACAAATGCCTTGAATCCCTTATAAATAAGTTACTATACAG | 826 |
| core CNT strand 23 | TTGAGATAGAATGACATATTTAACAACGTAAACACACGAAGG | 827 |
| core CNT strand 24 | TATGCGATCATTGATCGAGCCAGTAATATTGAAATTAAAACACTGCAT | 828 |
| | CA | |
| core CNT strand 25 | TATGTACAGGCTGCACAATAAACAACATCTTTTTCCATACAG | 829 |
| core CNT strand 26 | AGATTGTGCTTCTGATCAACAATAGATATGCTGATCCTCAGAGCTTTC | 830 |
| | CT | |
| core CNT strand 27 | TTTTGAATATCATTAACCAATCAATAATAGACTACTAAAGGG | 831 |
| core CNT strand 28 | TGAGCATATCAGCAACTGGCATGATTAAGACTGATAATTGT | 832 |
| core CNT strand 29 | GATATTCCGCGTTTTCAGAACCGCCACCAATAAGATTTGCGG | 833 |
| core CNT strand 30 | GTGAATAGCATCAACCCTCATTTTCAGGAATATCAACGAGGG | 834 |
| core CNT strand 31 | AGCAAACATCAAGAGTATTTTTTATCTTGACATTTGAAAGAGGAC | 835 |
| core CNT strand 32 | TATAATGAGTACCTCGGCATTTTCGGTCCTCAGAGCAGGGTGGTTTTT | 836 |
| | CACAGAC | |
| core CNT strand 33 | ACCTGTTTCGTGCCAGCTGCACATTCCAAACTAATGCAGATACATAAT | 837 |
| | TGTGTCTG | |
| core CNT strand 34 | GAGAGATGTTTTCCCCAGACGACGATAAGAAAGATGTAGATT | 838 |
| core CNT strand 35 | GAGCAAAAGGGGGAAAGAAGTTTTGCCAACGTTAAATAACCT | 839 |
| core CNT strand 36 | CCAAGCTTAATGCCGGATTTTTTGAGGGTAGCGGCCGGAGACAGT | 840 |
| core CNT strand 37 | TTATCCGTAGAGGAATTAGCAAGGCCGGTGATATTCTCACTGCCCGCT | 841 |
| | TATATGA | |
| core CNT strand 38 | CAGTCACTACTTCTGTAATGGGATAGGTGCCATCACACGCTG | 842 |
| core CNT strand 39 | AGATAGAGATGATGCCGTGCATCTGCCATAAATTTGCTACAG | 843 |
| core CNT strand 40 | AAAATTATGGATTATTTTTTTTTACATTGGCATGCAACAGGAAAA | 844 |
| core CNT strand 41 | CCGAAAGCGAATTGCTGAACCTCAAATATCAAAAAAAATCG | 845 |
| core CNT strand 42 | AAATCAGGTCTTTTACCCTGACTATTAACCAGAACTTTTTGAGTAGTA | 846 |
| | ACTGAT | |
| core CNT strand 43 | ATAAGAACCAACATCAGTTCAGAAAACGGGTTTAAAACGGAG | 847 |
| core CNT strand 44 | ATTCTACTAAATTTAATGGTAGAGAATATCGTCATAAATATTTTTAAG | 848 |
| | TCTTTGA | |
| core CNT strand 45 | CCAATAACCAGTCATTTTTTGGACGTTTCATTTGGTGGCGCGAGCT | 849 |
| core CNT strand 46 | GAAGGGCGATCTTTTGCGGGCCTCTTCATCGTAAATTTTTACTAGCAT | 850 |
| | GATTTC | |
| core CNT strand 47 | GAGAAAAGTTCAGCGCCATTCGCCATTCCCCGGTTACTTTTG | 851 |
| core CNT strand 48 | CGTTAGTAACTATATGTAAAAGTCCTGAGCTTTCCGGCACCATAAGCA | 852 |
| | AAAGCTA | |
| core CNT strand 49 | TCGCACAAACGTTATTTTTTATATTTTTTGCTTTGTACGAGCACGT | 853 |
| core CNT strand 50 | AGAAACCACCATTTAGGAGCGGAATTAAAGAATACTTTTTGTGGCACA | 854 |
| | GTTCTT | |
| core CNT strand 51 | CTGTCCAACAGGAAATCATAGG | 855 |
| core CNT strand 52 | GAGTGATAGATTAAGACGCTAGTACCGAACTCGTATTAAATTCGCCAT | 856 |
| | AATCAGT | |
| core CNT strand 53 | TTTAGAAGTATTTTGACTTTACAAACAGAACCACCTTTTTAGCAGAAG | 857 |
| | ATTTTT | |
| core CNT strand 54 | AAGGAACTAAAGCATTTTTTTCACCTTATTCATTTTCAATTACC | 858 |
| core CNT strand 55 | TTTGCCATCTTTTTCATAATCAAAATCCCTCAGAGTTTTTCCGCCACC | 859 |
| | CCAGTG | |
| core CNT strand 56 | CATCGATAGCATTTACCGTAATCAGTAAGGAGGTTTTTTTGAGGCAGG | 860 |
| | TGGGAA | |
| core CNT strand 57 | AGGTGAATTATTTTCCGTCACCGACTTCCGTTCCATTTTTGTAAGCGT | 861 |
| | CGAGTC | |
| core CNT strand 58 | ATATGGTATAAACATTTTTGTTAATGCCTCCTGTTTGATTTGGTGGTT | 862 |
| core CNT strand 59 | AAAAGTTTTTCCTGTTTCCATTAAACGGGTAAAATACGTAATCATCGC | 863 |
| | ATTGGGC | |
| core CNT strand 60 | AGGCAGAAAATAAGGAAAGAGGCAAAAGCGATTATATTACCT | 864 |
| core CNT strand 61 | TCATCTTTTTTTCTGACCTAATAGTAGTAGCATTAACATCCAGCCTTT | 865 |
| | TCAATCA | |
| core CNT strand 62 | GAACGCGAAGACAATAGCAAAATTAAGCTGTACCAAACAGGA | 866 |
| core CNT strand 63 | GGTTGTTTTTGGTTATAAATCAGAGCGGGAGCTAAACAGGAGCAATAC | 867 |
| | ACAATAT | |
| core CNT strand 64 | TAAGTATTTAAGAACGCATAACCGATATGCCCTTCACCGGAA | 868 |
| core CNT strand 65 | CAATATTTTTGCTATCTCGCTGAGGCTTGCAGGGAGTTAAAGACCAAC | 869 |
| | AGAACCG | |
| core CNT strand 66 | CCTCAGAAAGAATTGCAGCGAAAGACAGCCGGAACTCATTCA | 870 |
| core CNT strand 67 | CTCCTTTGCTTAATTAAATATGCAACTAGGGGAGACCAGAAC | 871 |
| core CNT strand 68 | AATACTTTTTCACATTCTATAACAGTTGATTCCCAATTCTGTGAGAAA | 872 |
| | TATTTTT | |
| core CNT strand 69 | AGAGGCTGGAACAAGATACATTTCGCAAAATGCAAGAGTCTG | 873 |
| core CNT strand 70 | GTACCGACCTGTGTATACGAGCCGGAAGTGAGCTATAAAGCC | 874 |
| core CNT strand 71 | CTTCCTTTTTTGTAGCCGAAAAACCGTCTATCAGGCAAGTGCAGCCAT | 875 |
| | GATTCAC | |
| core CNT strand 72 | ATTTGTATGCCACTCGGAATCATAATTAGAATCGCCCATAAATCAA | 876 |
| core CNT strand 73 | ATGTTTAGACTTTTATAGCGT | 877 |
| core CNT strand 74 | CGGGAGAAATAAATAAATATATTTTAGTGACGACGGCAACTGTTGG | 878 |
| core CNT strand 75 | CAGTATCGGCTTTTAGGAAGA | 879 |
| core CNT strand 76 | CGTTGTAGGCCGATCTTTTTAACCTCCGATGTAGATTGCGGAACAA | 880 |
| core CNT strand 77 | AAGCTGCGAGGCGCAGACGGTACCCTCAGAGTTAAGCCCAATCTCAGA | 881 |
| | AAAGACTT | |
| core CNT strand 78 | AAATTTTGTGTCGAAATCCGCGGCTACAGGTAATTGAGCGCTGATAGC | 882 |
| | AAAGCAAA | |
| core CNT strand 79 | ATTAGAGCTCATAGGCTGGCTGACCTTCTCCAACACCTTATTAGCG | 883 |
| core CNT strand 80 | CACCACCTTAGCGTAGGTCATTTCGCCAAAAGGAAAATCAAG | 884 |
| core CNT strand 81 | AACGCTTAAGGATAAAAATTTTATATTTGGGAAGAAAAATCTGAGGGG | 885 |
| | GCGCCAGC | |
| core CNT strand 82 | TCGACTCCTACCGTTCTAGCTGATAAATTGCATGCACCAATGAAAC | 886 |
| core CNT strand 83 | AGAATGGCAGCAAATTCGTAATCTGTGAGCGAGTATTGGGAA | 887 |
| core CNT strand 84 | ATTCTGGGGCCTTGCTGGTAACCACCACTAACCAATAGGAACCACGTT | 888 |
| | GGATTGTT | |
| core CNT strand 85 | TGATTTTAGTAATAACATCACACTATGGGTTAAAATTCGCATGTTTGA | 889 |
| | GATTCCTG | |
| core CNT strand 86 | GAAAAGGTGCAAACTGGCTCATTATCTGCGGAATAAAGTACC | 890 |
| core CNT strand 87 | ATAACGTGCATAATATTTAAATTGTTCCAGCCAACAAGAAAA | 891 |
| core CNT strand 88 | AATGGTTAAACAGTACATAAATCAATATTAAAAATACCGAACATTCGA | 892 |
| | CCACTCATCGA | |
| core CNT strand 89 | TAGCAACGACCTGCCCCTGACGAGAAACTAGTCAGAGCCCAATAG | 893 |
| core CNT strand 90 | ACAATGAGCCACCCTAATTCGAGCTTCAAAGCGAACTTTTAGACCGGA | 894 |
| core CNT strand 91 | AGATGAACGGTGTTTTTCACTCAGAACCGCCACCATAGCCCGGTCAGG | 895 |
| core CNT strand 92 | GAAGTTTTTAATGACCGCCAGCATTGACGCGACAGTTACGAGGCA | 896 |
| core CNT strand 93 | GTTTAGCTTAGAACATCGATGAACGGTAGCTATTATAATAGTAAA | 897 |
| core CNT strand 94 | TTGAGATTCGTTTACAGTCACGACGTTGTAAAACGATTTTGGCCAGTG | 898 |
| core CNT strand 95 | CAAATCACCATCATCCAGTCCAGACGATTGGCCTAAACGTCCTGCAGG | 899 |
| core CNT strand 96 | CACTATTTTAAAGAACGTGGACTCCATGAGTCTCTGAATTTAGAGCCA | 900 |
| | TACAACCCGTC | |
| core CNT strand 97 | GGCGCGTTTGCCTGTGACCTGAAAGCGTTCATCATGGGACGACGA | 901 |
| core CNT strand 98 | TTCGCGTATTGACCGAATAATGGAAGGGTTAGAACCTTTTACCATATC | 902 |
| core CNT strand 99 | AAACAGGGAATCTTACCAACGGGTTTTGTTAGCGA | 903 |
| core CNT strand 100 | CAGATAGCGAGAGGTAGGATTAGCGGGGGAAAGTATGCAGCA | 904 |
| core CNT strand 101 | TAAAACACCTTTGCCGGGTATTAAACCAGAGAAGAAGTGTTTTTATAT | 905 |
| | GT | |
| core CNT strand 102 | CCCCAAAAAAACATTATGACCAAAGAATAGAACGC | 906 |
| core CNT strand 103 | ATTGTGAACCAAGCGCGAAACCTAAAACGCGTTAA | 907 |
| core CNT strand 104 | TGGGCGCCATTTTTACCCGCCGCGCTTAAACTATCCCAACAG | 908 |
| core CNT strand 105 | TATCAAACGGTACGCCAGAATCCGAGTAATAGCCC | 909 |
| core CNT strand 106 | TTATTAACGAACTGAAAGAGT | 910 |
| core CNT strand 107 | CTAAAATAGCCAGCATGATGAAACAAAC | 911 |
| core CNT strand 108 | AGCGTAATTTACAGGCGTCTTTCCAGAGGCTATTAATTAACTT | 912 |
| core CNT strand 109 | TCTGAGCGGCTGTAGTTTGAGTAACATTGGCTATAATTAAC | 913 |
| core CNT strand 110 | TGCCTGATGCCTGAGTAATGTACCATTACATTATTACAGGTAAAACCA | 914 |
| | ATGGGTAA | |
| core CNT strand 111 | AATCGGTAATAAAGGCAAATC | 915 |
| core CNT strand 112 | CAATAATTGGCAACAGTTTATTTTGTCACAATCAATTTTAAAATTC | 916 |
| core CNT strand 113 | CGCAGAGTCGGCAAAGTAAC | 917 |
| core CNT strand 114 | GGGTCAGAGGGCGACGTCAGATGACCCTCAATCAAGCGCCAA | 918 |
| core CNT strand 115 | ATTTTCAACCTTTTTTGCTTTGAATACCCAAAAGATTTAACG | 919 |
| core CNT strand 116 | TAACAGTGGTTTAACATTCAACCGATTG | 920 |
| core CNT strand 117 | GAGAAACAAGAAATGAAGGTA | 921 |
| core CNT strand 118 | ACGCTCATGGAAAGGAACAAATACAT | 922 |
| core CNT strand 119 | AGAAATAAAGACGCTCAATCGTCTGAAATTTGCACTTATTCATTAA | 923 |
| core CNT strand 120 | GAACACCCTCGTCACGTATAAAGCC | 924 |
| core CNT strand 121 | TGAGTTTGAACAAATTTTTTGTCAGAGGAGGCTTTTGAGGAC | 925 |
| core CNT strand 122 | CCAGACTTTTTTAGTAAATGAATTTTTTTGTTTAATTTTTTCGTCAAA | 926 |
| | AATGCCAGTTACAAA | |
| core CNT strand 123 | GATCGTCCAATCATAAATCAACGTAACAAGCCCGACCGCCAC | 927 |
| core CNT strand 124 | ACACCGCTAATAGACAACAGTTTCAGCG | 928 |
| core CNT strand 125 | ATCAGCTATCGTAAGCAATTCATCAATA | 929 |
| core CNT strand 126 | GAGGCCACCTGAGAGTCAATAGTGAATTCCAAGAACCGAACG | 930 |
| core CNT strand 127 | CATTTTTAACGGATTGGTTGAGAGGAGTGTACTGGTAGGGAGGTGCGT | 931 |
| | AG | |
| core CNT strand 128 | CACCAACAAAGTACTTTCAACTTTAATCCTTTAAAGTAATTT | 932 |
| core CNT strand 129 | GCGGGACTAACGAAGAGAATAACATAACAGCCCTCAAGCAA | 933 |
| core CNT strand 130 | GCAAGGCCTGTAATGATAATCAGAAAAGGCAAAGCTAATGCA | 934 |
| core CNT strand 131 | ATTTTAGTCACGCATAGTCTTTAATGCGTTTTAAACTTTCCTTATCAT | 935 |
| | T | |
| core CNT strand 132 | AACCCACGCCACCAAAAGATTAAGAGGA | 936 |
| core CNT strand 133 | CCCCCAGAATACACACCGACCGTGTGATGGCATTTATCCCCCTCAAAT | 937 |
| | G | |
| core CNT strand 134 | CAGGCGGTAGCTCAATGGGCGCCCACCACCCTCAGATTTTCATTTAAT | 938 |
| | TG | |
| core CNT strand 135 | TAGTTTGGTAGGTAGGCTATCAGGTCATATTAAGTAATAGCG | 939 |
| core CNT strand 136 | GCTTAGATGATAAGCAGACTGTAGCGCG | 940 |
| core CNT strand 137 | GCTGTTTGCTCGAAATCACCAGTAGCAC | 941 |
| core CNT strand 138 | AGAAACGAAACCGAGATACCGATAGTTGCACGCTGTTATTCTGAAACA | 942 |
| | T | |
| core CNT strand 139 | TAACAGTGGTTTAACATTCAACCGATTG | 943 |
| core CNT strand 140 | AACTAACTTTGCAATGTGCTGCAAGGCG | 944 |
| core CNT strand 141 | CAATCGCCCTGTTTGTGCCGGAAACCAG | 945 |
| core CNT strand 142 | TATTCACACAATTCCCGTTGCGCACAAACAAATAAACATTACCTCCCC | 946 |
| | GG | |
| core CNT strand 143 | CGCGTAATATCCAGGTAATAAAAGGGACTAATCCTGTGTAGA | 947 |

CNT-binding handles on CDOs

| | | |
|---|---|---|
| CNT handles FRONT 0 | CCGCCTCACCGGAAAATAGGTTTTTAAGTGGTAAGTGG | 948 |
| CNT handles FRONT 1 | AGACGTTGGCAACAGCTGATTATTCGGTTACCGAATTTTAAGTGGTAA | 949 |
| | GTGG | |
| CNT handles FRONT 2 | GCCCTTTAGCCCGGCCAGAGCCACAAGAGAAGGATGTTGATATTTTAA | 950 |
| | GTGGTAAGTGG | |
| CNT handles FRONT 3 | GAGACTCCTCCACCGCCTGGCCCTGTCGCCCAAAGTAAGTTTTAAGTG | 951 |
| | GTAAGTGG | |
| CNT handles FRONT 4 | CCAGAAGGCACCAGGCGGATAAGTGCCGTTTTTAAGTGGTAAGTGG | 952 |
| CNT handles FRONT 5 | AGCGGTCCGCCGACAAGTTATTTTAAGTGGTAAGTGG | 953 |
| CNT handles FRONT 6 | TGAATTTCTTAAGCGAAAACCCTGCCTATTTCGCGGAATAATATAAAT | 954 |
| | TTTAAGTGGTAAGTGG | |
| CNT handles FRONT 7 | TTTTGCTCAGTAAAGACACCAGAACCTAGTTTGCCCCAGCAGACAGCT | 955 |
| | TGGAAACGTTTTAAGTGGTAAGTGG | |
| CNT handles FRONT 8 | AAACGTAGAATTTTTATACATATTTTAAGTGGTAAGTGG | 956 |
| CNT handles FRONT 9 | GAACCCATGTTTTTGTAACACTTTTAAGTTGGAAGAGG | 957 |
| CNT handles FRONT | TAAAGACTTTTTCGCTTAACTTTTTAAGTTGGAAGAGG | 958 |
| 10 | | |
| CNT handles FRONT | ATTAGACACGCCTGGCTTATCCGAAATTCTTACCACAGTACATTTTAA | 959 |
| 11 | GTTGGAAGAGG | |
| CNT handles FRONT | ATCAGATAGGCGTTAAGCCTTAAATCAATTATCCTGAAGCGCTTTTAA | 960 |
| 12 | GTTGGAAGAGG | |
| CNT handles FRONT | GAACGCGATAGAAGTAGCATTCCACAGTTTTAAGTTGGAAGAGG | 961 |
| 13 | | |
| CNT handles FRONT | ACCTCCCCCAATAGCATAGTTTTTTAAGTTGGAAGAGG | 962 |
| 14 | | |
| CNT handles FRONT | ATAGCTTTTTGATAGCTATAACCTTGCTTCTGTAAATCGTCCCTAATT | 963 |
| 15 | TGAAAATTTTTAAGTTGGAAGAGG | |
| CNT handles FRONT | GAATCCTTGTCGTAGGAAGTTTTGTCGTCTTTTTTTAAGTTGGAAGAG | 964 |
| 16 | G | |
| CNT handles BOT 0 | TGCTAAAGTATCCAAATAAGAAACGATTTCTGTATACATTTGAGGATT | 965 |
| | TTAGGTAGTGTGTTG | |
| CNT handles BOT 1 | ATAAACAGCCATAATTACCTTAAAACATTTTAGGTAGTGTGTTG | 966 |
| CNT handles BOT 2 | TTTTCACCAACTTTTTAGAGCCTTTTAGGTAGTGTGTTG | 967 |
| CNT handles BOT 3 | ATCCCATGAAAATCTACATTTAAGTATTATTTTAGGTAGTGTGTTG | 968 |
| CNT handles BOT 4 | GAGCCTTACAACTAAAGGAATGAGTGAGGGAGCACTTTTAGGTAGTGT | 969 |
| | GTTG | |
| CNT handles BOT 5 | TGCGAATAAAGGCTCCAAAAGATCAAGAAGTGCCATTTTAGGTAGTGT | 970 |
| | GTTG | |
| CNT handles BOT 6 | CGCTGAGATCTTTAAATAGAAAGCCTTATTACGCAAGGTTATTTTTAG | 971 |
| | GTAGTGTGTTG | |
| CNT handles BOT 7 | ATCGGTTAAAGAAGAGCAAATTTTTAGGTAGTGTGTTG | 972 |
| CNT handles BOT 8 | GTTGGCAAATTTTTCAGTTGATTTTAGGTAGTGTGTTG | 973 |

L1-L4 hybridization with CNT

| | | |
|---|---|---|
| L1 wrapped CNTs | GATGCGAGGCTATTCTGTGTGTGTGTGTGTGTGTGTGTGTGTGTGTGT | 974 |
| | GTGTGTGT | |
| L2 binds L1 | AGAATAGCCTCGCATCCCACTTACCACTTA | 975 |
| L3 binds L1 | AGAATAGCCTCGCATCCAACACACTACCTA | 976 |
| L4 binds L1 | AGAATAGCCTCGCATCCCTCTTCCAACTTA | 977 |

TABLE 13
Handle sequences for CNT 3-WJ design. The core cube sequences were
redesigned for CNT hybridization. Two designs were tested: one using identical sequences
and another using different sequences on each side of the CDO. The same L1-L4 handles
were used as in the U-shape design.

| | | |
|---|---|---|
| | | SEQ ID |
| Label | DNA Sequence (5′→3′) | NO |

CDO cores

| | | |
|---|---|---|
| core CNT strand 0 | GAACAAGCAATTTTTCCGTTTTTATTTTCAAAAAC | 978 |
| core CNT strand 1 | AGCAGCCCGATCTAAATCATTACCGCGCGATTTTCCCTTA | 979 |
| core CNT strand 2 | AACTACAGGGAGAAACCCAGCTACAATTGATTAGTATTCTAA | 980 |
| core CNT strand 3 | TGCGTTATACGTTGCTATTTTATGAGGAAGTTTAGTATCATA | 981 |
| core CNT strand 4 | AACGCTCAACTTTTTGTAGGGCTTAATTGACTAGA | 982 |
| core CNT strand 5 | GACAAAAGGTTTTTTAAGTAATTCTGTCCATAATT | 983 |
| core CNT strand 6 | ATAATATCCCTTTTTTCCTAATTTACGAGCGCTTA | 984 |
| core CNT strand 7 | TAACAACCTGCAACAAACAAAATTAATTCCAAAAAAATAATT | 985 |
| core CNT strand 8 | CAAATATATTACCCAAGGGAACCGAACTGGCCGCTGCAAGAA | 986 |
| core CNT strand 9 | GCGGATTAGGCTTGTCCATGTTACTTAGCATCGGAGAGAGAT | 987 |
| core CNT strand 10 | GCCGCCAGGCGGTTTGCGTATCATGTTTTGCTGAA | 988 |
| core CNT strand 11 | TTTGCCTAGAGCCGATCGGCCAACGCGCAAGTACGGCGGATG | 989 |
| core CNT strand 12 | TAGTAAGAGCTTTTTACACTATCATAACCCTTAGG | 990 |
| core CNT strand 13 | CGCCAGGCTACAAAAAGATTCAAAAGGGCGAACGATCATCAG | 991 |
| core CNT strand 14 | TGGCGAACAAGAGACCTCATATATTTTAATGGTCATAAAACG | 992 |
| core CNT strand 15 | TCCTCATACTCACATTAATTGACACAACGAAATTG | 993 |
| core CNT strand 16 | TTAGAGCAAAGCGCGGGTGCCTAATGAGCATAAAGGGTCATA | 994 |
| core CNT strand 17 | CAACATTAAAATTGTAAAGCCACGTCAAAGGGCAGCTTTCAT | 995 |
| core CNT strand 18 | GGATTCTCCGTTTTTGGGAACAAACGGCGGCTGGC | 996 |
| core CNT strand 19 | TGGATTAACGACCAAACAATATTACCGCTAGCGGTAAAATAA | 997 |
| core CNT strand 20 | ATTATCAACCCTTCAGTAGAAGAACTCAATGCGCCTTGTTAA | 998 |
| core CNT strand 21 | AATAAGTATAGCCCGAGATAGCGCCTGAACATCGG | 999 |
| core CNT strand 22 | AGACAAATGCCTTGAATCCCTTATAAATAAGTTACTATACAG | 1000 |
| core CNT strand 23 | TTGAGATAGAATGACATATTTAACAACGTAAACACACGAAGG | 1001 |
| core CNT strand 24 | TATGCGATCATTGATCGAGCCAGTAATATTGAAATTAAAACACTGCAT | 1002 |
| | CA | |
| core CNT strand 25 | TATGTACAGGCTGCACAATAAACAACATCTTTTTCCATACAG | 1003 |
| core CNT strand 26 | AGATTGTGCTTCTGATCAACAATAGATATGCTGATCCTCAGAGCTTTC | 1004 |
| | CT | |
| core CNT strand 27 | TTTTGAATATCATTAACCAATCAATAATAGACTACTAAAGGG | 1005 |
| core CNT strand 28 | TGAATTTTTTTGTTTAATTTTTTCGTCAAAAATGCCAGTTACAAA | 1006 |
| core CNT strand 29 | TGAGCATATCAGCAACTGGCATGATTAAGACTGATAATTGT | 1007 |
| core CNT strand 30 | GATATTCCGCGTTTTCAGAACCGCCACCAATAAGATTTGCGG | 1008 |
| core CNT strand 31 | GTGAATAGCATCAACCCTCATTTTCAGGAATATCAACGAGGG | 1009 |
| core CNT strand 32 | AGCAAACATCAAGAGTATTTTTTATCTTGACATTTGAAAGAGGAC | 1010 |
| core CNT strand 33 | TATAATGAGTACCTCGGCATTTTCGGTCCTCAGAGCAGGGTGGTTTTT | 1011 |
| | CACAGAC | |
| core CNT strand 34 | ACCTGTTTCGTGCCAGCTGCACATTCCAAACTAATGCAGATACATAAT | 1012 |
| | TGTGTCTG | |
| core CNT strand 35 | GAGAGATGTTTTCCCCAGACGACGATAAGAAAGATGTAGATT | 1013 |
| core CNT strand 36 | GAGCAAAAGGGGGAAAGAAGTTTTGCCAACGTTAAATAACCT | 1014 |
| core CNT strand 37 | CCAAGCTTAATGCCGGATTTTTTGAGGGTAGCGGCCGGAGACAGT | 1015 |
| core CNT strand 38 | TTATCCGTAGAGGAATTAGCAAGGCCGGTGATATTCTCACTGCCCGCT | 1016 |
| | TATATGA | |
| core CNT strand 39 | CAGTCACTACTTCTGTAATGGGATAGGTGCCATCACACGCTG | 1017 |
| core CNT strand 40 | AGATAGAGATGATGCCGTGCATCTGCCATAAATTTGCTACAG | 1018 |
| core CNT strand 41 | AAAATTATGGATTATTTTTTTTTACATTGGCATGCAACAGGAAAA | 1019 |
| core CNT strand 42 | CCGAAAGCGAATTGCTGAACCTCAAATATCAAAAAAAATCG | 1020 |
| core CNT strand 43 | TGAGTTTGAACAAATTTTTTGTCAGAGGAGGCTTTTGAGGACTAAA | 1021 |
| core CNT strand 44 | AAATCAGGTCTTTTACCCTGACTATTAACCAGAACTTTTTGAGTAGTA | 1022 |
| | ACTGAT | |
| core CNT strand 45 | ATAAGAACCAACATCAGTTCAGAAAACGGGTTTAAAACGGAG | 1023 |
| core CNT strand 46 | ATTCTACTAAATTTAATGGTAGAGAATATCGTCATAAATATTTTTAAG | 1024 |
| | TCTTTGA | |
| core CNT strand 47 | CCAATAACCAGTCATTTTTTGGACGTTTCATTTGGTGGCGCGAGCT | 1025 |
| core CNT strand 48 | GAAGGGCGATCTTTTGCGGGCCTCTTCATCGTAAATTTTTACTAGCAT | 1026 |
| | GATTTC | |
| core CNT strand 49 | GAGAAAAGTTCAGCGCCATTCGCCATTCCCCGGTTACTTTTG | 1027 |
| core CNT strand 50 | CGTTAGTAACTATATGTAAAAGTCCTGAGCTTTCCGGCACCATAAGCA | 1028 |
| | AAAGCTA | |
| core CNT strand 51 | TCGCACAAACGTTATTTTTTATATTTTTTGCTTTGTACGAGCACGT | 1029 |
| core CNT strand 52 | AGAAACCACCATTTAGGAGCGGAATTAAAGAATACTTTTTGTGGCACA | 1030 |
| | GTTCTT | |
| core CNT strand 53 | CTGTCCAACAGGAAATCATAGG | 1031 |
| core CNT strand 54 | GAGTGATAGATTAAGACGCTAGTACCGAACTCGTATTAAATTCGCCAT | 1032 |
| | AATCAGT | |
| core CNT strand 55 | TTTAGAAGTATTTTGACTTTACAAACAGAACCACCTTTTTAGCAGAAG | 1033 |
| | ATTTTT | |
| core CNT strand 56 | AAGGAACTAAAGCATTTTTTTCACCTTATTCATTTTCAATTACC | 1034 |
| core CNT strand 57 | TTTGCCATCTTTTTCATAATCAAAATCCCTCAGAGTTTTTCCGCCACC | 1035 |
| | CCAGTG | |
| core CNT strand 58 | CATCGATAGCATTTACCGTAATCAGTAAGGAGGTTTTTTTGAGGCAGG | 1036 |
| | TGGGAA | |
| core CNT strand 59 | AGGTGAATTATTTTCCGTCACCGACTTCCGTTCCATTTTTGTAAGCGT | 1037 |
| | CGAGTC | |
| core CNT strand 60 | ATATGGTATAAACATTTTTGTTAATGCCTCCTGTTTGATTTGGTGGTT | 1038 |
| core CNT strand 61 | ATAGCTTTTTGATAGCTATAACCTTGCTTCTGTAAATCGTCCCTAATT | 1039 |
| | TGAAAAT | |
| core CNT strand 62 | ATCAGATAGGCGTTAAGCCTTAAATCAATTATCCTGAAGCGC | 1040 |
| core CNT strand 63 | AAAAGTTTTTCCTGTTTCCATTAAACGGGTAAAATACGTAATCATCGC | 1041 |
| | ATTGGGC | |
| core CNT strand 64 | AGGCAGAAAATAAGGAAAGAGGCAAAAGCGATTATATTACCT | 1042 |
| core CNT strand 65 | TCATCTTTTTTTCTGACCTAATAGTAGTAGCATTAACATCCAGCCTTT | 1043 |
| | TCAATCA | |
| core CNT strand 66 | GAACGCGAAGACAATAGCAAAATTAAGCTGTACCAAACAGGA | 1044 |
| core CNT strand 67 | GGTTGTTTTTGGTTATAAATCAGAGCGGGAGCTAAACAGGAGCAATAC | 1045 |
| | ACAATAT | |
| core CNT strand 68 | TAAGTATTTAAGAACGCATAACCGATATGCCCTTCACCGGAA | 1046 |
| core CNT strand 69 | CAATATTTTTGCTATCTCGCTGAGGCTTGCAGGGAGTTAAAGACCAAC | 1047 |
| | AGAACCG | |
| core CNT strand 70 | CCTCAGAAAGAATTGCAGCGAAAGACAGCCGGAACTCATTCA | 1048 |
| core CNT strand 71 | CTCCTTTGCTTAATTAAATATGCAACTAGGGGAGACCAGAAC | 1049 |
| core CNT strand 72 | AATACTTTTTCACATTCTATAACAGTTGATTCCCAATTCTGTGAGAAA | 1050 |
| | TATTTTT | |
| core CNT strand 73 | AGAGGCTGGAACAAGATACATTTCGCAAAATGCAAGAGTCTG | 1051 |
| core CNT strand 74 | GTACCGACCTGTGTATACGAGCCGGAAGTGAGCTATAAAGCC | 1052 |
| core CNT strand 75 | CTTCCTTTTTTGTAGCCGAAAAACCGTCTATCAGGCAAGTGCAGCCAT | 1053 |
| | GATTCAC | |
| core CNT strand 76 | ATTAGACACGCCTGGCTTATCCGAAATTCTTACCACAGTACA | 1054 |
| core CNT strand 77 | GAACCCATGTTTTTGTAACAC | 1055 |
| core CNT strand 78 | ATTTGTATGCCACTCGGAATCATAATTAGAATCGCCCATAAATCAA | 1056 |
| core CNT strand 79 | ATGTTTAGACTTTTATAGCGT | 1057 |
| core CNT strand 80 | CGGGAGAAATAAATAAATATATTTTAGTGACGACGGCAACTGTTGG | 1058 |
| core CNT strand 81 | CAGTATCGGCTTTTAGGAAGA | 1059 |
| core CNT strand 82 | CGTTGTAGGCCGATCTTTTTAACCTCCGATGTAGATTGCGGAACAA | 1060 |
| core CNT strand 83 | AAGCTGCGAGGCGCAGACGGTACCCTCAGAGTTAAGCCCAATCTCAGA | 1061 |
| | AAAGACTT | |
| core CNT strand 84 | AAATTTTGTGTCGAAATCCGCGGCTACAGGTAATTGAGCGCTGATAGC | 1062 |
| | AAAGCAAA | |
| core CNT strand 85 | ATTAGAGCTCATAGGCTGGCTGACCTTCTCCAACACCTTATTAGCG | 1063 |
| core CNT strand 86 | CACCACCTTAGCGTAGGTCATTTCGCCAAAAGGAAAATCAAG | 1064 |
| core CNT strand 87 | AACGCTTAAGGATAAAAATTTTATATTTGGGAAGAAAAATCTGAGGGG | 1065 |
| | GCGCCAGC | |
| core CNT strand 88 | TCGACTCCTACCGTTCTAGCTGATAAATTGCATGCACCAATGAAAC | 1066 |
| core CNT strand 89 | AGAATGGCAGCAAATTCGTAATCTGTGAGCGAGTATTGGGAA | 1067 |
| core CNT strand 90 | ATTCTGGGGCCTTGCTGGTAACCACCACTAACCAATAGGAACCACGTT | 1068 |
| | GGATTGTT | |
| core CNT strand 91 | TGATTTTAGTAATAACATCACACTATGGGTTAAAATTCGCATGTTTGA | 1069 |
| | GATTCCTG | |
| core CNT strand 92 | GACTTTTTCGCTTAACTGAACACCCTCGTCACGTATAAAGCC | 1070 |
| core CNT strand 93 | GAAAAGGTGCAAACTGGCTCATTATCTGCGGAATAAAGTACC | 1071 |
| core CNT strand 94 | ATAACGTGCATAATATTTAAATTGTTCCAGCCAACAAGAAAA | 1072 |
| core CNT strand 95 | AATGGTTAAACAGTACATAAATCAATATTAAAAATACCGAACATTCGA | 1073 |
| | CCACTCATCGA | |
| core CNT strand 96 | GAATCCTTGTCGTAGGAAGTTTTGTCGTCTTTCCAGACTTTTTTAGTA | 1074 |
| | AA | |
| core CNT strand 97 | TAGCAACGACCTGCCCCTGACGAGAAACTAGTCAGAGCCCAATAG | 1075 |
| core CNT strand 98 | ACAATGAGCCACCCTAATTCGAGCTTCAAAGCGAACTTTTAGACCGGA | 1076 |
| core CNT strand 99 | AGATGAACGGTGTTTTTCACTCAGAACCGCCACCATAGCCCGGTCAGG | 1077 |
| core CNT strand 100 | GAAGTTTTTAATGACCGCCAGCATTGACGCGACAGTTACGAGGCA | 1078 |
| core CNT strand 101 | GTTTAGCTTAGAACATCGATGAACGGTAGCTATTATAATAGTAAA | 1079 |
| core CNT strand 102 | TTGAGATTCGTTTACAGTCACGACGTTGTAAAACGATTTTGGCCAGTG | 1080 |
| core CNT strand 103 | CAAATCACCATCATCCAGTCCAGACGATTGGCCTAAACGTCCTGCAGG | 1081 |
| core CNT strand 104 | CACTATTTTAAAGAACGTGGACTCCATGAGTCTCTGAATTTAGAGCCA | 1082 |
| | TACAACCCGTC | |
| core CNT strand 105 | GGCGCGTTTGCCTGTGACCTGAAAGCGTTCATCATGGGACGACGA | 1083 |
| core CNT strand 106 | TTCGCGTATTGACCGAATAATGGAAGGGTTAGAACCTTTTACCATATC | 1084 |
| core CNT strand 107 | AAACAGGGAATCTTACCAACGGGTTTTGTTAGCGA | 1085 |
| core CNT strand 108 | CAGATAGCGAGAGGTAGGATTAGCGGGGGAAAGTATGCAGCA | 1086 |
| core CNT strand 109 | TAAAACACCTTTGCCGGGTATTAAACCAGAGAAGAAGTGTTTTTATAT | 1087 |
| | GT | |
| core CNT strand 110 | CCCCAAAAAAACATTATGACCAAAGAATAGAACGC | 1088 |
| core CNT strand 111 | ATTGTGAACCAAGCGCGAAACCTAAAACGCGTTAA | 1089 |
| core CNT strand 112 | TGGGCGCCATTTTTACCCGCCGCGCTTAAACTATCCCAACAG | 1090 |
| core CNT strand 113 | TATCAAACGGTACGCCAGAATCCGAGTAATAGCCC | 1091 |
| core CNT strand 114 | TTATTAACGAACTGAAAGAGT | 1092 |
| core CNT strand 115 | CTAAAATAGCCAGCATGATGAAACAAAC | 1093 |
| core CNT strand 116 | ACCTCCCCCAATAGCATAGTT | 1094 |
| core CNT strand 117 | AGCGTAATTTACAGGCGTCTTTCCAGAGGCTATTAATTAACTT | 1095 |
| core CNT strand 118 | TCTGAGCGGCTGTAGTTTGAGTAACATTGGCTATAATTAAC | 1096 |
| core CNT strand 119 | TGCCTGATGCCTGAGTAATGTACCATTACATTATTACAGGTAAAACCA | 1097 |
| | ATGGGTAA | |
| core CNT strand 120 | AATCGGTAATAAAGGCAAATC | 1098 |
| core CNT strand 121 | GAGGTGAAGATAATGGGATTT | 1099 |
| core CNT strand 122 | GGCTTTTGACGGAAAGTAAAAC | 1100 |
| core CNT strand 123 | GAAAAATTTGAGGAGTATGTTAGC | 1101 |
| core CNT strand 124 | AGTGCCCGTTTACCATATCTGGTCA | 1102 |
| core CNT strand 125 | TACTGGTAGGGAGGTGCGTAGTTTTAGGACACA | 1103 |
| core CNT strand 126 | GTCAATGGCGGTCACAATTTCATTTGATTATTT | 1104 |
| core CNT strand 127 | AATATTGATGATACTGTTGTTCCAGTTTTACCTA | 1105 |
| core CNT strand 128 | GTATCACCGTACTTTTTTAGGAGGTTTAGTACCAATAG | 1106 |
| core CNT strand 129 | CCGAACAAATGACAACAACCAAGAGAGTTTAAGAGGCT | 1107 |
| core CNT strand 130 | CATAAAGGAACGGAATACTTTTTCCAAAAGTTGCTTTCGAGG | 1108 |
| core CNT strand 131 | GATCGTCCAATCATAAATCAACGTAACAAGCCCGACCGCCAC | 1109 |
| core CNT strand 132 | GAACGCGATAGAAGTAGCATTCCACAGA | 1110 |
| core CNT strand 133 | ACACCGCTAATAGACAACAGTTTCAGCG | 1111 |
| core CNT strand 134 | ATCAGCTATCGTAAGCAATTCATCAATA | 1112 |
| core CNT strand 135 | GAGGCCACCTGAGAGTCAATAGTGAATTCCAAGAACCGAACG | 1113 |
| core CNT strand 136 | CATTTTTAACGGATTGGTTGAGAGGAGTGTACTGGTAGGGAGGTGCGT | 1114 |
| | AG | |
| core CNT strand 137 | CACCAACAAAGTACTTTCAACTTTAATCCTTTAAAGTAATTT | 1115 |
| core CNT strand 138 | GCGGGACTAACGAAGAGAATAACATAACAGCCCTCAAGCAA | 1116 |
| core CNT strand 139 | GCAAGGCCTGTAATGATAATCAGAAAAGGCAAAGCTAATGCA | 1117 |
| core CNT strand 140 | ATTTTAGTCACGCATAGTCTTTAATGCGTTTTAAACTTTCCTTATCAT | 1118 |
| | T | |
| core CNT strand 141 | AACCCACGCCACCAAAAGATTAAGAGGA | 1119 |
| core CNT strand 142 | CCCCCAGAATACACACCGACCGTGTGATGGCATTTATCCCCCTCAAAT | 1120 |
| | G | |
| core CNT strand 143 | CAGGCGGTAGCTCAATGGGCGCCCACCACCCTCAGATTTTCATTTAAT | 1121 |
| | TG | |
| core CNT strand 144 | TAGTTTGGTAGGTAGGCTATCAGGTCATATTAAGTAATAGCG | 1122 |
| core CNT strand 145 | GCTTAGATGATAAGCAGACTGTAGCGCG | 1123 |
| core CNT strand 146 | GCTGTTTGCTCGAAATCACCAGTAGCAC | 1124 |
| core CNT strand 147 | AGAAACGAAACCGAGATACCGATAGTTGCACGCTGTTATTCTGAAACA | 1125 |
| | T | |
| core CNT strand 148 | AACTAACTTTGCAATGTGCTGCAAGGCG | 1126 |
| core CNT strand 149 | CAATCGCCCTGTTTGTGCCGGAAACCAG | 1127 |
| core CNT strand 150 | TATTCACACAATTCCCGTTGCGCACAAACAAATAAACATTACCTCCCC | 1128 |
| | GG | |
| core CNT strand 151 | CGCGTAATATCCAGGTAATAAAAGGGACTAATCCTGTGTAGA | 1129 |

CNT-binding handles on CDOs (same sequences)

| | | |
|---|---|---|
| CNT handles 0 | AGCGGTCCGCCGACAAGTTATTTTAAGTGGTAAGTGG | 1130 |
| CNT handles 1 | CGCAGAGTCGGCAAAGTAACTTTTAAGTGGTAAGTGG | 1131 |
| CNT handles 2 | CCGCCTCACCGGAAAATAGGTTTTTAAGTGGTAAGTGG | 1132 |
| CNT handles 3 | ATCGGTTAAAGAAGAGCAAATTTTTAAGTGGTAAGTGG | 1133 |
| CNT handles 4 | GTTGGCAAATTTTTCAGTTGATTTTAAGTGGTAAGTGG | 1134 |
| CNT handles 5 | GAGAAACAAGAAATGAAGGTATTTTAAGTGGTAAGTGG | 1135 |
| CNT handles 6 | AAACGTAGAATTTTTATACATATTTTAAGTGGTAAGTGG | 1136 |
| CNT handles 7 | TTTTCACCAACTTTTTAGAGCCTTTTAAGTGGTAAGTGG | 1137 |
| CNT handles 8 | ACGCTCATGGAAAGGAACAAATACATTTTTAAGTGGTAAGTGG | 1138 |
| CNT handles 9 | ATAAACAGCCATAATTACCTTAAAACATTTTAAGTGGTAAGTGG | 1139 |
| CNT handles 10 | CATTTTTAACGGATTGGTTGAGAGGAGTGTTTTAAGTGGTAAGTGG | 1140 |
| CNT handles 11 | ATCCCATGAAAATCTACATTTAAGTATTATTTTAAGTGGTAAGTGG | 1141 |
| CNT handles 12 | CCAGAAGGCACCAGGCGGATAAGTGCCGTTTTTAAGTGGTAAGTGG | 1142 |
| CNT handles 13 | TGCGAATAAAGGCTCCAAAAGATCAAGAAGTGCCATTTTAAGTGGTAA | 1143 |
| | GTGG | |
| CNT handles 14 | AGACGTTGGCAACAGCTGATTATTCGGTTACCGAATTTTAAGTGGTAA | 1144 |
| | GTGG | |
| CNT handles 15 | GAGACTCCTCCACCGCCTGGCCCTGTCGCCCAAAGTAAGTTTTAAGTG | 1145 |
| | GTAAGTGG | |
| CNT handles 16 | GCCCTTTAGCCCGGCCAGAGCCACAAGAGAAGGATGTTGATATTTTAA | 1146 |
| | GTGGTAAGTGG | |
| CNT handles 17 | GGGTCAGAGGGCGACGTCAGATGACCCTCAATCAAGCGCCAATTTTAA | 1147 |
| | GTGGTAAGTGG | |
| CNT handles 18 | ATTTTCAACCTTTTTTGCTTTGAATACCCAAAAGATTTAACGTTTTAA | 1148 |
| | GTGGTAAGTGG | |
| CNT handles 19 | CGCTGAGATCTTTAAATAGAAAGCCTTATTACGCAAGGTTATTTTTAA | 1149 |
| | GTGGTAAGTGG | |
| CNT handles 20 | AGAAATAAAGACGCTCAATCGTCTGAAATTTGCACTTATTCATTAATT | 1150 |
| | TTAAGTGGTAAGTGG | |
| CNT handles 21 | CAATAATTGGCAACAGTTTATTTTGTCACAATCAATTTTAAAATTCTT | 1151 |
| | TTAAGTGGTAAGTGG | |
| CNT handles 22 | TGCTAAAGTATCCAAATAAGAAACGATTTCTGTATACATTTGAGGATT | 1152 |
| | TTAAGTGGTAAGTGG | |
| CNT handles 23 | TGAATTTCTTAAGCGAAAACCCTGCCTATTTCGCGGAATAATATAAAT | 1153 |
| | TTTAAGTGGTAAGTGG | |
| CNT handles 24 | TTTTGCTCAGTAAAGACACCAGAACCTAGTTTGCCCCAGCAGACAGCT | 1154 |
| | TGGAAACGTTTTAAGTGGTAAGTGG | |

CNT-binding handles on CDOs (different sequences)

| | | |
|---|---|---|
| CNT handles FRONT 0 | CCGCCTCACCGGAAAATAGGTTTTTAAGTGGTAAGTGG | 1155 |
| CNT handles FRONT 1 | AGACGTTGGCAACAGCTGATTATTCGGTTACCGAATTTTAAGTGGTAA | 1156 |
| | GTGG | |
| CNT handles FRONT 2 | GCCCTTTAGCCCGGCCAGAGCCACAAGAGAAGGATGTTGATATTTTAA | 1157 |
| | GTGGTAAGTGG | |
| CNT handles FRONT 3 | GAGACTCCTCCACCGCCTGGCCCTGTCGCCCAAAGTAAGTTTTAAGTG | 1158 |
| | GTAAGTGG | |
| CNT handles FRONT 4 | CCAGAAGGCACCAGGCGGATAAGTGCCGTTTTTAAGTGGTAAGTGG | 1159 |
| CNT handles FRONT 5 | AGCGGTCCGCCGACAAGTTATTTTAAGTGGTAAGTGG | 1160 |
| CNT handles FRONT 6 | TGAATTTCTTAAGCGAAAACCCTGCCTATTTCGCGGAATAATATAAAT | 1161 |
| | TTTAAGTGGTAAGTGG | |
| CNT handles FRONT 7 | TTTTGCTCAGTAAAGACACCAGAACCTAGTTTGCCCCAGCAGACAGCT | 1162 |
| | TGGAAACGTTTTAAGTGGTAAGTGG | |
| CNT handles FRONT 8 | AAACGTAGAATTTTTATACATATTTTAAGTGGTAAGTGG | 1163 |
| CNT handles BOT 0 | TGCTAAAGTATCCAAATAAGAAACGATTTCTGTATACATTTGAGGATT | 1164 |
| | TTAGGTAGTGTGTTG | |
| CNT handles BOT 1 | ATAAACAGCCATAATTACCTTAAAACATTTTAGGTAGTGTGTTG | 1165 |
| CNT handles BOT 2 | TTTTCACCAACTTTTTAGAGCCTTTTAGGTAGTGTGTTG | 1166 |
| CNT handles BOT 3 | ATCCCATGAAAATCTACATTTAAGTATTATTTTAGGTAGTGTGTTG | 1167 |
| CNT handles BOT 4 | GAGCCTTACAACTAAAGGAATGAGTGAGGGAGCACTTTTAGGTAGTGT | 1168 |
| | GTTG | |
| CNT handles BOT 5 | TGCGAATAAAGGCTCCAAAAGATCAAGAAGTGCCATTTTAGGTAGTGT | 1169 |
| | GTTG | |
| CNT handles BOT 6 | CGCTGAGATCTTTAAATAGAAAGCCTTATTACGCAAGGTTATTTTTAG | 1170 |
| | GTAGTGTGTTG | |
| CNT handles BOT 7 | ATCGGTTAAAGAAGAGCAAATTTTTAGGTAGTGTGTTG | 1171 |
| CNT handles BOT 8 | GTTGGCAAATTTTTCAGTTGATTTTAGGTAGTGTGTTG | 1172 |
| CNT handles RIGHT 0 | CAATAATTGGCAACAGTTTATTTTGTCACAATCAATTTTAAAATTCTT | 1173 |
| | TT AAGTTGGAAGAGG | |
| CNT handles RIGHT 1 | CGCAGAGTCGGCAAAGTAACTTTTAAGTTGGAAGAGG | 1174 |
| CNT handles RIGHT 2 | GGGTCAGAGGGCGACGTCAGATGACCCTCAATCAAGCGCCAATTTTAA | 1175 |
| | GTTGGAAGAGG | |
| CNT handles RIGHT 3 | ATTTTCAACCTTTTTTGCTTTGAATACCCAAAAGATTTAACGTTTTAA | 1176 |
| | GTTGGAAGAGG | |
| CNT handles RIGHT 4 | TAACAGTGGTTTAACATTCAACCGATTGTTTTAAGTTGGAAGAGG | 1177 |
| CNT handles RIGHT 5 | CATTTTTAACGGATTGGTTGAGAGGAGTGTTTTAAGTTGGAAGAGG | 1178 |
| CNT handles RIGHT 6 | GAGAAACAAGAAATGAAGGTATTTTAAGTTGGAAGAGG | 1179 |
| CNT handles RIGHT 7 | ACGCTCATGGAAAGGAACAAATACATTTTTAAGTTGGAAGAGG | 1180 |
| CNT handles RIGHT 8 | AGAAATAAAGACGCTCAATCGTCTGAAATTTGCACTTATTCATTAATT | 1181 |
| | TTAAGTTGGAAGAGG | |

1. A cube DNA origami nanostructure, comprising:
a ssDNA scaffold folded into a cubic frame, comprising:
six helical bundles (HB) defining six edges of the cubic frame; and
a plurality of staple strands, each staple strand being positioned proximate to a center of an edge of the cubic frame.

2. The nanostructure of claim 1, wherein the plurality of staple strands comprises pairs of complementary sequences configured to promote binding between two edges of two cube DNA origami nanostructures.

3. The nanostructure of claim 1, wherein the HBs comprise 6-12 helical turns.

4. The nanostructure of claim 1, wherein the cube DNA origami nanostructure comprises mitered vertices.

5. The nanostructure of claim 1, wherein the plurality of staple strands comprises a plurality of types of orthogonal staple strands.

6. The nanostructure of claim 1, wherein:
the cubic frame comprises six faces, each face comprising four edges;
the plurality of staple strands comprises four types of orthogonal staple strands; and
each one of the four edges of each face comprises one of the four types of orthogonal staple strands such that the four edges of each face comprise all four types of orthogonal staple strands.

7. The nanostructure of claim 1, wherein the melting temperature of the plurality of staple strands is below 55° C.

8. The nanostructure of claim 1, further comprising carbon nanotubes, gold nanoparticles, proteins, or other molecules.

9. The nanostructure of claim 1, further comprising carbon nanotubes.

10. The nanostructure of claim 9, further comprising a positioning DNA strand, wherein:
the positioning DNA strand is located on an exterior surface of the carbon nanotubes; and
the positioning DNA strand is complementary to another sequence within the nanostructure.

11. A polycube assembly, comprising:
a plurality of the cube DNA origami nanostructures of claim 1, wherein:
each of the plurality of the cube DNA origami nanostructures belongs to one of a plurality of cube types;
the plurality of staple strands of the cube DNA origami nanostructures of each of the plurality of cube types independently comprise a set of staple strands;
each of the cube types has a unique set of staple strands; and
the plurality of the staple strands of the plurality of cube types are designed to promote binding between the plurality of cube types, whereby the cube DNA origami nanostructures assemble into a specific 3D configuration.

12. The polycube assembly of claim 11, wherein:
each cube DNA origami nanostructure comprises a set of six faces comprising a top, a bottom, a left, a right, a front, and a back;
each face comprises four edges; and
each edge of each face of each of the plurality of cube types has a unique sequence.

13. The polycube assembly of claim 12, wherein each edge of each face of each of the plurality of cube types has a sequence selected from SEQ ID NO: 1-1181.

14. The polycube assembly of claim 11, further comprising one or more blocker strands configured to prevent aggregation of the cube DNA origami nanostructures.

15. The polycube assembly of claim 11, wherein the plurality of cube types comprises 2-19 cube types.

16. The polycube assembly of claim 11, wherein each of the cube DNA origami nanostructures of each of the plurality of cube types comprises a unique set of sequences selected from SEQ ID NO: 1-1181.

17. The polycube assembly of claim 11, wherein the specific 3D configuration is selected from the group consisting of: a 3D cross, a hollow 3×3×3 cube, a human only polycube, a wolf only polycube, a multifarious werewolf polycube, fractal Menger cube M₂, a simple cubic crystal lattice, and a solid 4×4×4 assembly.

18. The polycube assembly of claim 11, further comprising carbon nanotubes, gold nanoparticles, proteins, or other molecules.

19. (canceled)

20. The polycube assembly of claim 19, further comprising a positioning DNA strand, wherein:
the positioning DNA strand is located on an exterior surface of the carbon nanotubes; and
the positioning DNA strand is complementary to another sequence within the polycube assembly.

21. A method of making a three-dimensional (3D) DNA nanocube capable of self-assembly into larger 3D multicomponent structures, the method comprising:
obtaining one or more DNA origami monomers comprising staple strands; and
incubating the DNA origami monomers for in a solution a period of time and at a temperature sufficient for self-assembly into the DNA nanocube.

22-46. (canceled)
